(19) United States
(12) Patent Application Publication
(10) Pub. No.: US 2009/0062286 A1
(43) Pub. Date: Mar. 5, 2009

(54) Crystal Structure of SMYD3 Protein

(76) Inventors: Kenneth William Foreman, Farmingdale, NY (US); Frances E. Park, San Diego, CA (US); Lee Arnold, East Islip, NY (US)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/115,089

(22) Filed: May 5, 2008

Related U.S. Application Data
(60) Provisional application No. 60/915,969, filed on May 4, 2007.

(51) Int. Cl. A61K 31/41; C12N 9/10; G06G 7/58; G01N 33/53; C07D 257/04; A61K 31/421; A61K 31/519; A61P 35/00; A61K 31/5375; A61K 31/433; C07D 263/04; C07D 285/135; C07D 487/04; C07D 265/30

(52) U.S. Cl. 514/237.8; 435/193; 703/11; 435/7.1; 548/253; 548/215; 548/138; 544/263; 544/165; 514/381; 514/374; 514/363; 514/252.16

(57) ABSTRACT

The invention relates to SMYD3 methyltransferase (SMYD3), SMYD3 binding pockets or SMYD3-like binding pockets. The invention relates to a computer comprising a data storage medium encoded with the structure coordinates of such binding pockets. The invention also relates to methods of using the structure coordinates to solve the structure of homologous proteins or protein complexes. The invention relates to methods of using the structure coordinates to screen for and design compounds that bind to SMYD3 methyltransferase protein, complexes of SMYD3 methyltransferase protein, homologues thereof, or SMYD3-like protein or protein complexes.

### RELATED APPLICATIONS

This application claims the benefit of U.S. Ser. No. 60/915,969, filed May 4, 2007 the contents of which is incorporated herein by reference in its entirety.

### TECHNICAL FIELD OF THE INVENTION

The present invention relates to human SMYD3 methyltransferase (SMYD3), SMYD3 binding pockets or SMYD3-like binding pockets. The present invention provides a computer comprising a data storage medium encoded with the structure coordinates of such binding pockets. This invention also relates to methods of using the structure coordinates to solve the structure of homologous proteins or protein complexes. In addition, this invention relates to methods of using the structure coordinates to screen for and design compounds, including inhibitory compounds, that bind to SMYD3 protein, SMYD3 protein complexes, homologues thereof, or SMYD3-like protein or SMYD3-like protein complexes. The invention also relates to crystallizable compositions and crystals comprising SMYD3 domain.

### BACKGROUND OF THE INVENTION

The SMYD3 methyltransferase (SMYD3) is a lysine methyltransferase that is believed to play a role in liver, colon, and breast cancers. It has also been associated with spermatogenesis. The SMYD3 methyltransferase (SMYD3) is a lysine methyltransferase that is believed to play a role in liver, colon, and breast cancers. It has also been associated with spermatogenesis. SMYD3 is a SET domain histone methyltransferase that can modify lysine 4 of histone H3 and thereby contribute to transcriptional activation of target genes. SMYD3 also has a MYND type zinc finger domain that could play a role in either DNA sequence recognition or protein-protein interaction. SMYD3 was found to physically associate with heat shock protein Hsp90; furthermore this association was shown to be essential for SMYD3's methyltransferase activity towards histone H3. SMYD3 was initially identified by virtue of its overexpression in colon and liver cancers (Hamamoto, R., Furukawa, Y., Morita, M., Iimura, Y., Silva, F. P., Li, M., Yagyu, R., and Nakamura, Y. (2004). SMYD3 encodes a histone methyltransferase involved in the proliferation of cancer cells. Nature Cell Biology 6, 731-740.) but was also later found to be elevated in the great majority of breast cancers (Hamamoto, R., Silva, F. P., Tsuge, M., Nishidate, T., Katagiri, T., Nakamura, Y., and Furukawa, Y. (2006). Enhanced SMYD3 expression is essential for the growth of breast cancer cells. Cancer Science 97, 113-118.). Knockdown of SMYD3 by siRNA in breast, colon, and liver cancer cell lines brought about apoptosis and inhibited proliferation of these cells underscoring the important role for the elevated level of SMYD3 in these cancer types. This elevated expression was later linked to a variable number of tandem repeats polymorphism in the SMYD3 regulatory region that creates a third binding site for the E2F-1 transcription factor in addition to the two commonly present in the more widespread allele (Tsuge, M., Hamamoto, R., Silva, F. P., Ohnishi, Y., Chayama, K., Kamatani, N., Furukawa, Y., and Nakamura, Y. (2005). A variable number of tandem repeats polymorphism in an E2F-1 binding element in the 5′ flanking region of SMYD3 is a risk factor for human cancers. Nature Genetics 37, 1104-1107.). The homozygosity with respect to the allele with three tandem repeats was associated with an increased risk in a cohort of Japanese patients with colorectal cancer, hepatocellular carcinoma, and breast cancer. This association might be specific to Asian cancer patients since a similar study on German breast cancer patients failed to demonstrate such an association (Frank, B., Hemminki, K., Wappenschmidt, B., Klaes, R., Meindl, A., Schmutzler, R. K., Bugert, P., Untch, M., Bartram, C. R., and Burwinkel, B. (2006). Variable number of tandem repeats polymorphism in the SMYD3 promoter region and the risk of familial breast cancer. International Journal of Cancer 118, 2917-2918.).

The oncogenic activity of SMYD3 likely derives from the myriad of genes it regulates and which influence cell proliferation and differentiation. Among these genes were the pro-tumorigenic genes Wnt10B, PIK3CB, PIK3CB, CRKL, CDK2, Cyclin G1, Shh, and CutL1 (Hamamoto, R., Furukawa, Y., Morita, M., Iimura, Y., Silva, F. P., Li, M., Yagyu, R., and Nakamura, Y. (2004). SMYD3 encodes a histone methyltransferase involved in the proliferation of cancer cells. Nature Cell Biology 6, 731-740.). Many of the target genes could be regulated directly via binding of SMYD3 to DNA control elements in these genes. An in vitro selection procedure identified the sequence CCCTCC as a likely candidate recognition sequence. The demonstration that the siRNA knockdown of SMYD3 impairs cancer cell growth in vitro and in vivo (Hamamoto, R., Furukawa, Y., Morita, M., Iimura, Y., Silva, F. P., Li, M., Yagyu, R., and Nakamura, Y. (2004). SMYD3 encodes a histone methyltransferase involved in the proliferation of cancer cells. Nature Cell Biology 6, 731-740; Hamamoto, R., Silva, F. P., Tsuge, M., Nishidate, T., Katagiri, T., Nakamura, Y., and Furukawa, Y. (2006). Enhanced SMYD3 expression is essential for the growth of breast cancer cells. Cancer Science 97, 113-118; Xu, J. Y., Chen, L. B., Xu, J. Y., Yang, Z., Xu, R. H., and Wei, H. Y. (2006). [Experimental research of therapeutic effect on hepatocellular carcinoma of targeting SMYD3 gene inhibition by RNA interference]. Zhonghua wai ke za zhi [Chinese Journal of Surgery] 44, 481-484.) makes SMYD3 an attractive target for molecularly targeted therapy of breast, colon, and liver cancers. This could likely be achieved by small molecules that inhibit the methyltransferase activity via interacting with the sites on the SMYD3 protein for binding the S-adenosyl methionine cofactor (SAM), the Hsp90 chaperone, the histone H3 substrate, and other SMYD3-interacting proteins (such as the RNA helicase HELZ) or novel allosteric sites.

We determined the X-ray crystal structure of SMYD3 in order to enable structure-guided inhibitor design. The binding site for SAM was visualized clearly from co-crystals with the SAM analog Sinefungin. The binding mode of SAM is consistent with what has been previously seen with other SET domain-containing methyltransferases. 15 different lysine methytransferase structures are published in the PDB (2 structures each of human Set8 [2BQZ, 1ZKK] and Neurospora DIM-5 [1PEG, 1ML9], 4 of human Set9 [2F69, 1XQH, 1N6A, 1N6C], 5 of pea plant Lsmt [2H21, 2H23, 2H2E, 2H2J, 1MLV], and 1 each of yeast Dot1p [1U2Z] and human euchromatic histone methyltransferase 1 [2IGQ].

### SUMMARY OF THE INVENTION

The present invention provides the first time the crystal structure of the SMYD3 methyltransferase domain. This structure elucidates the key residues for S-adenosyl-methionine (SAM) binding and the binding region for its substrates. The structure also presents a rationale for the structure-based design of small molecule SMYD3 binders as therapeutic agents, thus addressing the need for novel drugs for the treatment of cancer and/or male infertility or fertility and related conditions.

The present invention also provides molecules comprising SMYD3 binding pockets, or SMYD3-like binding pockets that have similar three-dimensional shapes. In one embodiment, the molecules are SMYD3 or SMYD3-like proteins, protein complexes, or homologues thereof. In another embodiment, the molecules are SMYD3 domains or homologues thereof. In another embodiment, the molecules are in crystalline form.

The invention provides crystallizable compositions and crystal compositions comprising the domain of human SMYD3 or a homologue thereof with or without a chemical entity.

The invention provides a computer comprising a machine-readable storage medium, comprising a data storage material encoded with machine-readable data, wherein the data defines the binding pockets or domains according to the structure coordinates of molecules or molecular complexes of SMYD3 or SMYD3-like proteins, protein complexes or homologues thereof. The invention also provides a computer comprising the data storage medium. Such storage medium when read and utilized by a computer programmed with appropriate software can display, on a computer screen or similar viewing device, a three-dimensional graphical representation of such binding pockets or domains. In one embodiment, the structure coordinates of said molecules or molecular complexes are produced by homology modeling of the coordinates of FIG. 1A.

The invention also provides methods for designing, selecting, evaluating and identifying and/or optimizing compounds that bind to the molecules or molecular complexes or their binding pockets. Such compounds are potential binders of SMYD3, SMYD3-like proteins or their homologues.

The invention also provides a method for determining at least a portion of the three-dimensional structure of molecules or molecular complexes which contain at least some structurally similar features to SMYD3, particularly SMYD3 homologues. This is achieved by using at least some of the structure coordinates obtained from a SMYD3 domain.

The invention provides a crystal comprising a domain of a SMYD3 methyltransferase protein or a homologue thereof, wherein the domain of the SMYD3 methyltransferase protein is selected from the group consisting of amino acid residues X-Y of SEQ ID NO:1, where X=1, 2, or 7 and Y=419 or 428, and optionally other chemical entities are present. Alternatively, the domain of the SMYD3 methyltransferase protein comprises amino acid residues 1-428 of SEQ ID NO:1, and optionally other chemical entities are present.

The invention provides a crystallizable composition comprising a domain of a SMYD3 methyltransferase protein or a homologue thereof, wherein the domain of the SMYD3 methyltransferase protein is selected from the group consisting of amino acid residues X-Y of SEQ ID NO:1, where X=1, 2, or 7 and Y=419 or 428, and optionally other chemical entities are present. Alternatively, the domain of the SMYD3 methyltransferase protein comprises amino acid residues 1-428 of SEQ ID NO:1, and optionally other chemical entities are present.

The invention provides a computer comprising:

(a) a machine-readable data storage medium, comprising a data storage material encoded with machine-readable data, wherein the data defines a binding pocket or domain selected from the group consisting of:

(i) a set of amino acid residues which are identical to human SMYD3 methyltransferase amino acid residues R14, N132, Y124, and N205 according to FIG. 1A, wherein the root mean square deviation of the backbone atoms between the set of amino acid residues and the SMYD3 amino acid residues is not greater than about 2.0 Å;

(ii) a set of amino acid residues comprising at least three amino acid residues which are identical to human SMYD3 methyltransferase amino acid residues R14, N16, Y124, E130, N132, N181, N205, H206, and F259 according to FIG. 1A, wherein the root mean square deviation of the backbone atoms between the at least three amino acid residues and the SMYD3 amino acid residues which are identical is not greater than about 2.0 Å;

(iii) a set of amino acid residues comprising at least five amino acid residues which are identical to human SMYD3 methyltransferase amino acid residues R14, N16, Y124, E130, N132, N181, N205, H206, and F259 according to FIG. 1A, wherein the root mean square deviation of the backbone atoms between the at least five amino acid residues and the SMYD3 amino acid residues which are identical is not greater than about 2.0 Å;

(iv) a set of amino acid residues comprising at least five amino acid residues which are identical to human SMYD3 methyltransferase amino acid residues R14, G15, N16, G17, Y124, E130, N132, K135, C180, N181, S182, F183, T184, I201, S202, L203, L204, N205, H206, S207, C208, I214, I237, C238, Y239, L240, D241, R249, L253, Q256, Y257, F259, C261, D262, C263, R265, C266 according to FIG. 1A, wherein the root mean square deviation of the backbone atoms between the at least five amino acid residues and the SMYD3 amino acid residues which are identical is not greater than about 2.0 Å; and

(v) a set of amino acid residues comprising at least six amino acid residues which are identical to human SMYD3 methyltransferase amino acid residues R14, G15, N16, G17, Y124, E130, N132, K135, C180, N181, S182, F183, T184, I201, S202, L203, L204, N205, H206, S207, C208, I214, I237, C238, Y239, L240, D241, R249, L253, Q256, Y257, F259, C261, D262, C263, R265, C266 according to FIG. 1A, wherein the root mean square deviation of the backbone atoms between the at least six amino acid residues and the SMYD3 amino acid residues which are identical is not greater than about 2.0 Å; and

(vi) a set of amino acid residues that are identical to SMYD3 amino acid residues according to FIG. 1A, wherein the root mean square deviation between the set of amino acid residues and the SMYD3 amino acid residues is not more than about 2.0 Å;

(vii) a set of amino acid residues that are identical to SMYD3 amino acid residues according to FIG. 1A, wherein the root mean square deviation between the set of amino acid residues and the SMYD3 amino acid residues is not more than about 3.0 Å;

(b) a working memory for storing instructions for processing the machine-readable data;

(c) a central processing unit coupled to the working memory and to the machine-readable data storage medium for processing the machine-readable data and a means for generating three-dimensional structural information of the binding pocket or domain; and

(d) output hardware coupled to the central processing unit for outputting said three-dimensional structural information of the binding pocket or domain, or information produced using the three-dimensional structural information of the binding pocket or domain.

For example, the binding pocket is produced by homology modeling of the structure coordinates of the SMYD3 methyltransferase amino acid residues according to FIG. 1A. The means for generating three-dimensional structural information is for example provided by means for generating a three-dimensional graphical representation of the binding pocket or domain.

The output hardware is for example, a display terminal, a printer, CD or DVD recorder, ZIP™ or JAZ™ drive, a disk drive, or other machine-readable data storage device.

The invention provides a method of using a computer for selecting an orientation of a chemical entity that interacts favorably with a binding pocket or domain selected from the group consisting of:

(i) a set of amino acid residues which are identical to human SMYD3 methyltransferase amino acid residues R14, N132, Y124, and N205 according to FIG. 1A, wherein the root mean square deviation of the backbone atoms between the set of amino acid residues and the SMYD3 amino acid residues is not greater than about 2.0 Å;

(ii) a set of amino acid residues comprising at least three amino acid residues which are identical to human SMYD3 methyltransferase amino acid residues R14, N16, Y124, E130, N132, N181, N205, H206, and F259 according to FIG. 1A, wherein the root mean square deviation of the backbone atoms between the at least three amino acid residues and the SMYD3 amino acid residues which are identical is not greater than about 2.0 Å;

(iii) a set of amino acid residues comprising at least five amino acid residues which are identical to human SMYD3 methyltransferase amino acid residues R14, N16, Y124, E130, N132, N181, N205, H206, and F259 according to FIG. 1A, wherein the root mean square deviation of the backbone atoms between the at least five amino acid residues and the SMYD3 amino acid residues which are identical is not greater than about 2.0 Å;

(iv) a set of amino acid residues comprising at least five amino acid residues which are identical to human SMYD3 methyltransferase amino acid residues R14, G15, N16, G17, Y124, E130, N132, K135, C180, N181, S182, F183, T184, I201, S202, L203, L204, N205, H206, S207, C208, I214, I237, C238, Y239, L240, D241, R249, L253, Q256, Y257, F259, C261, D262, C263, R265, C266 according to FIG. 1A, wherein the root mean square deviation of the backbone atoms between the at least five amino acid residues and the SMYD3 amino acid residues which are identical is not greater than about 2.0 Å; and

(v) a set of amino acid residues comprising at least six amino acid residues which are identical to human SMYD3 methyltransferase amino acid residues R14, G15, N16, G17, Y124, E130, N132, K135, C180, N181, S182, F183, T184, I201, S202, L203, L204, N205, H206, S207, C208, I214, I237, C238, Y239, L240, D241, R249, L253, Q256, Y257, F259, C261, D262, C263, R265, C266 according to FIG. 1A, wherein the root mean square deviation of the backbone atoms between the at least six amino acid residues and the SMYD3 amino acid residues which are identical is not greater than about 2.0 Å; and

(vi) a set of amino acid residues that are identical to SMYD3 amino acid residues according to FIG. 1A, wherein the root mean square deviation between the set of amino acid residues and the SMYD3 amino acid residues is not more than about 2.0 Å;

(vii) a set of amino acid residues that are identical to SMYD3 amino acid residues according to FIG. 1A, wherein the root mean square deviation between the set of amino acid residues and the SMYD3 amino acid residues is not more than about 3.0 Å;

the method comprising the steps of:
a. providing the structure coordinates of the binding pocket or domain on a computer comprising means for generating three-dimensional structural information from the structure coordinates;
b. employing computational means to dock a first chemical entity in the binding pocket or domain;
c. quantifying the association between the chemical entity and all or part of the binding pocket or domain for different orientations of the chemical entity; and
d. selecting the orientation of the chemical entity with the most favorable interaction based on the quantified association.

Optionally, the method further comprises the step of (e) generating a three-dimensional graphical representation of the binding pocket or domain prior to step (b). The energy minimization, molecular dynamics simulations, or rigid-body minimizations combinations thereof, or similar induced-fit manipulations are performed simultaneously with or following step (b). Optionally the method further comprises the steps of:

(e) repeating steps (b) through (d) with a second chemical entity; and

(f) selecting of at least one of said first or second chemical entity that interacts more favorably with said-binding pocket or domain based on said quantified association of said first or second chemical entity.

The invention provides a method of using a computer for selecting an orientation of a chemical entity with a favorable shape complementarity in a binding pocket selected from the group consisting of:

(i) a set of amino acid residues which are identical to human SMYD3 methyltransferase amino acid residues R14, N132, Y124, and N205 according to FIG. 1A, wherein the root mean square deviation of the backbone atoms between the set of amino acid residues and the SMYD3 amino acid residues is not greater than about 2.0 Å;

(ii) a set of amino acid residues comprising at least three amino acid residues which are identical to human SMYD3 methyltransferase amino acid residues R14, N16, Y124, E130, N132, N181, N205, H206, and F259 according to FIG. 1A, wherein the root mean square deviation of the backbone atoms between the at least three amino acid residues and the SMYD3 amino acid residues which are identical is not greater than about 2.0 Å;

(iii) a set of amino acid residues comprising at least five amino acid residues which are identical to human SMYD3 methyltransferase amino acid residues R14, N16, Y124, E130, N132, N181, N205, H206, and F259 according to FIG. 1A, wherein the root mean square deviation of the backbone atoms between the at least five amino acid residues and the SMYD3 amino acid residues which are identical is not greater than about 2.0 Å;

(iv) a set of amino acid residues comprising at least five amino acid residues which are identical to human SMYD3 methyltransferase amino acid residues R14, G15, N16, G17, Y124, E130, N132, K135, C180, N181, S182, F183, T184, I201, S202, L203, L204, N205, H206, S207, C208, I214, I237, C238, Y239, L240, D241, R249, L253, Q256, Y257, F259, C261, D262, C263, R265, C266 according to FIG. 1A, wherein the root mean square deviation of the backbone atoms between the at least five amino acid residues and the SMYD3 amino acid residues which are identical is not greater than about 2.0 Å; and

(v) a set of amino acid residues comprising at least six amino acid residues which are identical to human SMYD3 methyltransferase amino acid residues R14, G15, N16, G17, Y124, E130, N132, K135, C180, N181, S182, F183, T184, I201, S202, L203, L204, N205, H206, S207, C208, I214, I237, C238, Y239, L240, D241, R249, L253, Q256, Y257, F259, C261, D262, C263, R265, C266 according to FIG. 1A, wherein the root mean square deviation of the backbone atoms between the at least six amino acid residues and the SMYD3 amino acid residues which are identical is not greater than about 2.0 Å; and

(vi) a set of amino acid residues that are identical to SMYD3 amino acid residues according to FIG. 1A, wherein the root mean square deviation between the set of amino acid residues and the SMYD3 amino acid residues is not more than about 2.0 Å;

(vii) a set of amino acid residues that are identical to SMYD3 amino acid residues according to FIG. 1A, wherein the root mean square deviation between the set of amino acid residues and the SMYD3 amino acid residues is not more than about 3.0 Å;

the method comprising the steps of:
a. providing the structure coordinates of the binding pocket and all or part of the substrate binding pocket therein on a computer comprising means for generating three-dimensional structural information from the structure coordinates;
b. employing computational means to dock a first chemical entity in the binding pocket;
c. quantitating the contact score of the chemical entity in different orientations; and
d. selecting the orientation with the highest contact score.

In various aspects, the method further comprises the steps of:

(e) repeating steps (b) through (d) with a second chemical entity; and

(f) selecting of at least one of said first or second chemical entity that has a higher contact score based on the quantitated contact score of the first or second chemical entity.

Optionally the method further comprises the step of: generating a three-dimensional graphical representation of the binding pocket and all or part of the substrate binding pocket therein prior to step (b).

The invention provides a method for identifying a candidate binder of a molecule or molecular complex comprising a binding pocket or domain selected from the group consisting of:

(i) a set of amino acid residues which are identical to human SMYD3 methyltransferase amino acid residues R14, N132, Y124, and N205 according to FIG. 1A, wherein the root mean square deviation of the backbone atoms between the set of amino acid residues and the SMYD3 amino acid residues is not greater than about 2.0 Å;

(ii) a set of amino acid residues comprising at least three amino acid residues which are identical to human SMYD3 methyltransferase amino acid residues R14, N16, Y124, E130, N132, N181, N205, H206, and F259 according to FIG. 1A, wherein the root mean square deviation of the backbone atoms between the at least three amino acid residues and the SMYD3 amino acid residues which are identical is not greater than about 2.0 Å;

(iii) a set of amino acid residues comprising at least five amino acid residues which are identical to human SMYD3 methyltransferase amino acid residues R14, N16, Y124, E130, N132, N181, N205, H206, and F259 according to FIG. 1A, wherein the root mean square deviation of the backbone atoms between the at least five amino acid residues and the SMYD3 amino acid residues which are identical is not greater than about 2.0 Å;

(iv) a set of amino acid residues comprising at least five amino acid residues which are identical to human SMYD3 methyltransferase amino acid residues R14, G15, N16, G17, Y124, E130, N132, K135, C180, N181, S182, F183, T184, I201, S202, L203, L204, N205, H206, S207, C208, I214, I237, C238, Y239, L240, D241, R249, L253, Q256, Y257, F259, C261, D262, C263, R265, C266 according to FIG. 1A, wherein the root mean square deviation of the backbone atoms between the at least five amino acid residues and the SMYD3 amino acid residues which are identical is not greater than about 2.0 Å; and

(v) a set of amino acid residues comprising at least six amino acid residues which are identical to human SMYD3 methyltransferase amino acid residues R14, G15, N16, G17, Y124, E130, N132, K135, C180, N181, S182, F183, T184, I201, S202, L203, L204, N205, H206, S207, C208, I214, I237, C238, Y239, L240, D241, R249, L253, Q256, Y257, F259, C261, D262, C263, R265, C266 according to FIG. 1A, wherein the root mean square deviation of the backbone atoms between the at least six amino acid residues and the SMYD3 amino acid residues which are identical is not greater than about 2.0 Å; and

(vi) a set of amino acid residues that are identical to SMYD3 amino acid residues according to FIG. 1A, wherein the root mean square deviation between the set of amino acid residues and the SMYD3 amino acid residues is not more than about 2.0 Å;

(vii) a set of amino acid residues that are identical to SMYD3 amino acid residues according to FIG. 1A, wherein the root mean square deviation between the set of amino acid residues and the SMYD3 amino acid residues is not more than about 3.0 Å;

comprising the steps of:
a. using a three-dimensional structure of the binding pocket or domain to design, select or optimize a plurality of chemical entities;
b. contacting each chemical entity with the molecule or the molecular complex;
c. monitoring the effect of the catalytic activity of the molecule or molecular complex by each chemical entity; and
d. selecting a chemical entity based on the modulatory effect of the chemical entity on the catalytic activity of the molecule or molecular complex.

Whether one monitors and selects a chemical with an inhibitory or stimulatory effect on the catalytic activity will depend on the intended use of the selected chemical. For example, an inhibitor may be desirable as a treatment for certain cancers.

The invention provides a method of designing a compound or complex that interacts with a binding pocket or domain selected from the group consisting of:

(i) a set of amino acid residues which are identical to human SMYD3 methyltransferase amino acid residues R14, N132, Y124, and N205 according to FIG. 1A, wherein the root mean square deviation of the backbone atoms between the set of amino acid residues and the SMYD3 amino acid residues is not greater than about 2.0 Å;

(ii) a set of amino acid residues comprising at least three amino acid residues which are identical to human SMYD3 methyltransferase amino acid residues R14, N16, Y124, E130, N132, N181, N205, H206, and F259 according to FIG. 1A, wherein the root mean square deviation of the backbone atoms between the at least three amino acid residues and the SMYD3 amino acid residues which are identical is not greater than about 2.0 Å;

(iii) a set of amino acid residues comprising at least five amino acid residues which are identical to human SMYD3 methyltransferase amino acid residues R14, N16, Y124, E130, N132, N181, N205, H206, and F259 according to FIG. 1A, wherein the root mean square deviation of the backbone atoms between the at least five amino acid residues and the SMYD3 amino acid residues which are identical is not greater than about 2.0 Å;

(iv) a set of amino acid residues comprising at least five amino acid residues which are identical to human SMYD3 methyltransferase amino acid residues R14, G15, N16, G17, Y124, E130, N132, K135, C180, N181, S182, F183, T184, I201, S202, L203, L204, N205, H206, S207, C208, I214, I237, C238, Y239, L240, D241, R249, L253, Q256, Y257, F259, C261, D262, C263, R265, C266 according to FIG. 1A, wherein the root mean square deviation of the backbone atoms between the at least five amino acid residues and the SMYD3 amino acid residues which are identical is not greater than about 2.0 Å; and

(v) a set of amino acid residues comprising at least six amino acid residues which are identical to human SMYD3 methyltransferase amino acid residues R14, G15, N16, G17, Y124, E130, N132, K135, C180, N181, S182, F183, T184, I201, S202, L203, L204, N205, H206, S207, C208, I214, I237, C238, Y239, L240, D241, R249, L253, Q256, Y257, F259, C261, D262, C263, R265, C266 according to FIG. 1A, wherein the root mean square deviation of the backbone atoms between the at least six amino acid residues and the SMYD3 amino acid residues which are identical is not greater than about 2.0 Å; and

(vi) a set of amino acid residues that are identical to SMYD3 amino acid residues according to FIG. 1A, wherein the root mean square deviation between the set of amino acid residues and the SMYD3 amino acid residues is not more than about 2.0 Å;

(vii) a set of amino acid residues that are identical to SMYD3 amino acid residues according to FIG. 1A, wherein the root mean square deviation between the set of amino acid residues and the SMYD3 amino acid residues is not more than about 3.0 Å;

comprising the steps of:
a. providing the structure coordinates of the binding pocket or domain on a computer comprising means for generating three-dimensional structural information from the structure coordinates;
b. using the computer to dock a first chemical entity in part of the binding pocket or domain;
c. docking at least a second chemical entity in another part of the binding pocket or domain;
d. quantifying the association between the first or second chemical entity and part of the binding pocket or domain;
e. repeating steps (b) to (d) with another first and second chemical entity,
f. selecting a first and a second chemical entity based on the quantified association of both the first and second chemical entity;
g. optionally, visually inspecting the relationship of the selected first and second chemical entity to each other in relation to the binding pocket or domain on a computer screen using the three-dimensional graphical representation of the binding pocket or domain and the first and second chemical entity; and
h. assembling the selected first and second chemical entity into a compound or complex that interacts with said binding pocket or domain by model building.

The method provides a method of utilizing molecular replacement to obtain structural information about a molecule or a molecular complex of unknown structure,

wherein the molecule is sufficiently homologous to a domain of a SMYD3 protein, comprising the steps of:
a. crystallizing the molecule or molecular complex;
b. generating an X-ray diffraction pattern from the crystallized molecule or molecular complex; and
c. applying at least a portion of the structure coordinates set forth in FIG. 1A or a homology model thereof to the X-ray diffraction pattern to generate a three-dimensional electron density map of at least a portion of the molecule or molecular complex of unknown structure; and
d. generating a structural model of the molecule or molecular complex from the three-dimensional electron density map.

The molecule is selected from the group consisting of the SMYD3 methyltransferase protein, and a homologue of a domain of the SMYD3 methyltransferase protein.

The molecular complex is selected from the group consisting of the SMYD3 methyltransferase protein complex and a homologue of the SMYD3 complex.

The invention provides a method for identifying a candidate binder that interacts with a binding site of a SMYD3 methyltransferase protein or a homologue thereof, comprising the steps of:
a. obtaining a crystal comprising a domain of said SMYD3 methyltransferase protein or said homologue thereof, wherein the crystal is characterized with space group P_{1 21 1} and has unit cell parameters of a=58.175 Å, b=118.073 Å, c=82.901 Å, α=90.00, β=91.58, γ=90.00;
b. obtaining the structure coordinates of amino acids of the crystal of step (a), wherein the structure coordinates are set forth in FIG. 1A-1 to 1A-129;
c. generating a three-dimensional model of the domain of said SMYD3 methyltransferase protein or said homologue thereof using the structure coordinates of the amino acids obtained in step (b), a root mean square deviation from backbone atoms of said amino acids of not more than ±2.0 Å;
d. determining a binding site of the domain of said SMYD3 methyltransferase protein or said homologue thereof from said three-dimensional model; and
e. performing computer fitting analysis to identify the candidate binder which interacts with said binding site.

Optionally the method, further comprises the step of: (f) contacting the identified candidate binder with the domain of said SMYD3 methyltransferase protein or said homologue thereof in order to determine the effect of the binder on SMYD3 methyltransferase protein activity.

The binding site of the domain of said SMYD3 methyltransferase protein or said homologue thereof determined in step (d) comprises the structure coordinates according to FIG. 1A-1 to 1A-129 of amino acid residues R14, N132, Y124, and N205, wherein the root mean square deviation from the backbone atoms of said amino acids is not more than ±2.0 Å.

Alternatively, the binding site of the domain of said SMYD3 methyltransferase protein or said homologue thereof determined in step (d) comprises the structure coordinates according to FIG. 1A-1 to 1A-129 of amino acid residues R14, N16, Y124, E130, N132, N181, N205, H206, and F259, wherein the root mean square deviation from the backbone atoms of said amino acids is not more than ±2.0 Å.

The invention provides a method for identifying a candidate binder that interacts with a binding site of a domain of a SMYD3 methyltransferase protein or a homologue thereof, comprising the steps of:
a. obtaining a crystal comprising the domain of said SMYD3 methyltransferase protein or said homologue thereof, wherein the crystal is characterized with space group P_{1 21 1} and has unit cell parameters of a=58.175 Å, b=118.073 Å, c=82.901 Å, α=90.00, β=91.58, γ=90.00;

(b) obtaining the structure coordinates of amino acids of the crystal of step (a);

(c) generating a three-dimensional model of said SMYD3 methyltransferase protein or said homologue thereof using the structure coordinates of the amino acids generated in step (b), a root mean square deviation from backbone atoms of said amino acids of not more than ±2.0 Å;

(d) determining a binding site of the domain of said SMYD3 methyltransferase protein or said homologue thereof from said three-dimensional model; and

(e) performing computer fitting analysis to identify the candidate binder which interacts with said binding site.

Optionally, the method further comprises the step of:

(f) contacting the identified candidate binder with the domain of said SMYD3 methyltransferase protein or said homologue thereof in order to determine the effect of the binder on SMYD3 methyltransferase protein activity.

The binding site of the domain of said SMYD3 methyltransferase protein or said homologue thereof determined in step (d) comprises the structure coordinates according to FIG. 1A-1 to 1A-129 of amino acid residues R14, N132, Y124, and N205, wherein the root mean square deviation from the backbone atoms of said amino acids is not more than ±2.0 Å.

Alternatively, the binding site of the domain of said SMYD3 methyltransferase protein or said homologue thereof determined in step (d) comprises the structure coordinates according to FIG. 1A-1 to 1A-129 of amino acid residues R14, N16, Y124, E130, N132, N181, N205, H206, and F259, wherein the root mean square deviation from the backbone atoms of said amino acids is not more than ±2.0 Å.

The binding site of the domain of said SMYD3 methyltransferase protein or said homologue thereof determined in step (d) comprises the structure coordinates according to FIG. 1A-1 to 1A-129 of amino acid residues R14, G15, N16, G17, Y124, E130, N132, K135, C180, N181, S182, F183, T184, I201, S202, L203, L204, N205, H206, S207, C208, I214, I237, C238, Y239, L240, D241, R249, L253, Q256, Y257, F259, C261, D262, C263, R265, and C266, wherein the root mean square deviation from the backbone atoms of said amino acids is not more than ±2.0 Å.

The invention provides a method for identifying a candidate binder that interacts with a binding site of a domain of a SMYD3 methyltransferase protein or a homologue thereof, comprising the step of determining a binding site of the domain of said SMYD3 methyltransferase protein or the homologue thereof from a three-dimensional model to design or identify the candidate binder which interacts with said binding site.

The binding site of the domain of said SMYD3 methyltransferase protein or said homologue thereof determined comprises the structure coordinates according to FIG. 1A-1 to 1A-129 of amino acid residues R14, N132, Y124, and N205, wherein the root mean square deviation from the backbone atoms of said amino acids is not more than ±2.0 Å.

Alternatively the binding site of the domain of said SMYD3 methyltransferase protein or said homologue thereof determined comprises the structure coordinates according to FIG. 1A-1 to 1A-129 of amino acid residues R14, N16, Y124, E130, N132, N181, N205, H206, and F259, wherein the root mean square deviation from the backbone atoms of said amino acids is not more than ±2.0 Å.

The binding site of the domain of said SMYD3 methyltransferase protein or said homologue thereof determined comprises the structure coordinates according to FIG. 1A-1 to 1A-129 of amino acid residues F621, K644, A657, L658, E661, M664, L802, K805, S806, C807, V808, H809, R810, D811, C828, D829, F830, G831, and L832, wherein the root mean square deviation from the backbone atoms of said amino acids is not more than ±2.0 Å.

The invention provides a method for identifying a candidate binder of a molecule or molecular complex comprising a binding pocket or domain selected from the group consisting of:

(i) a set of amino acid residues which are identical to human SMYD3 methyltransferase amino acid residues R14, N132, Y124, and N205 according to FIG. 1A, wherein the root mean square deviation of the backbone atoms between the set of amino acid residues and the SMYD3 amino acid residues is not greater than about 2.0 Å;

(ii) a set of amino acid residues comprising at least three amino acid residues which are identical to human SMYD3 methyltransferase amino acid residues R14, N16, Y124, E130, N132, N181, N205, H206, and F259 according to FIG. 1A, wherein the root mean square deviation of the backbone atoms between the at least three amino acid residues and the SMYD3 amino acid residues which are identical is not greater than about 2.0 Å;

(iii) a set of amino acid residues comprising at least five amino acid residues which are identical to human SMYD3 methyltransferase amino acid residues R14, N16, Y124, E130, N132, N181, N205, H206, and F259 according to FIG. 1A, wherein the root mean square deviation of the backbone atoms between the at least five amino acid residues and the SMYD3 amino acid residues which are identical is not greater than about 2.0 Å;

(iv) a set of amino acid residues comprising at least five amino acid residues which are identical to human SMYD3 methyltransferase amino acid residues R14, G15, N16, G17, Y124, E130, N132, K135, C180, N181, S182, F183, T184, I201, S202, L203, L204, N205, H206, S207, C208, I214, I237, C238, Y239, L240, D241, R249, L253, Q256, Y257, F259, C261, D262, C263, R265, C266 according to FIG. 1A, wherein the root mean square deviation of the backbone atoms between the at least five amino acid residues and the SMYD3 amino acid residues which are identical is not greater than about 2.0 Å; and

(v) a set of amino acid residues comprising at least six amino acid residues which are identical to human SMYD3 methyltransferase amino acid residues R14, G15, N16, G17, Y124, E130, N132, K135, C180, N181, S182, F183, T184, I201, S202, L203, L204, N205, H206, S207, C208, I214, I237, C238, Y239, L240, D241, R249, L253, Q256, Y257, F259, C261, D262, C263, R265, C266 according to FIG. 1A, wherein the root mean square deviation of the backbone atoms between the at least six amino acid residues and the SMYD3 amino acid residues which are identical is not greater than about 2.0 Å; and

(vi) a set of amino acid residues that are identical to SMYD3 amino acid residues according to FIG. 1A, wherein the root mean square deviation between the set of amino acid residues and the SMYD3 amino acid residues is not more than about 2.0 Å;

(vii) a set of amino acid residues that are identical to SMYD3 amino acid residues according to FIG. 1A, wherein the root mean square deviation between the set of amino acid residues and the SMYD3 amino acid residues is not more than about 3.0 Å;

comprising the steps of:

(a) using a three-dimensional structure of the binding pocket or domain to design, select or optimize a plurality of chemical entities; and

(b) selecting said candidate binder based on the effect of said chemical entities on a domain of a SMYD3 methyltransferase protein or a domain of a SMYD3 methyltransferase protein homologue on the catalytic activity of the molecule or molecular complex.

The invention provides a method of using the crystals according to the invention in an binder screening assay comprising: (a) selecting a potential binder by performing rational drug design with a three-dimensional structure determined for the crystal, wherein said selecting is performed in conjunction with computer modeling; (b) contacting the potential binder with a methyltransferase; and (c) detecting the ability of the potential binder to modulate the activity of the methyltransferase.

The invention provides a method of preparing the crystals comprising the steps: (i) generating TOPO adapted plasmids which contain the target sequence that are optionally tagged with particular extensions off the N or C termini of the SMYD3-like methyltransferase sequence [such as His tag] that are known to be useful by those in the art of protein production and purification; (ii) transfecting in an expression system, such as E. Coli or baculovirus; (iii) inducing expression of the SMYD3-like methyltransferase protein product; (iv) screening for overexpression of particular constructs; (v) purifying the overexpressed proteins; (vi) placing the purified protein in a variety of initial conditions for crystallization; and (vii) refining conditions to improve diffraction quality of the crystals. The invention also relates to a method of obtaining a crystal of an SMYD3-like methyltransferase protein or homologue thereof, comprising the steps of a) optionally producing and purifying an SMYD3-like methyltransferase protein or homologue thereof; b) combining a crystallization solution with said SMYD3-like methyltransferase protein or homologue thereof to produce a crystallizable composition; and c) subjecting the composition to conditions which promote crystallization and obtaining said crystal. Other chemical entities that bind SMYD3-like methyltransferases may optionally be present at any stage.

The invention provides a set of coordinates as described in the associated crystal structure defining the 3-dimentional structure of the protein SMYD3 with the amino acid sequence 1-428 [SEQ ID NO:1].

The invention provides compounds in described below in the EXAMPLES, identified by any of the methods described above.

The invention provides a method of treating cancer and/or male infertility or fertility in a patient by administering one or more of compounds, described below in the EXAMPLES, with or without additional formulation or administration of other treatments (e.g. anticancer treatments, antidiabetics).

The invention provides a method for determining SMYD3 binding of any potential SMYD3 binder, including those identified by any of the methods above, comprising the steps (i) generating purified SMYD3 protein; (ii) generating pools of compounds whose components all have unique molecular weights and distinct chemotypes; (iii) contacting the protein with the pools; (iv) separating binders via a spin column; (v) separating any binders from the protein via chemical denaturation; (vi) detecting the amount and chemical nature of binders using mass spectrometry.

### BRIEF DESCRIPTION OF THE FIGURES

The following abbreviations are used in FIG. 1A:

“Atom type” refers to the element whose coordinates are measured. The first letter in the column defines the element.

“Resid” refers to the amino acid residue in the molecular model.

“X, Y, Z” define the atomic position of the element measured.

“B” is a thermal factor that measures movement of the atom around its atomic center.

“Occ” is an occupancy factor that refers to the fraction of the molecules in which each atom occupies the position specified by the coordinates. A value of “1” indicates that each atom has the same conformation, i.e., the same position, in the molecules.

FIG. 1A (1A-1 to 1A-129) lists the atomic coordinates for human SMYD3 (amino acid residues 1-428 of human SMYD3 protein (GenBank accession no. AAH31010; SEQ ID NO:1)) as derived from X-ray diffraction. Residues 1-3 and 423-428 were not included in the final model. The coordinates are shown in Protein Data Bank (PDB) format. Residues “SFG W”, “ZN W”, and “HOH W” represent adenosyl-ornithine, zinc, and water molecules, respectively.

FIG. 2A depicts the SMYD3 structure as a ribbon diagram. The crystals yielded a dimer in the unit cell. The biologically active arrangement is putatively the monomer.

FIG. 2B depicts a single monomer of SMYD3 as a ribbon diagram. “Dots” in the image represent zinc atoms. The group of helices in the lower right hand corner of the figure are part of the insert not present outside the SMYD family and is a structural feature unique to this protein when compared against the entire PDB.

FIG. 2C depicts the SMYD3 monomer as a surface

FIG. 2D show rigidly rotated views of 2B

FIG. 2E show rigidly rotated views of 2C.

FIG. 3A Fig. depicts the SAM binding site with adenosyl-ornithine bound. The Ca trace is represented by a ribbon diagram, while crystallographically resolved atoms from the protein within 5 A of adenosyl-ornithine are depicted in a ball-and-stick representation. Adenosyl-ornithine is depicted with capped sticks. Hydrogen bonds are denoted with a dashed line and residues making key interaction with adenosyl-ornithine are labeled.

FIG. 3B provides the same binding site in the same orientation, except without adenosyl-ornithine present.

FIG. 4 shows the amino acid sequence of human SMYD3 (SEQ ID NO:1).

FIG. 5 shows a diagram of a system used to carry out the instructions encoded by the storage media of FIG. 6.

FIG. 6 shows cross sections of magnetic (A) and optically-readable (B) data storage media.

### DETAILED DESCRIPTION OF THE INVENTION

In order that the invention described herein may be more fully understood, the following detailed description is set forth.

Throughout the specification, the word “comprise” or variations such as “comprises” or “comprising” will be understood to imply the inclusion of a stated integer or groups of integers but not the exclusion of any other integer or groups of integers.

The following abbreviations are used throughout the application:

A=Ala=Alanine T=Thr=Threonine V=Val=Valine C=Cys=Cysteine L=Leu=Leucine Y=Tyr=Tyrosine I=Ile=Isoleucine N=Asn=Asparagine P=Pro=Proline Q=Gln=Glutamine F=Phe=Phenylalanine D=Asp=Aspartic Acid W=Trp=Tryptophan E=Glu=Glutamic Acid M=Met=Methionine K=Lys=Lysine G=Gly=Glycine R=Arg=Arginine S=Ser=Serine H=His=Histidine

As used herein, the following definitions shall apply unless otherwise indicated.

The term “about” when used in the context of root mean square deviation (RMSD) values takes into consideration the standard error of the RMSD value, which is ±0.1 Å.

The term “associating with” refers to a condition of proximity between a chemical entity or compound, or portions thereof, and a binding pocket or binding site on a protein. The association may be non-covalent—wherein hydrogen bonding, hydrophobic, Van der Waals and electrostatic interactions, taken together, favor the juxtaposition—or it may be covalent.

The term “binding pocket” refers to a region of a molecule or molecular complex, that, as a result of its shape, favorably associates with a chemical entity. The term “pocket” includes, but is not limited to, cleft, channel or site. SMYD3, SMYD3-like molecules or homologues thereof may have binding pockets which include, but are not limited to, peptide or substrate binding and SAM-binding sites. The shape of a first binding pocket may be largely pre-formed before binding of a chemical entity, may be formed simultaneously with binding of a chemical entity, or may be formed by the binding of another chemical entity to a different binding pocket of the molecule, which in turn induces a change in shape of the first binding pocket

The term “catalytic active site” or “active site” refers to the portion of the protein to which nucleotide substrates bind. For example, the catalytic active site of SMYD3 is comprised of the residues in the cavity containing the adenosyl-ornithine.

The term “chemical entity” refers to chemical compounds, complexes of at least two chemical compounds, and fragments of such compounds or complexes. The chemical entity can be, for example, a ligand, substrate, nucleotide amino acid, non-naturally occurring nucleotide amino acid, amino acid, nucleotide, agonist, antagonist, binder, antibody, peptide, protein or drug. In one embodiment, the chemical entity is a binder or substrate for the active site of SMYD3 proteins or protein complexes, or homologues thereof. The first and second chemical entities referred to in the present invention may be identical or distinct from each other. When iterative steps of using first and second chemical entities are carried out, taken as a pair, the first and second chemical entities used in repeated steps should be different from the first and second chemical entities of the prior steps.

The term “complex” or “molecular complex” refers to a protein associated with a chemical entity.

The term “conservative substitutions” refers to residues that are physically or functionally similar to the corresponding reference residues. That is, a conservative substitution and its reference residue have similar size, shape, electric charge, chemical properties including the ability to form covalent or hydrogen bonds, or the like. Preferred conservative substitutions are those fulfilling the criteria defined for an accepted point mutation in Dayhoff et al., Atlas of Protein Sequence and Structure, 5: 345-352 (1978 & Supp.), which is incorporated herein by reference. Examples of conservative substitutions are substitutions including but not limited to the following groups: (a) valine, glycine; (b) glycine, alanine; (c) valine, isoleucine, leucine; (d) aspartic acid, glutamic acid; (e) asparagine, glutamine; (f) serine, threonine; (g) lysine, arginine, methionine; and (h) phenylalanine, tyrosine.

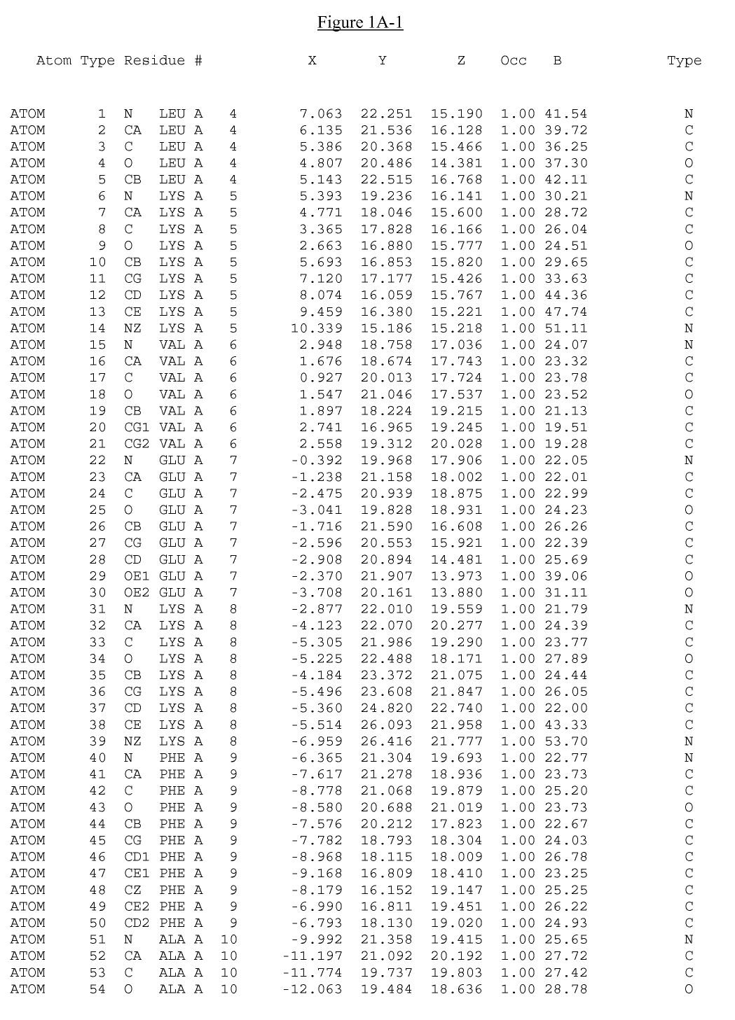

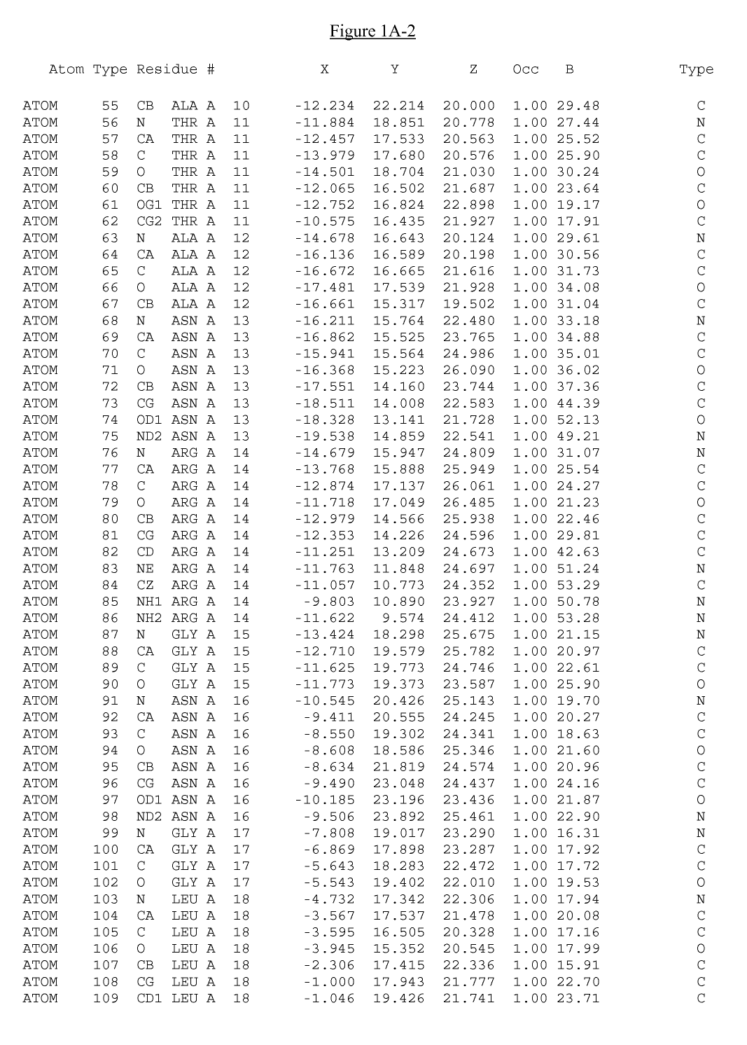

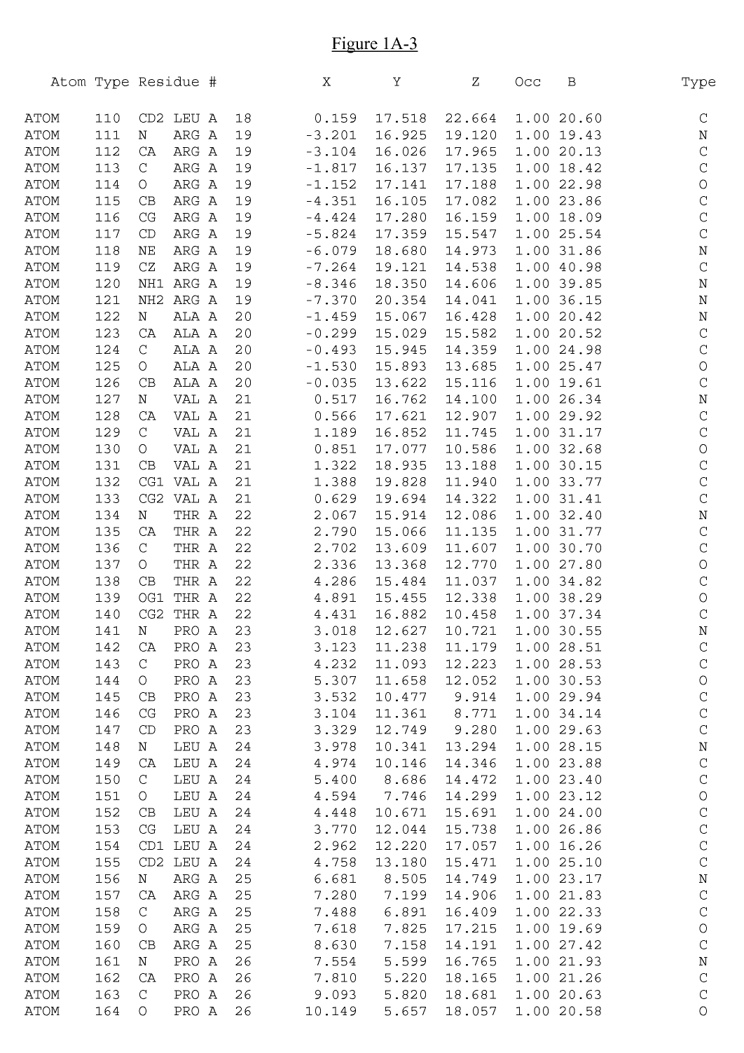

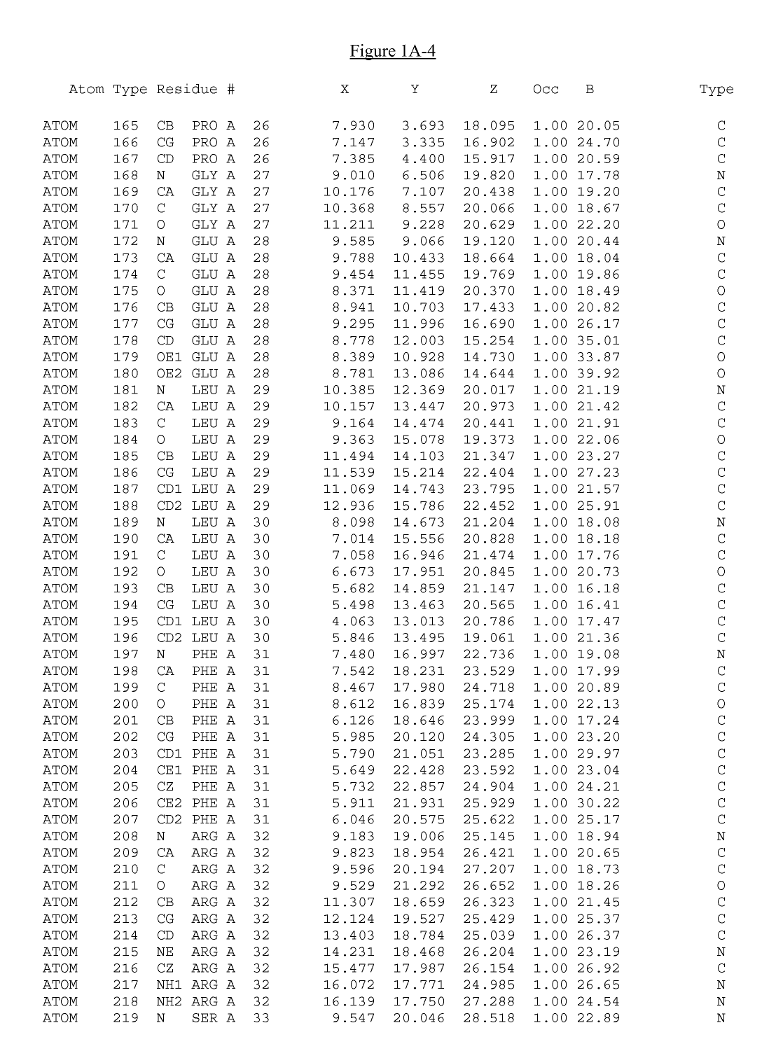

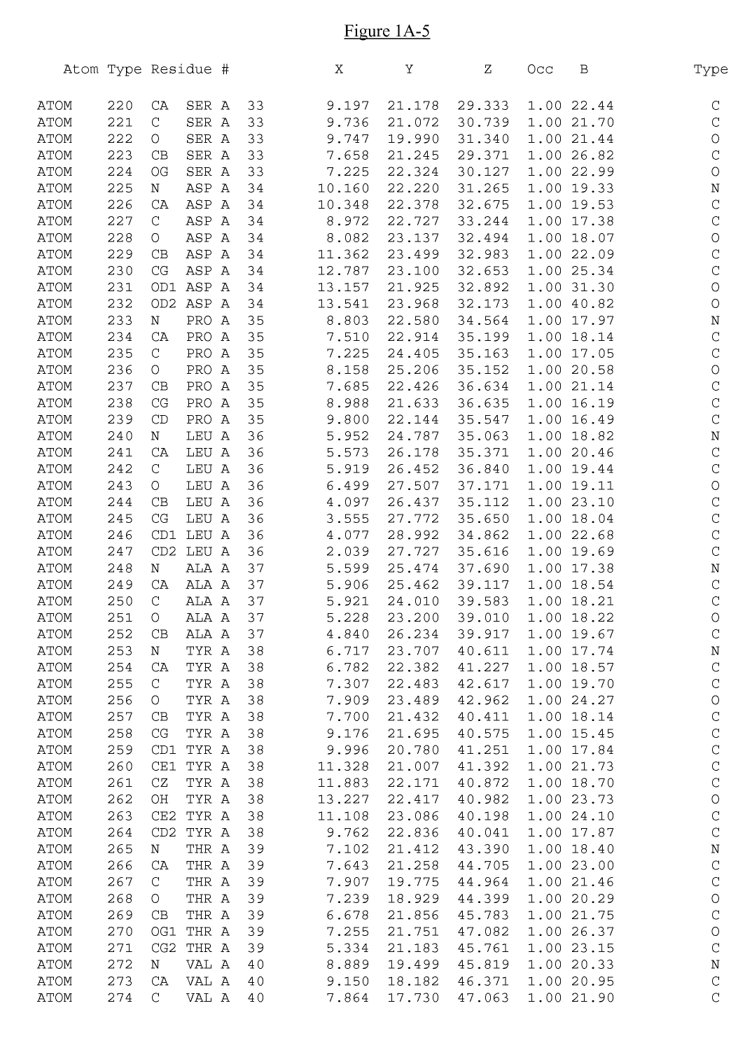

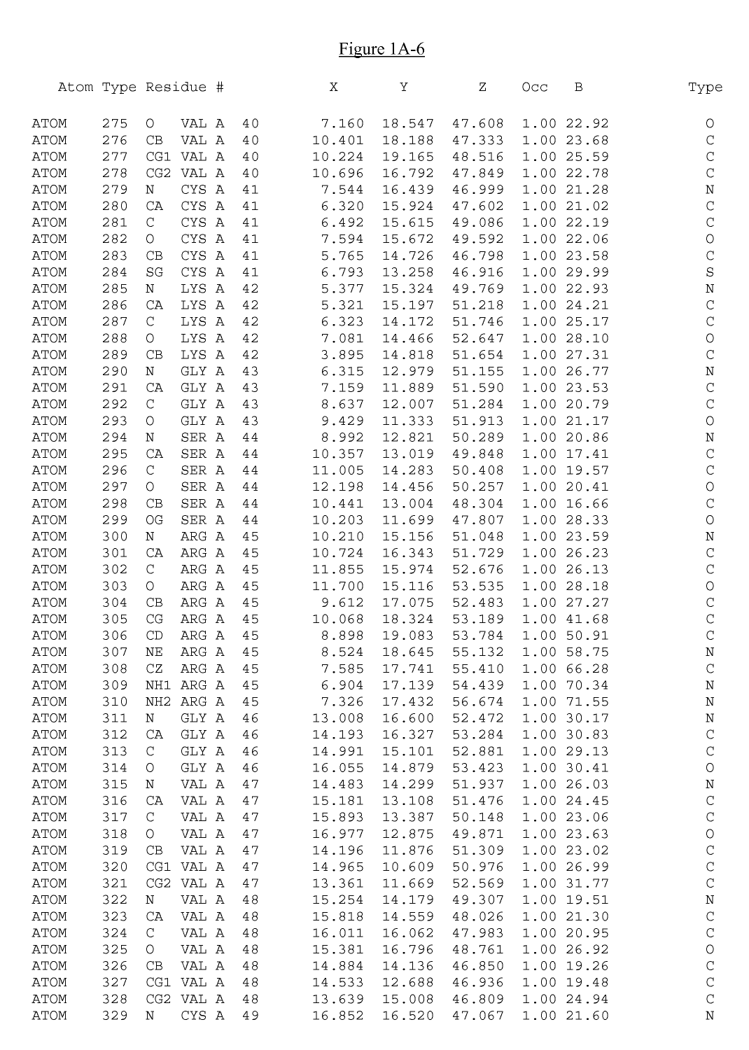

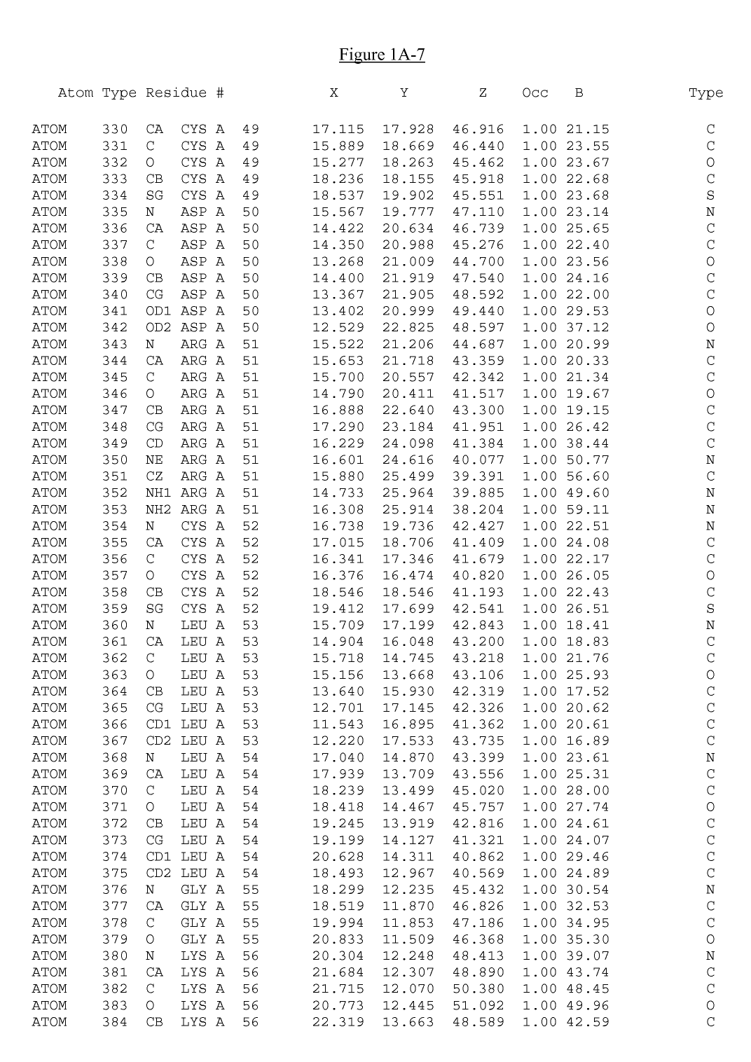

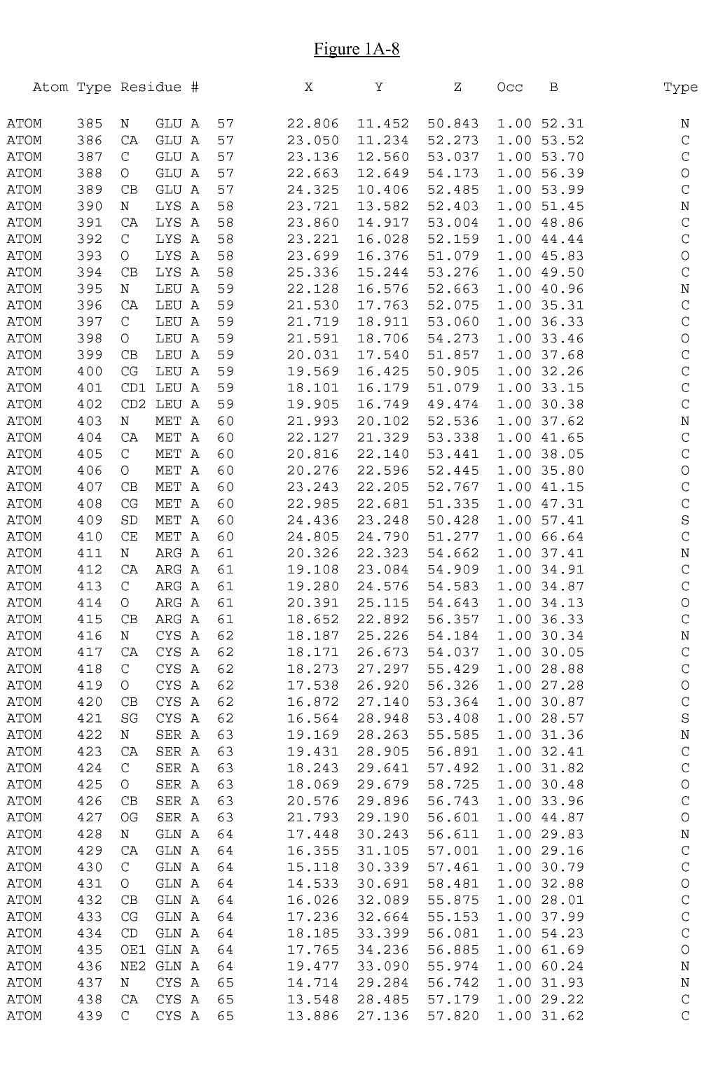

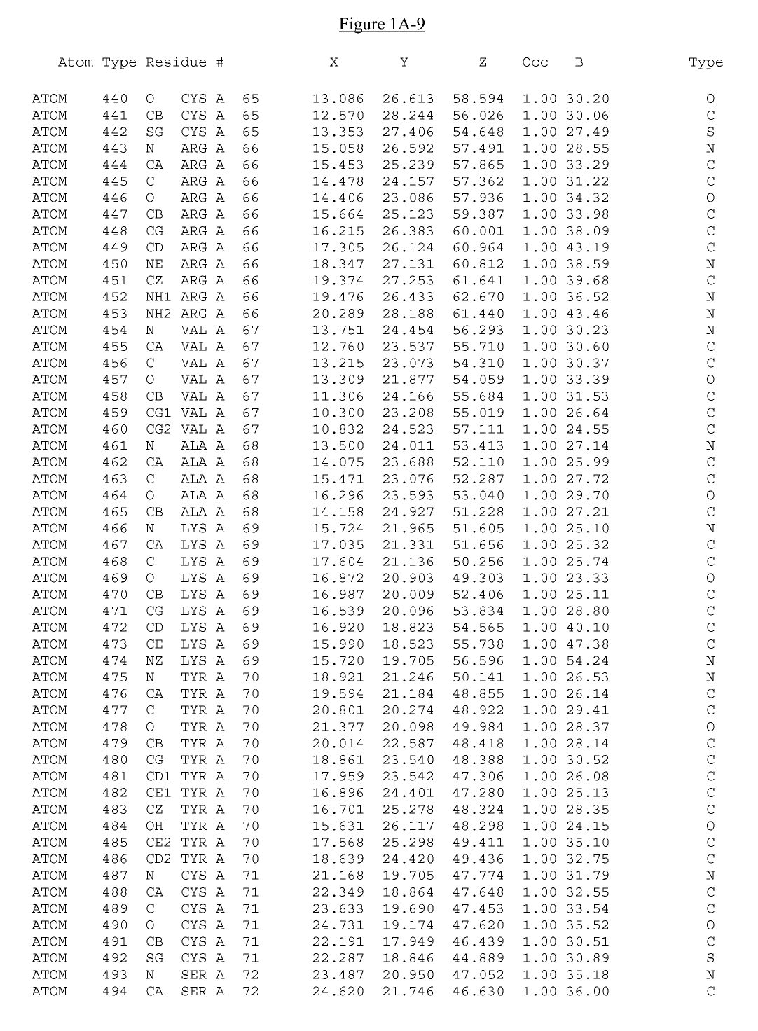

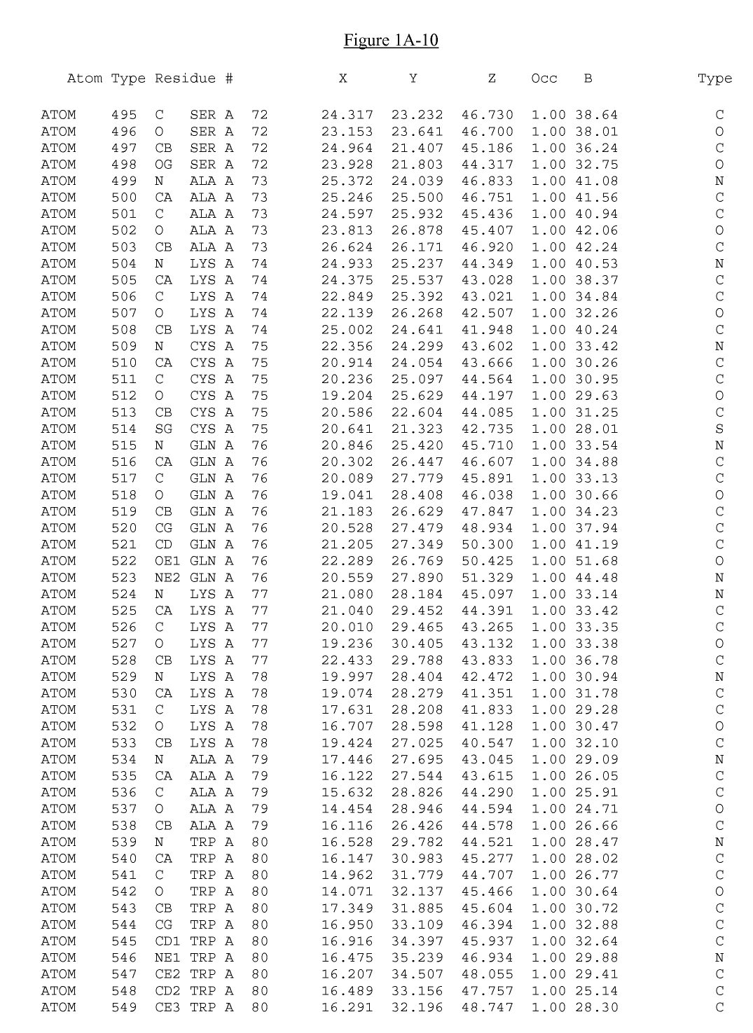

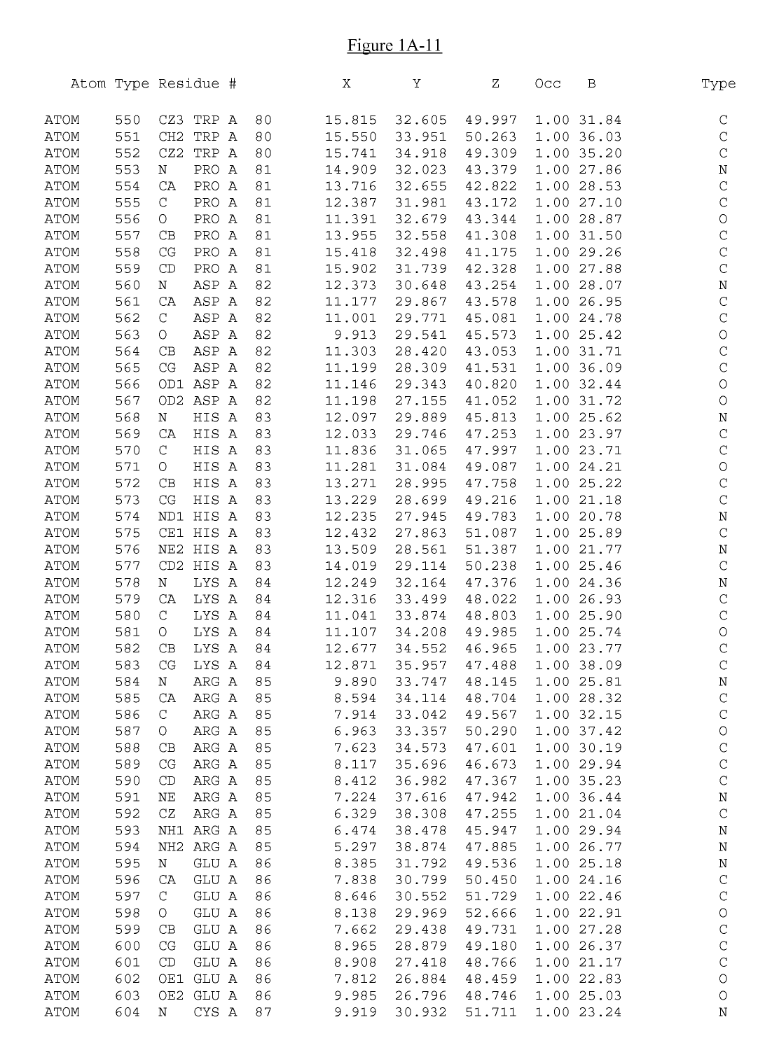

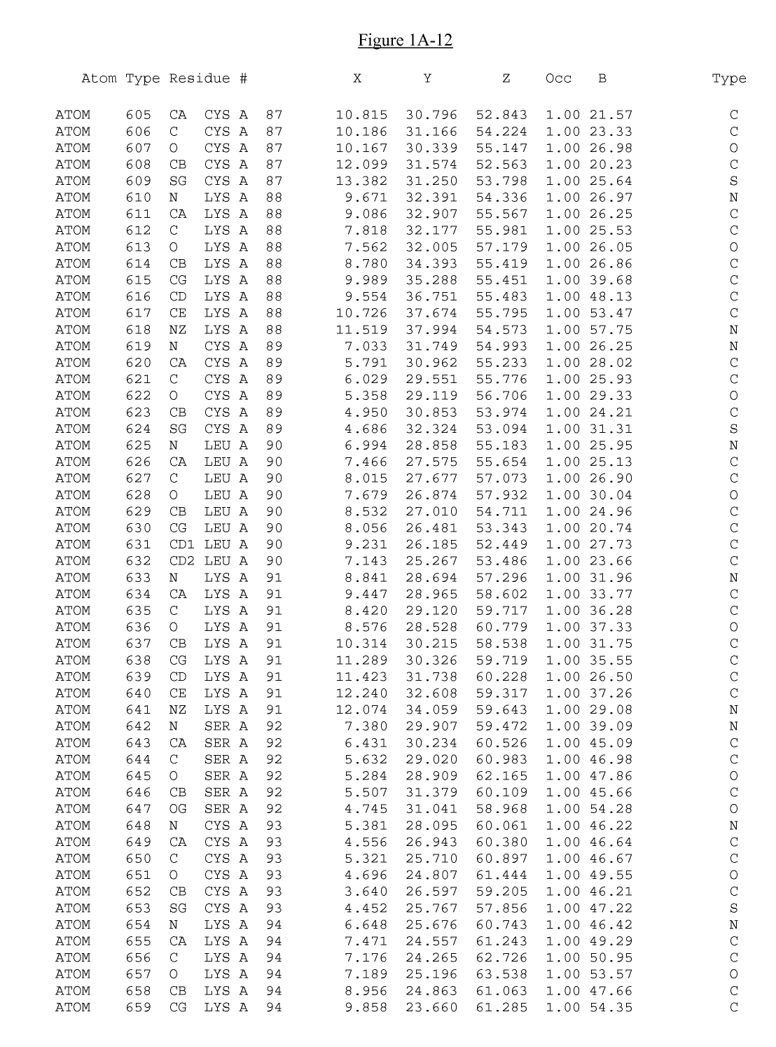

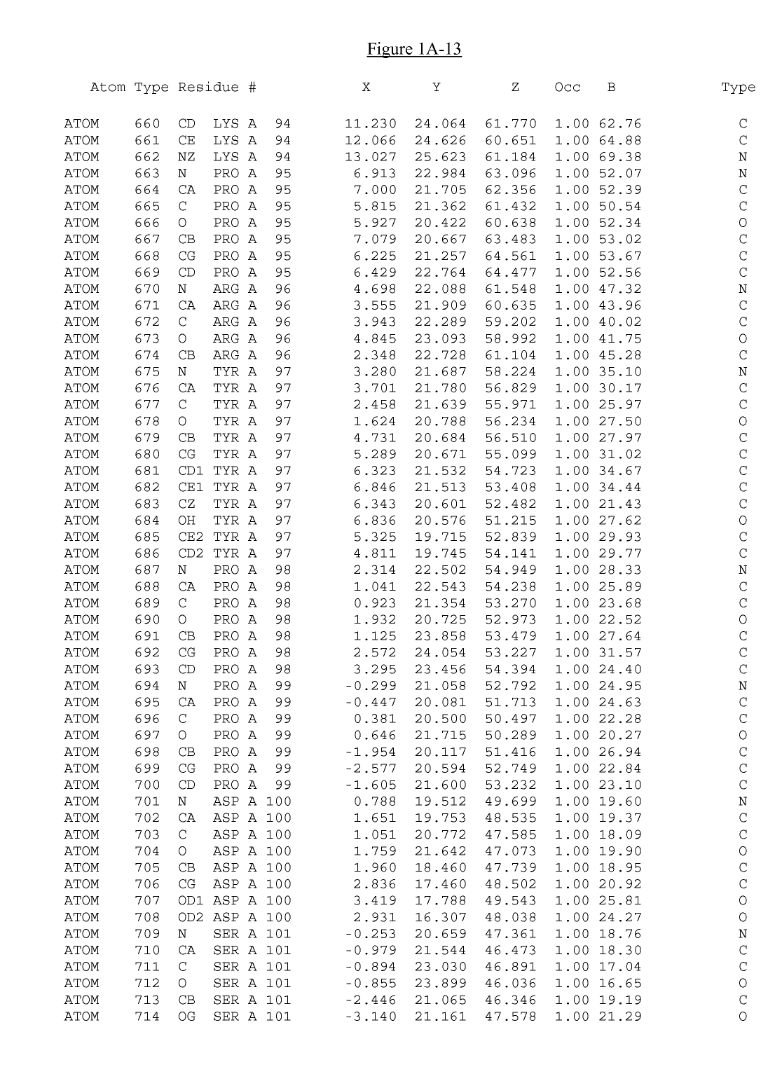

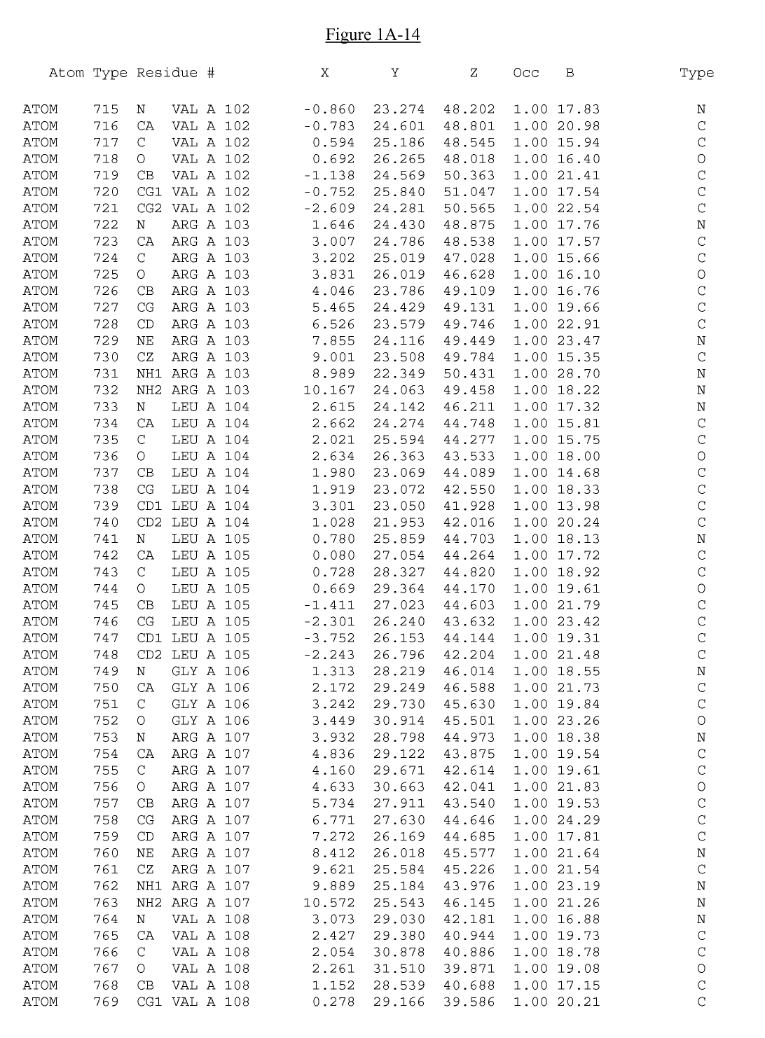

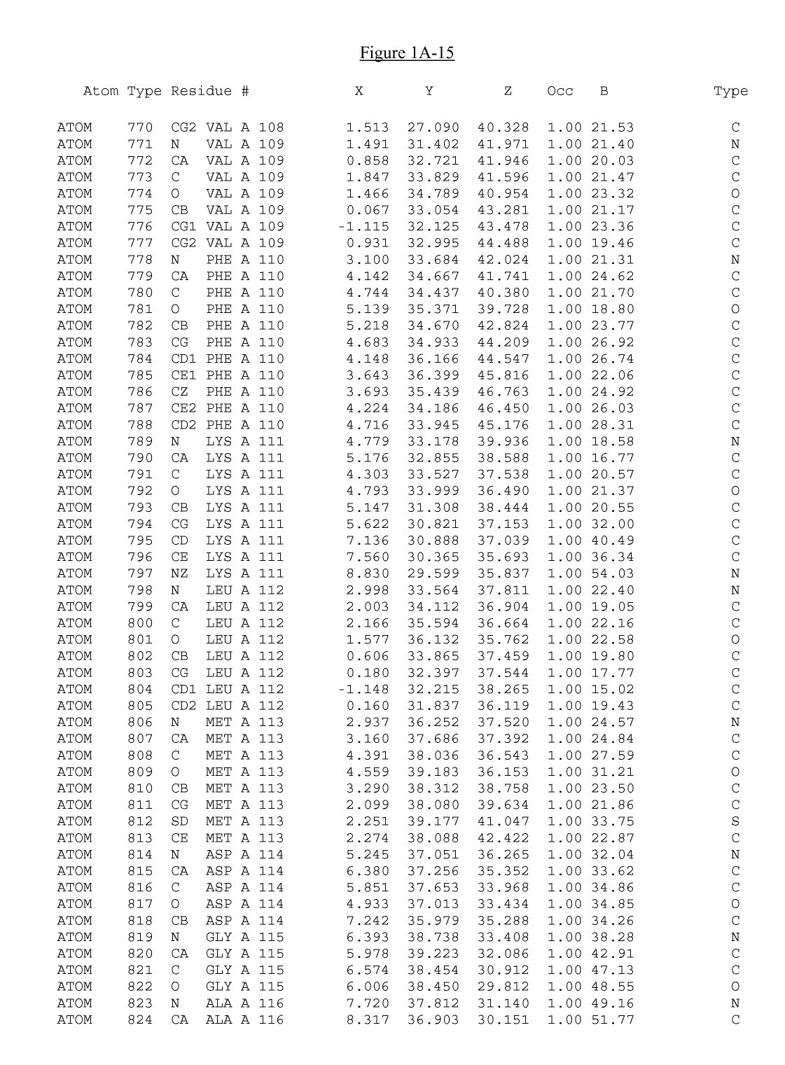

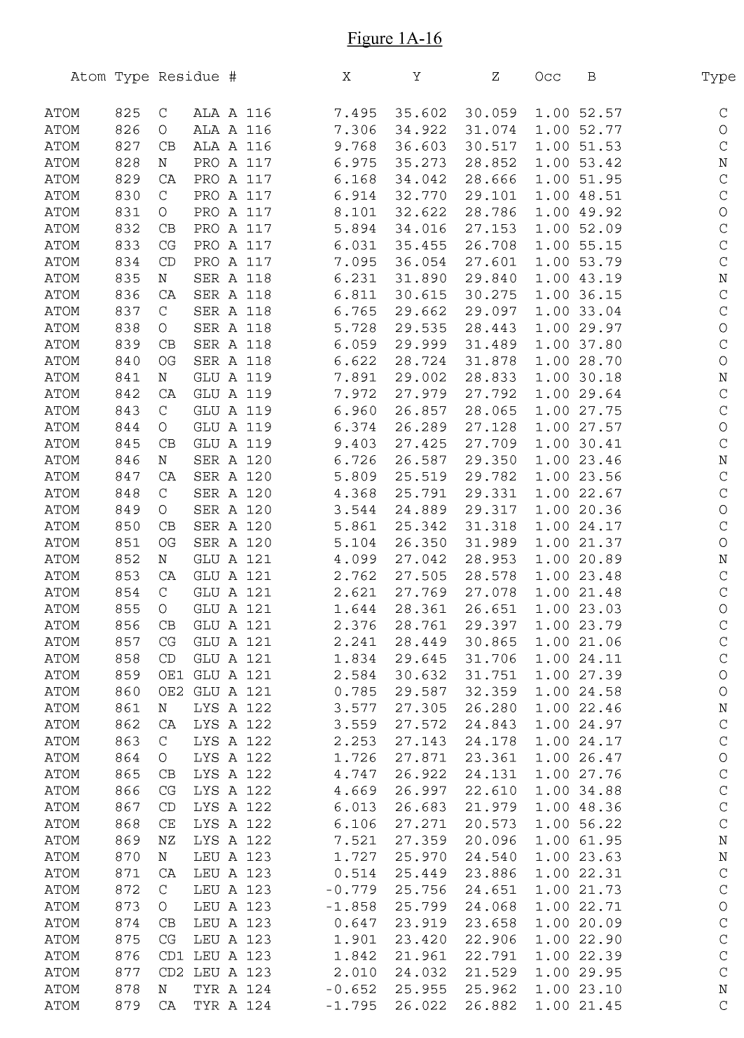

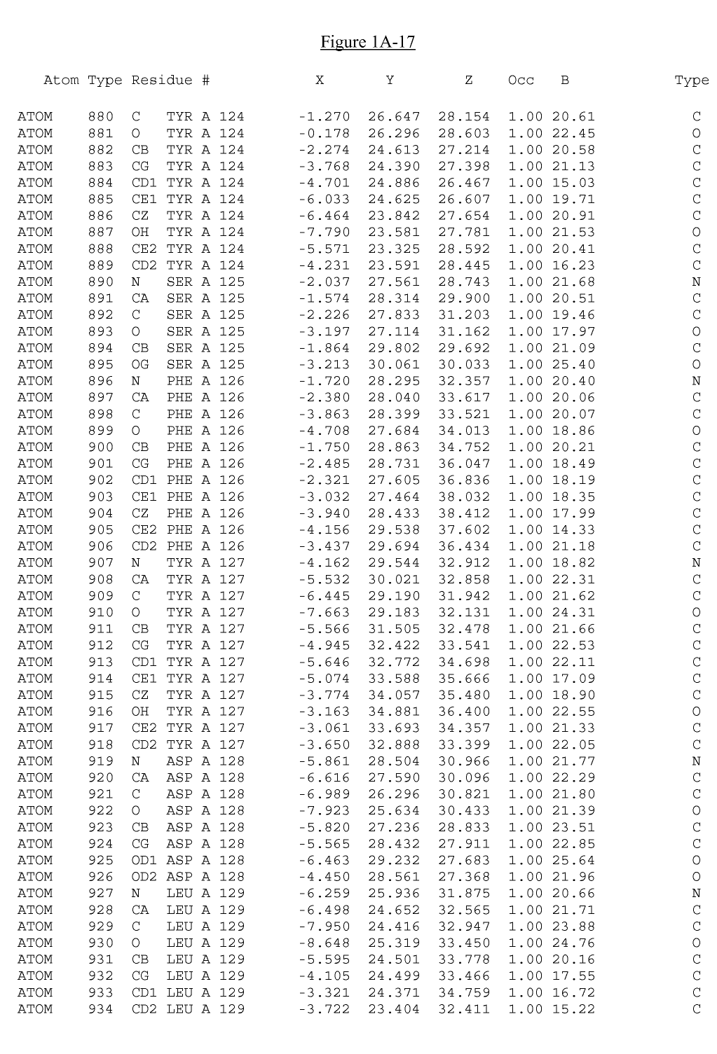

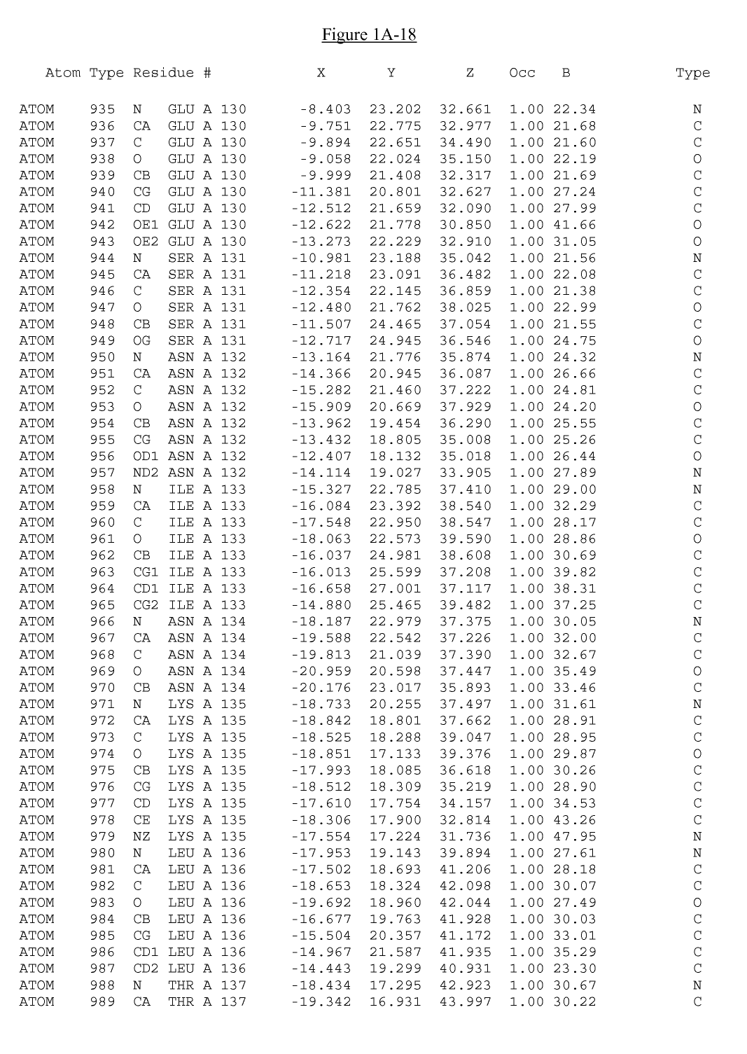

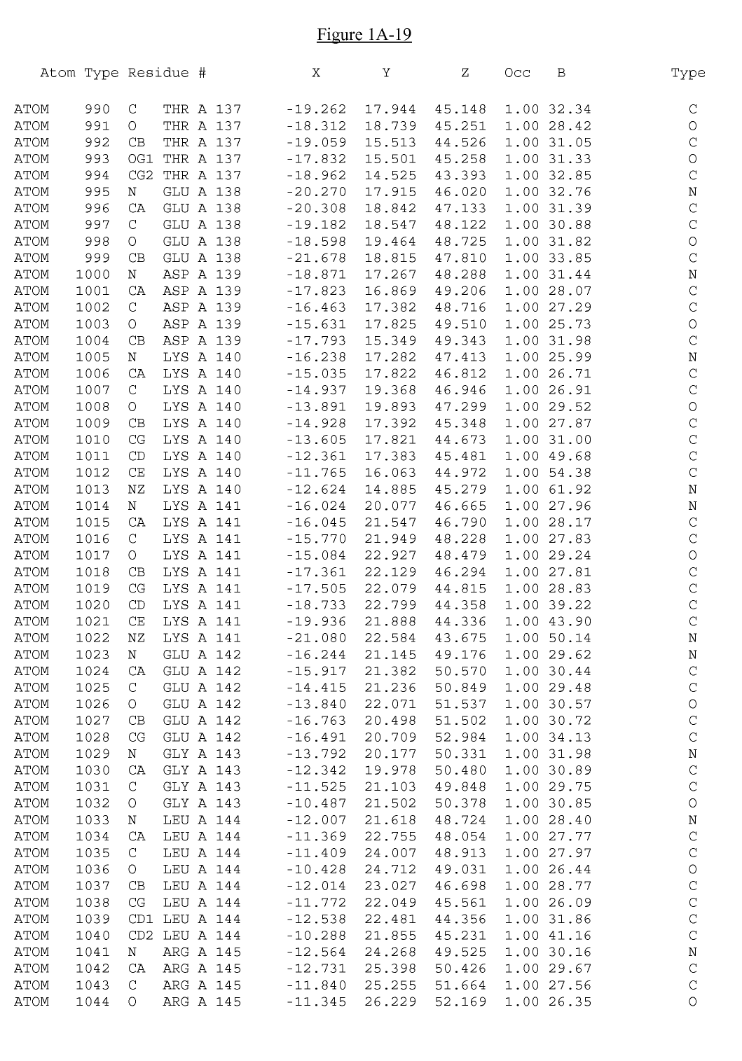

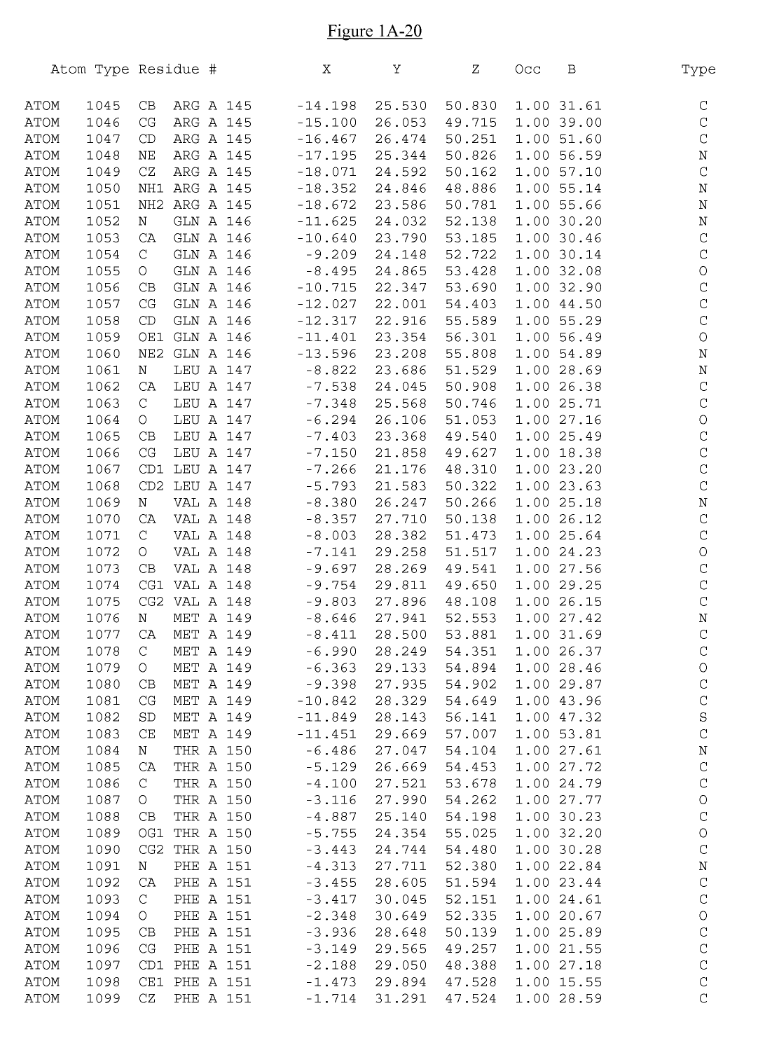

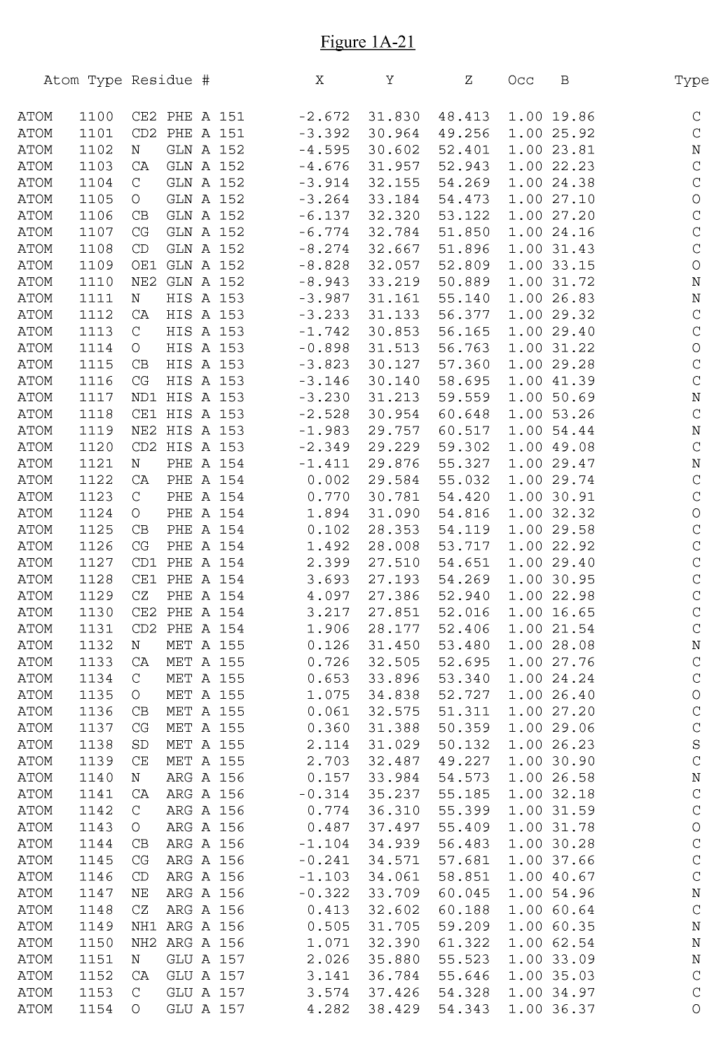

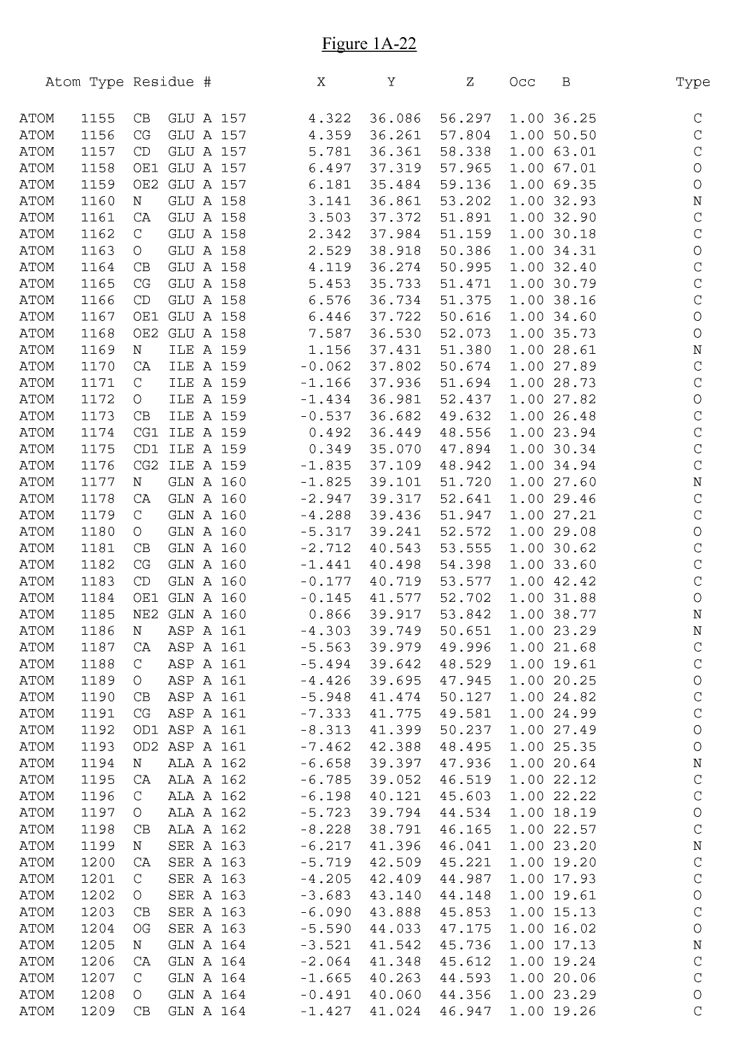

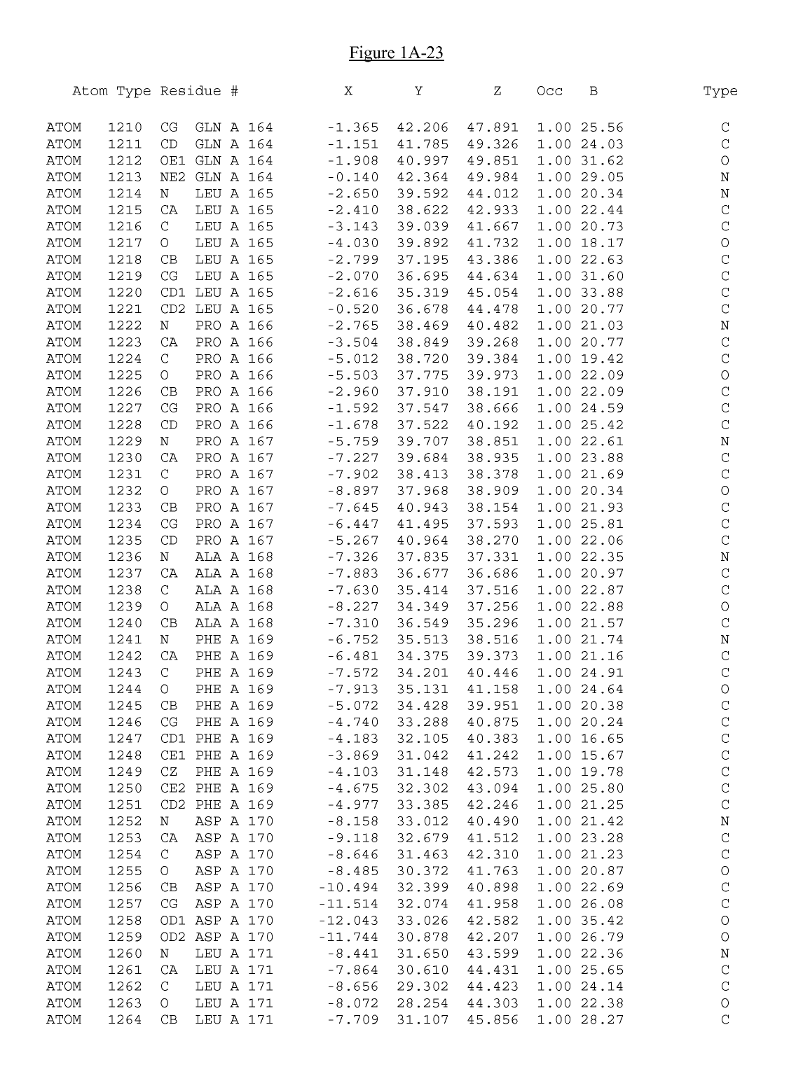

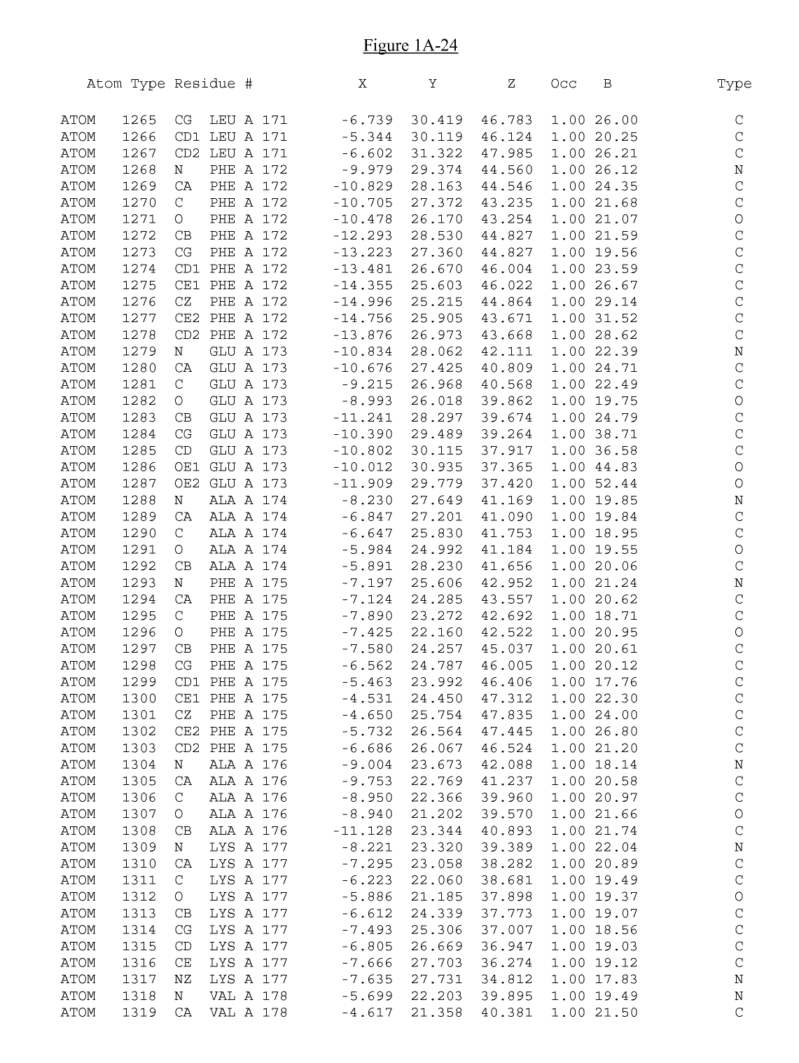

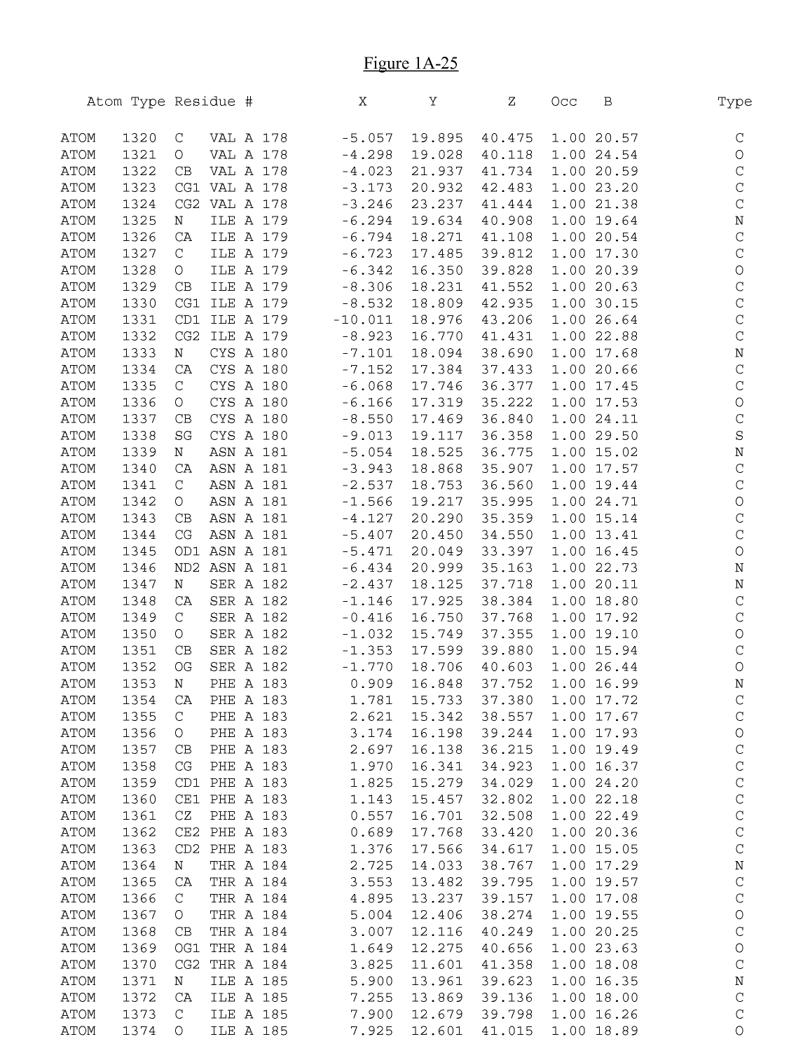

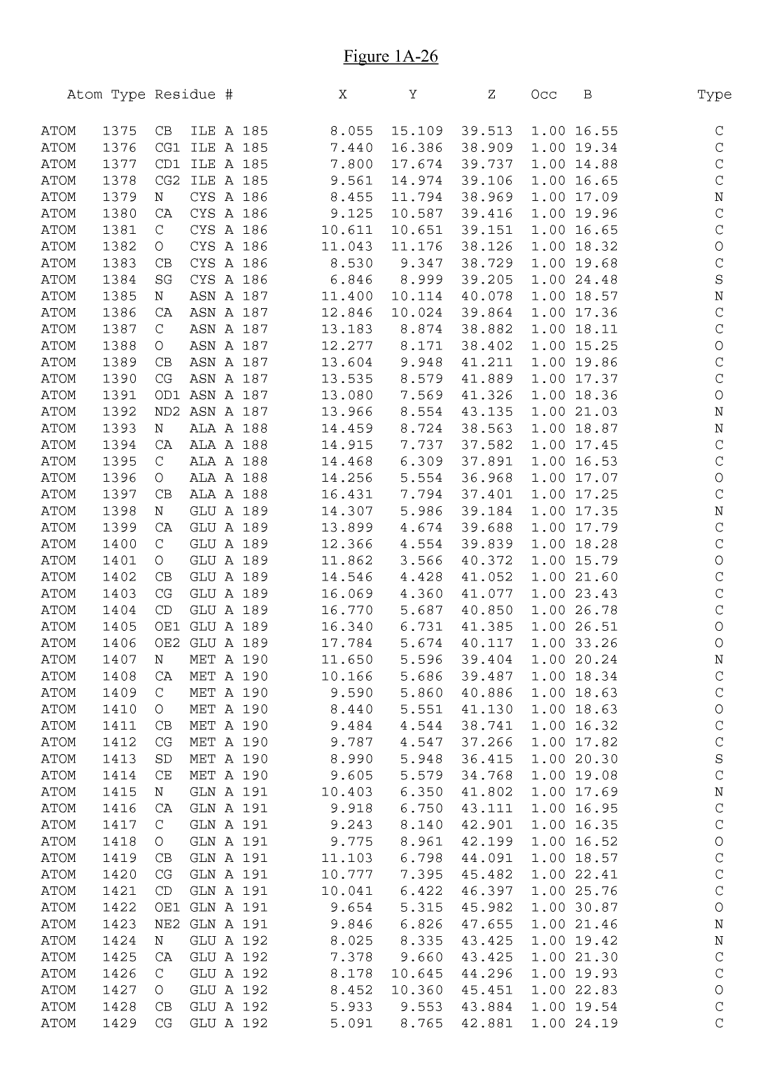

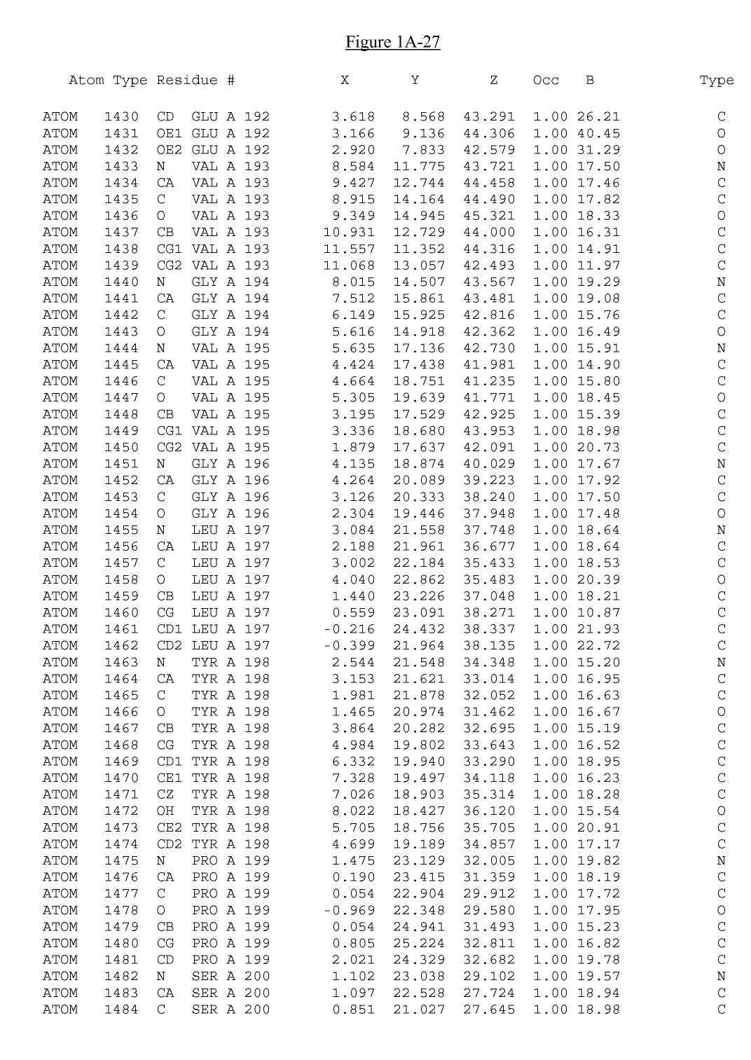

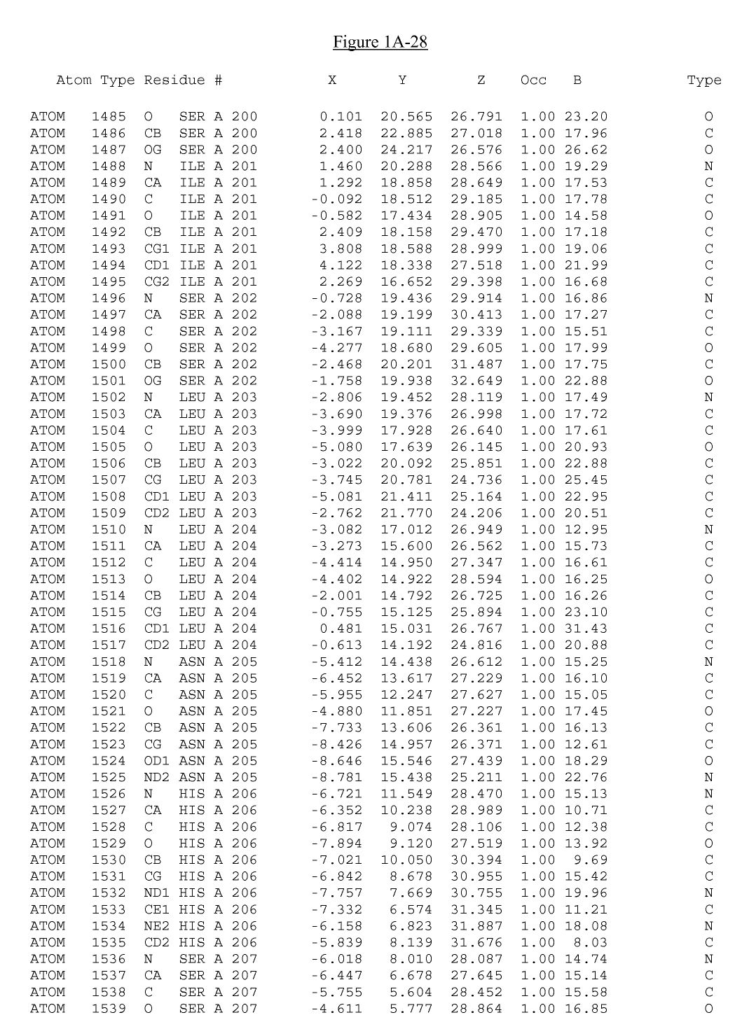

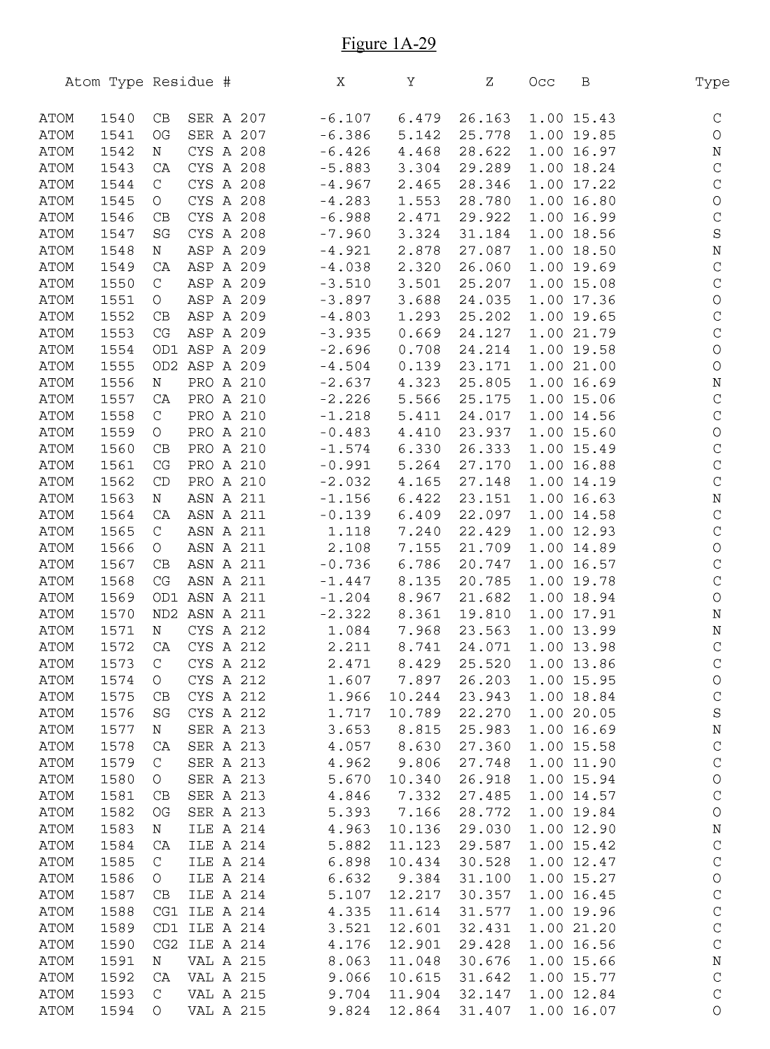

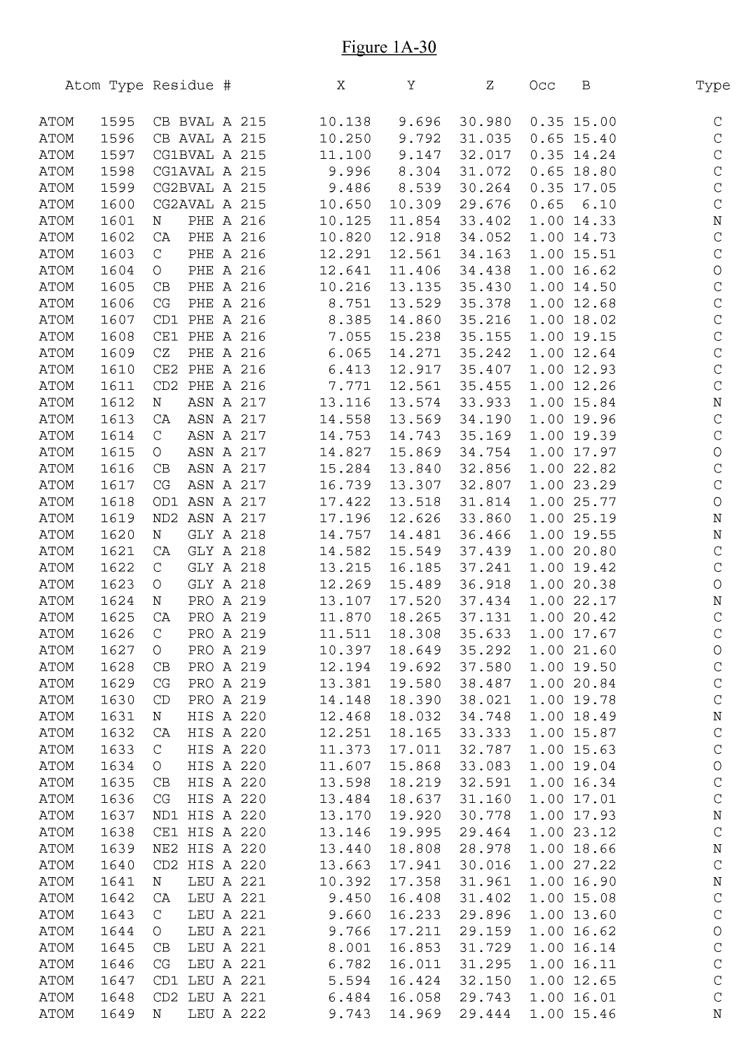

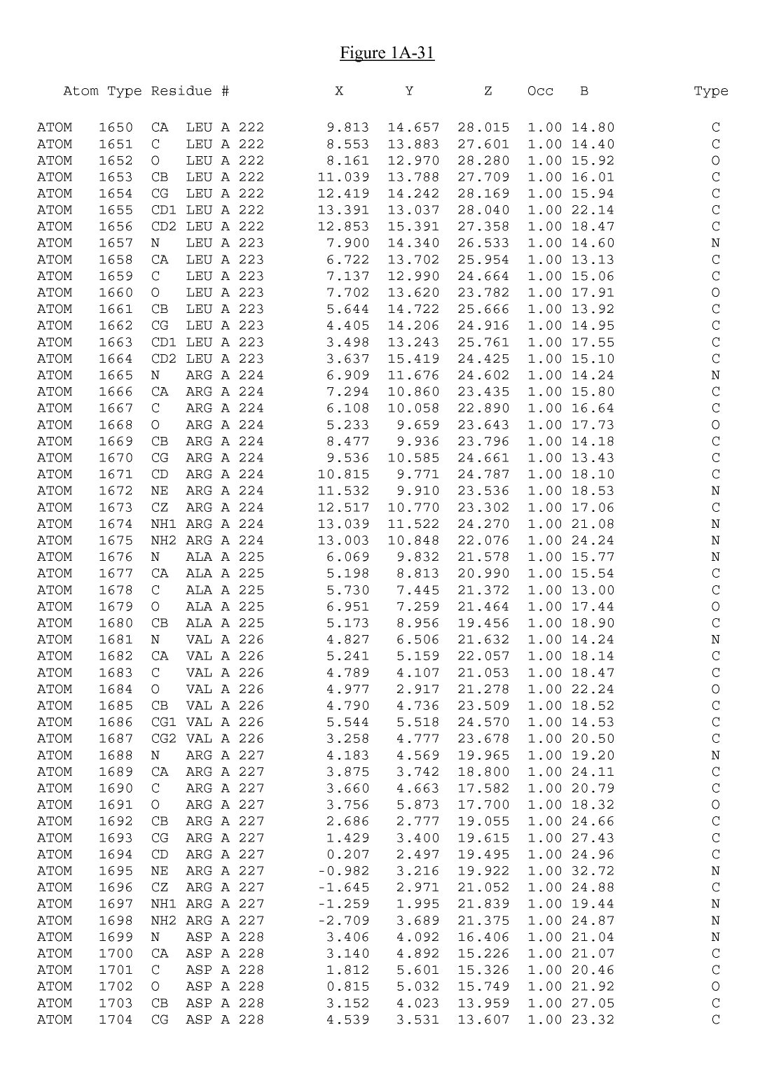

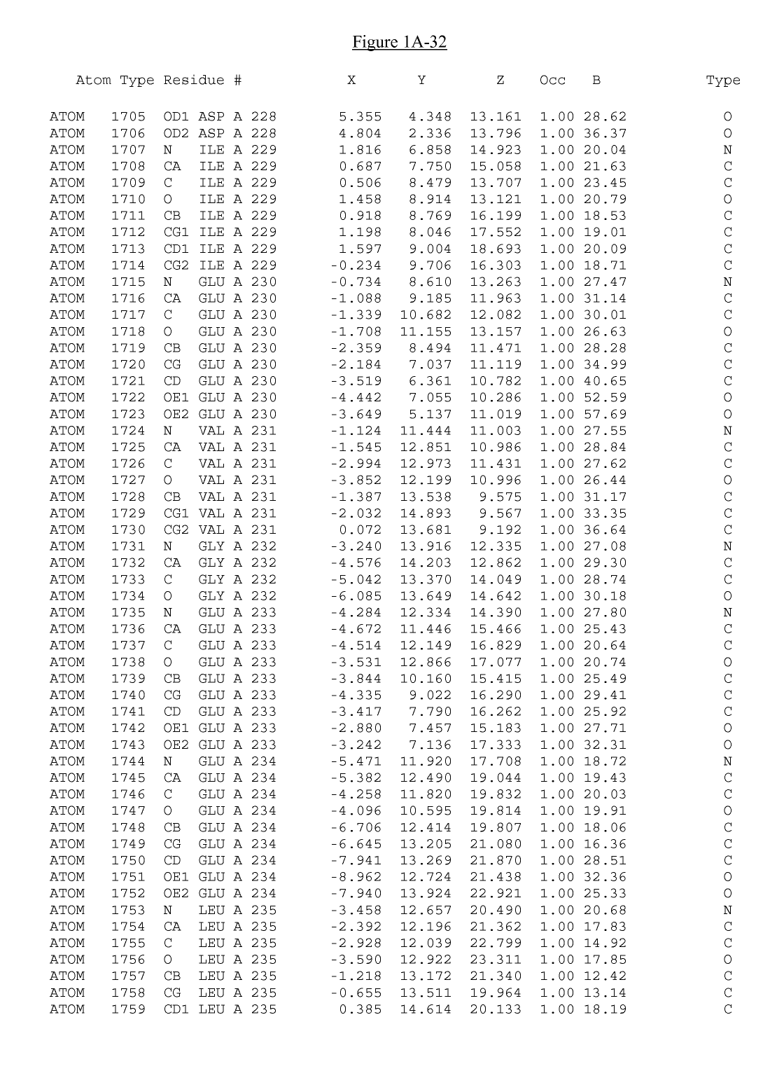

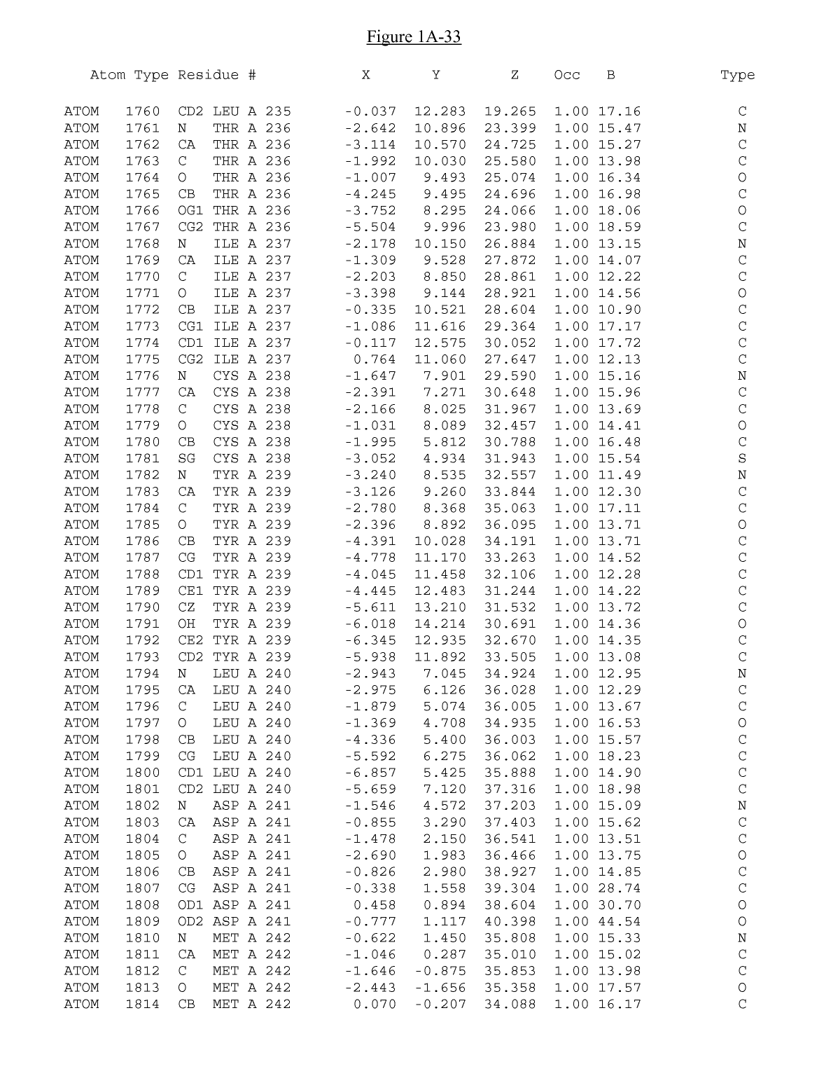

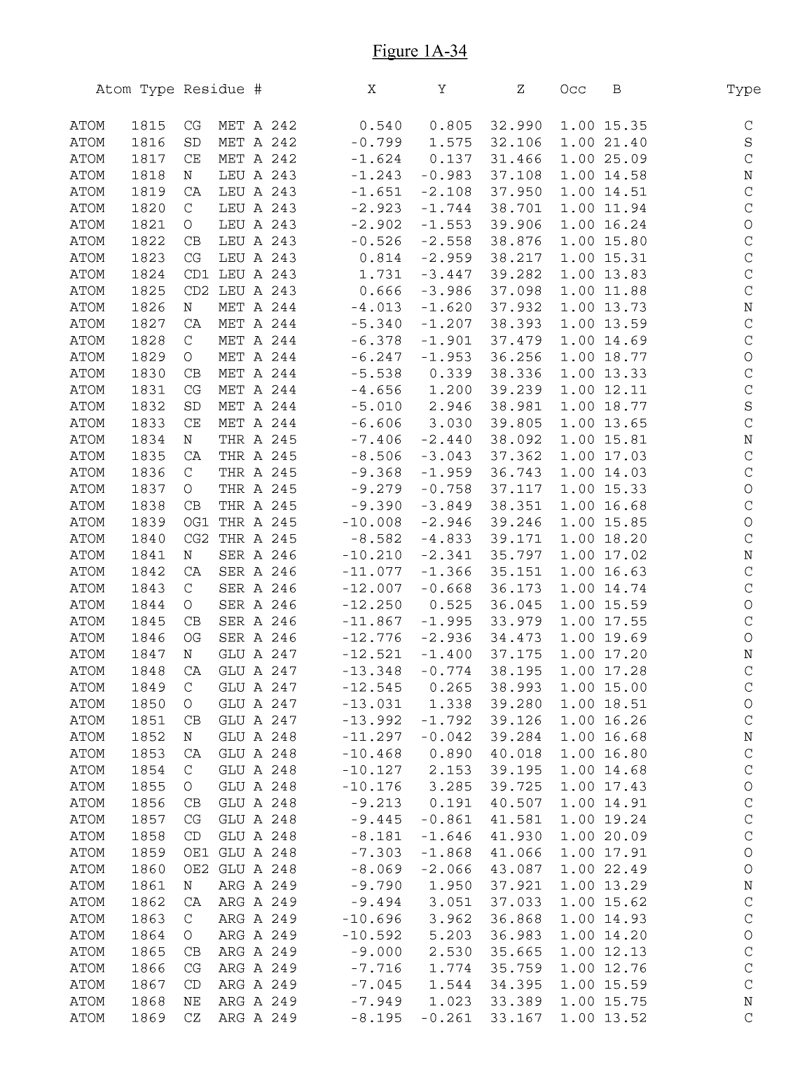

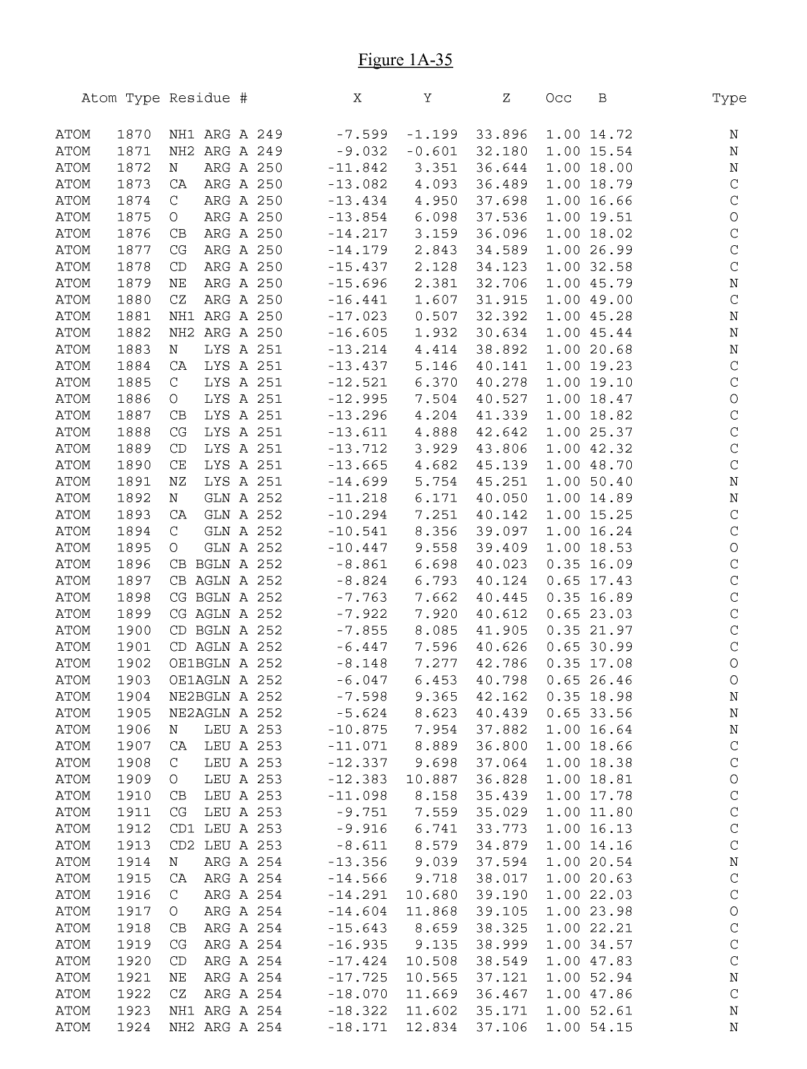

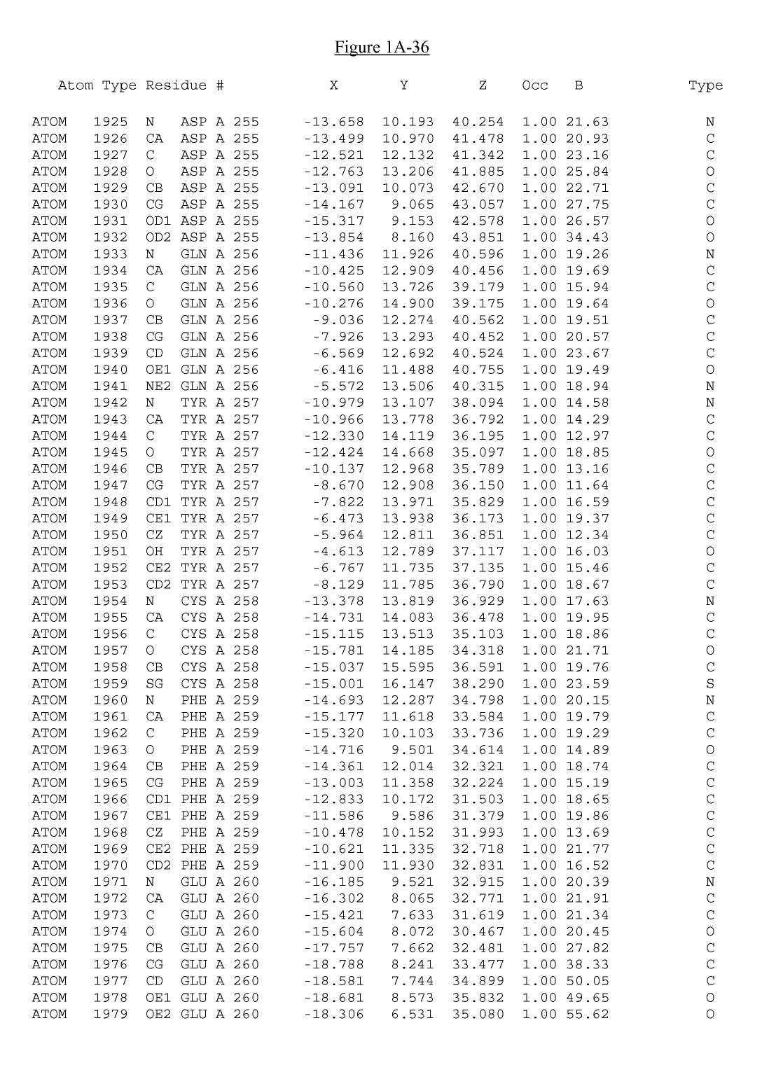

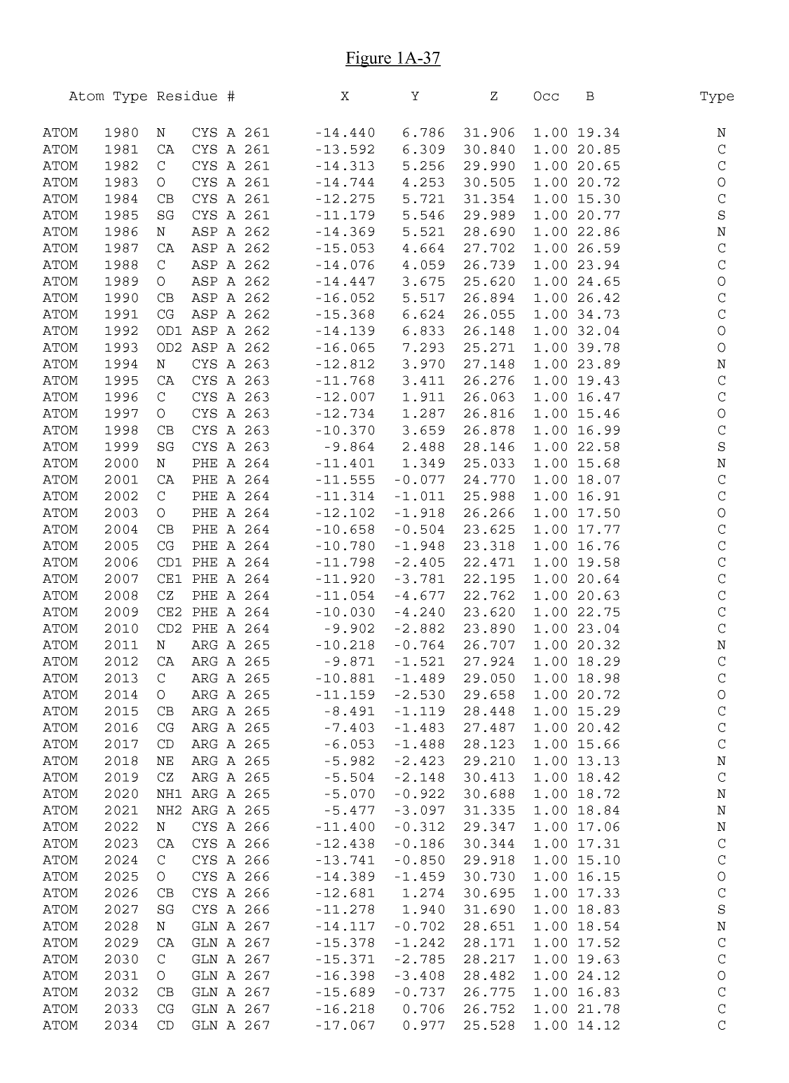

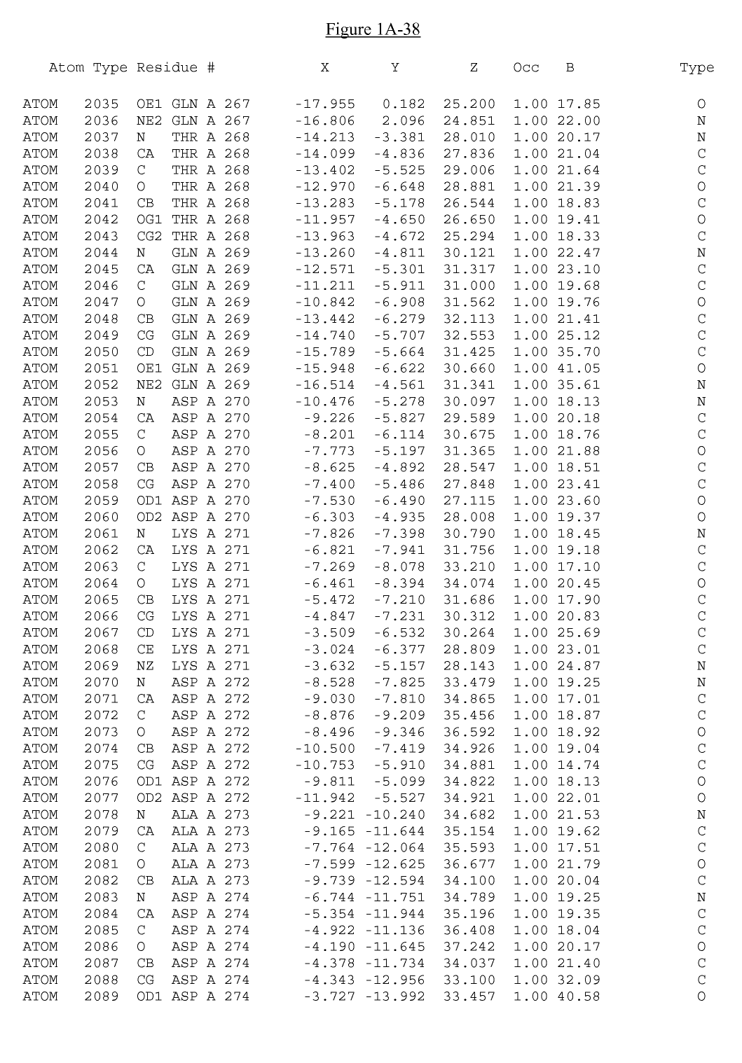

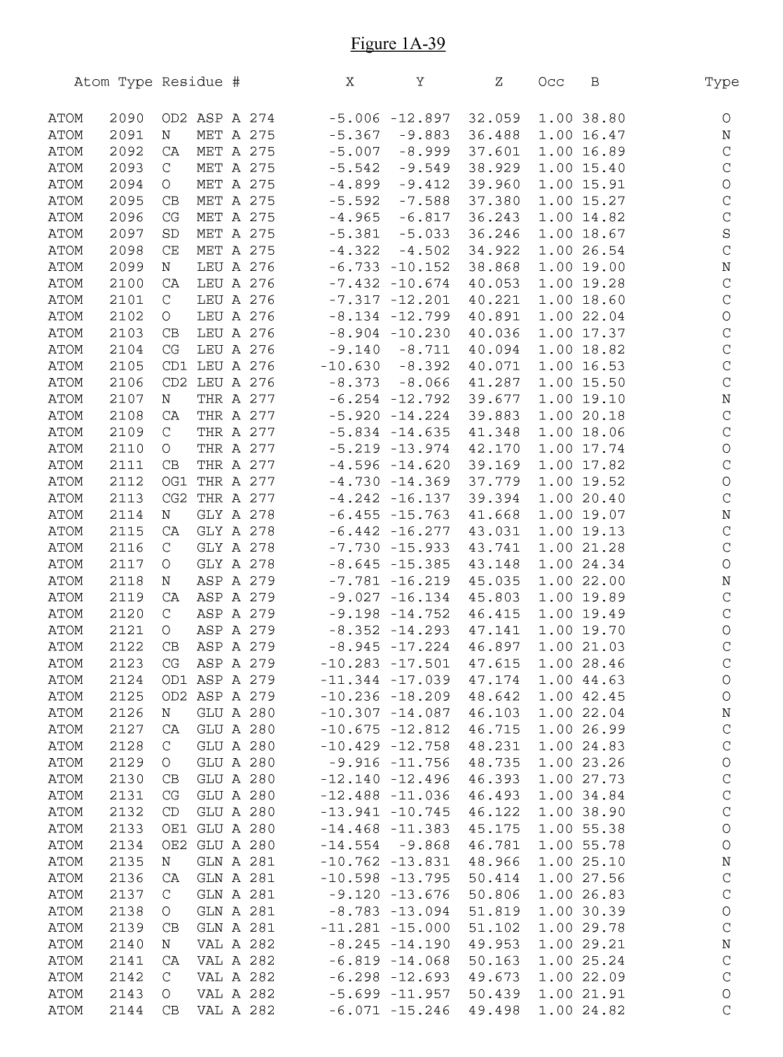

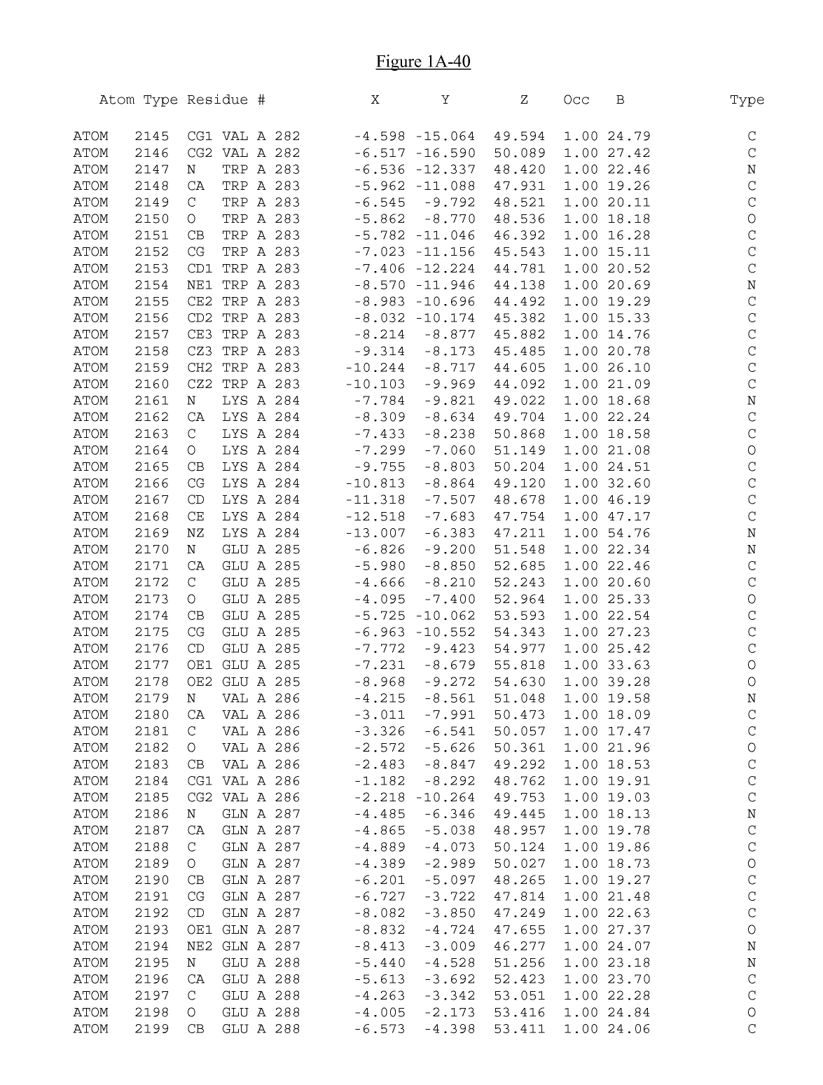

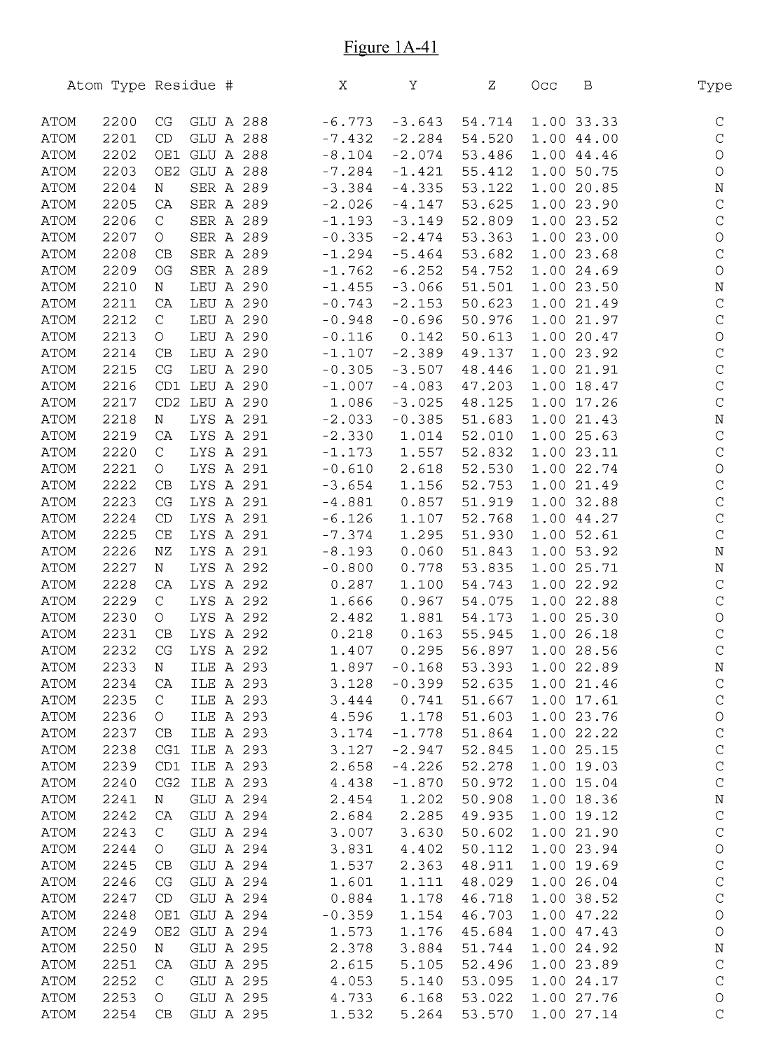

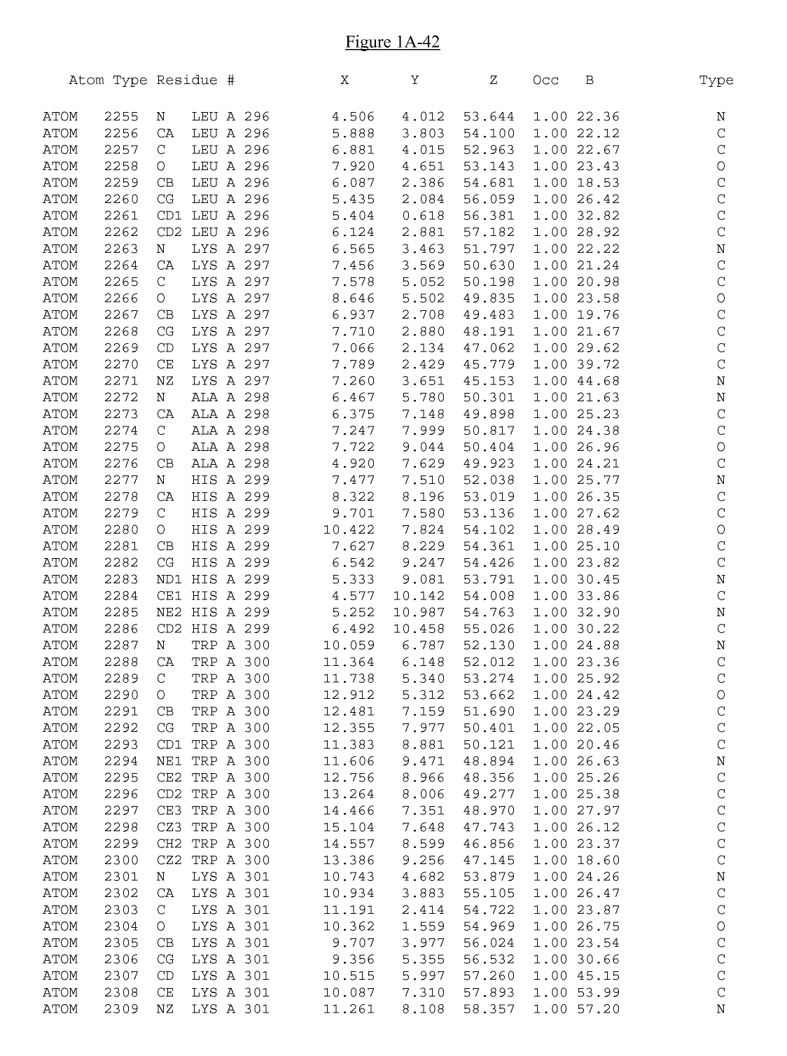

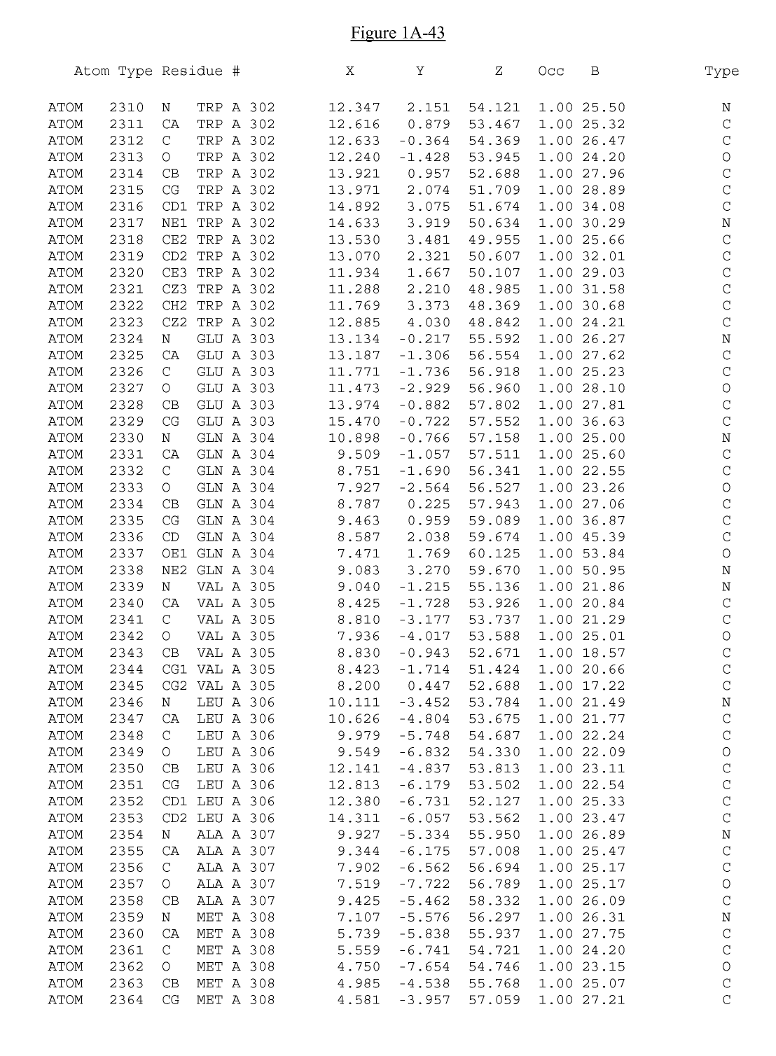

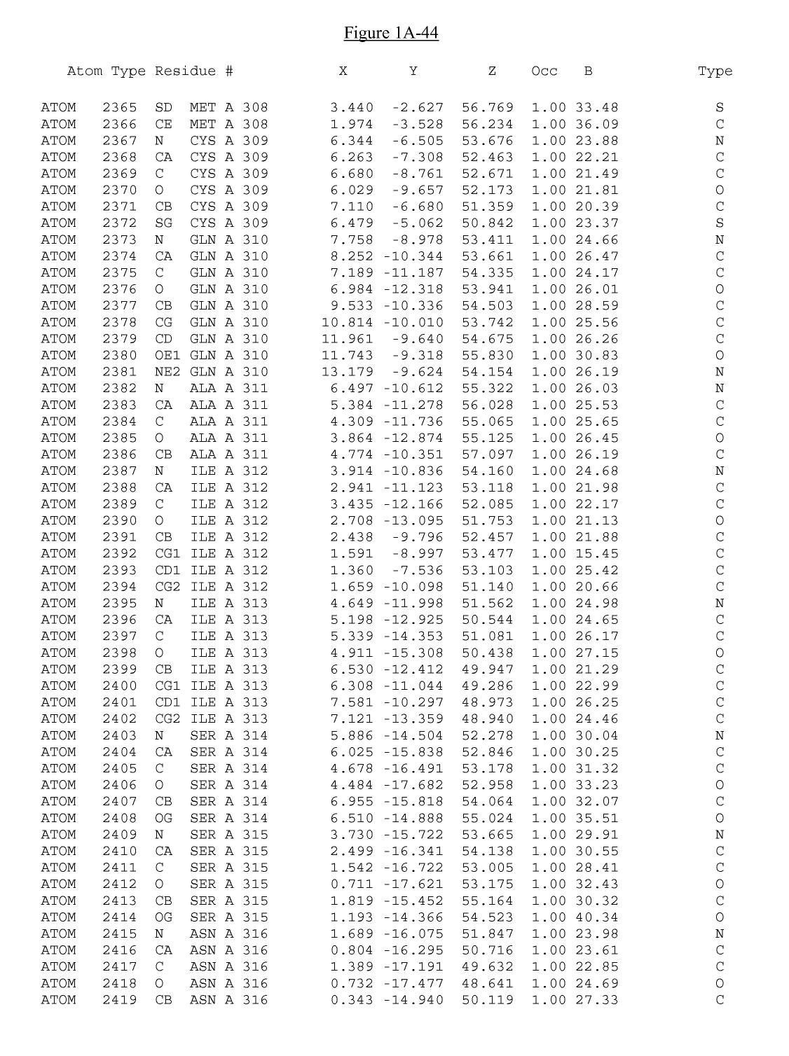

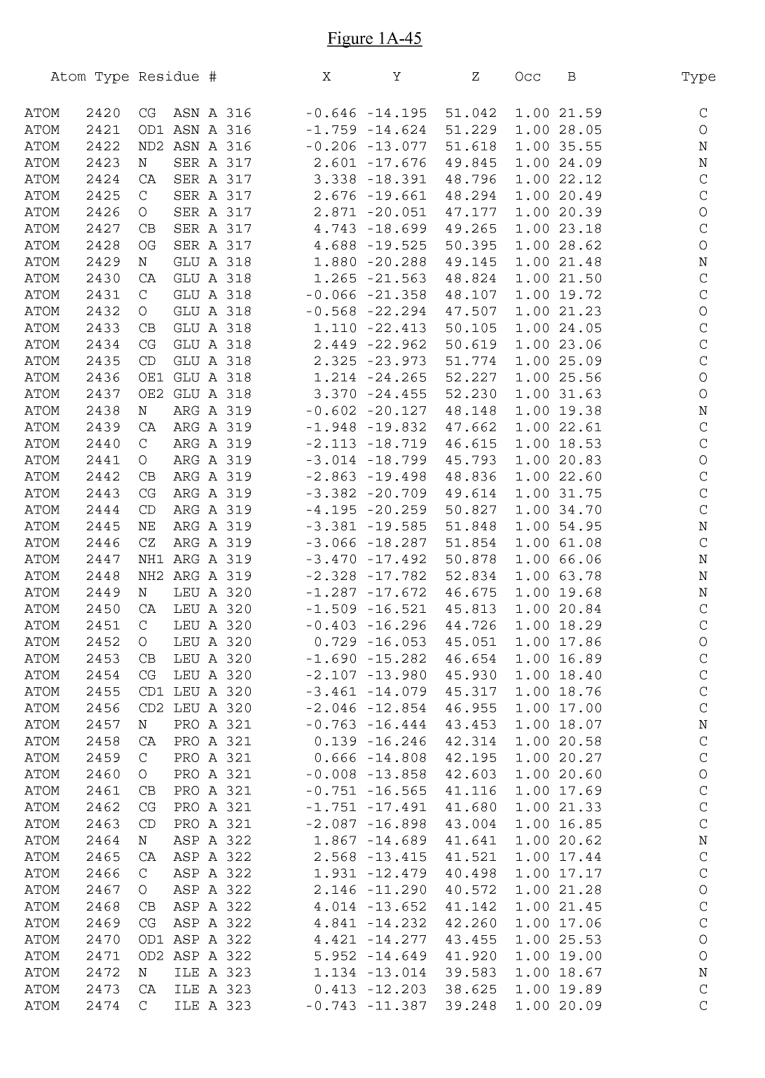

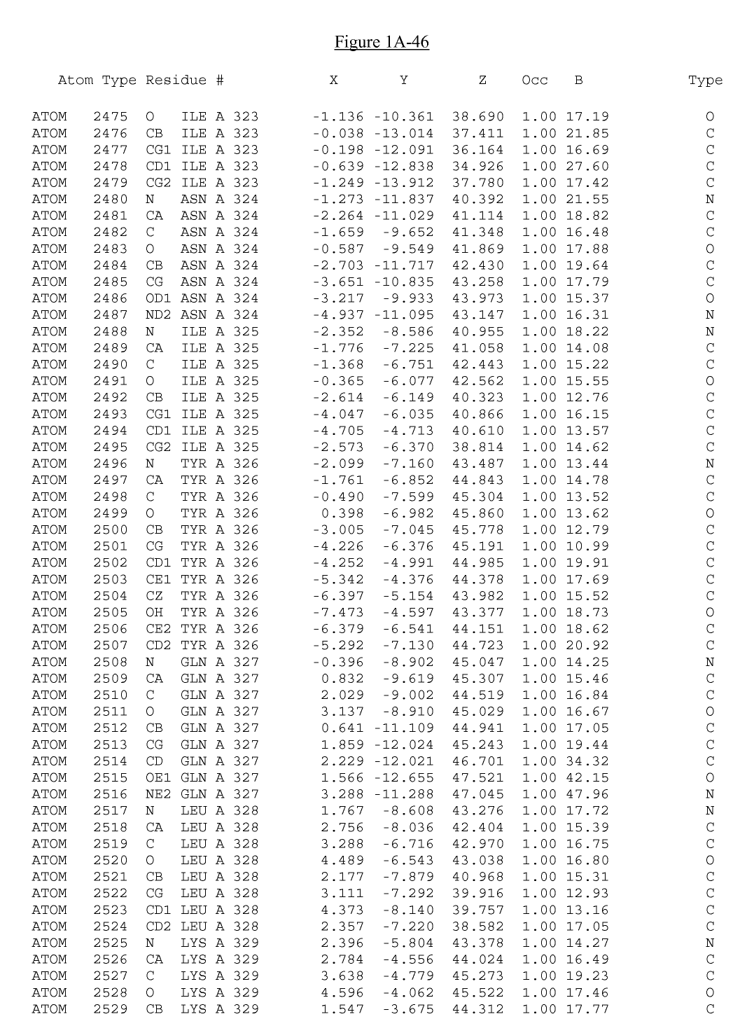

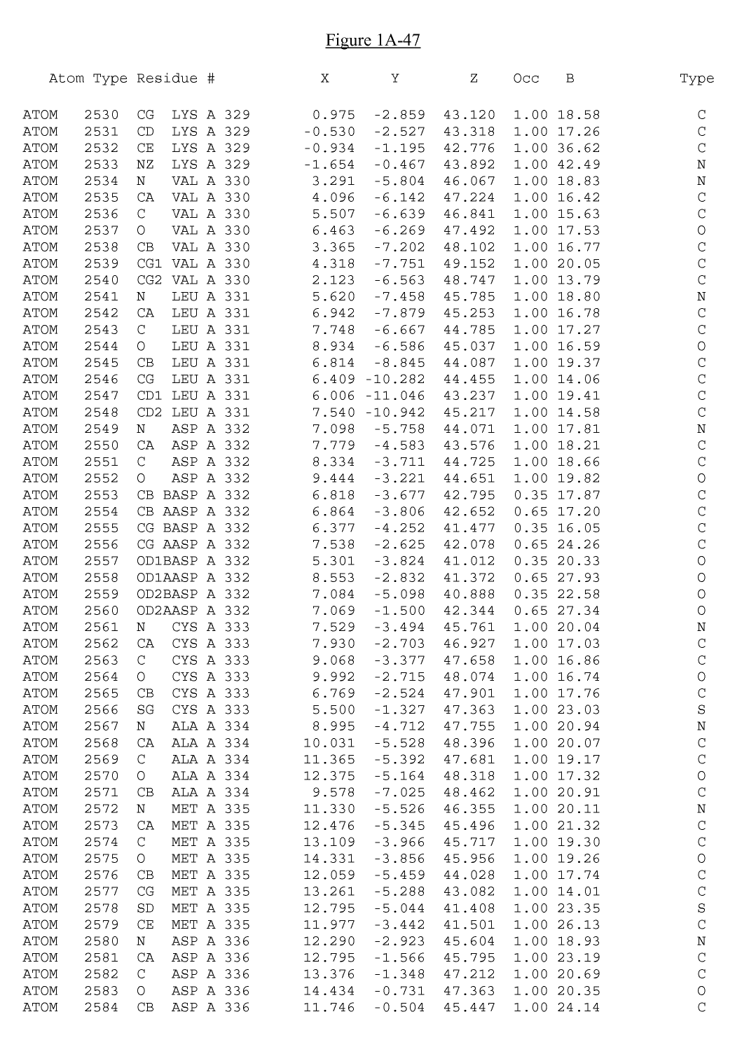

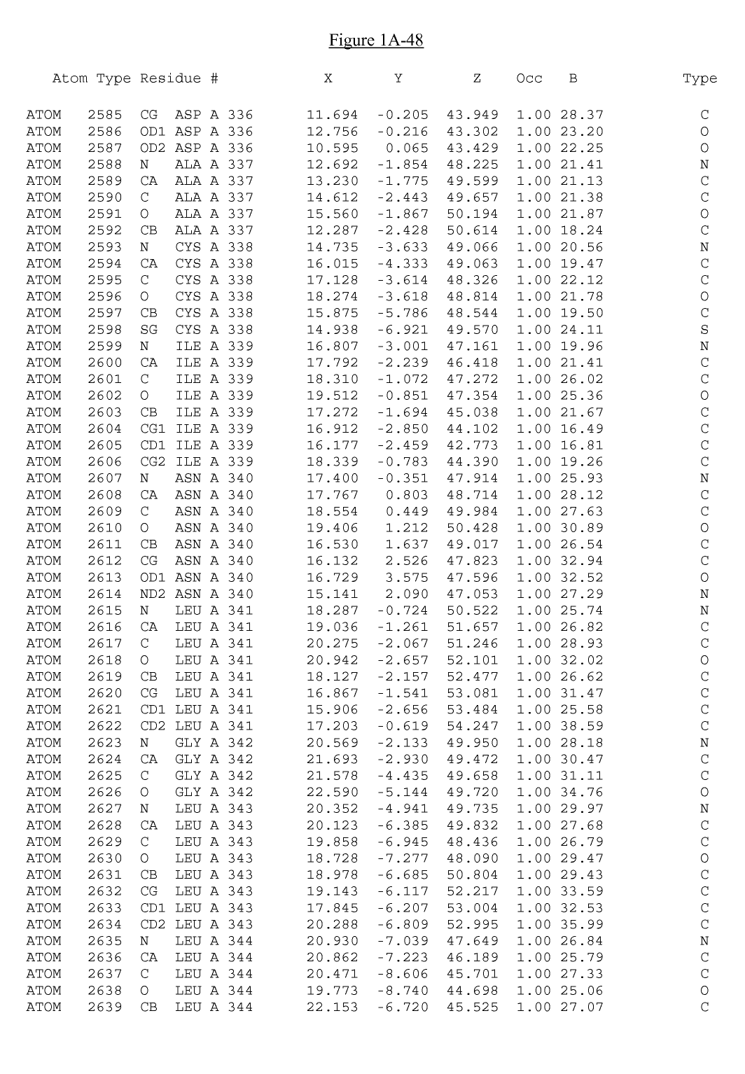

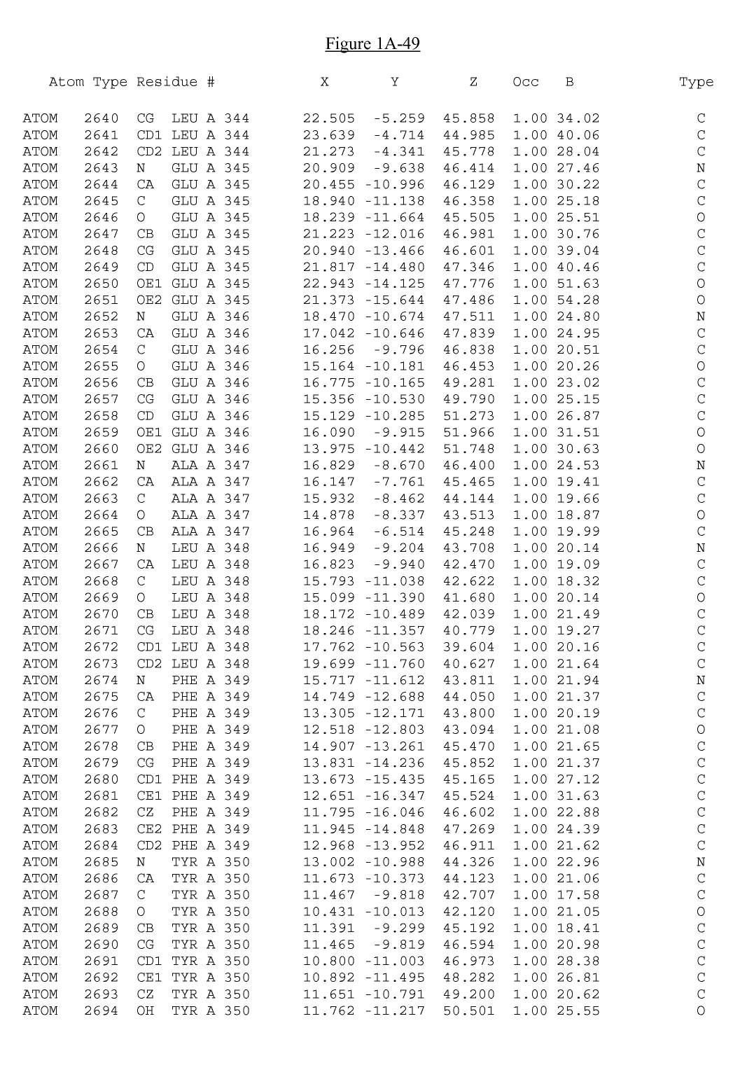

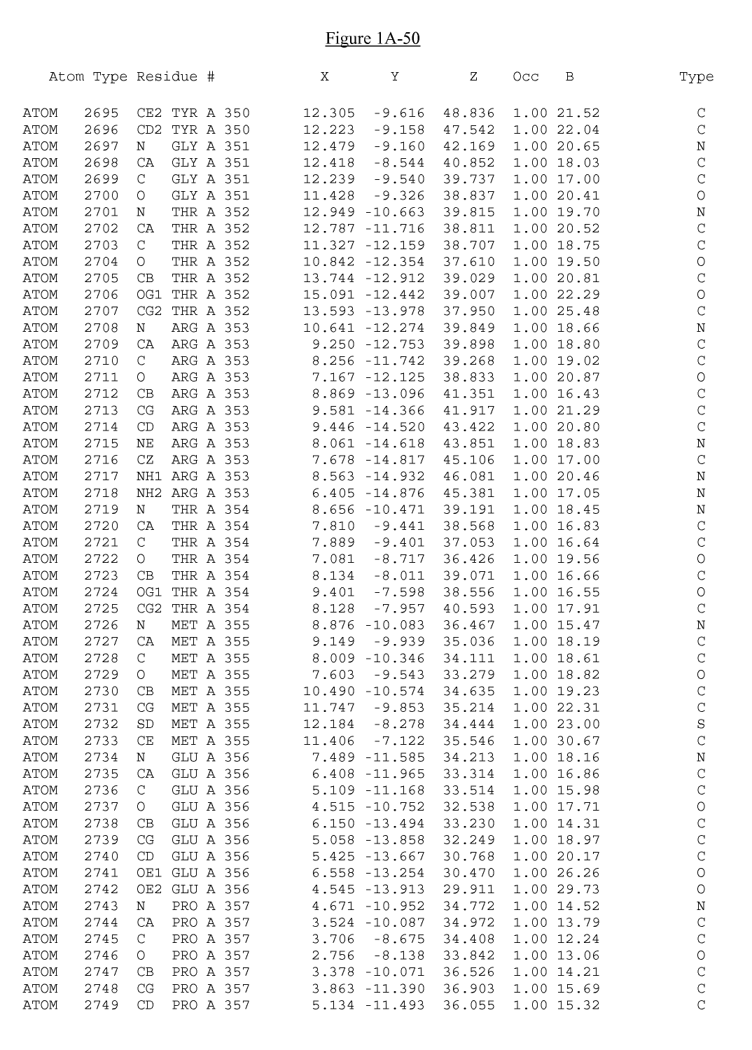

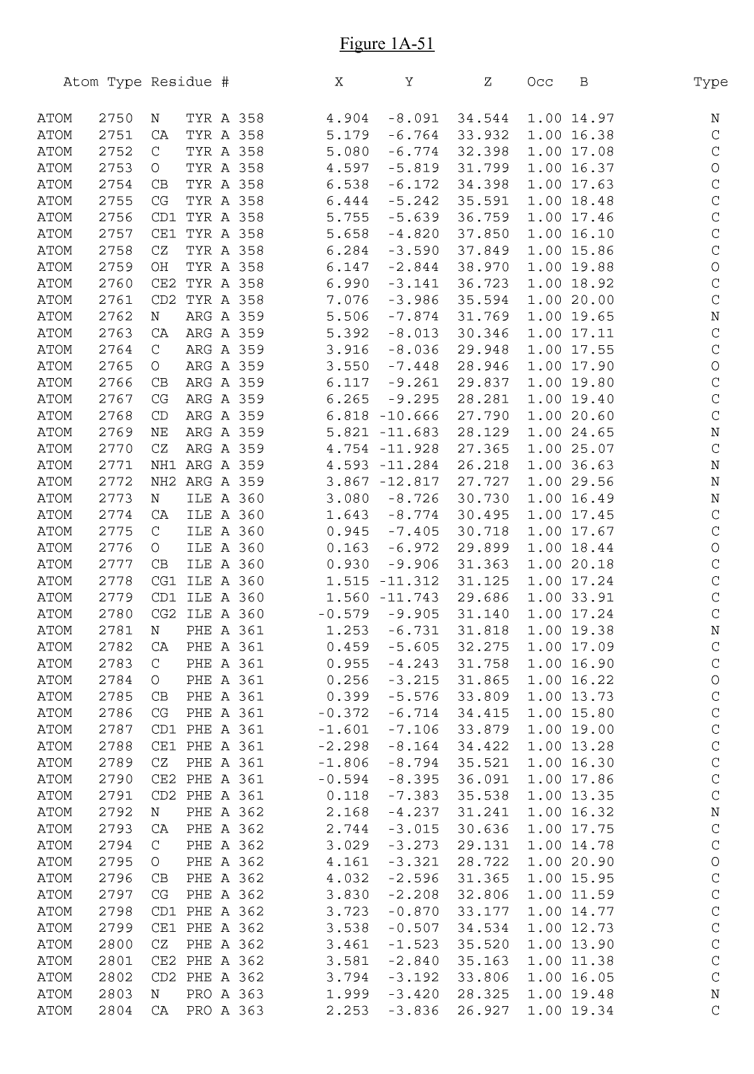

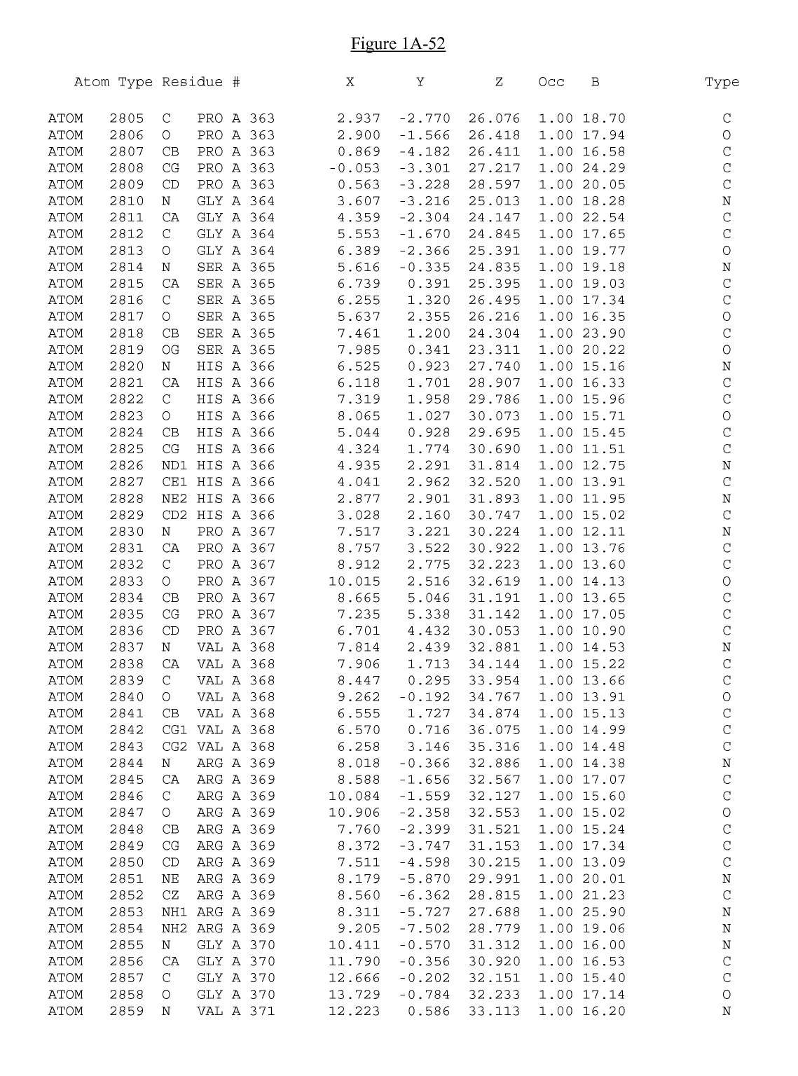

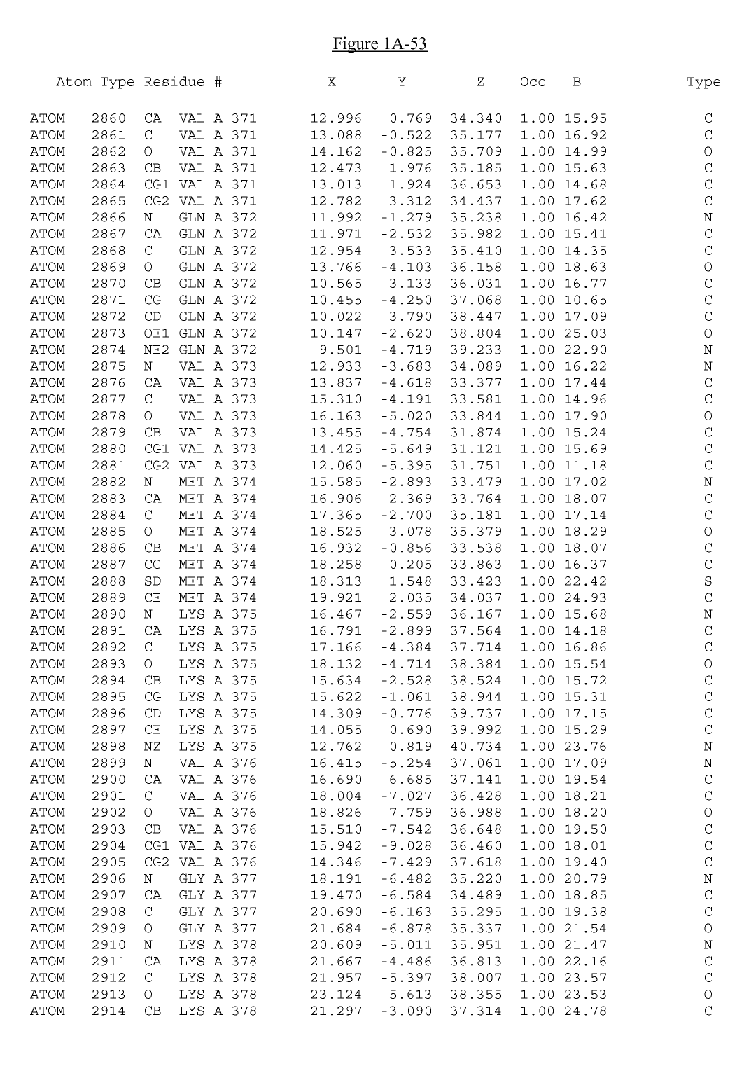

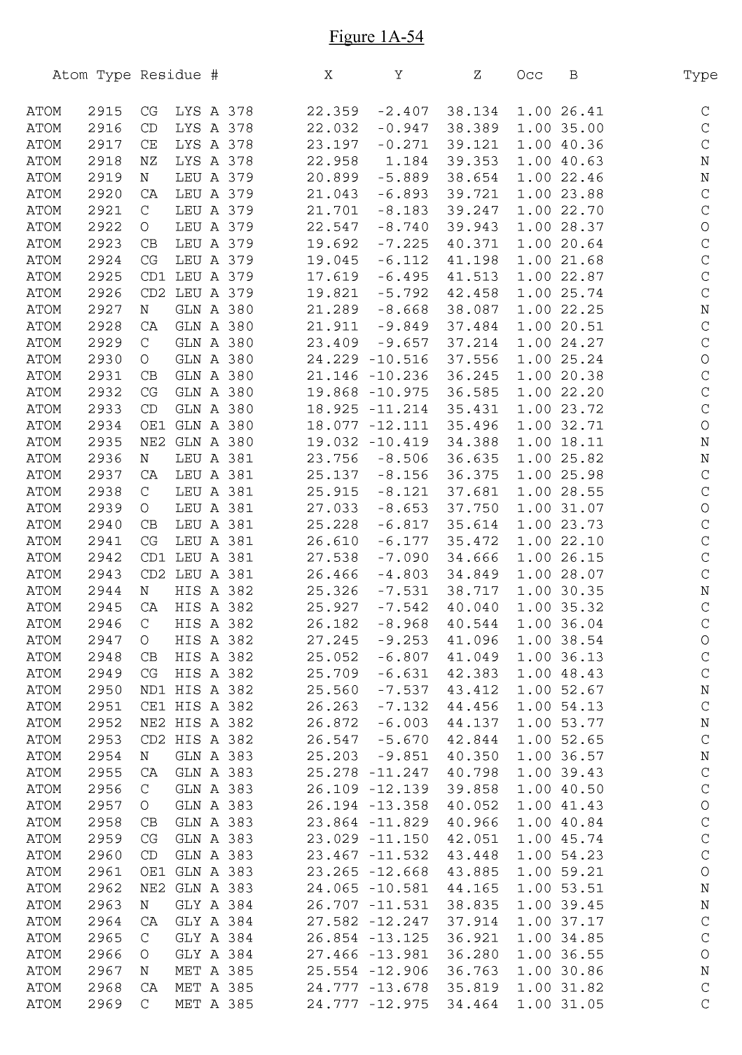

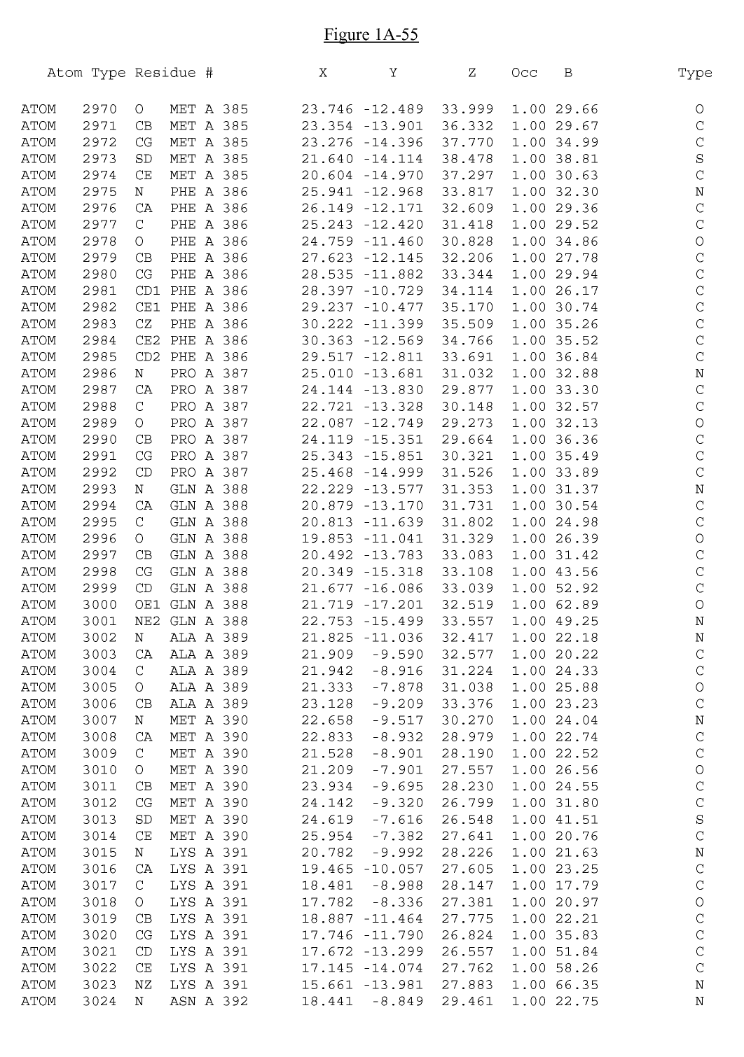

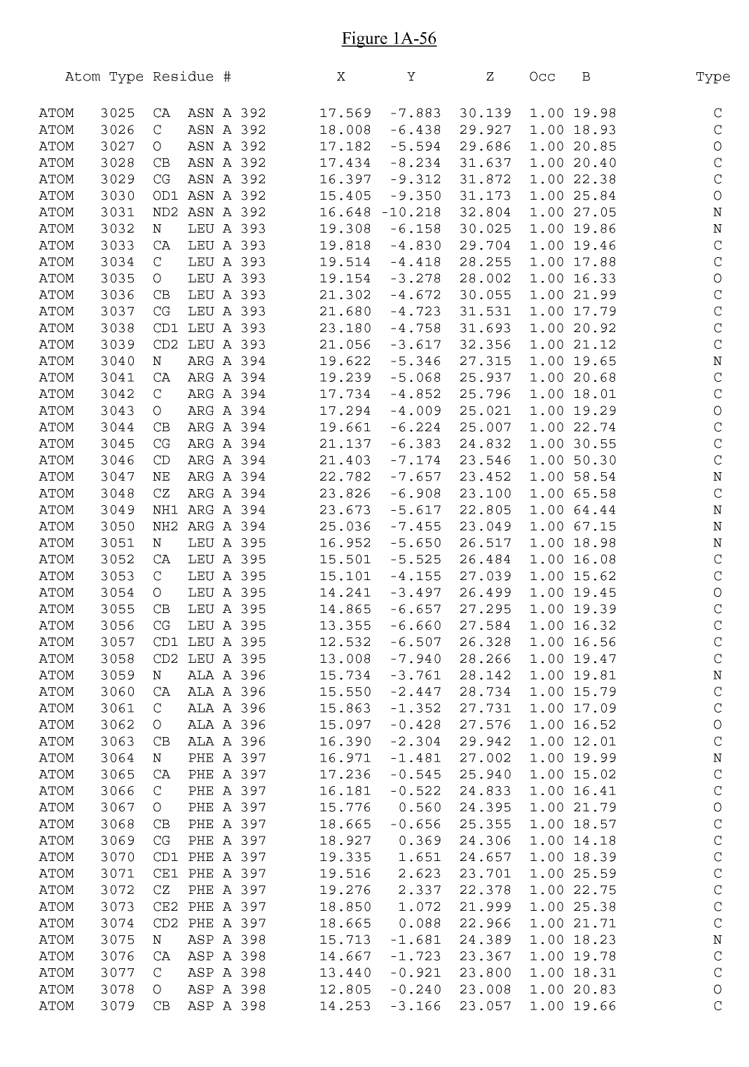

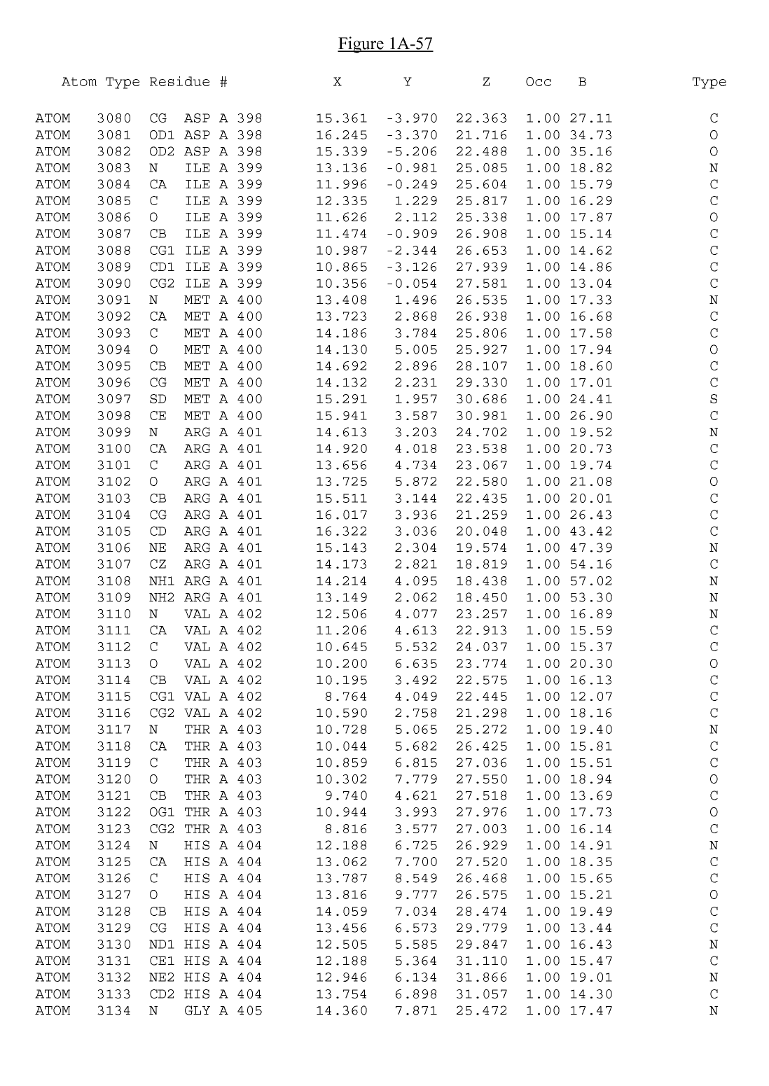

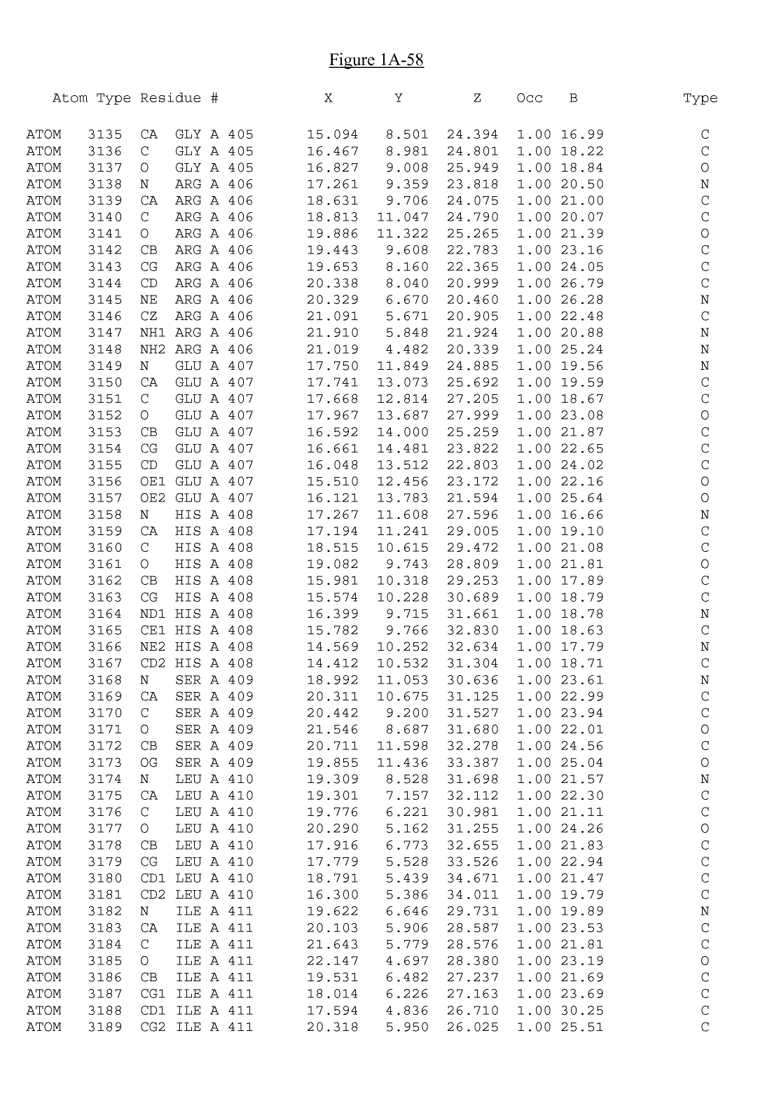

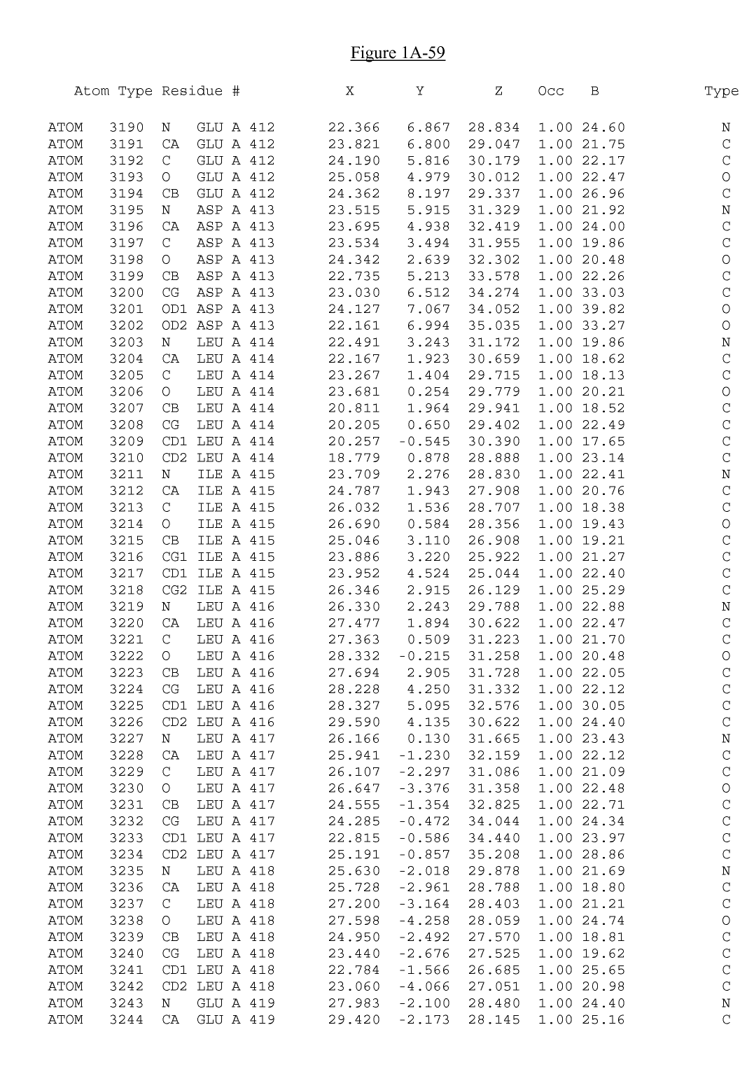

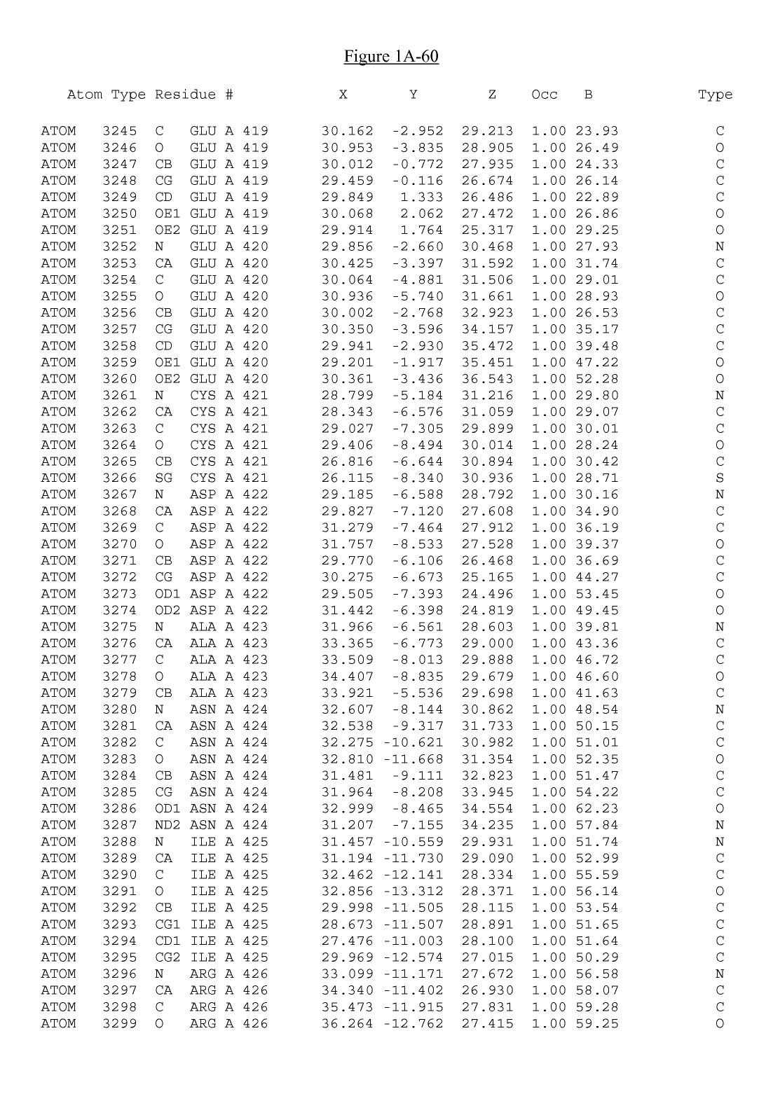

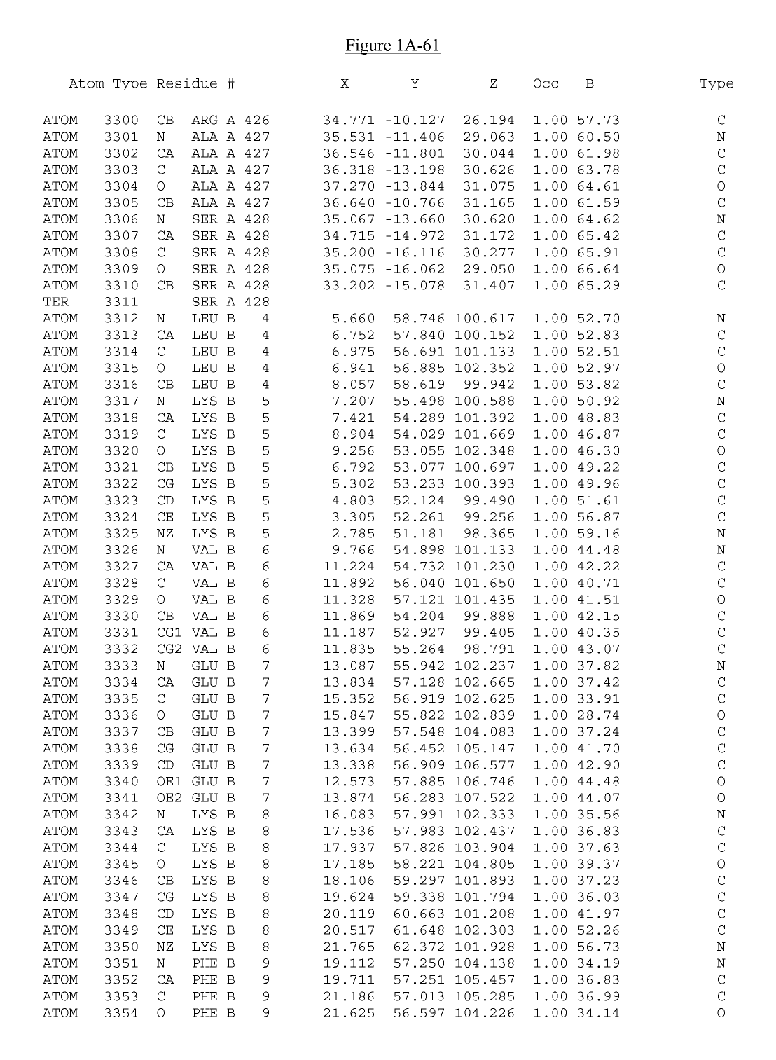

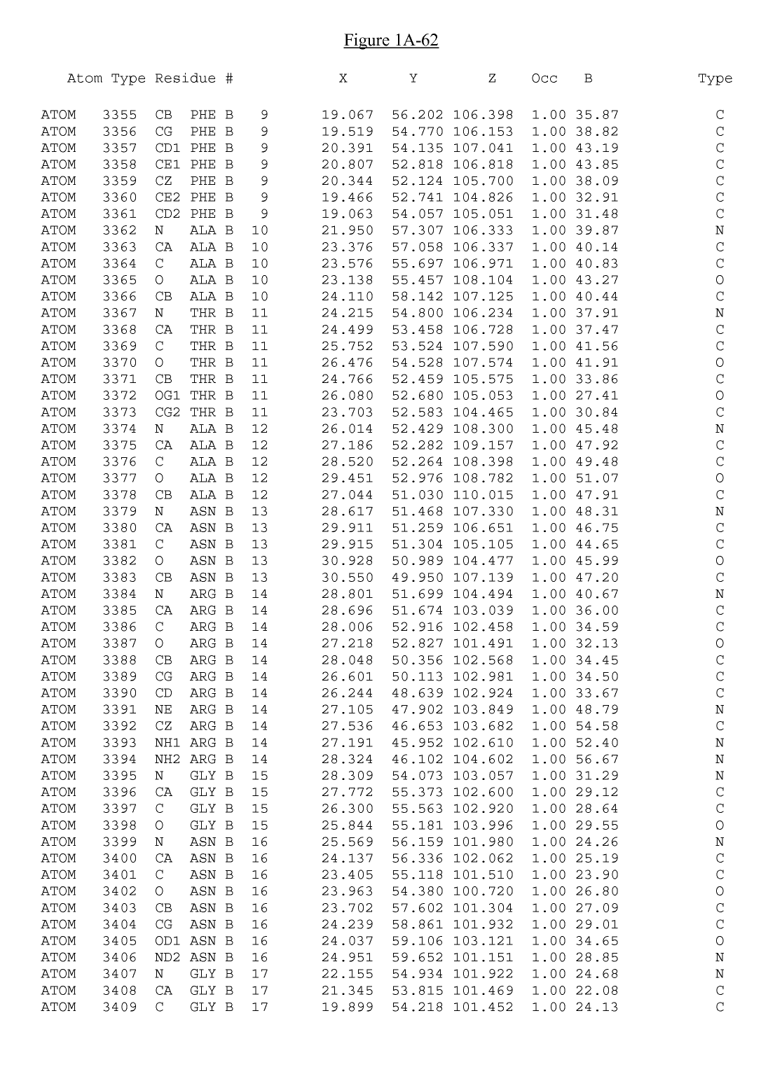

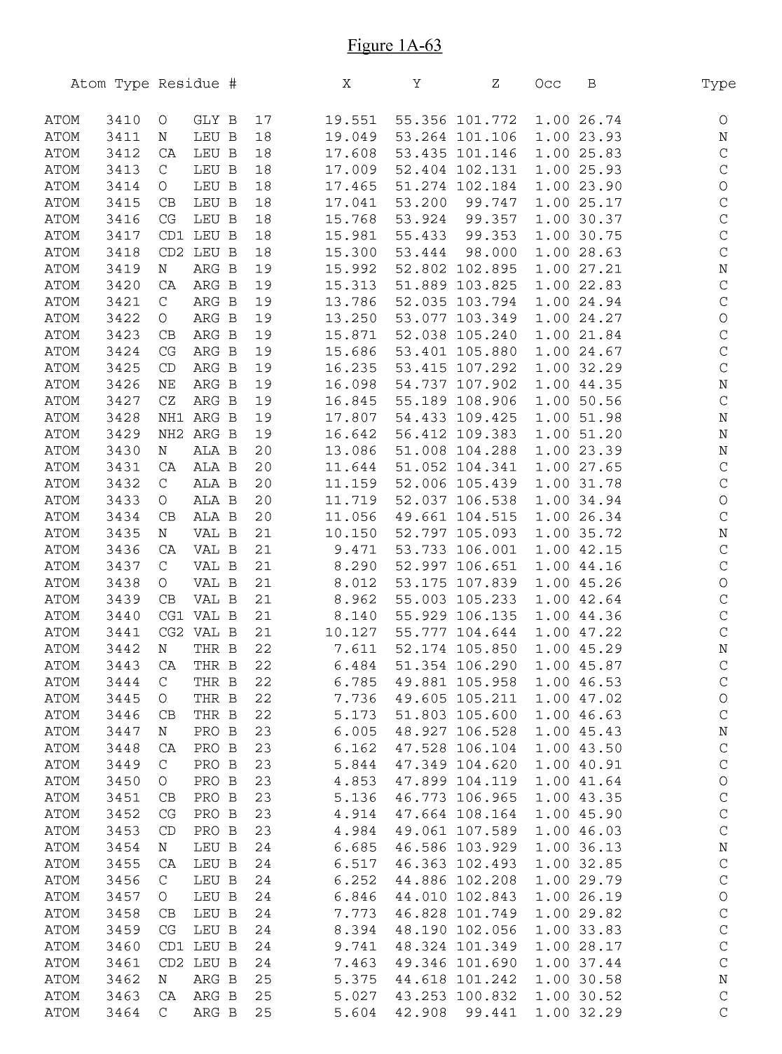

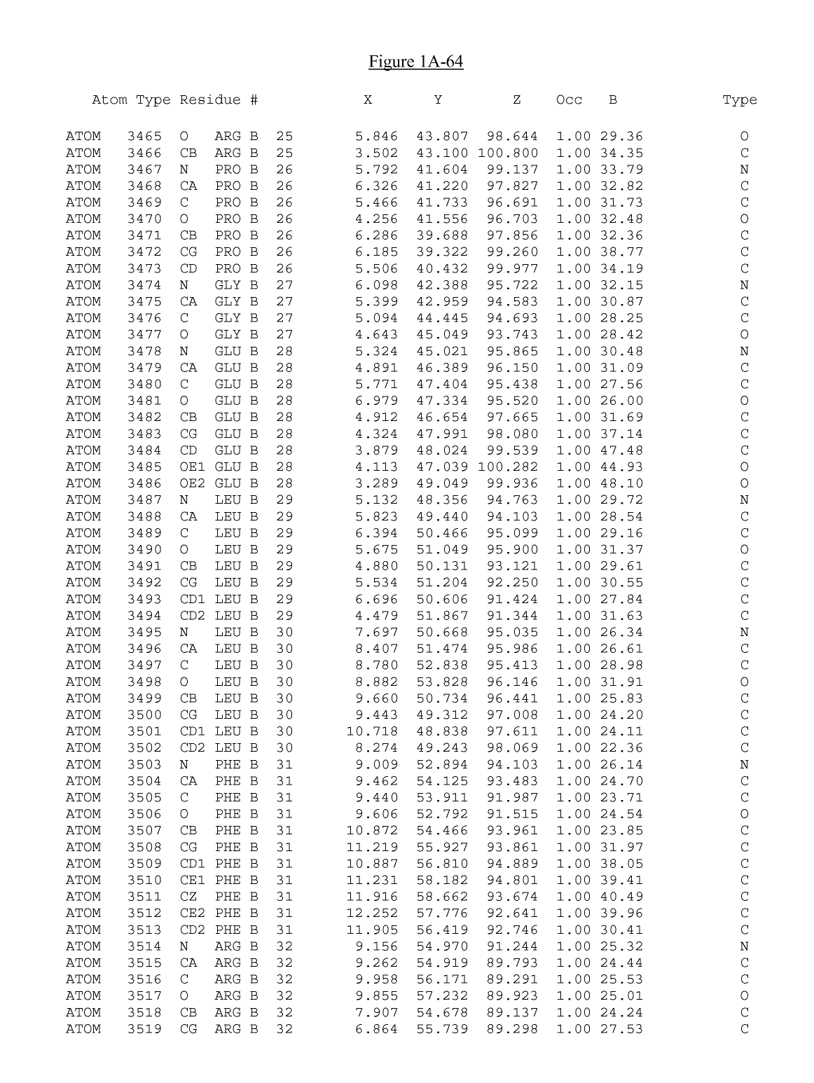

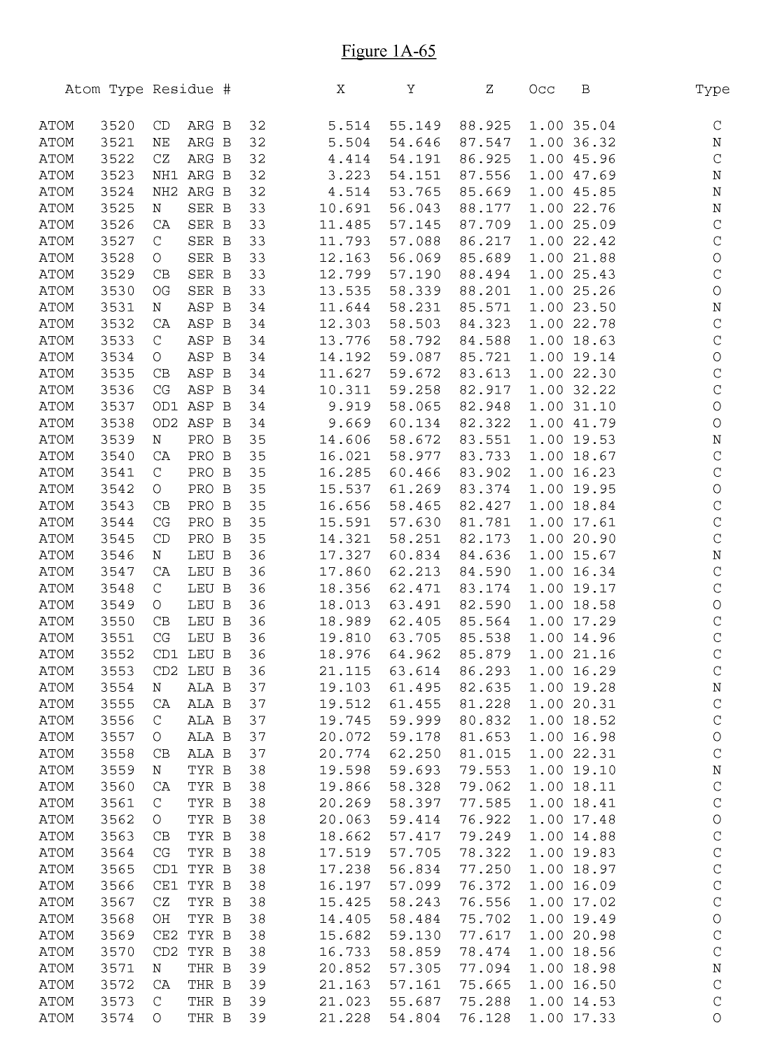

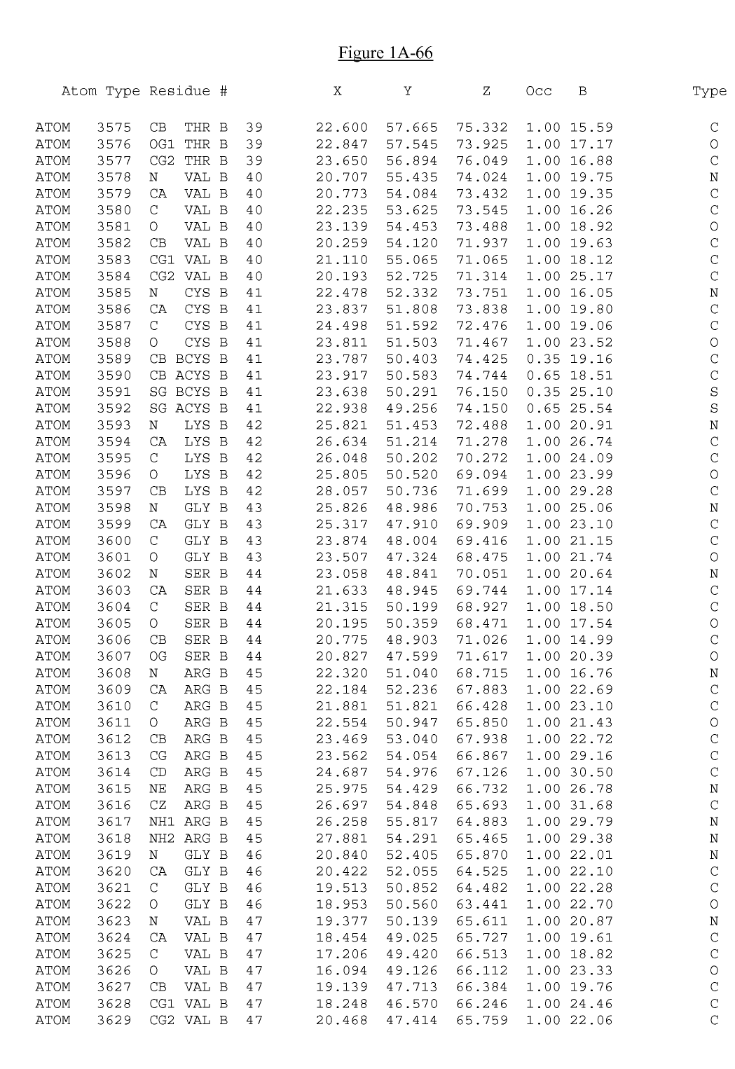

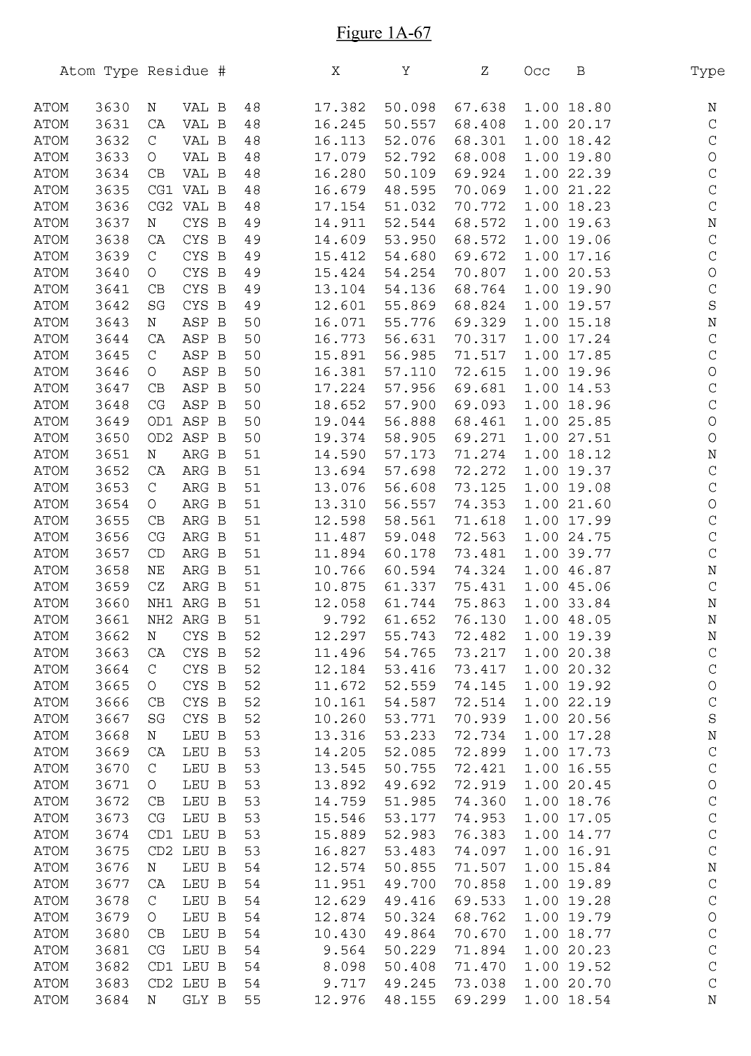

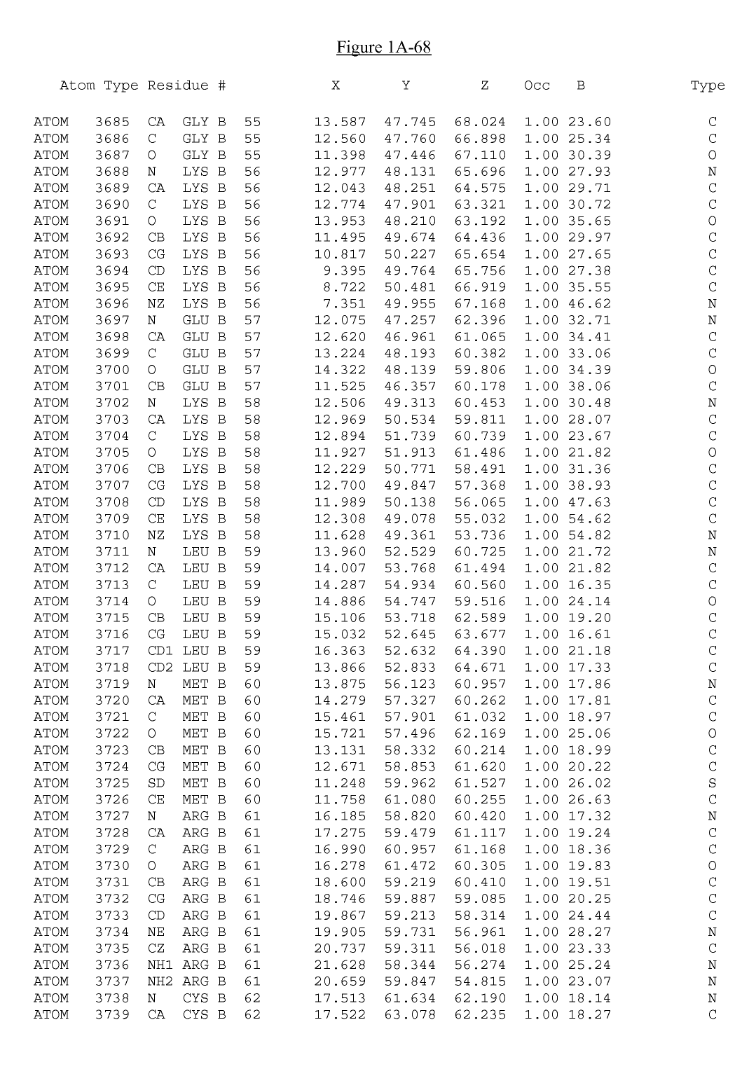

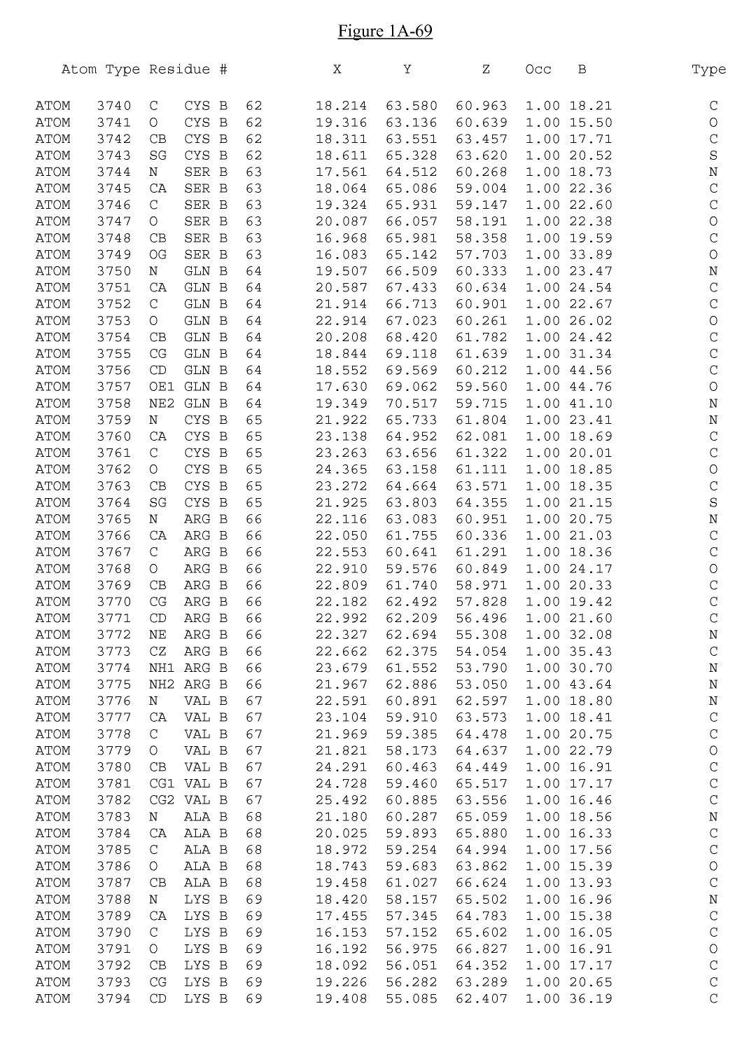

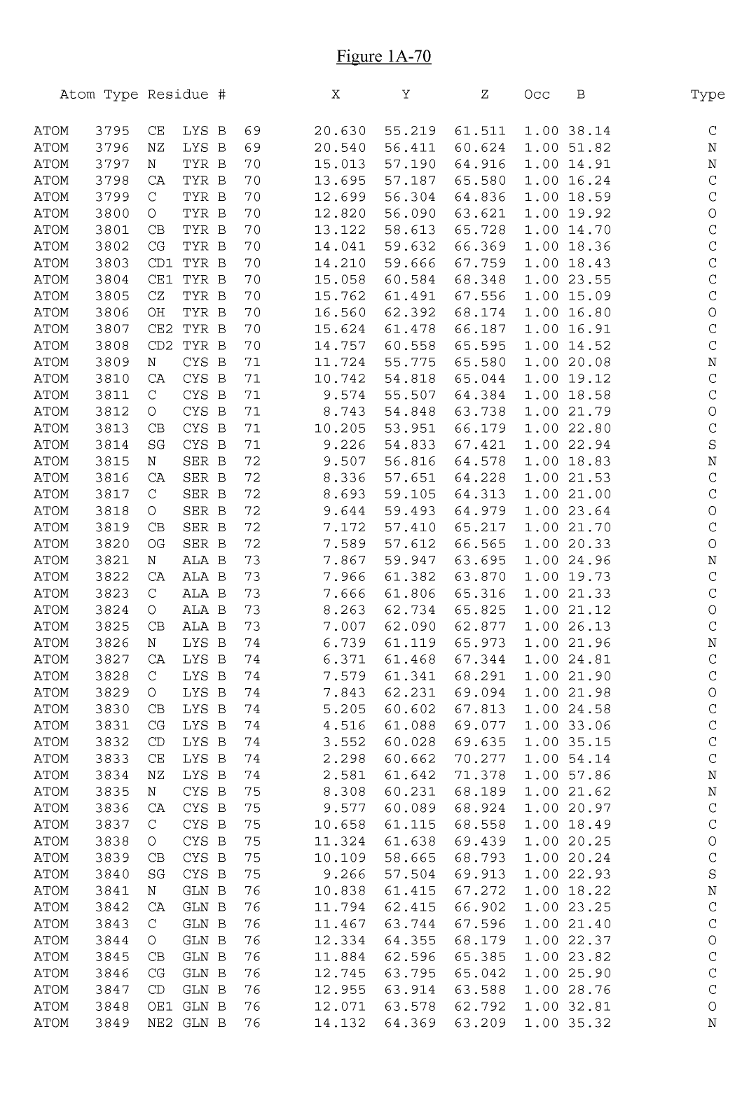

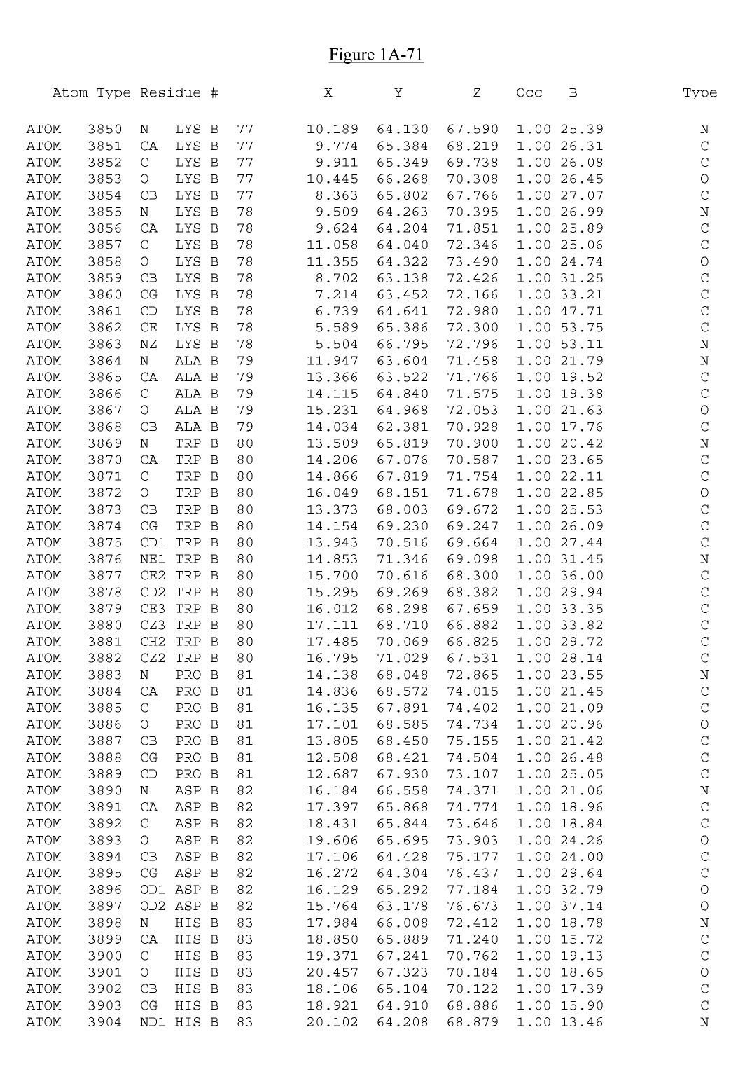

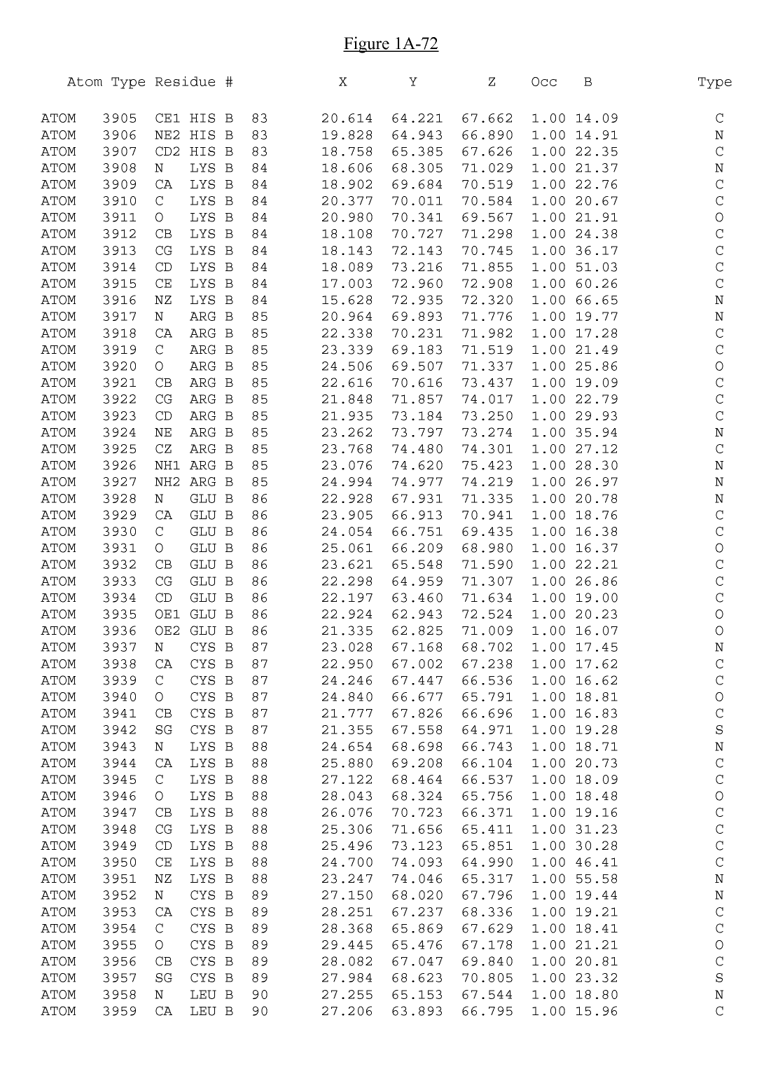

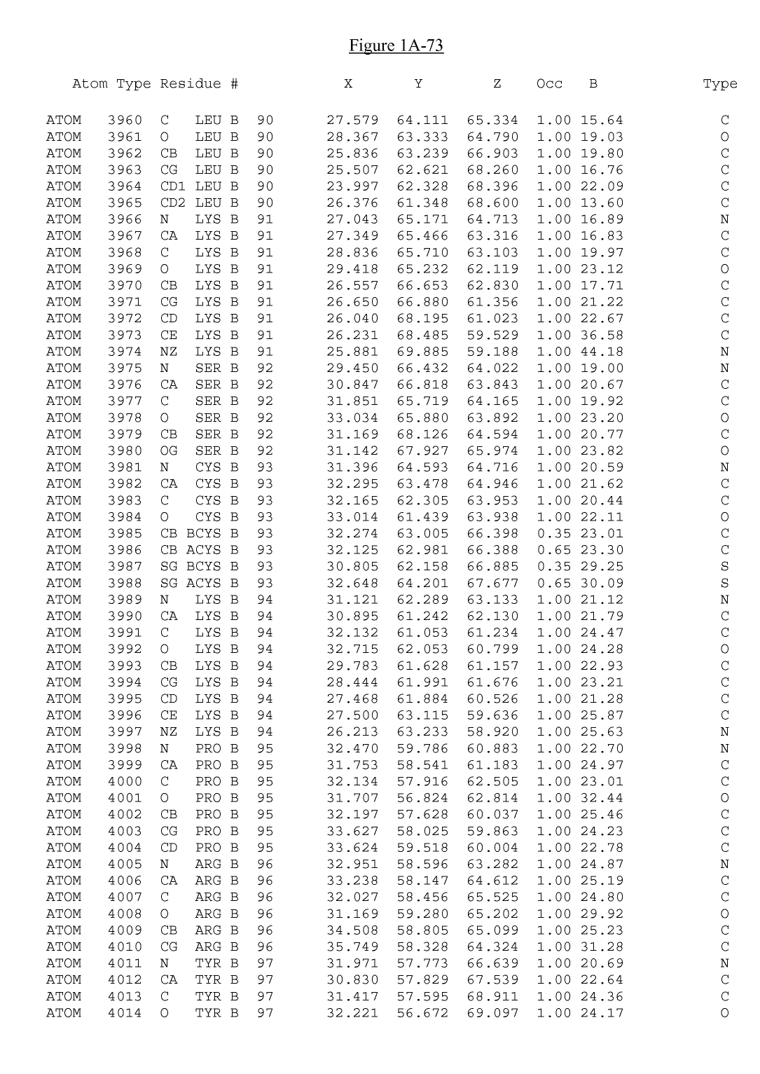

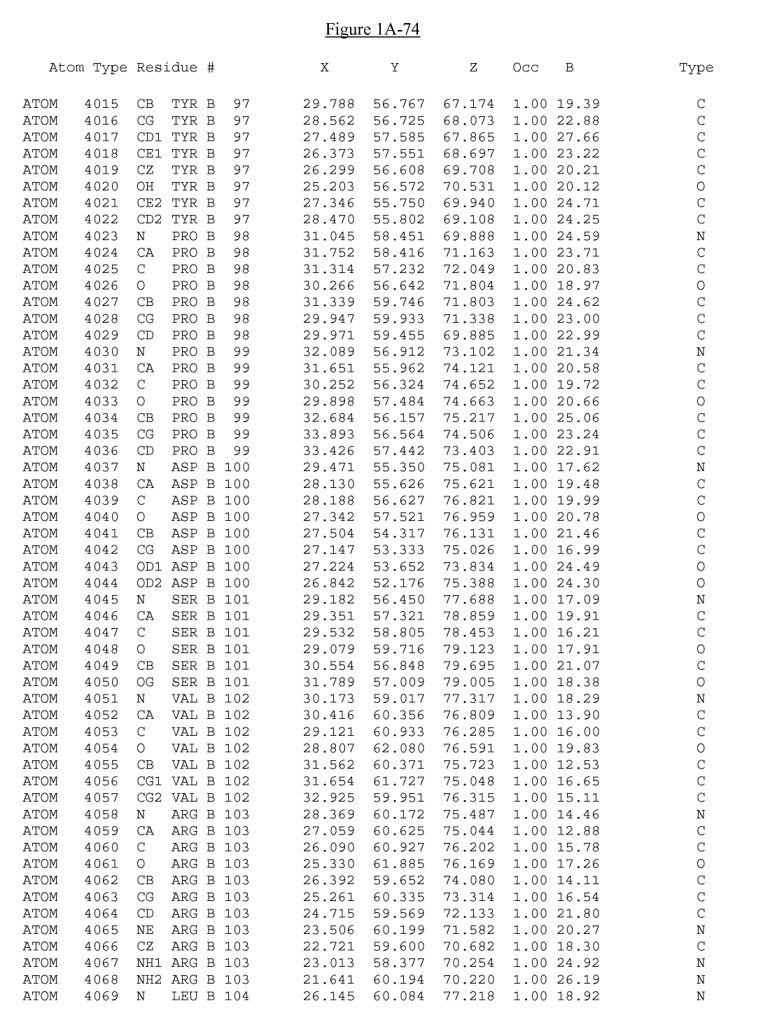

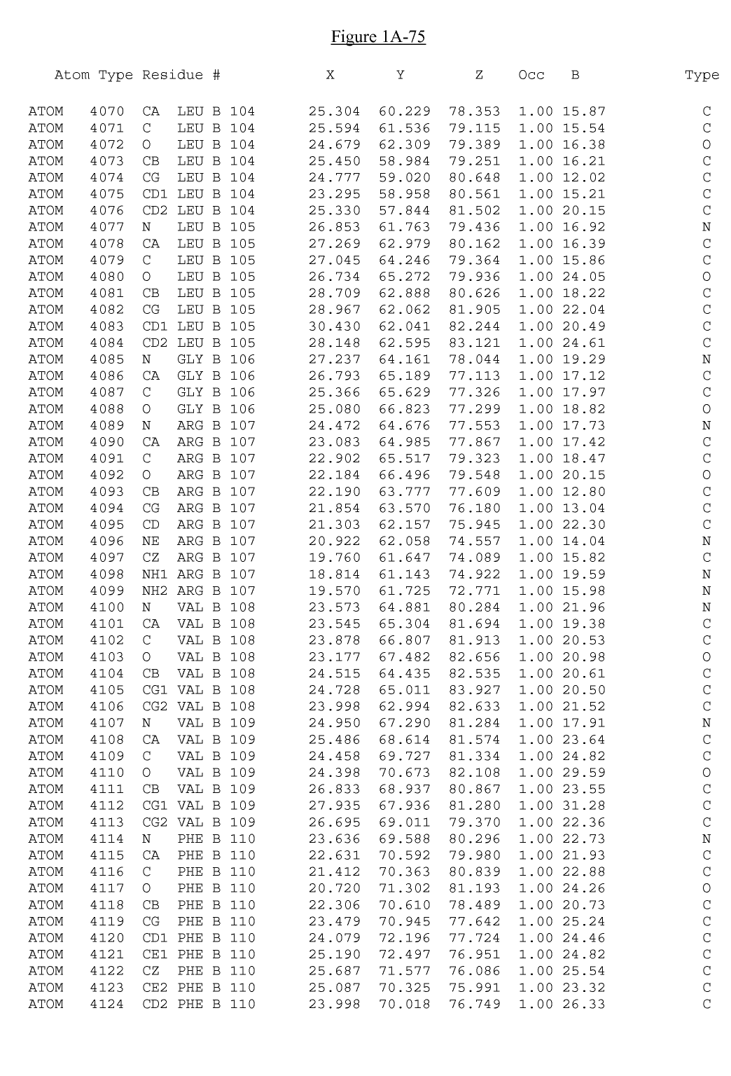

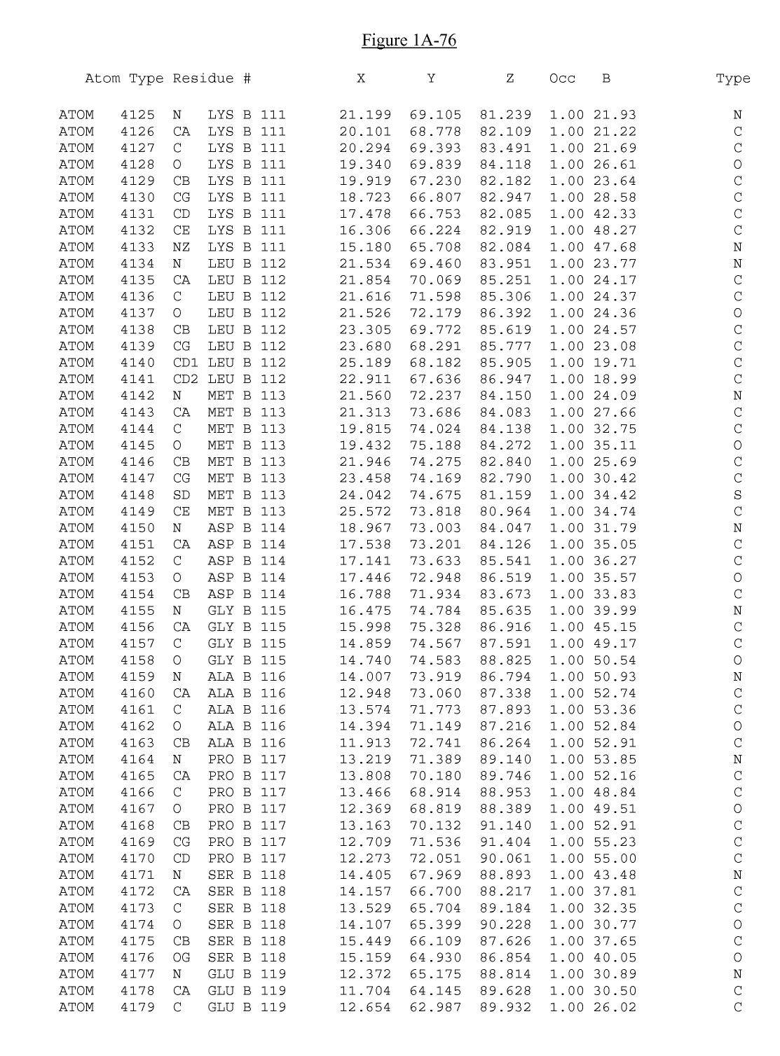

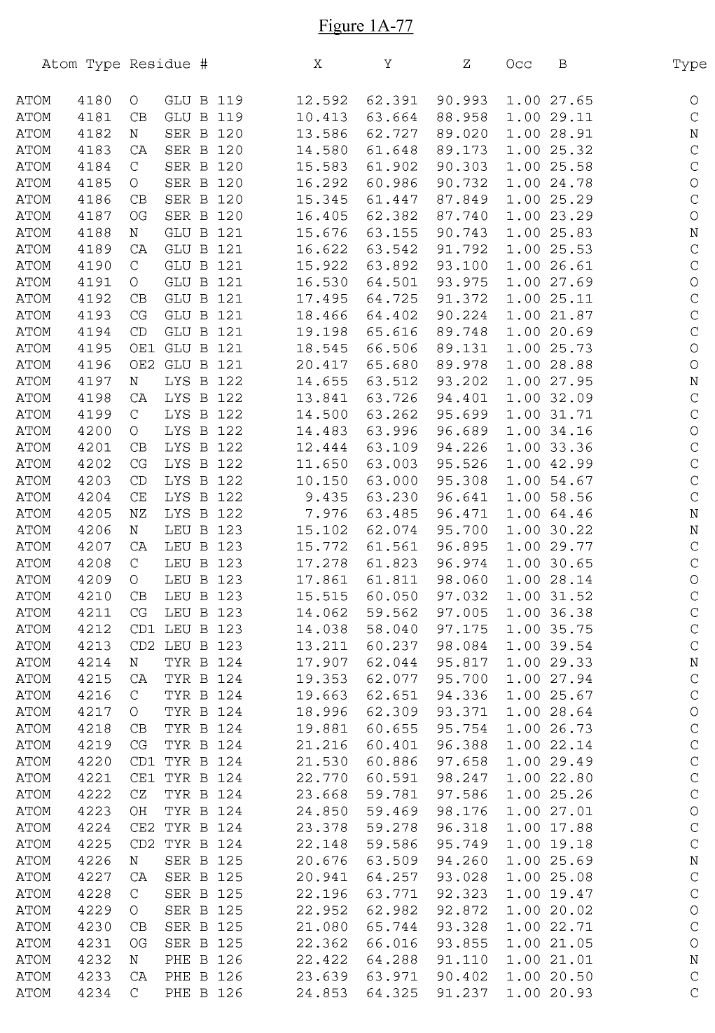

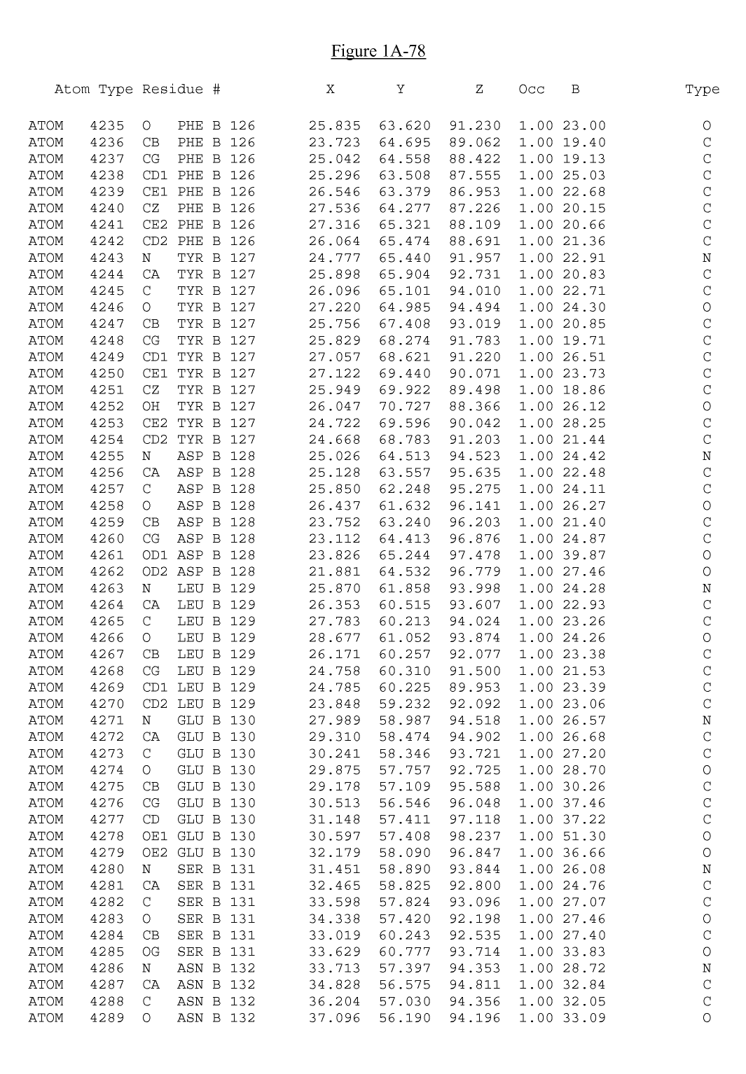

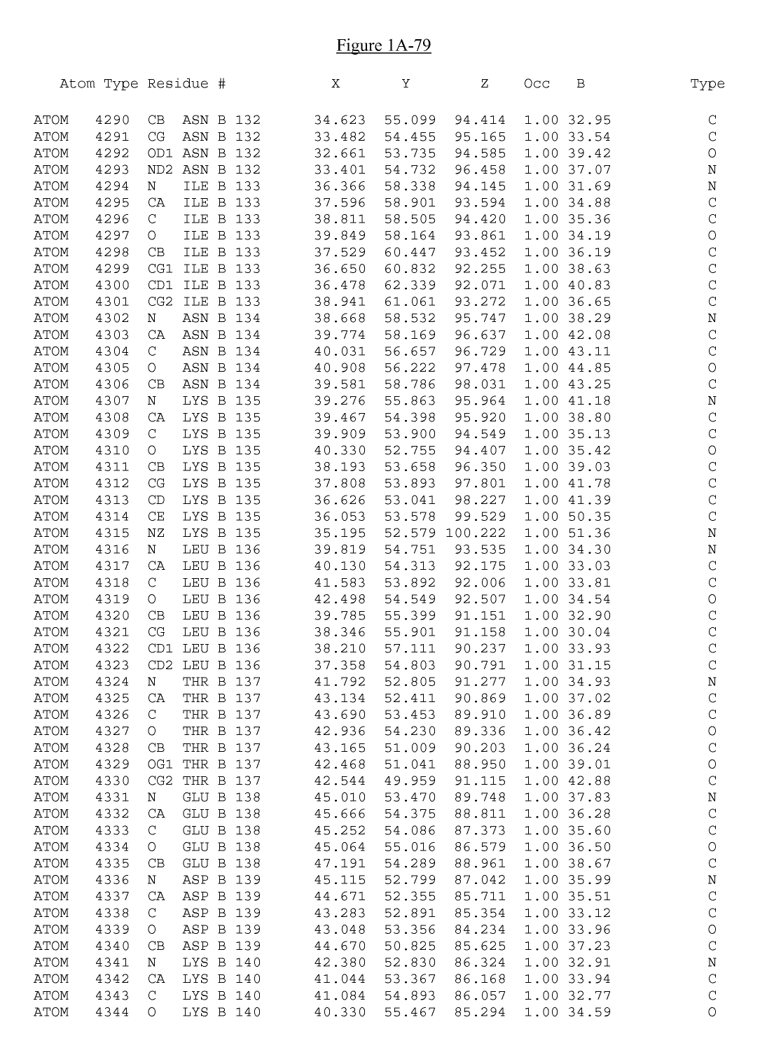

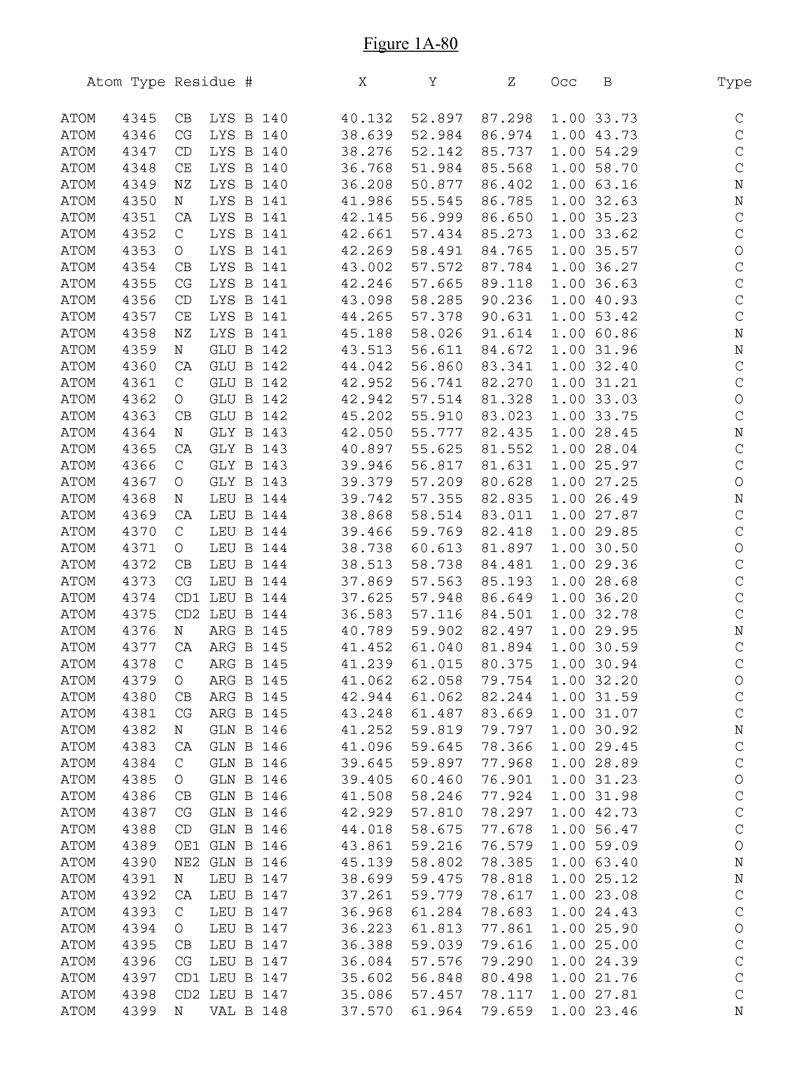

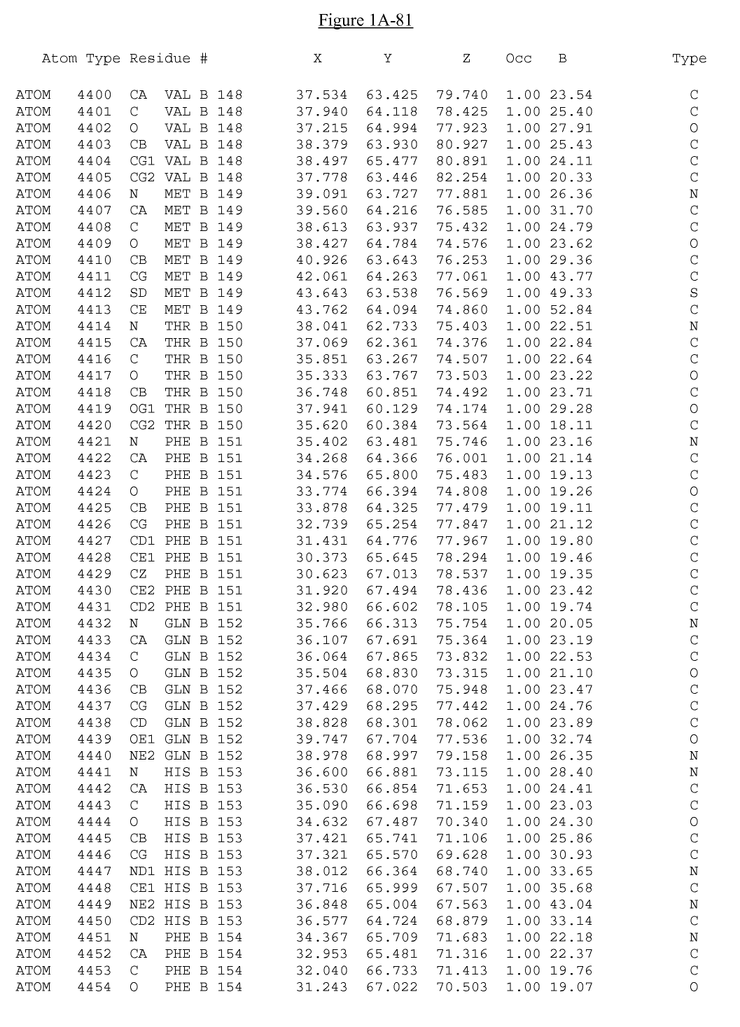

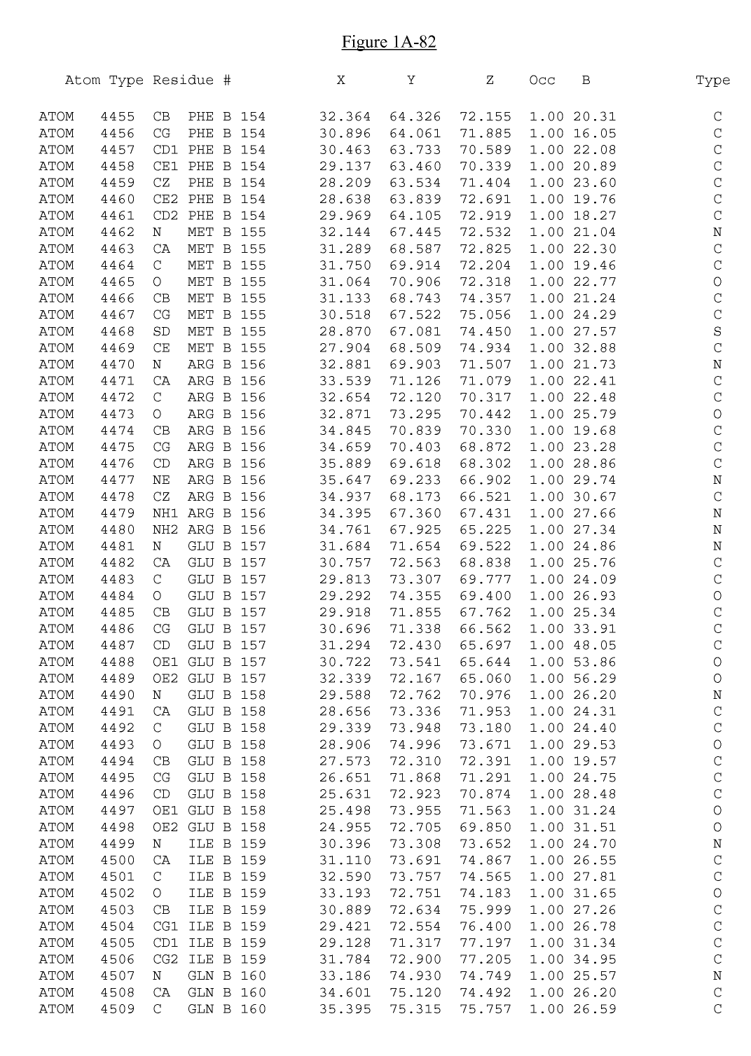

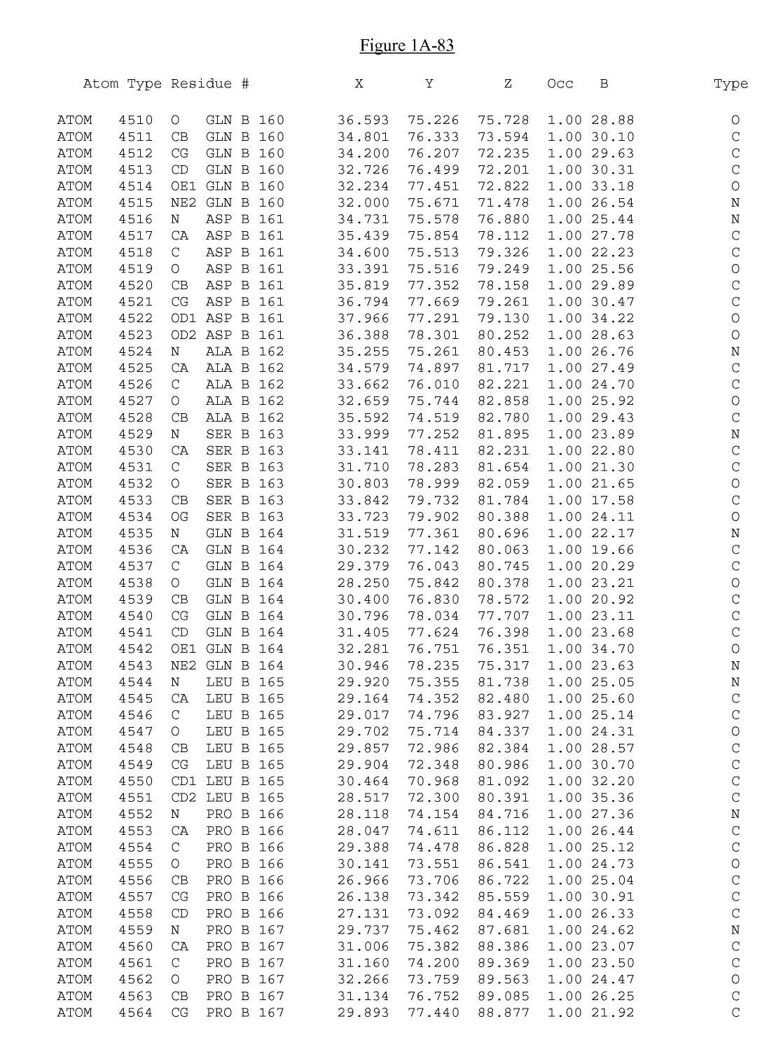

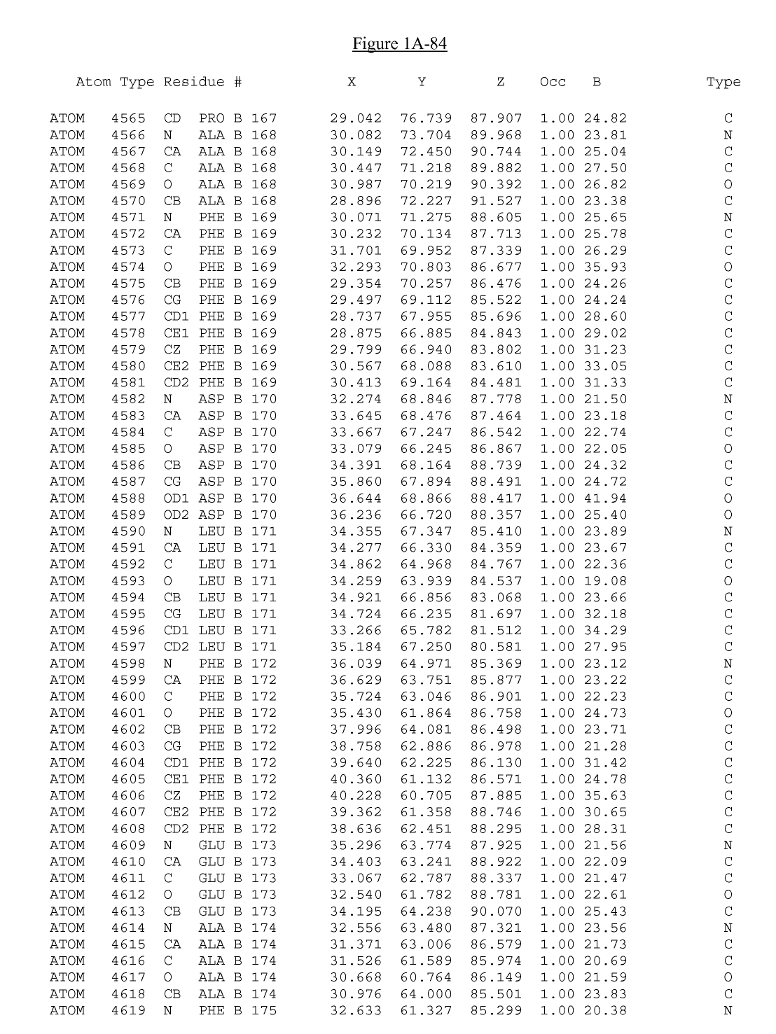

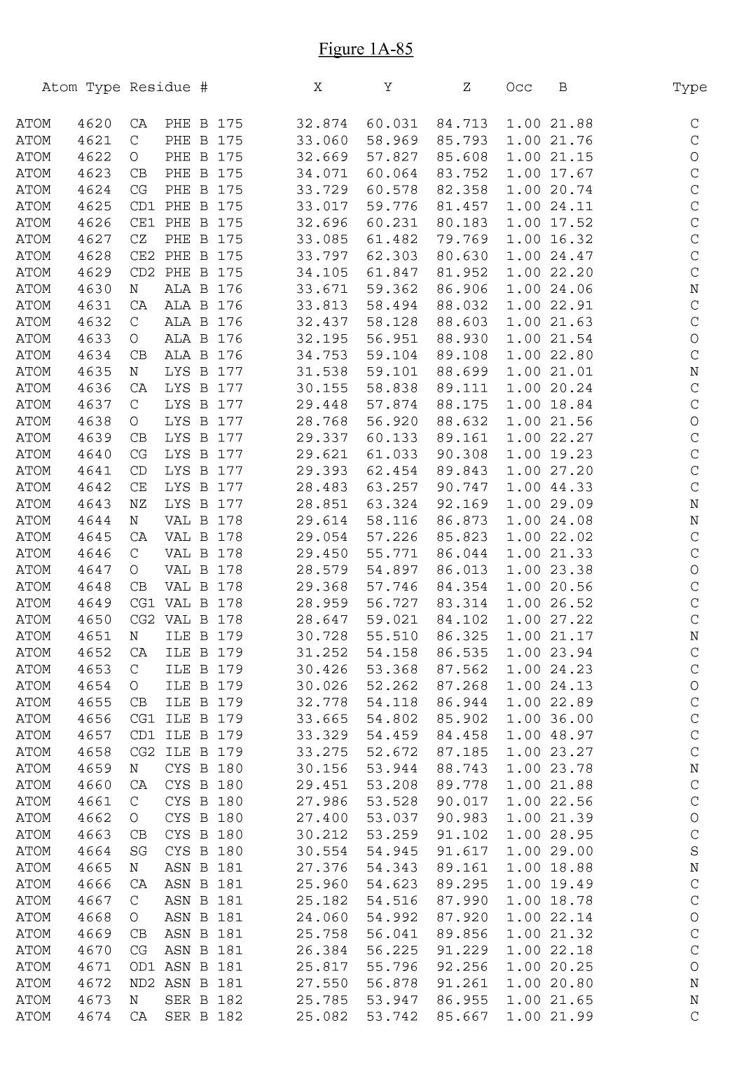

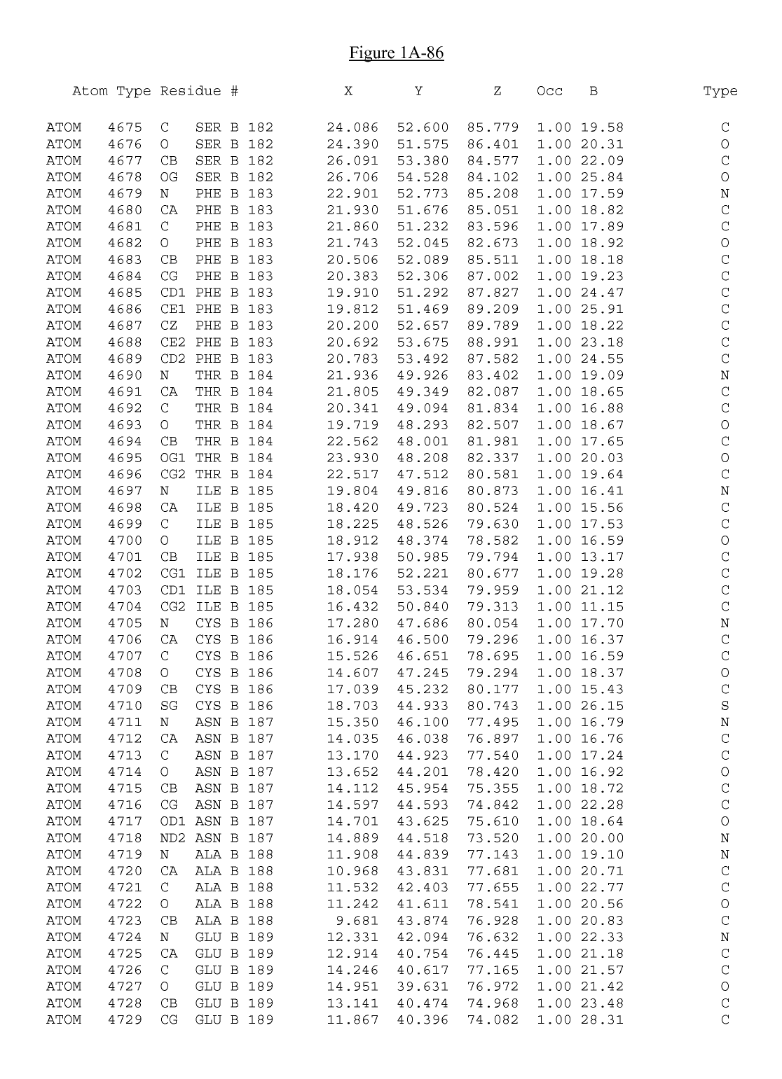

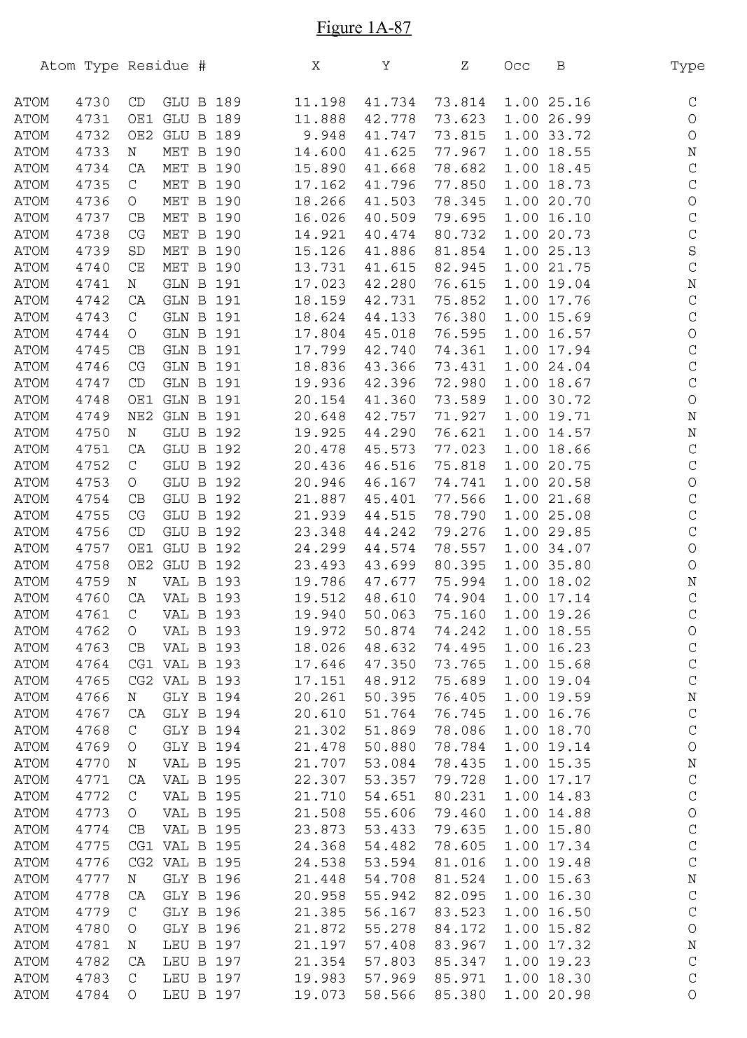

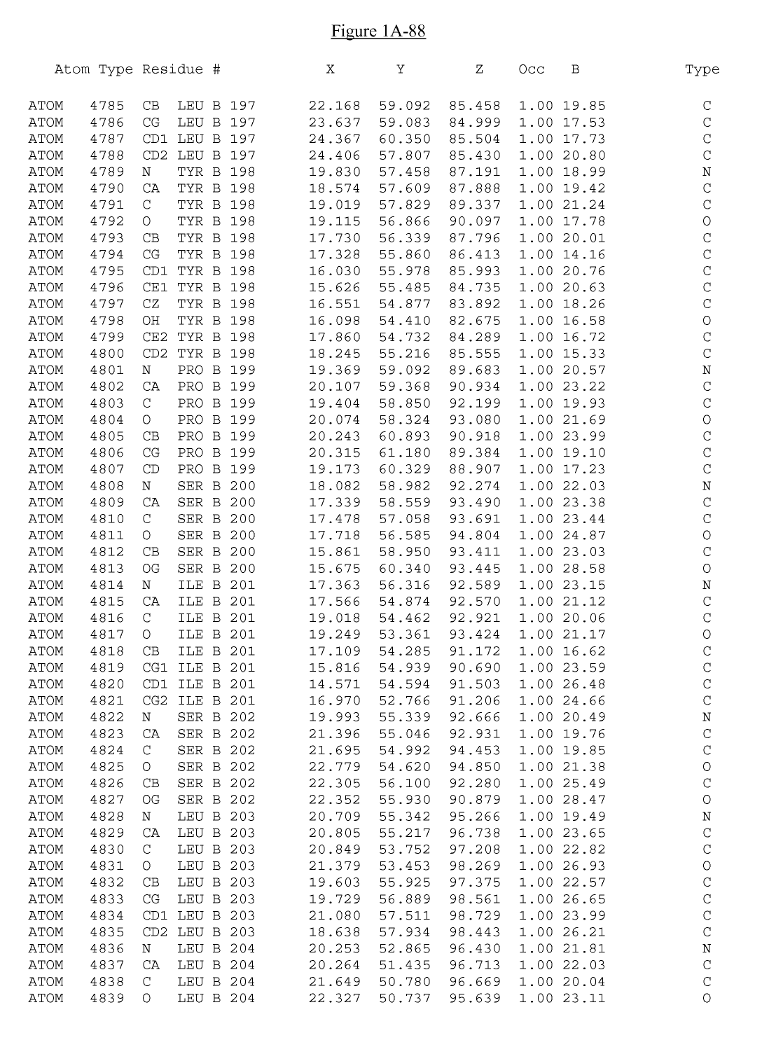

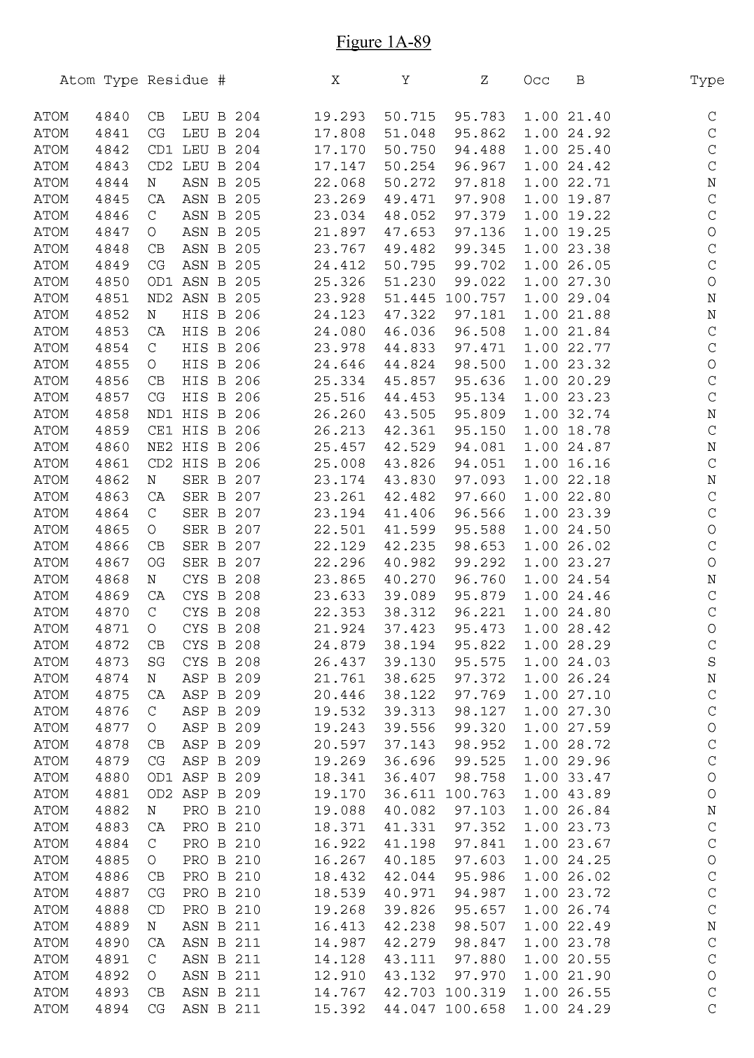

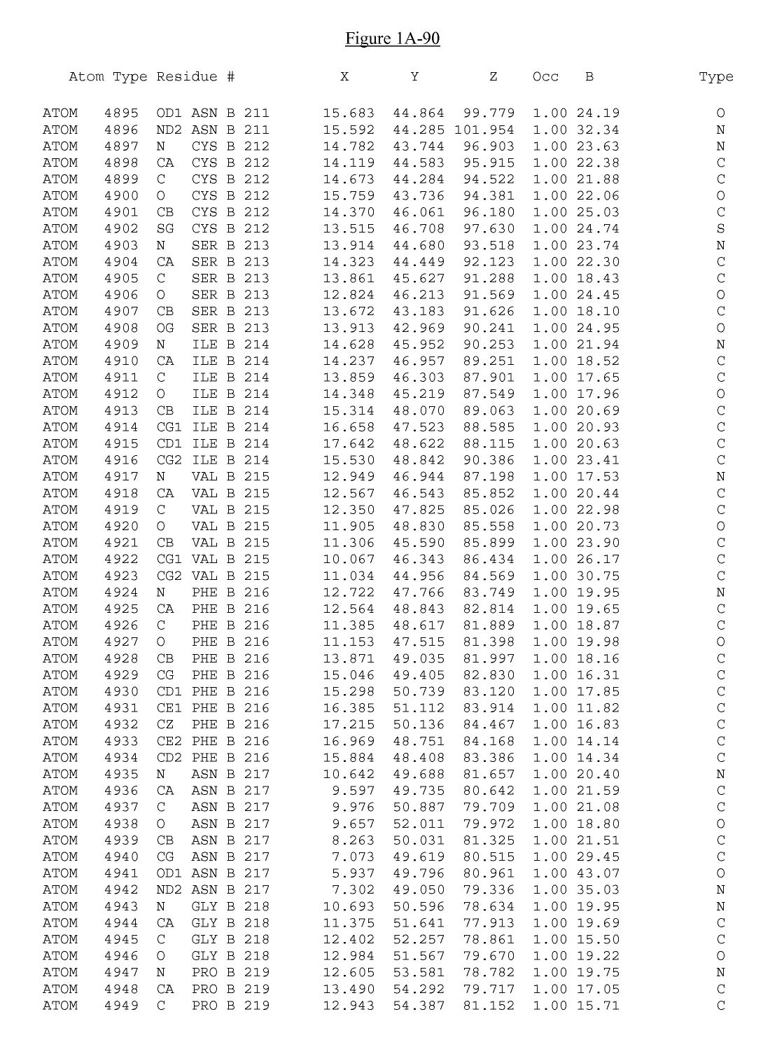

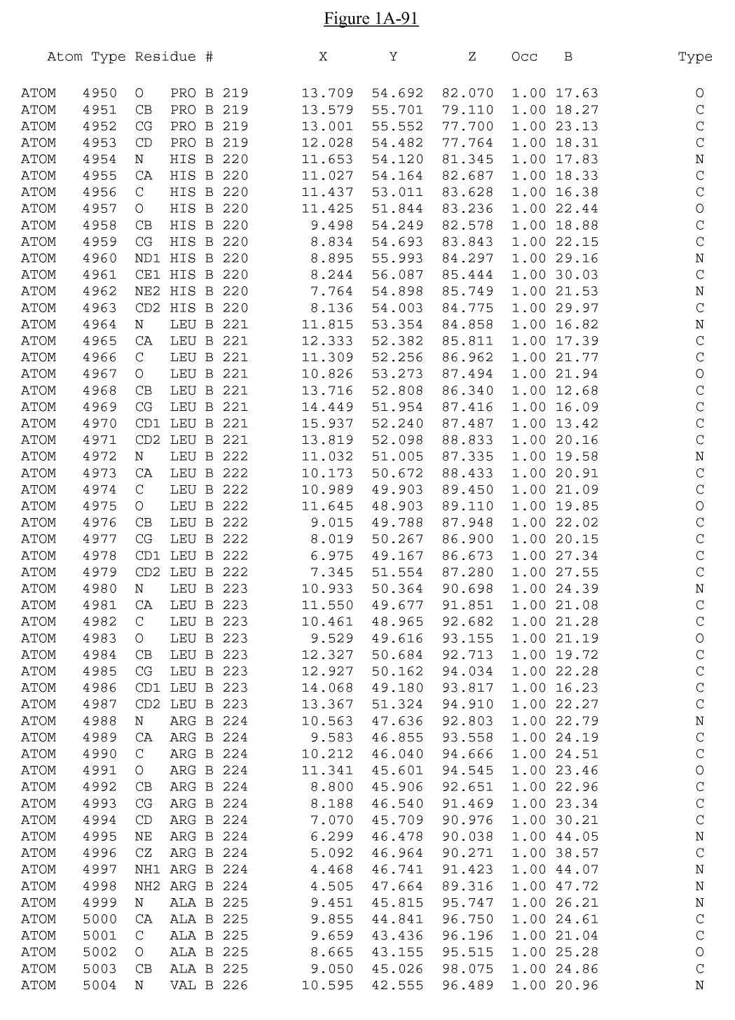

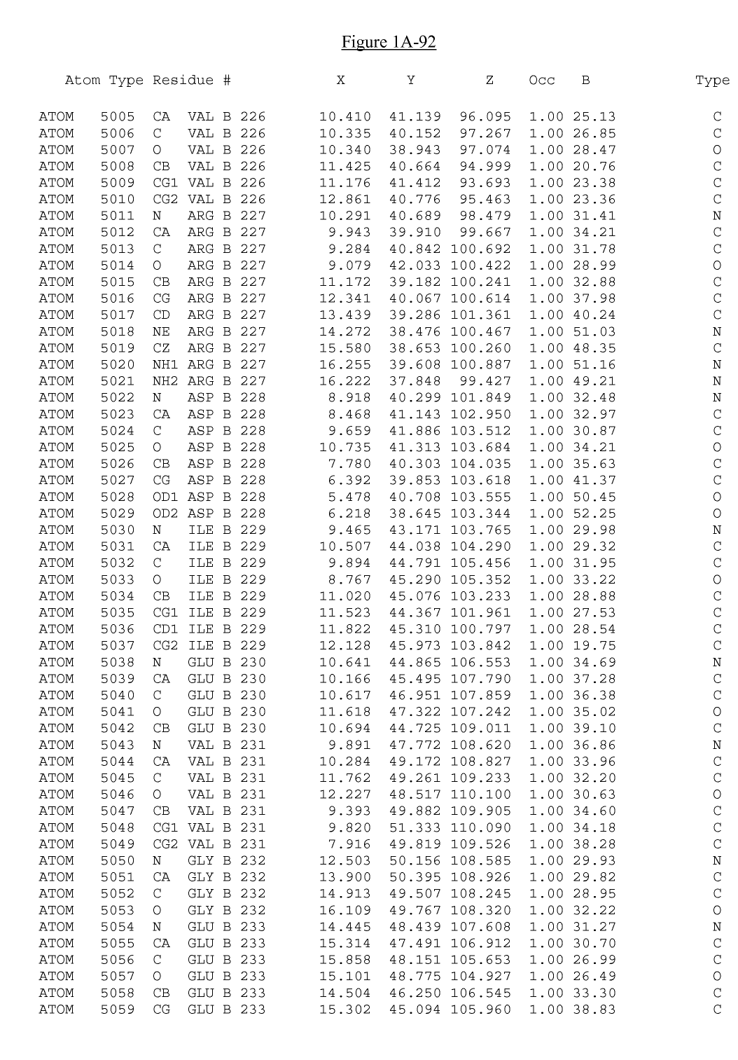

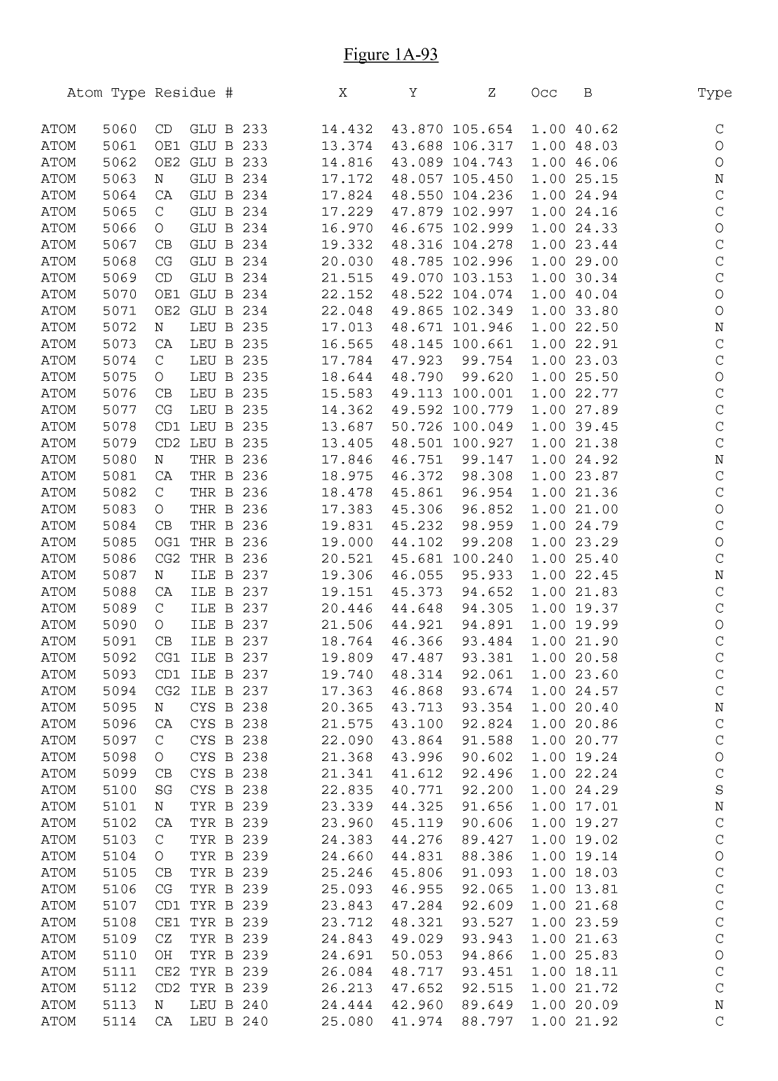

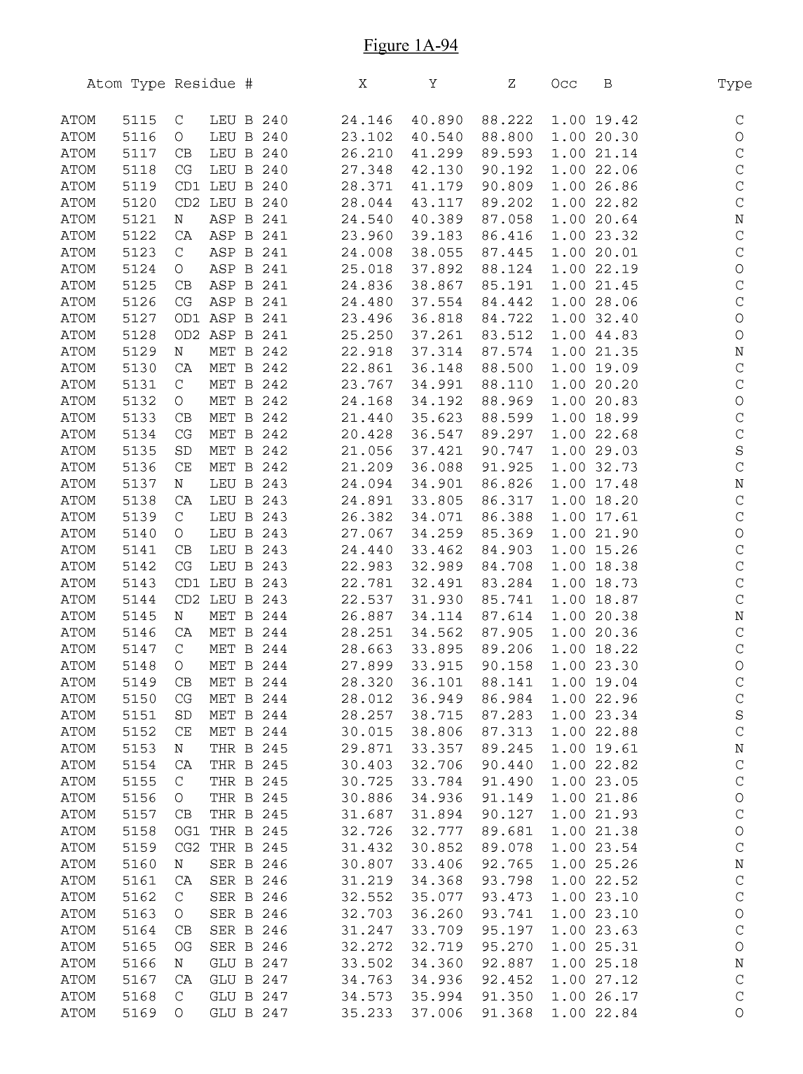

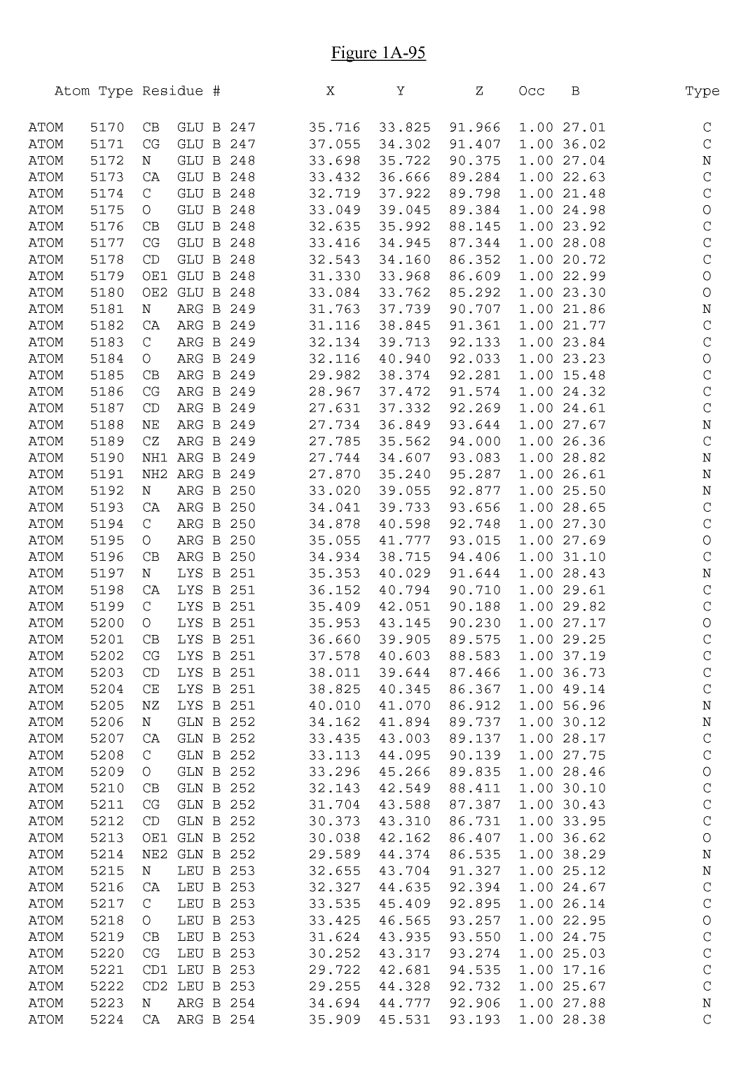

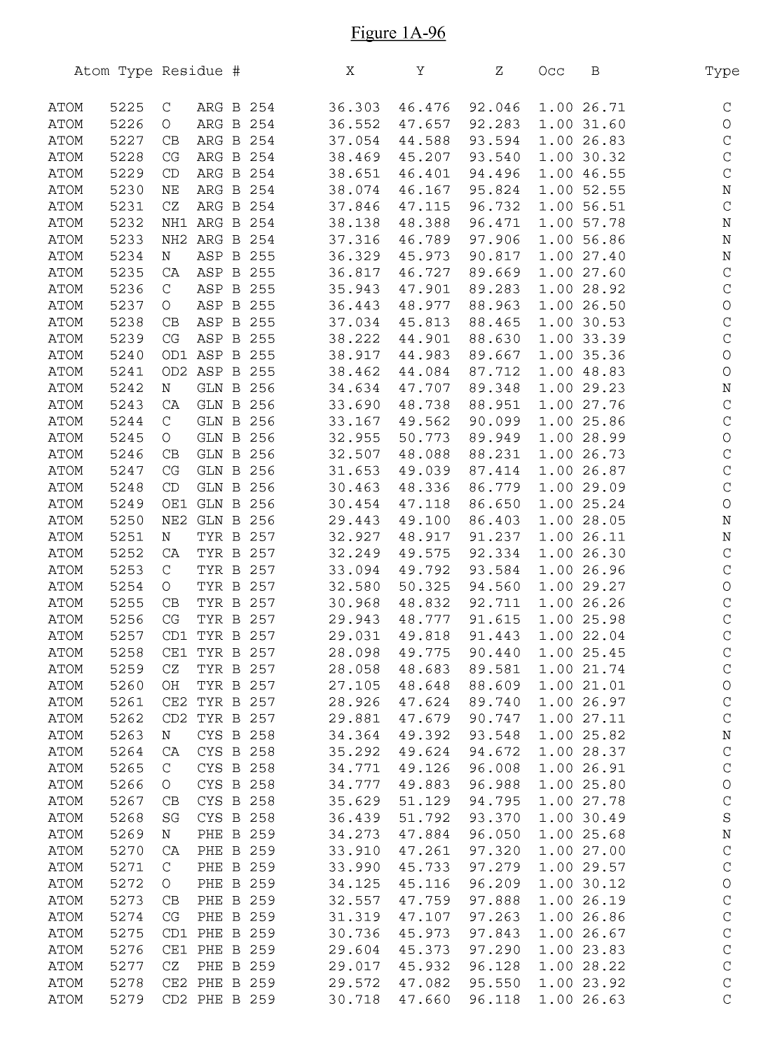

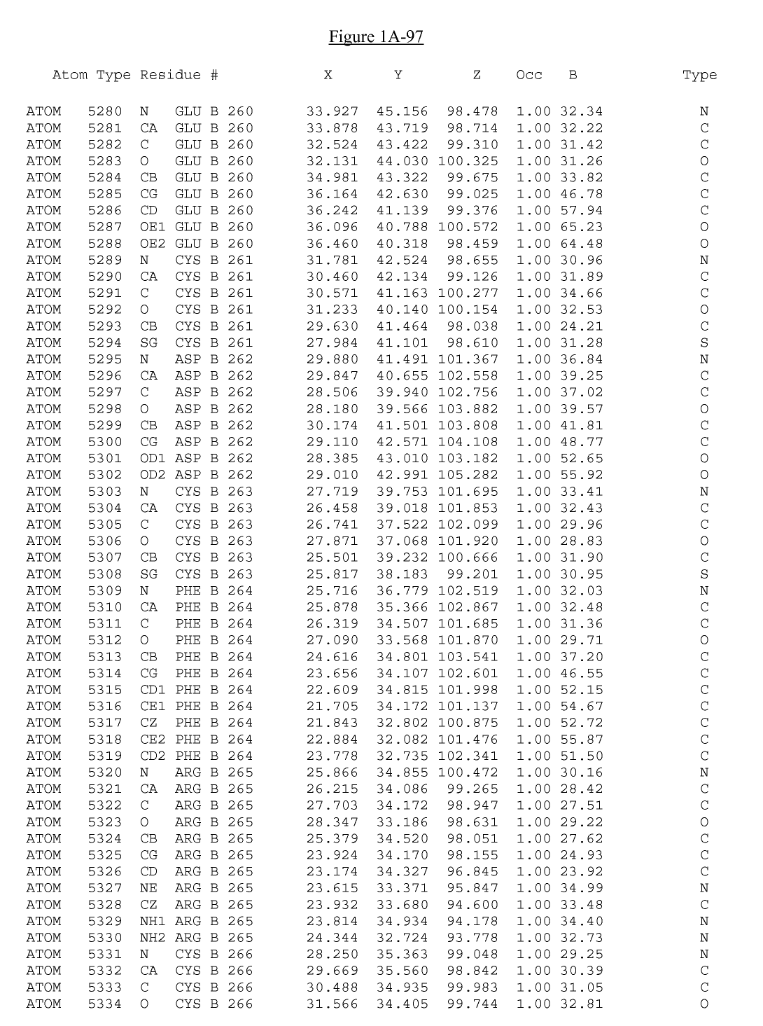

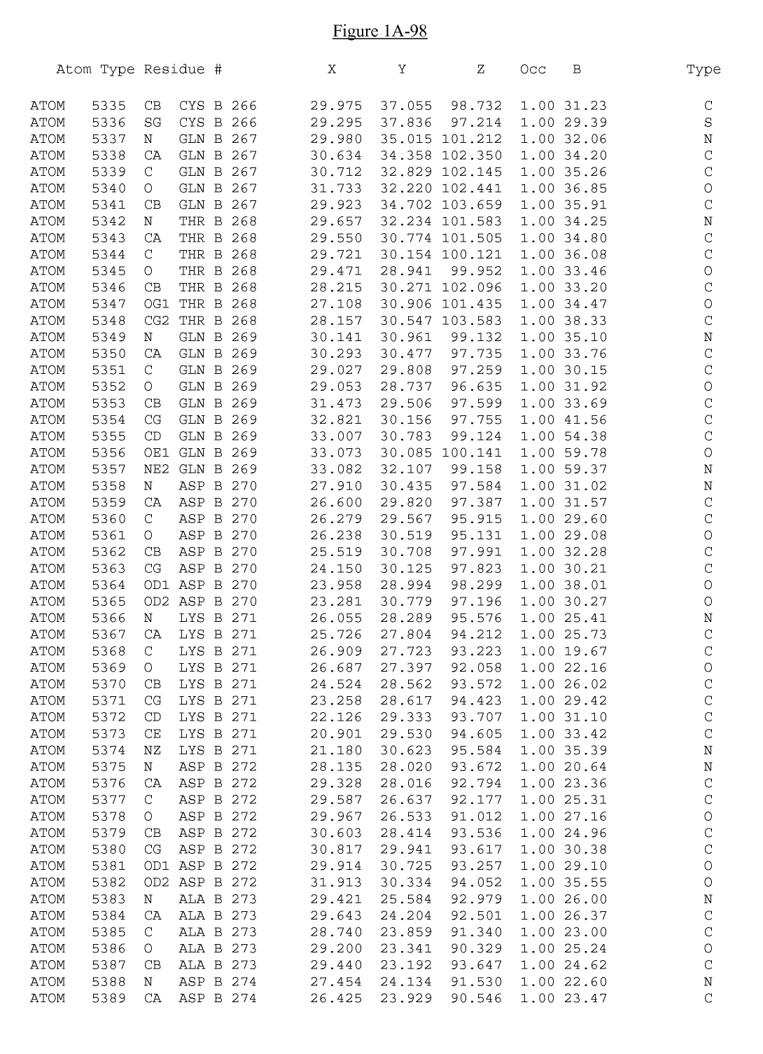

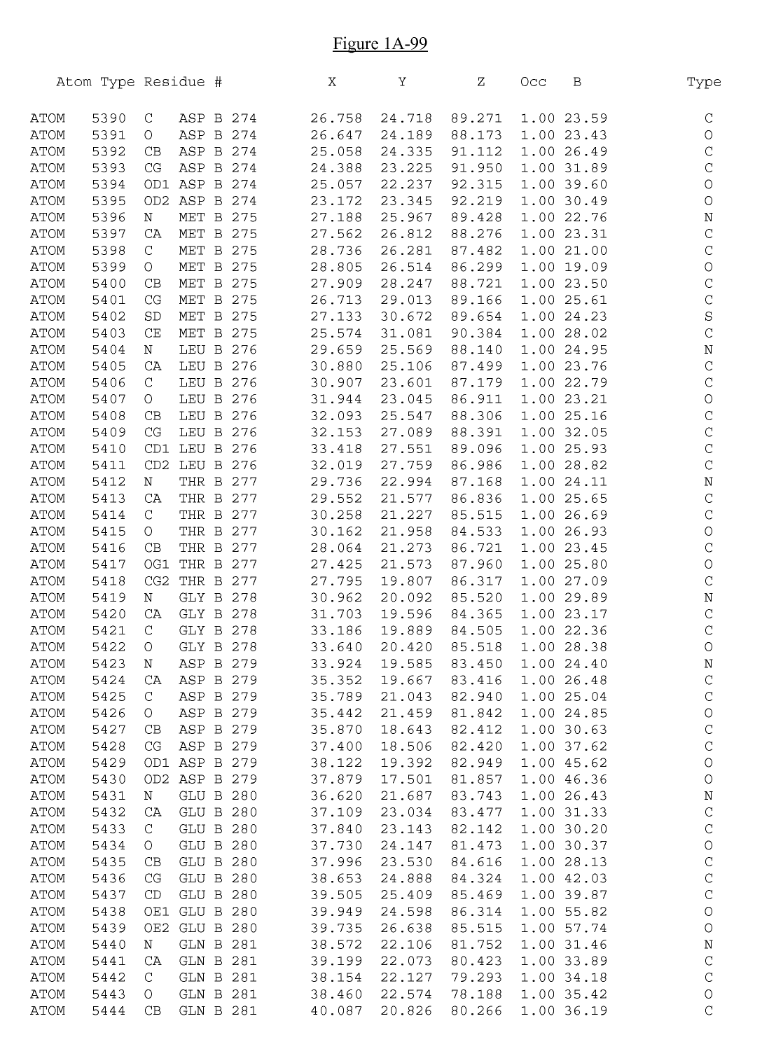

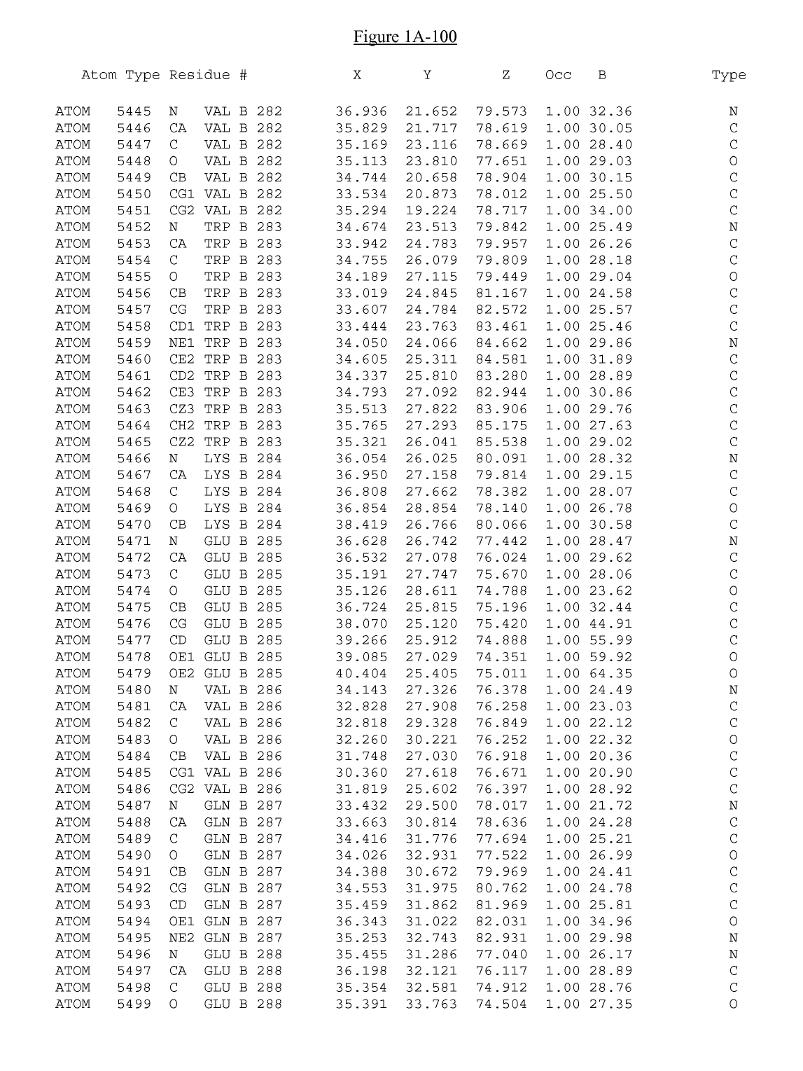

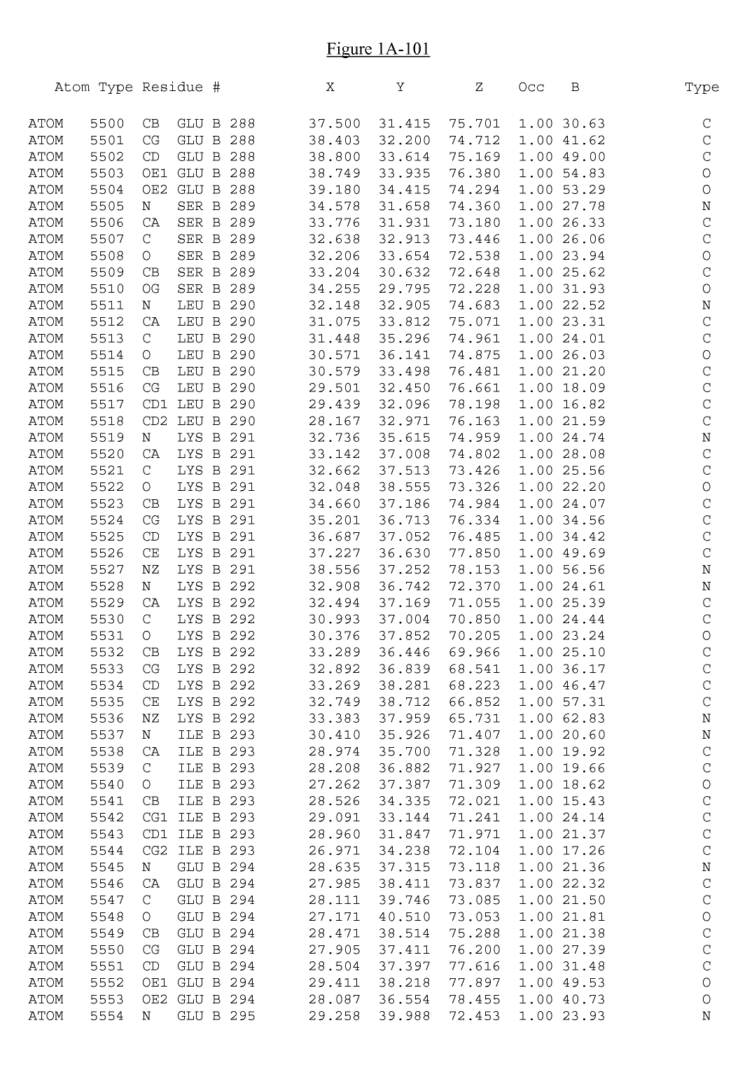

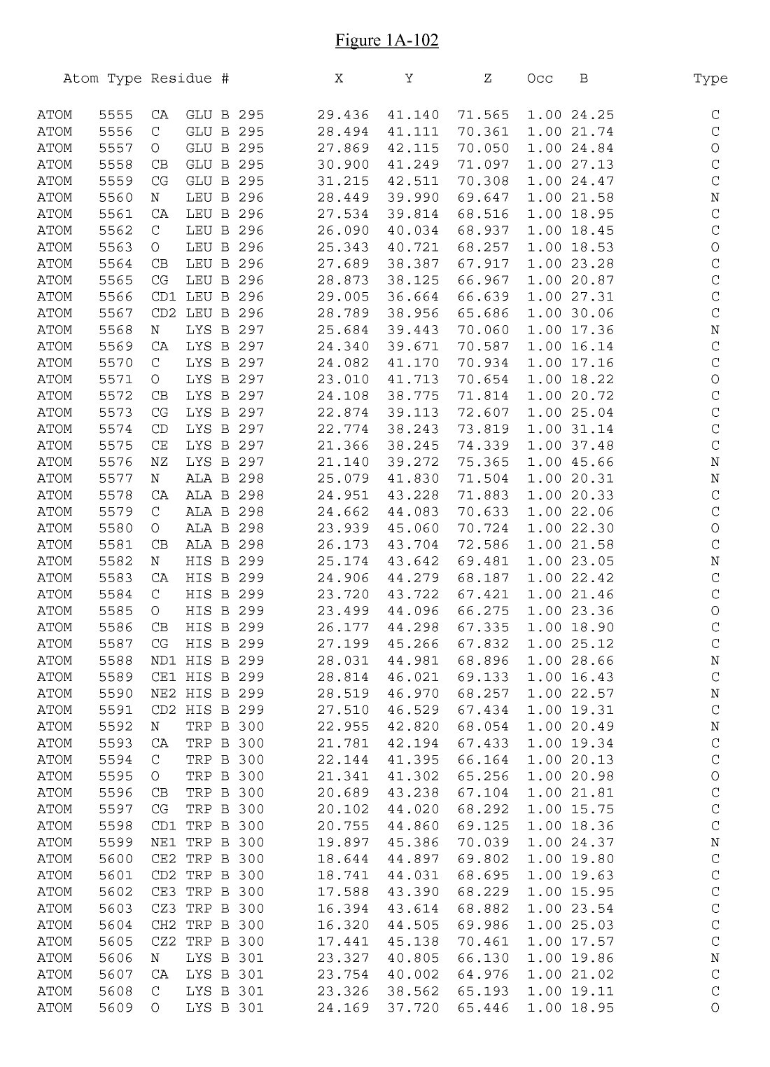

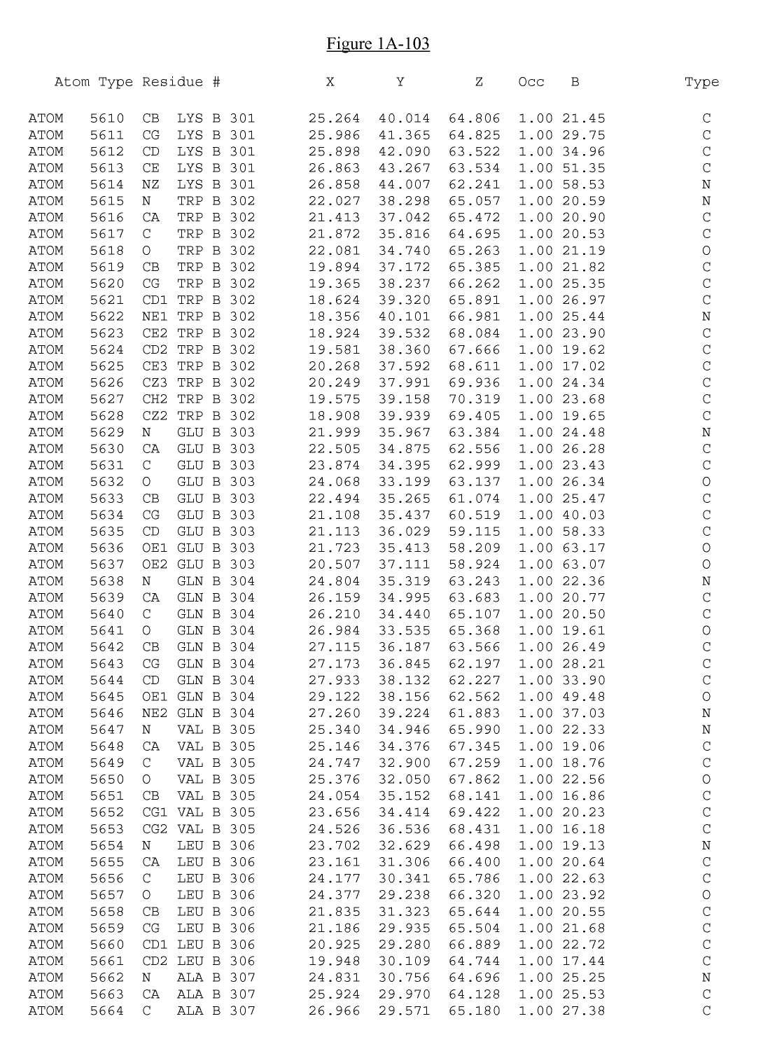

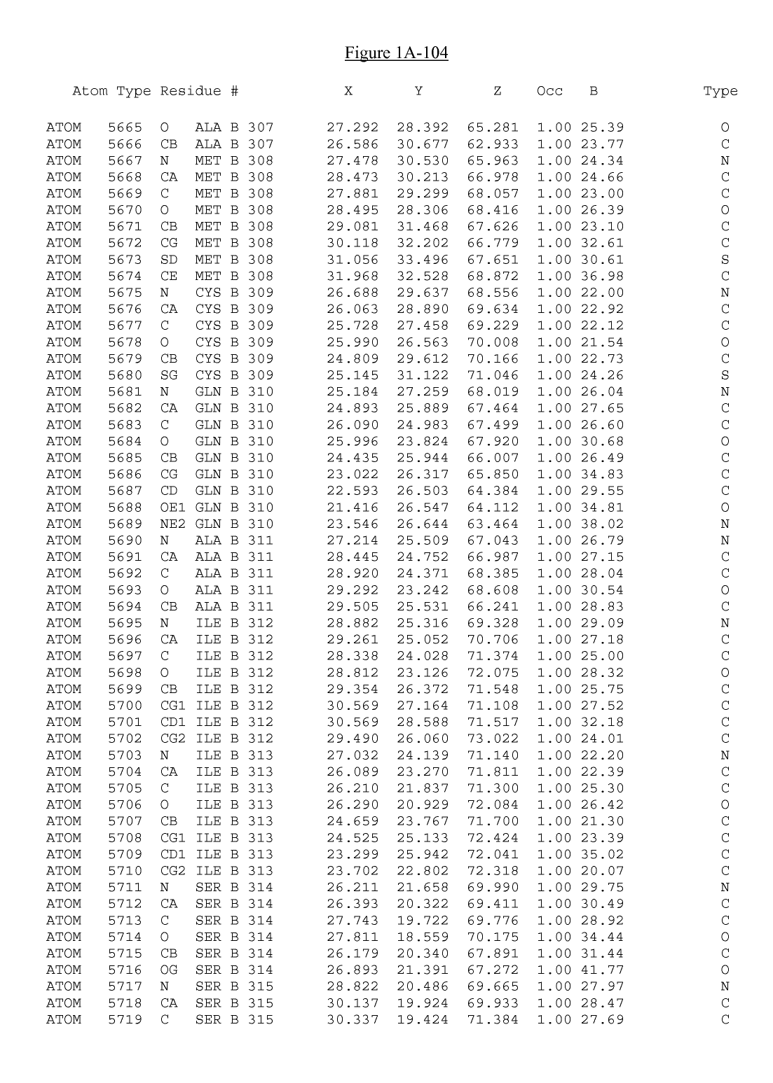

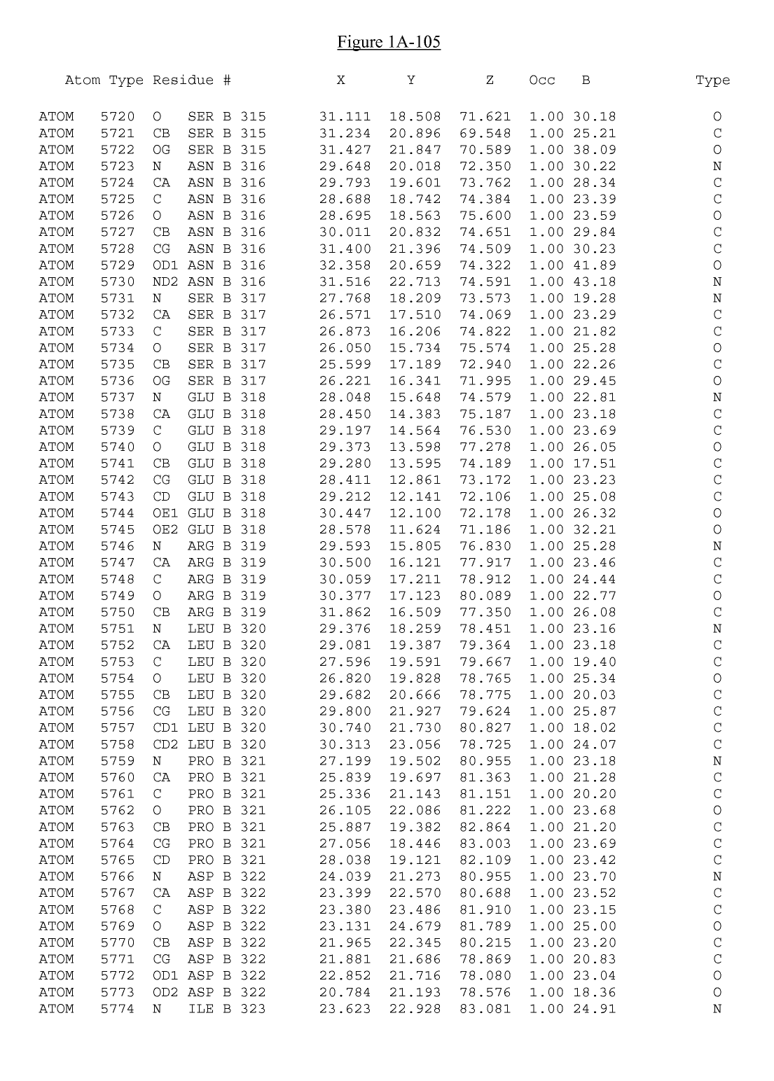

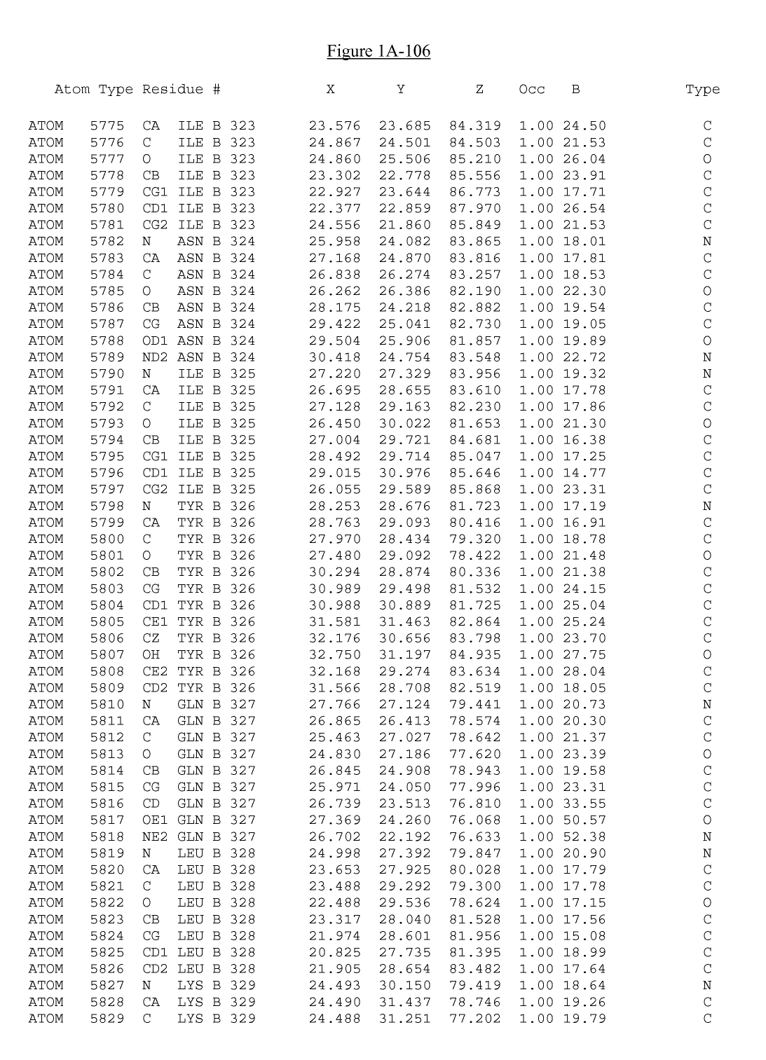

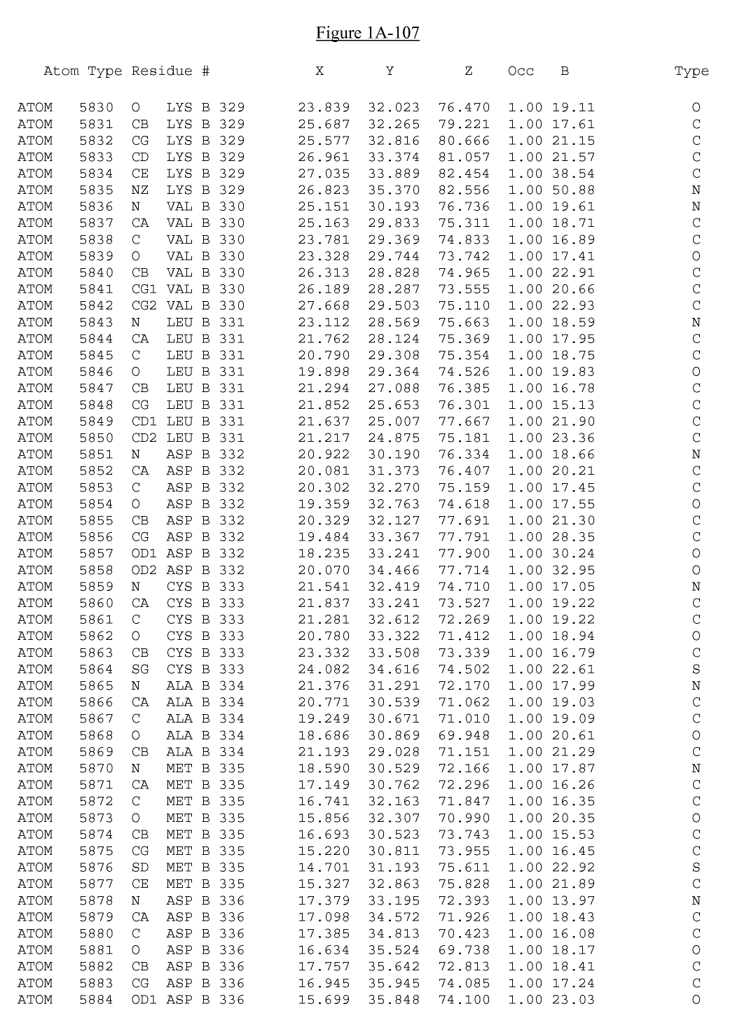

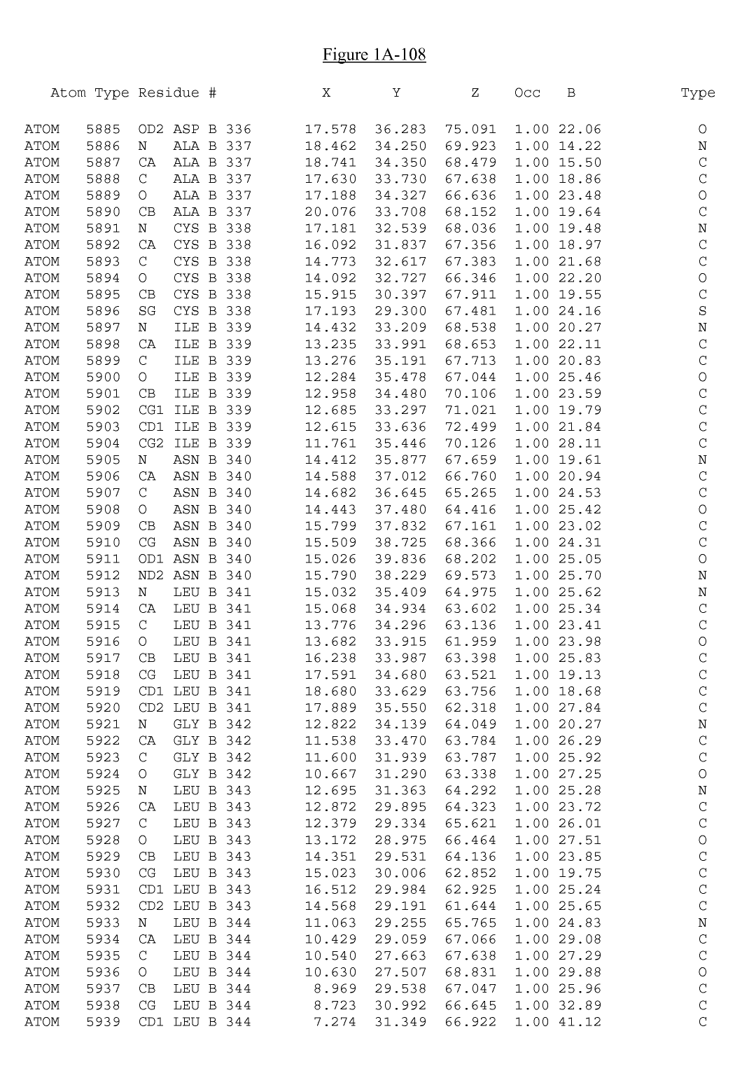

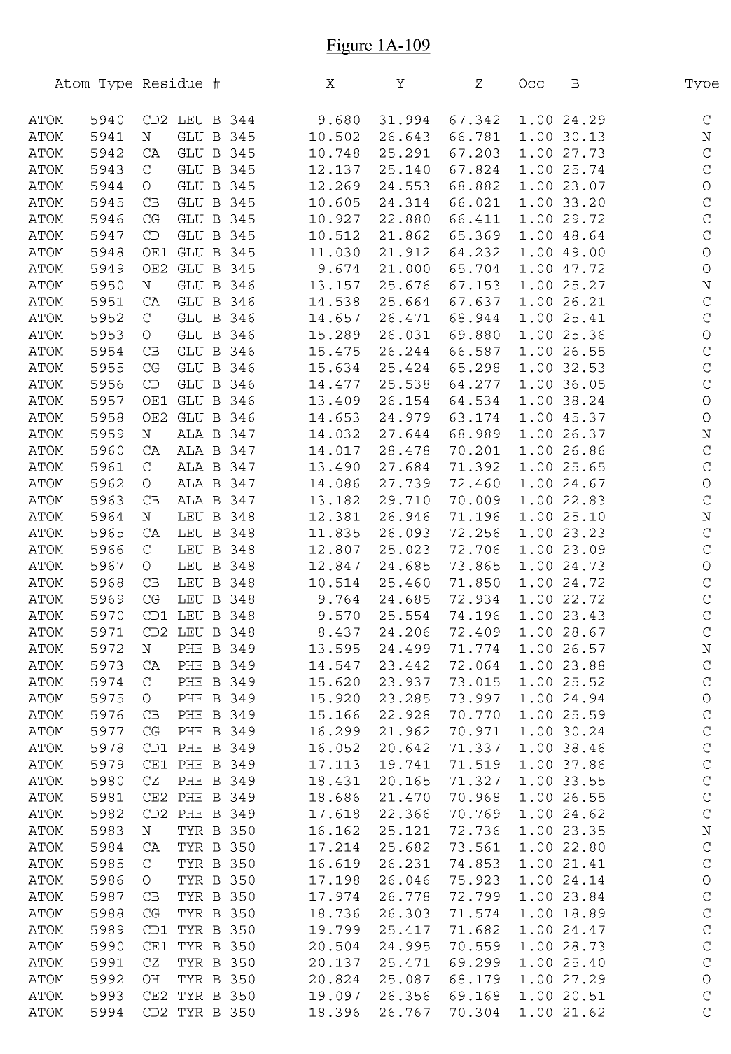

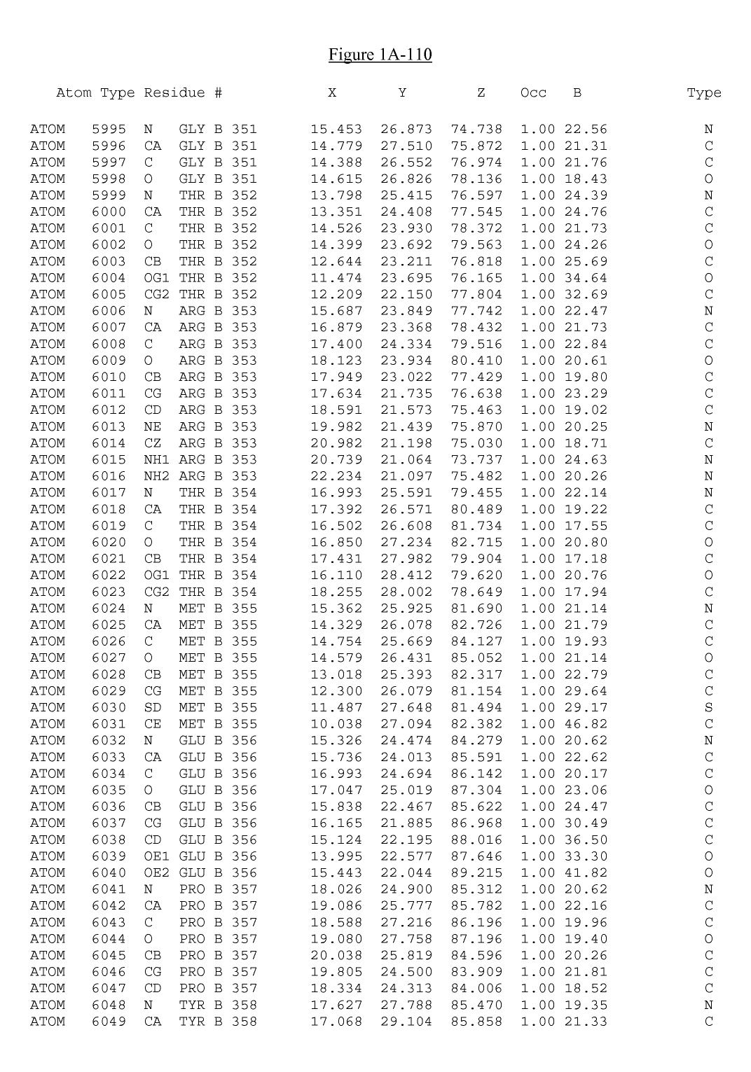

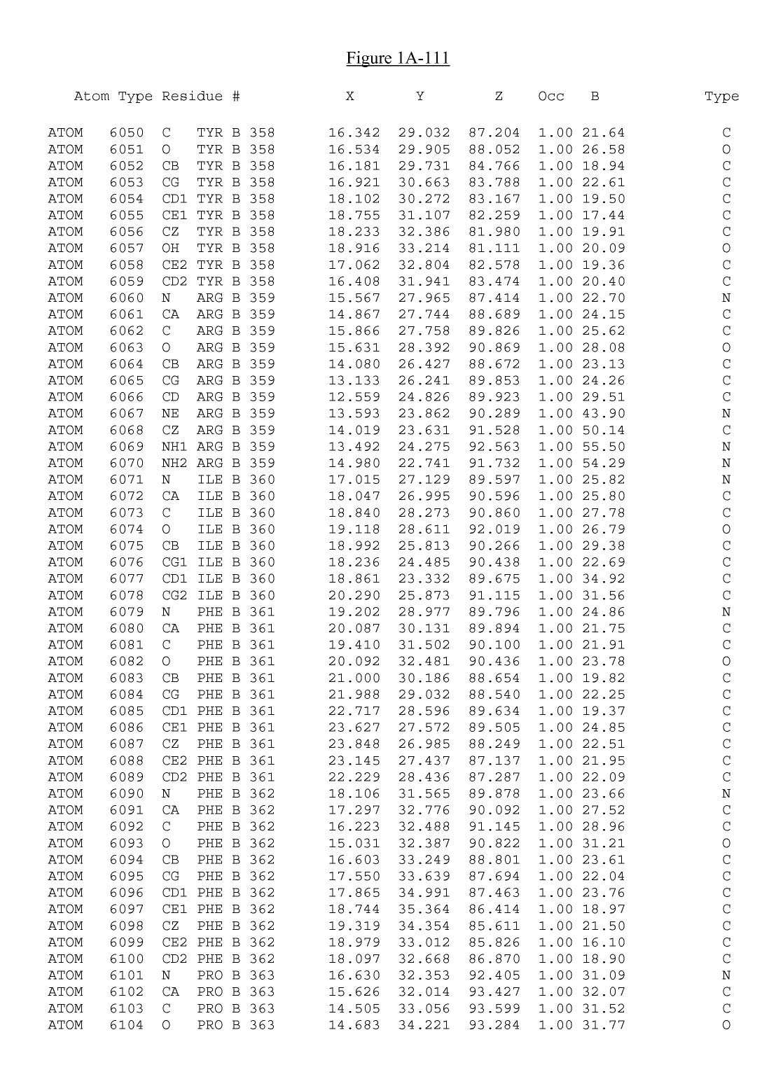

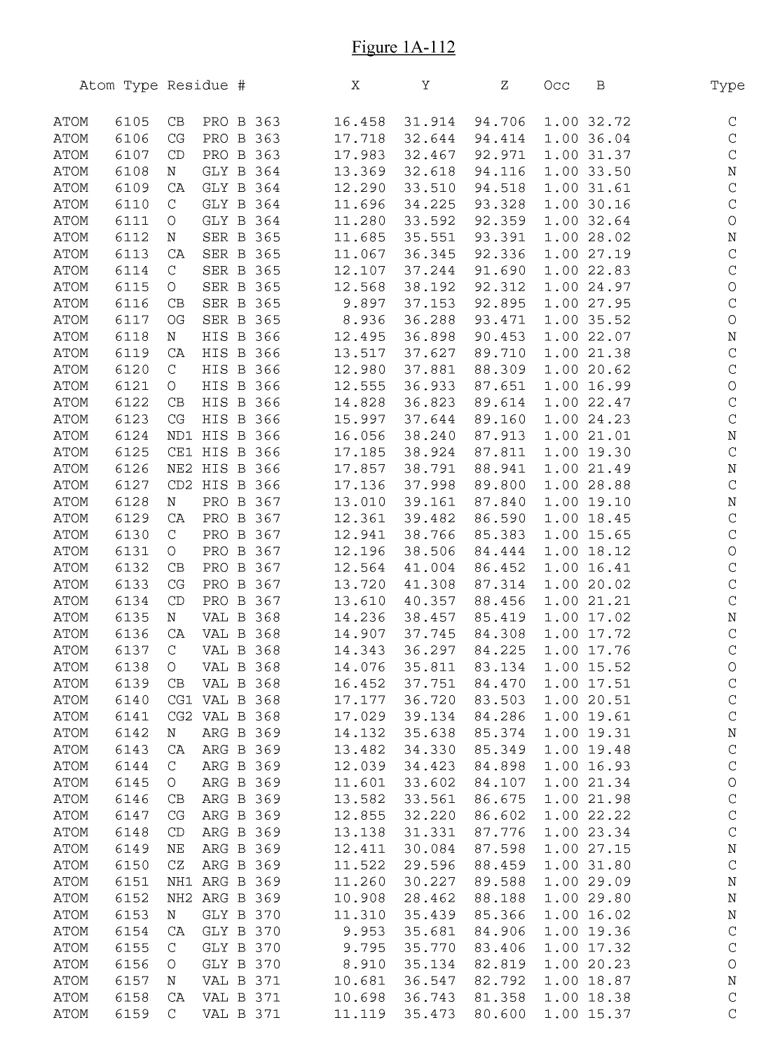

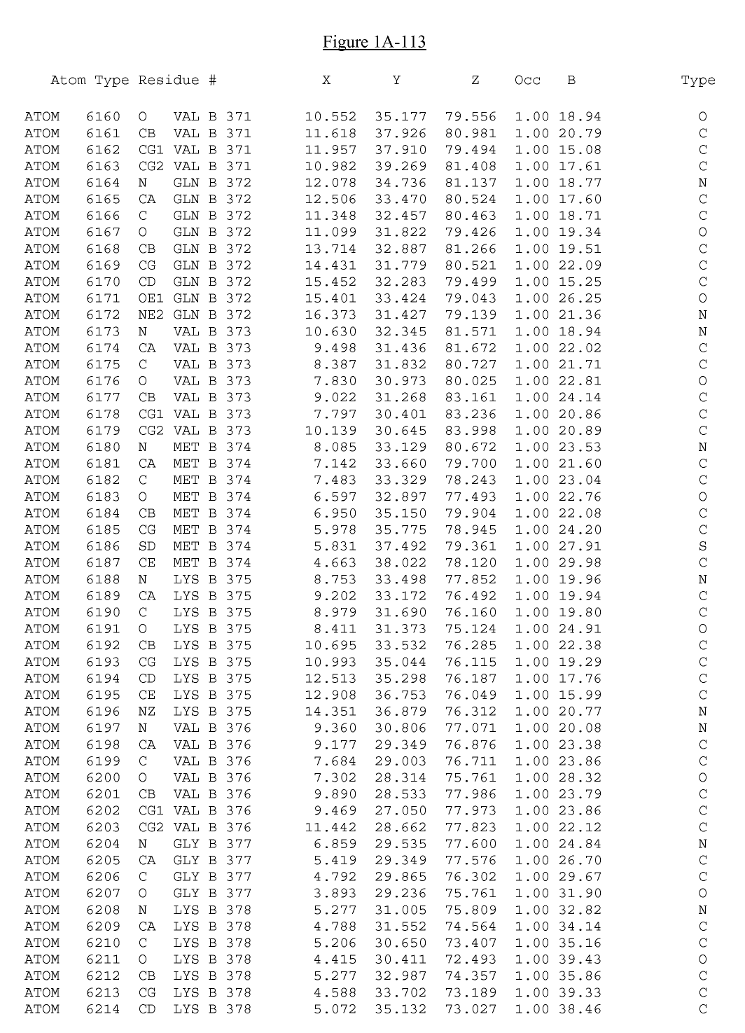

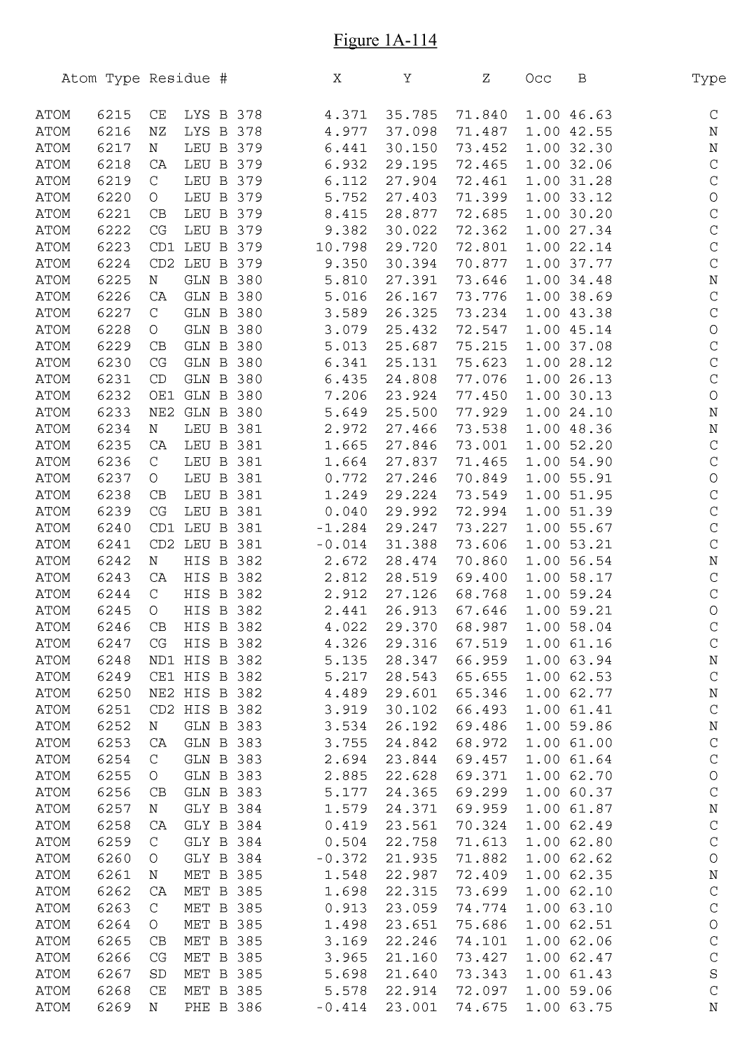

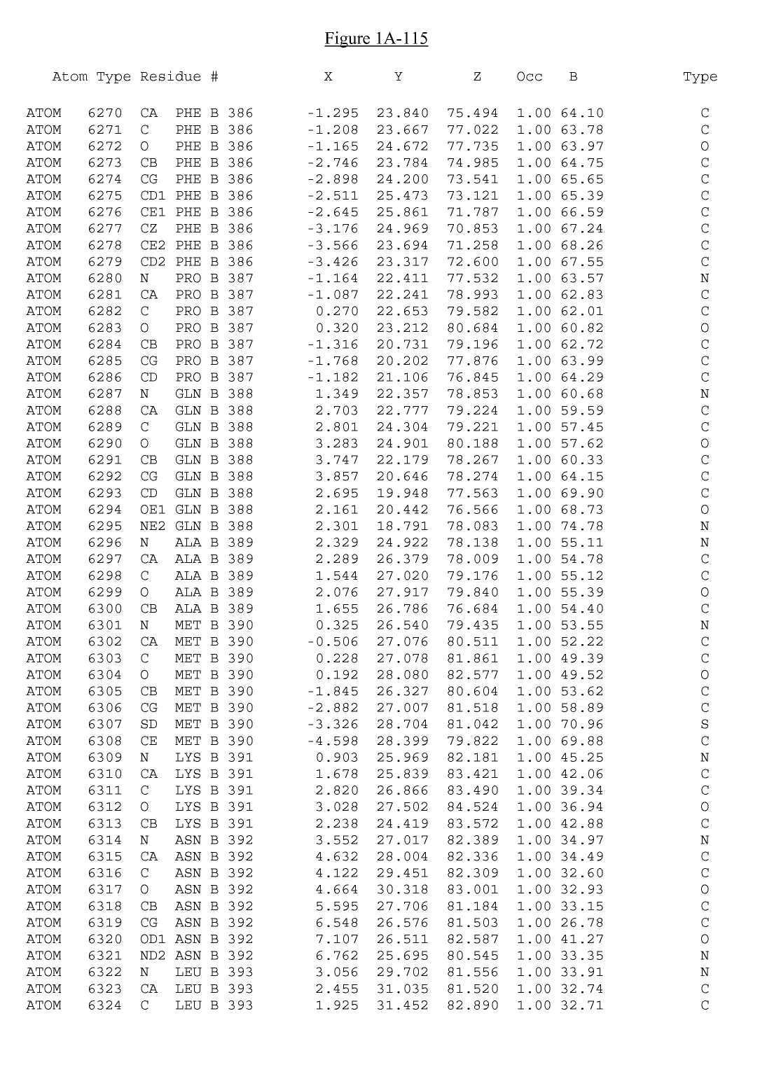

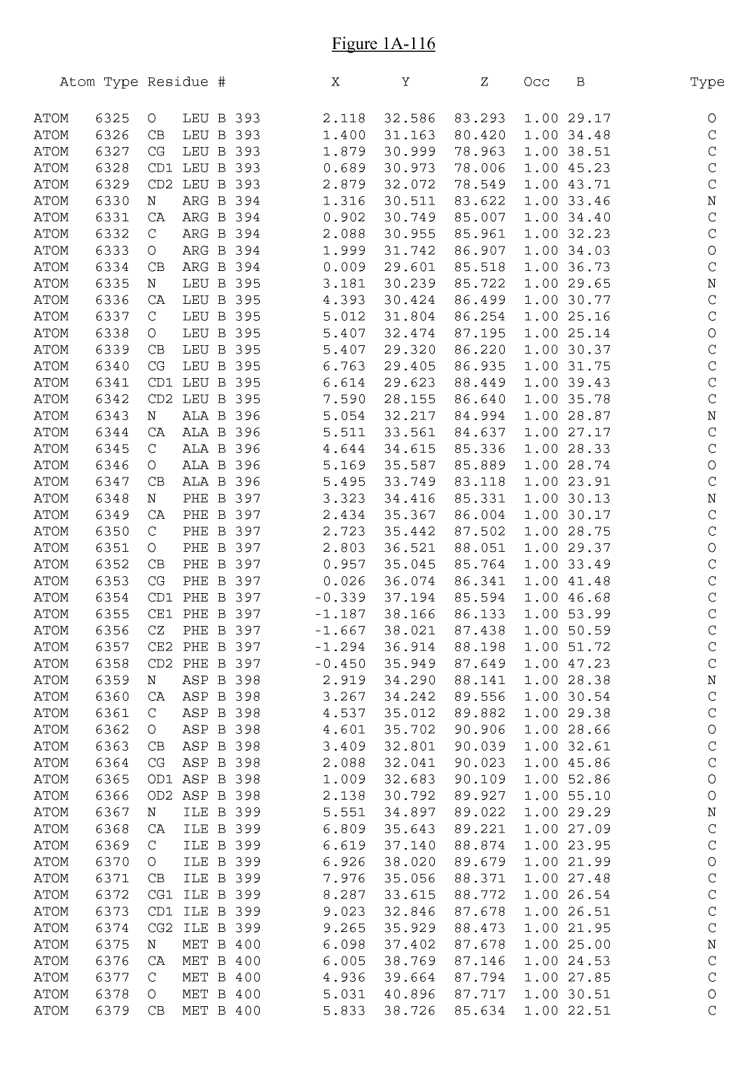

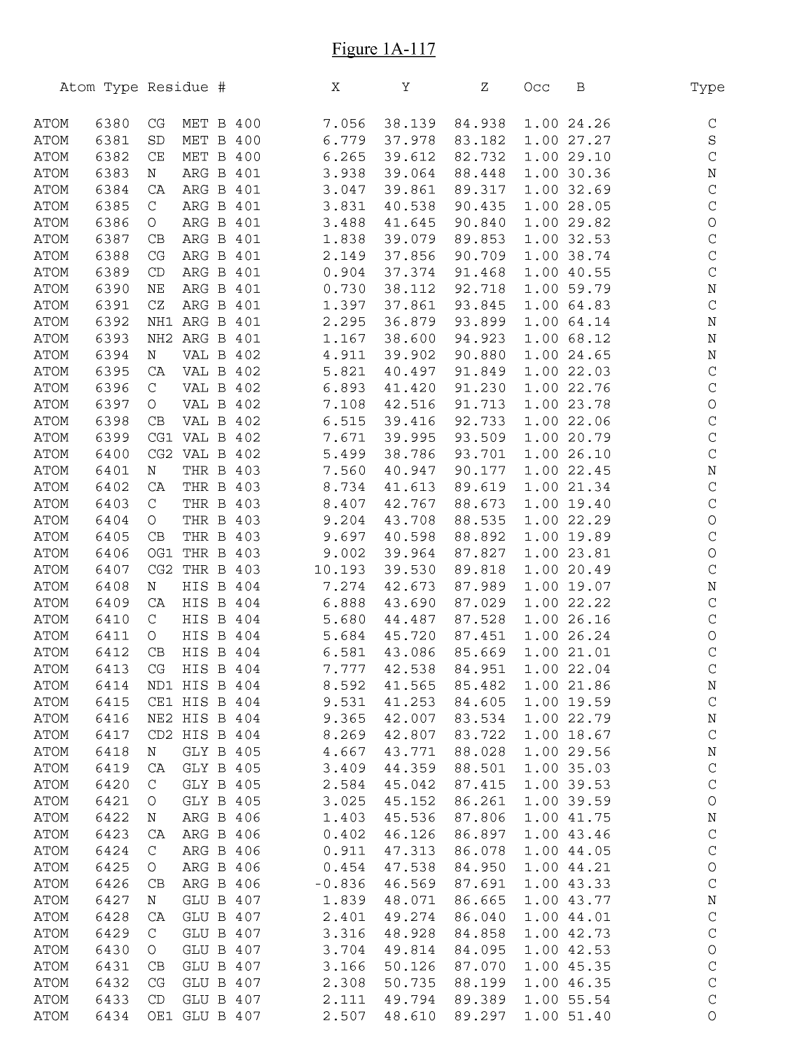

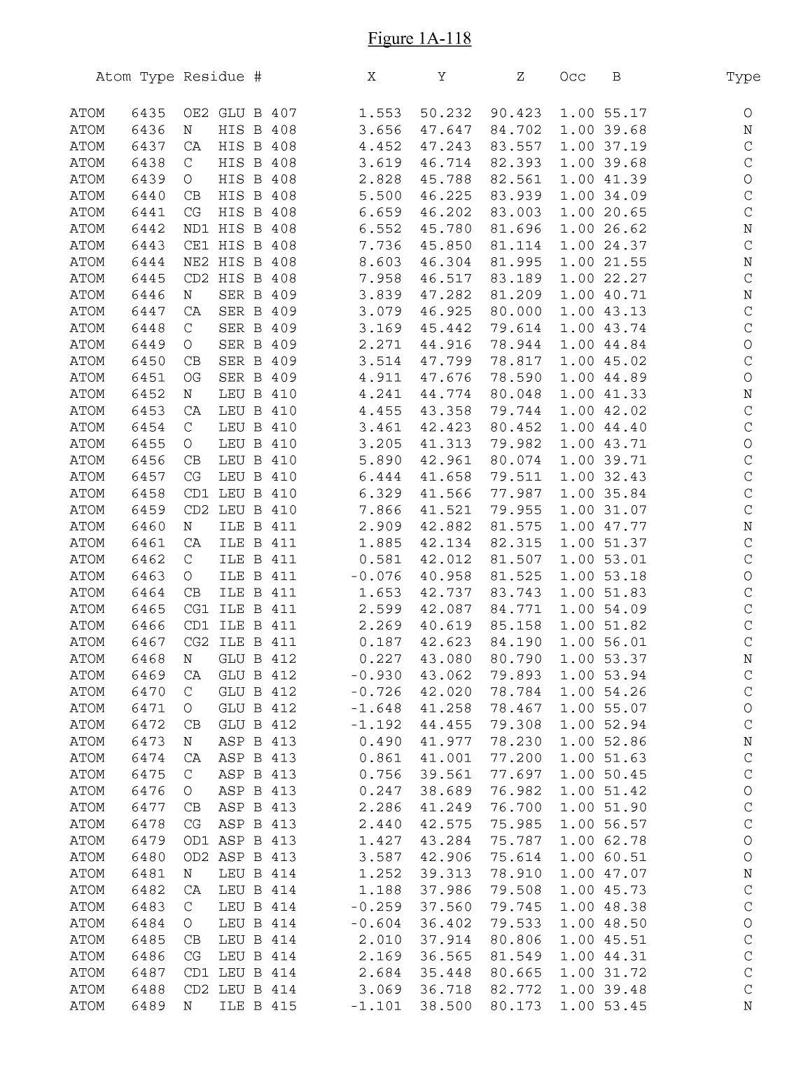

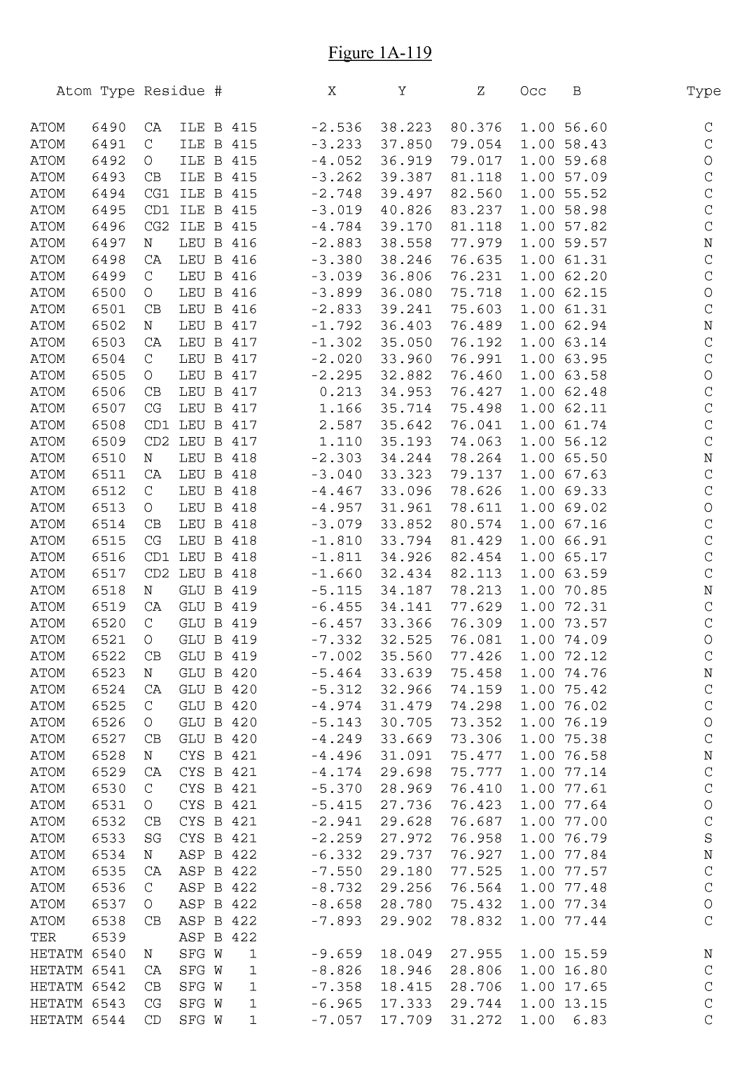

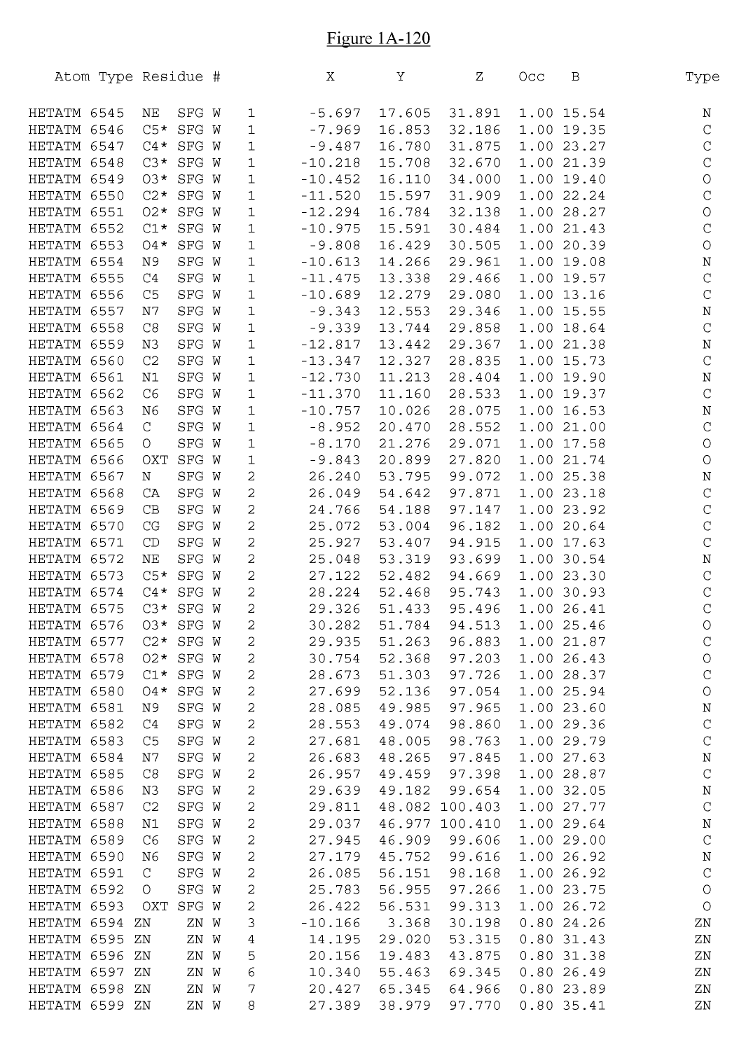

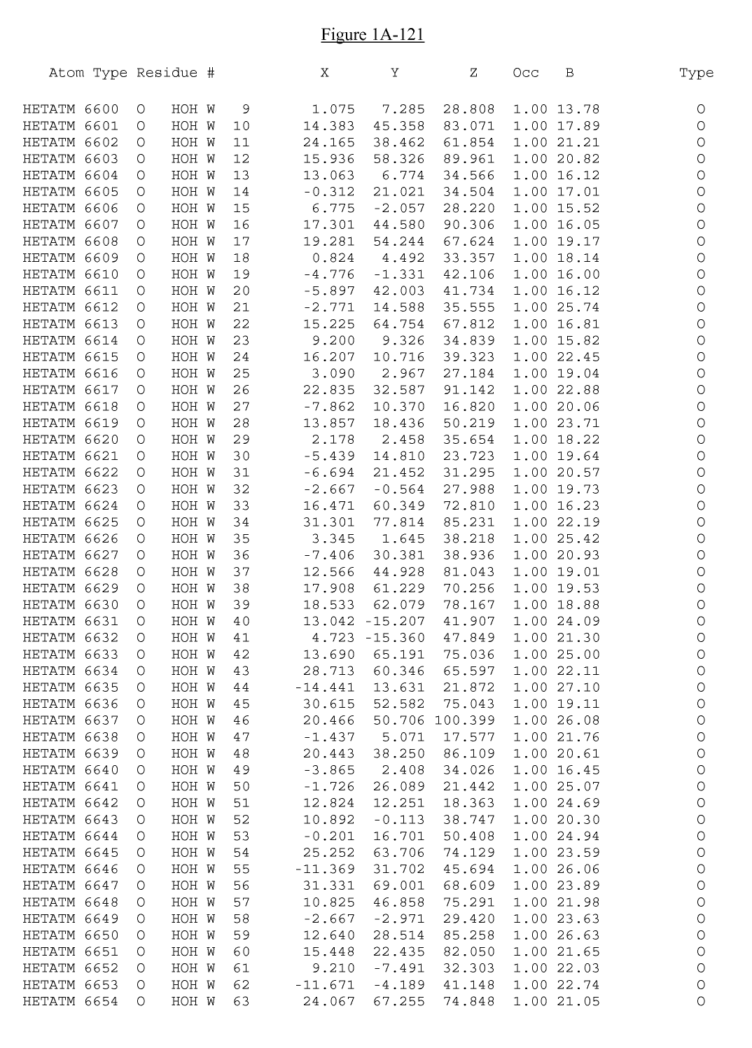

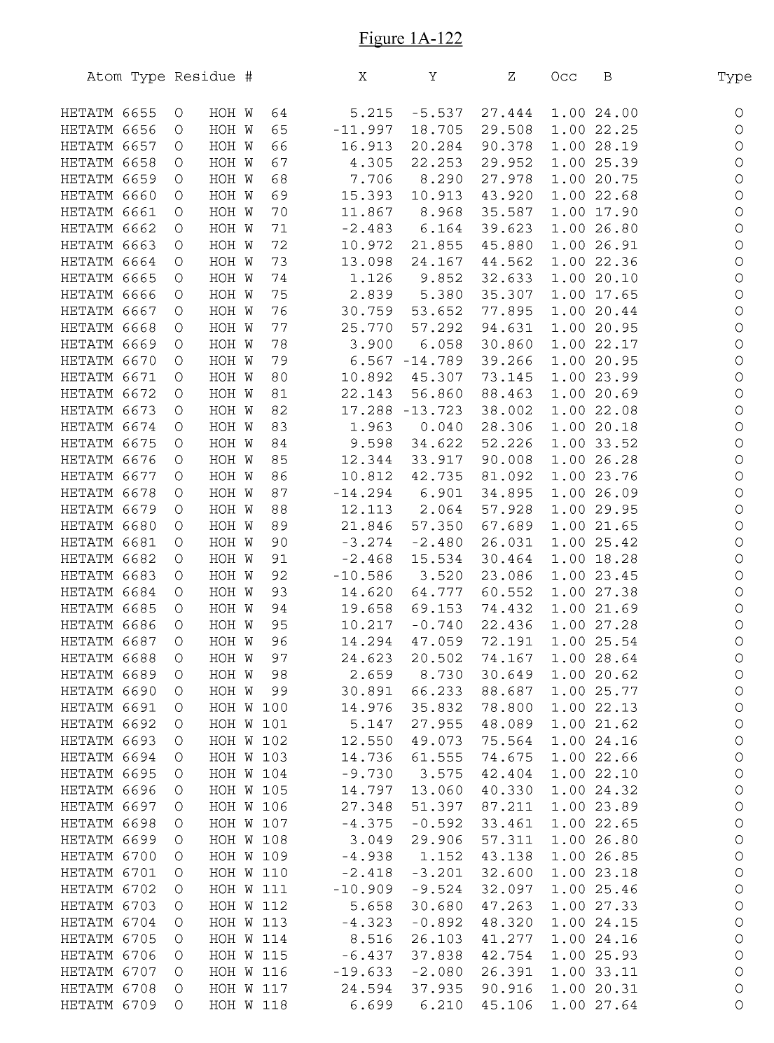

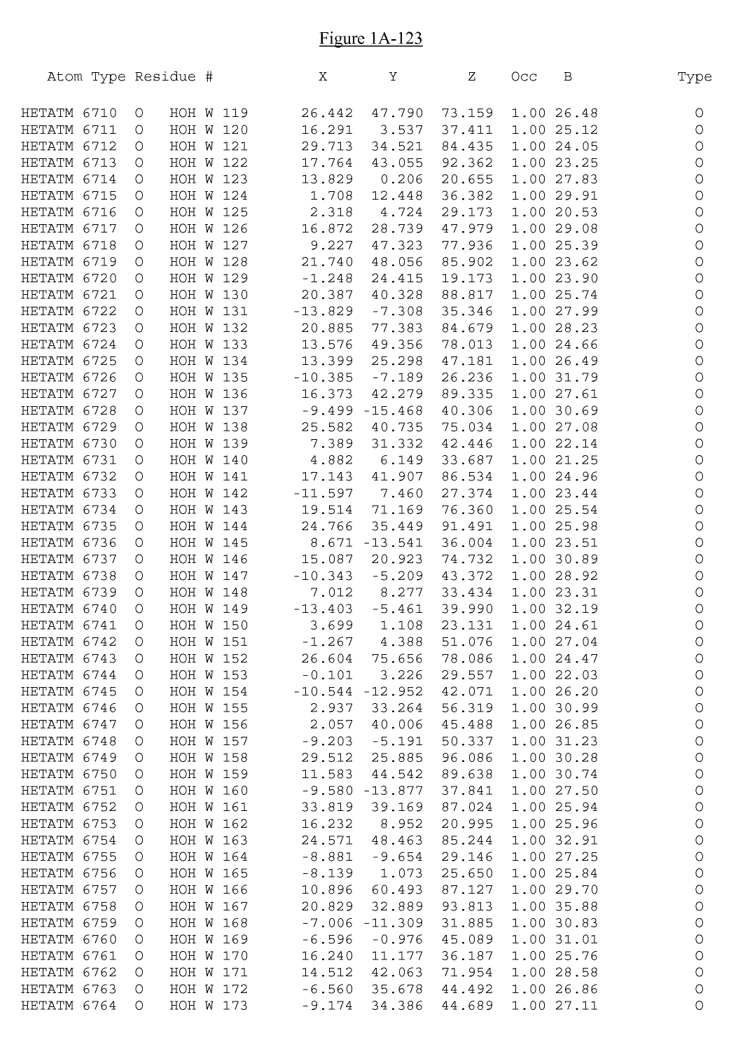

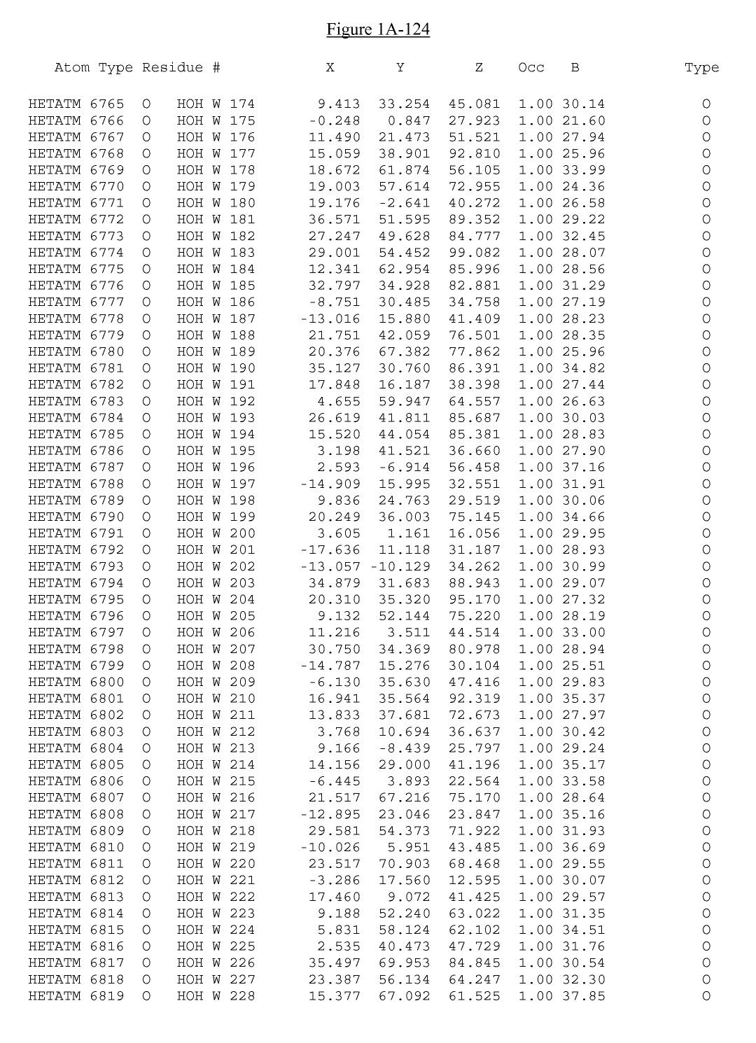

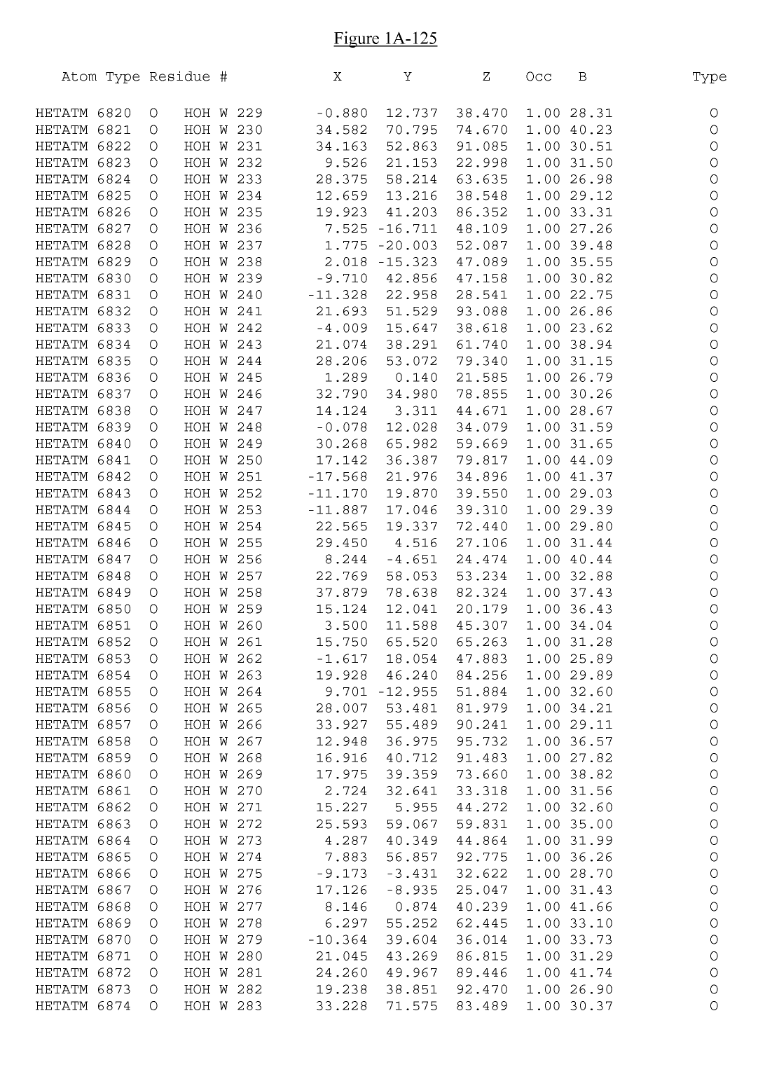

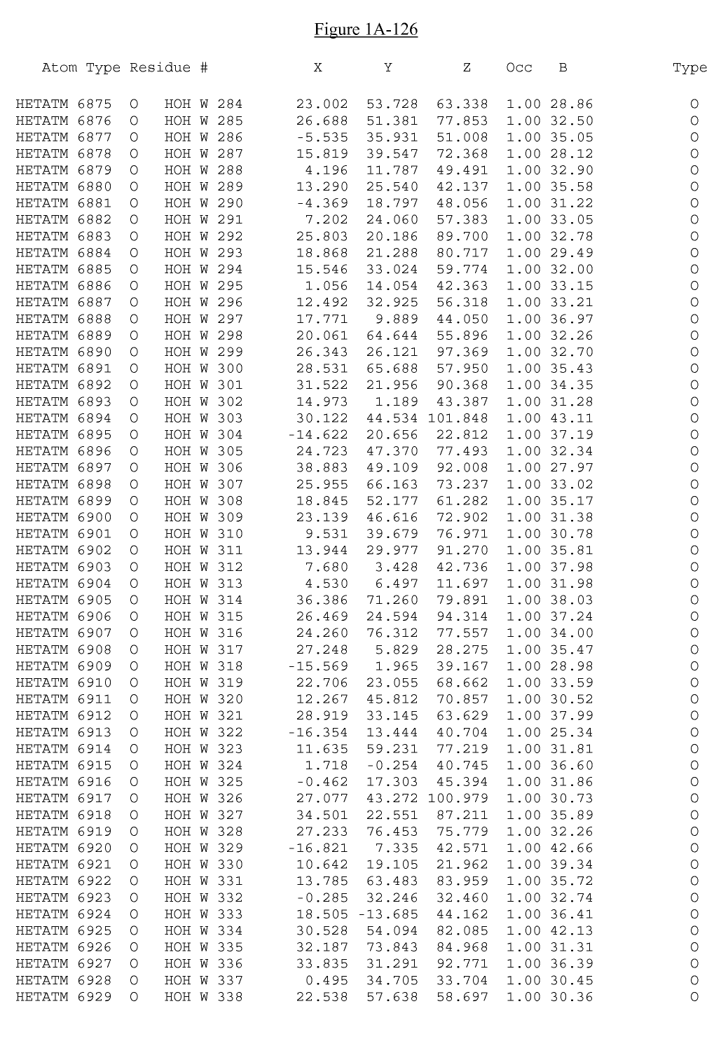

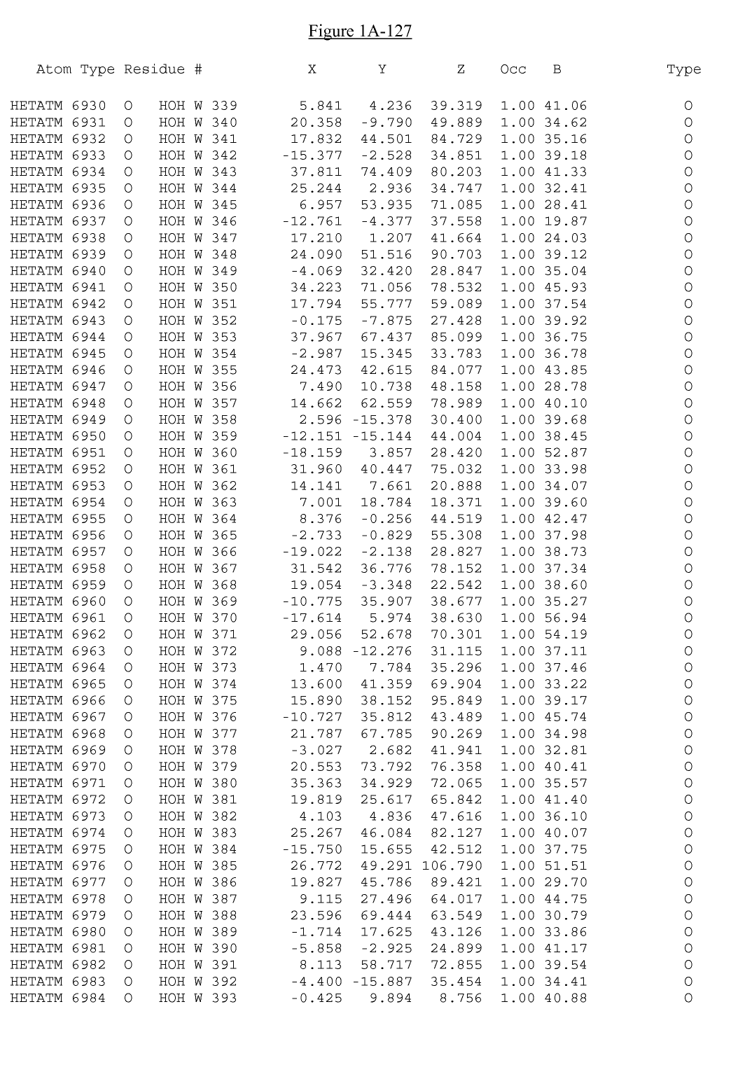

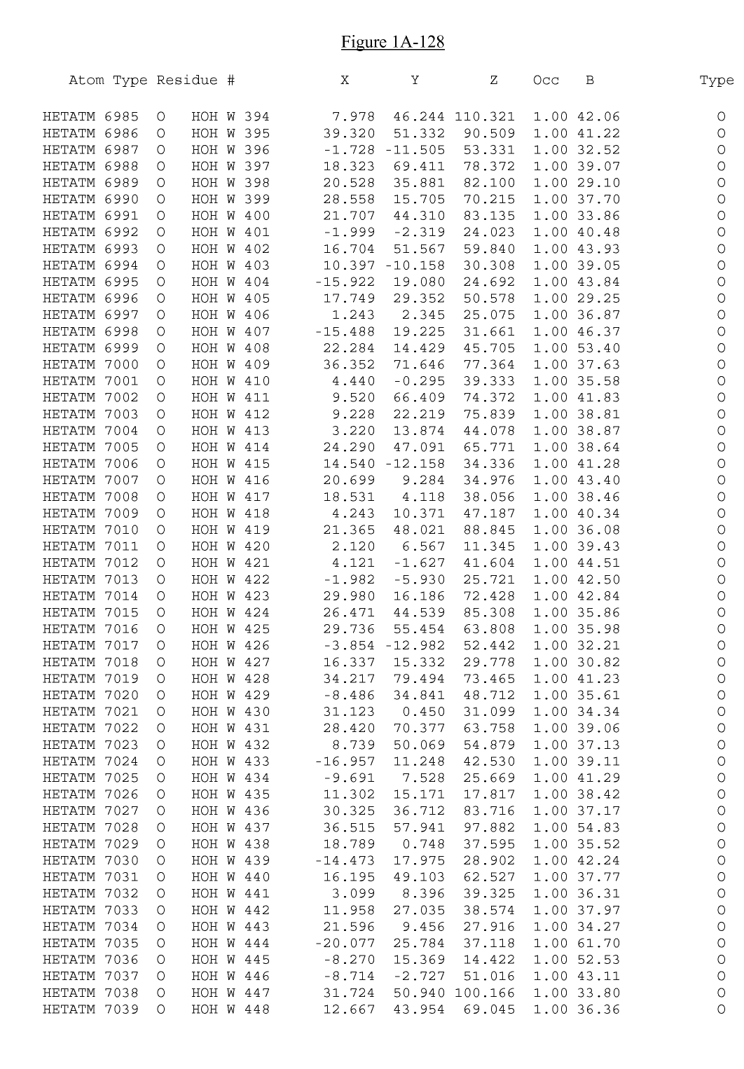

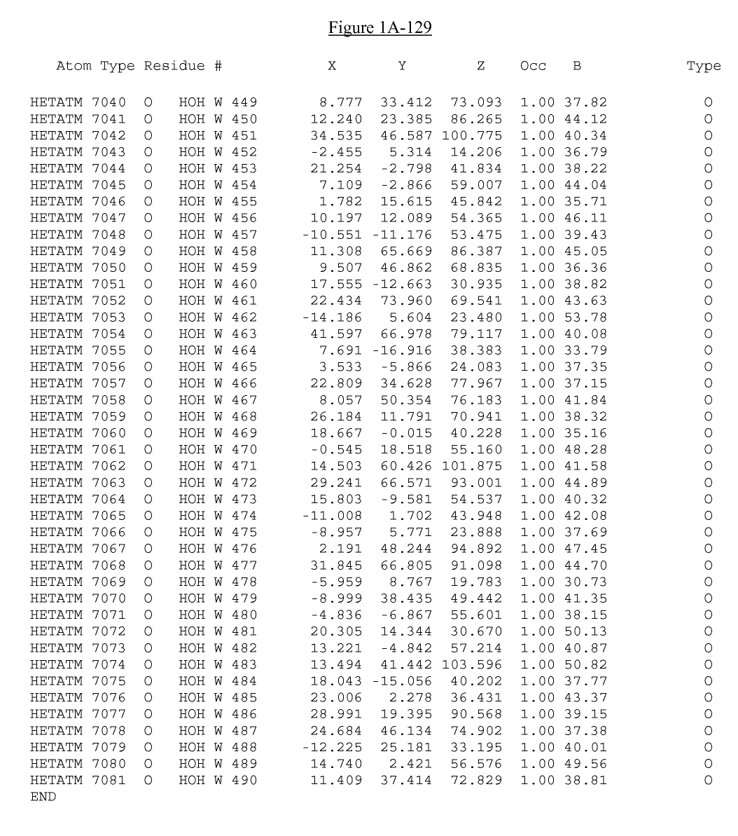

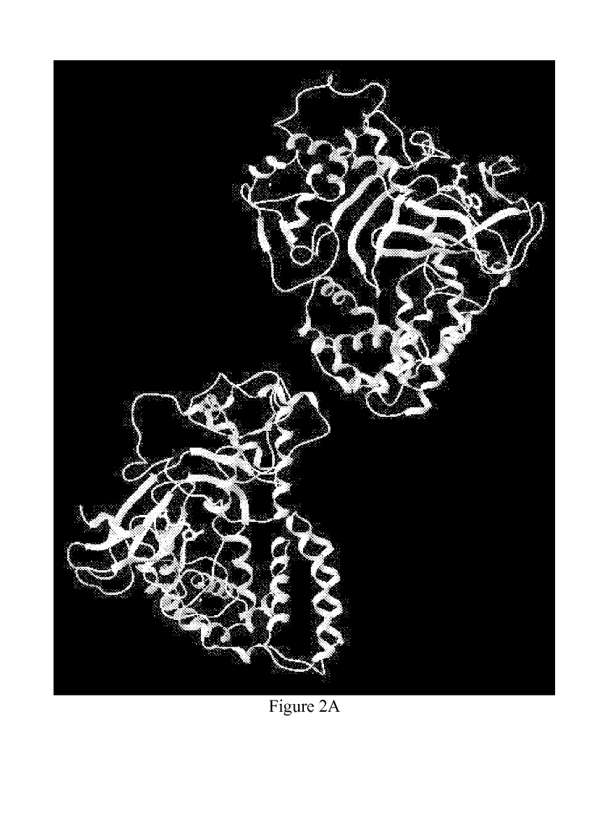

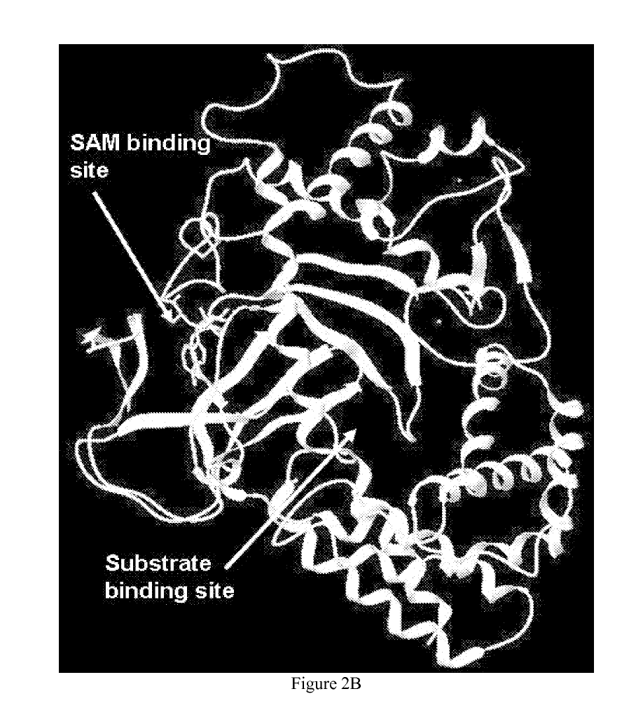

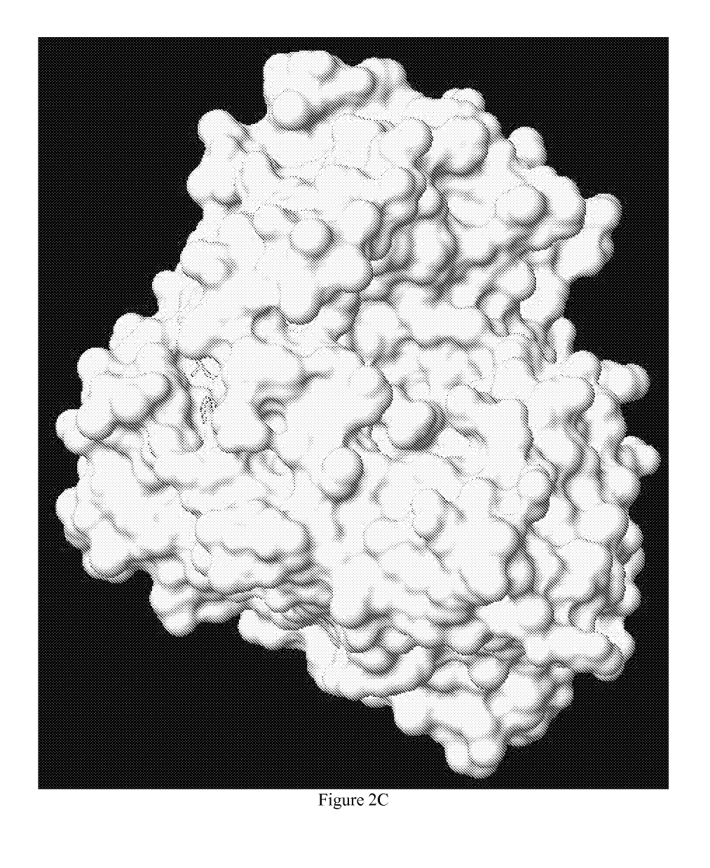

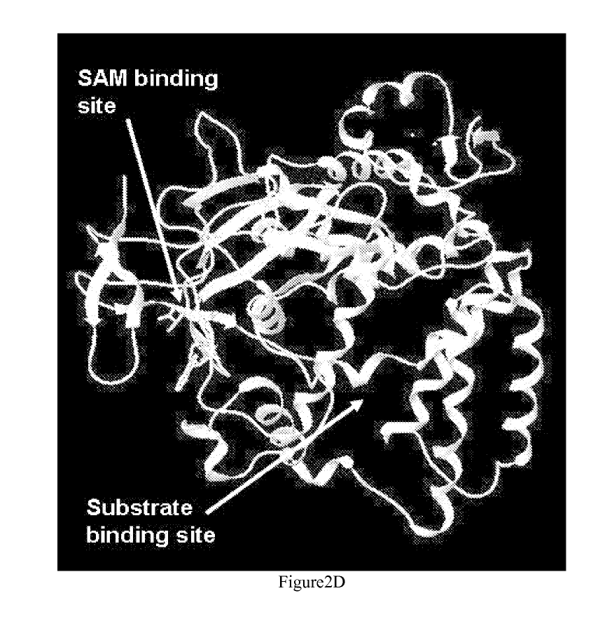

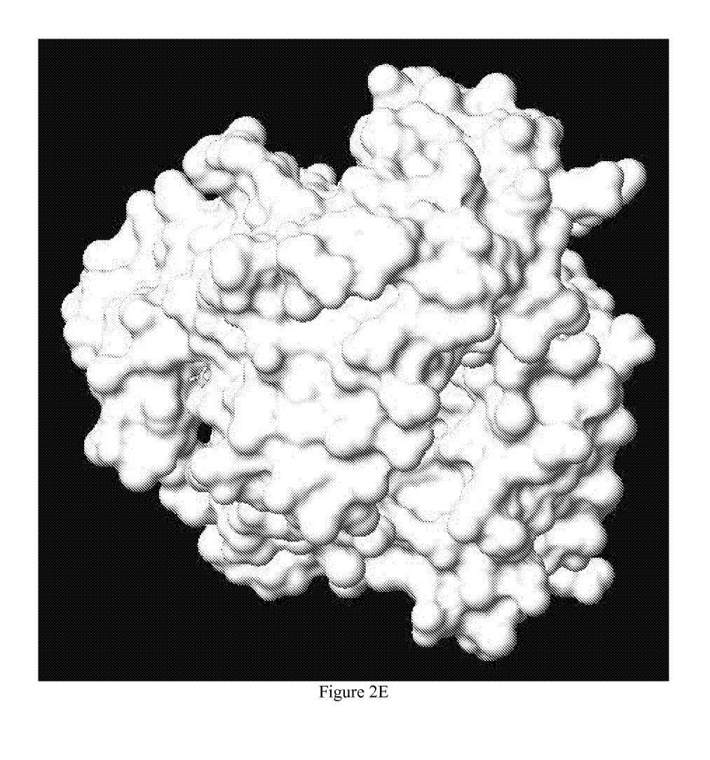

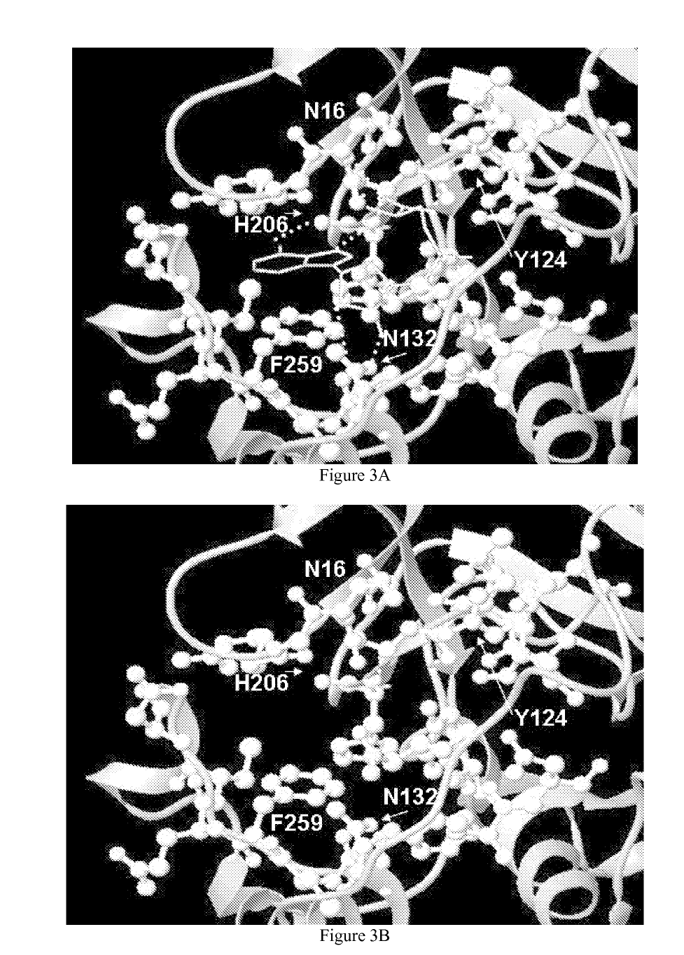

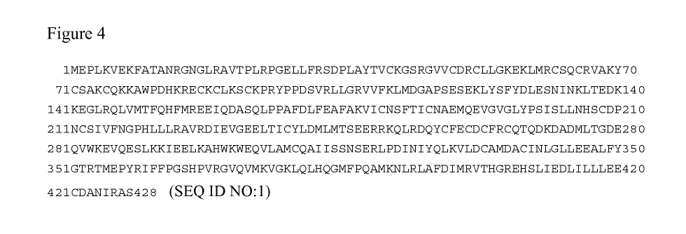

The term “contact score” refers to a measure of shape complementarity between the chemical entity and binding pocket, which is correlated with an RMSD value obtained from a least square superimposition between all or part of the atoms of the chemical entity and all or part of the atoms of the ligand bound (for example, SAM or some other binder) in the binding pocket according to FIG. 1 or 2. The docking process may be facilitated by the contact score or RMSD values. For example, if the chemical entity moves to an orientation with high RMSD, the system will resist the motion. A set of orientations of a chemical entity can be ranked by contact score. A lower RMSD value will give a higher contact score. See Meng et al. J. Comp. Chem., 4, 505-524 (1992).

The term “correspond to” or “corresponding amino acids”, when used in the context of the relationship between amino acid residues of any protein and SMYD3 amino acid residues, refers to particular amino acids or analogues thereof that align to amino acids in the human SMYD3 protein. Each of these amino acids may be an identical, mutated, chemically modified, conserved, conservatively substituted, functionally equivalent or homologous amino acid, when compared to the SMYD3 amino acid to which it could be aligned by those skilled in the art. For example, the following are examples of SMYD3 amino acid residues that correspond to SMYD1 amino acid residues: S182:G181 and A188:Q187 (the identity of the SMYD3 residue is listed first; its position is indicated using SMYD3 sequence numbering; and the identity of the SMYD1 residue is given at the end).

Methods for identifying a corresponding amino acid are known in the art and are based upon sequence, structural alignment, its functional position or a combination thereof, as compared to the SMYD3 protein. For example, corresponding amino acids may be identified by superimposing the backbone atoms of the amino acids in SMYD3 and another protein using well known software applications, such as QUANTA (Accelrys, Inc., San Diego, Calif. ©1998, 2000; Accelrys ©2001, 2002). The corresponding amino acids may also be identified using sequence alignment programs such as the “bestfit” program or CLUSTAL W Alignment Tool (Higgins D. G., et al., Methods Enzymol., 266: 383-402 (1996)).

The term “crystallization solution” refers to a solution which promotes crystallization comprising at least one agent, including a buffer, one or more salts, a precipitating agent, one or more detergents, sugars or organic compounds, lanthanide ions, a poly-ionic compound, and/or stabilizer.

The term “docking” refers to orienting, rotating, or translating a chemical entity in the binding pocket, domain, molecule or molecular complex or portion thereof based on distance geometry or energy. Docking may be performed by distance geometry methods that find sets of atoms of a chemical entity that match sets of sphere centers of the binding pocket, domain, molecule or molecular complex or portion thereof. See Meng et al. J. Comp. Chem., 4, 505-524 (1992). Sphere centers are generated by providing an extra radius of given length from the atoms (excluding hydrogen atoms) in the binding pocket, domain, molecule or molecular complex or portion thereof. Real-time interaction energy calculations, energy minimizations or rigid-body minimizations (Gschwend, et al., J. Mol. Recognition, 9:175-186 (1996)) can be performed during or after orientation of the chemical entity to facilitate docking. For example, interactive docking experiments can be designed to follow the path of least resistance. If the user in an interactive docking experiment makes a move to increase the energy, the system will resist that move. However, if that user makes a move to decrease energy, the system will favor that move by increased responsiveness. (Cohen, et al., J. Med. Chem. 33:889-894 (1990)). Docking can also be performed by combining a Monte Carlo search technique with rapid energy evaluation using molecular affinity potentials. See Goodsell and Olson, Proteins: Structure, Function and Genetics 8:195-202 (1990). Software programs that carry out docking functions include but are not limited to MATCHMOL (Cory et al., J. Mol. Graphics, 2, 39 (1984); MOLFIT (Redington, Comput. Chem., 16, 217 (1992)) and DOCK (Meng et al., supra). Other software, such as GLIDE (Sherman et al., Chem. Biol. Drug Des., 67, 83-84 (2006)) allow for the dynamic docking of a ligand to an “induced fit” conformation of a protein derived from the starting coordinates of a protein target by stripping back certain side chains near the binding site of the provided protein, docking into the stripped-back site, reintroducing the side chains, and relaxing the complex.

The term “domain” refers to a structural unit of the SMYD3 protein or homologue. The domain can comprise a binding pocket, a sequence or structural motif.

The term “full-length SMYD3” refers to the complete human SMYD3 protein, which includes an MYND domain and a SET domain (amino acid residues 1 to 428; GenBank accession no. AAH31010; SEQ ID NO:1). The protein includes an insert between the two domains not present in other members of the SMYD family.

The term “SMYD3-like” refers to all or a portion of a molecule or molecular complex that has a commonality of shape with all or a portion of the SMYD3 protein. For example, in the SMYD3-like SAM binding pocket, the commonality of shape is defined by a root mean square deviation of the structure coordinates of the backbone atoms between the amino acids in the SMYD3-like SAM binding pocket and the SMYD3 amino acids in the SMYD3 SAM binding pocket (as set forth in FIG. 1A). Compared to the amino acids of the SMYD3 binding pocket, the corresponding amino acid residues in the SMYD3-like binding pocket may or may not be identical. Depending on the set of SMYD3 amino acid residues that define the SMYD3 SAM binding pocket, one skilled in the art would be able to locate the corresponding amino acids that define a SMYD3-like binding pocket in a protein based on sequence or structural homology.

The term “SMYD3 protein complex” or “SMYD3 homologue complex” refers to a molecular complex formed by associating the SMYD3 protein or SMYD3 homologue with a chemical entity, for example, a ligand, a substrate, nucleotide amino acid, non-natural nucleotide amino acid, amino acid, an agonist or antagonist, binder, antibody, drug or compound.

The term “generating a three-dimensional structure” or “generating a three-dimensional representation” refers to converting the lists of structure coordinates into structural models or graphical representations in three-dimensional space. This can be achieved through commercially or publicly available software. A model of a three-dimensional structure of a molecule or molecular complex can thus be constructed on a computer screen by a computer that is given the structure coordinates and that comprises the correct software. The three-dimensional structure may be displayed or used to perform computer modeling or fitting operations. In addition, the structure coordinates themselves, without the displayed model, may be used to perform computer-based modeling and fitting operations.

The term “homologue of SMYD3 domain” or “SMYD3 domain homologue” refers to the domain of a protein that is at least 70%, 80%, 90%, 95%, 96%, 97%, 98%, 99% or greater than 99% identical in sequence to the corresponding domain of human SMYD3 protein and retains SMYD3 methyltransferase activity. In one embodiment, the homologue is at least 95%, 96%, 97%, 98% or 99% identical in sequence to the corresponding human SMYD3 domain, and has conservative mutations as compared to human SMYD3 domain. The homologue can be a SMYD3 domain from another species, or the foregoing human SMYD3 domain with mutations, conservative substitutions, additions, deletions or a combination thereof. Such animal species include, but are not limited to, mouse, rat, a primate such as monkey or other primates.

The term “homology model” refers to a structural model derived from known three-dimensional structure(s). Generation of the homology model, termed “homology modeling”, can include sequence alignment, residue replacement, residue conformation adjustment through energy minimization, or a combination thereof.

The term “interaction energy” refers to the energy determined for the interaction of a chemical entity and a binding pocket, domain, molecule or molecular complex or portion thereof. Interactions include but are not limited to one or more of covalent interactions, non-covalent interactions such as hydrogen bond, electrostatic, hydrophobic, aromatic, van der Waals interactions, and non-complementary electrostatic interactions such as repulsive charge-charge, dipole-dipole and charge-dipole interactions. As interaction energies are measured in negative values, the lower the value the more favorable the interaction.

The term “motif” refers to a group of amino acid residues in the SMYD3 protein or homologue that defines a structural compartment or carries out a function in the protein or homologue, for example, catalysis or structural stabilization, or methylation. The motif may be conserved in sequence, structure and function. The motif can be contiguous in primary sequence or three-dimensional space. An example of a motif includes but is not limited to the residues lining the SAM-binding site.

The term “part of a binding pocket” refers to less than all of the amino acid residues that define the binding pocket. The structure coordinates of amino acid residues that constitute part of a binding pocket may be specific for defining the chemical environment of the binding pocket, or useful in designing fragments of an binder that may interact with those residues. For example, the portion of amino acid residues may be key residues that play a role in ligand binding, or may be residues that are spatially related and define a three-dimensional compartment of the binding pocket The amino acid residues may be contiguous or non-contiguous in primary sequence. In one embodiment, part of the binding pocket has at least two amino acid residues, preferably at least three, eight, fourteen or fifteen amino acid residues.

The term “part of a SMYD3 protein” or “part of a SMYD3 homologue” refers to less than all of the amino acid residues of a SMYD3 protein or homologue. In one embodiment, part of the SMYD3 protein or homologue defines the binding pockets, domains, sub-domains, and motifs of the protein or homologue. The structure coordinates of amino acid residues that constitute part of a SMYD3 protein or homologue may be specific for defining the chemical environment of the protein, or useful in designing fragments of a binder that interacts with those residues. The portion of amino acid residues may also be spatially related residues that define a three-dimensional compartment of the binding pocket, motif, or domain. The amino acid residues may be contiguous or non-contiguous in primary sequence. For example, the portion of amino acid residues may be key residues that play a role in ligand or substrate binding, peptide binding, antibody binding, catalysis, structural stabilization or degradation.

The term “quantified association” refers to calculations of distance geometry and energy. Energy can include but is not limited to interaction energy, free energy and deformation energy. See Cohen, supra.

The term “root mean square deviation” or “RMSD” refers to the square root of the arithmetic mean of the squares of the deviations from the mean. It is a way to express the deviation or variation from a trend or object. For purposes of this invention, the “root mean square deviation” defines the variation in the backbone of a protein from the backbone of SMYD3, a binding pocket, a motif, a domain, or portion thereof, as defined by the structure coordinates of SMYD3 described herein. It would be readily apparent to those skilled in the art that the calculation of RMSD involves standard error of ±0.1 Å.

The term “soaked” refers to a process in which a crystal is transferred to a solution containing a compound of interest.

The term “structure coordinates” refers to Cartesian coordinates derived from mathematical equations related to the patterns obtained on diffraction of a monochromatic beam of X-rays by the atoms (scattering centers) of a protein or protein complex in crystal form. The diffraction data are used to calculate an electron density map of the repeating unit of the crystal. The electron density maps are then used to establish the positions of the individual atoms of the molecule or molecular complex.

The term “sub-domain” refers to a portion of a domain.

The term “substantially all of a SMYD3 binding pocket” or “substantially all of a SMYD3 protein” refers to all or almost all of the amino acids in the SMYD3 binding pocket or protein. For example, substantially all of a SMYD3 binding pocket can be 100%, 95%, 90%, 80%, or 70% of the residues defining the SMYD3 binding pocket or protein.

The term “substrate binding pocket” refers to the binding pocket for a substrate of SMYD3 or homologue thereof. A substrate is generally defined as the molecule upon which an enzyme performs catalysis. Natural substrates, synthetic substrates or peptides, or mimics of natural substrates of SMYD3 or homologue thereof may associate with the substrate binding pocket

The term “sufficiently homologous to SMYD3” refers to a protein that has a sequence identity of at least 25% compared to SMYD3 protein. In other embodiments, the sequence identity is at least 40%. In other embodiments, the sequence identity is at least 50%, 60%, 70%, 80%, 90%, 95%, 96%, 97%, 98% or 99%.

The term “three-dimensional structural information” refers to information obtained from the structure coordinates. Structural information generated can include the three-dimensional structure or graphical representation of the structure. Structural information can also be generated when subtracting distances between atoms in the structure coordinates, calculating chemical energies for a SMYD3 molecule or molecular complex or homologues thereof, calculating or minimizing energies for an association of a SMYD3 molecule or molecular complex, or homologues thereof to a chemical entity.

Crystallizable Compositions and Crystals of a SMYD3 Domain and Complexes Thereof

In one embodiment, the invention provides a crystallizable composition comprising a SMYD3 domain or its homologue. In another embodiment, the crystallizable composition further comprises a buffer that maintains pH between about 7.0 and 12.0 and 0.1-5 M magnesium chloride. In certain embodiments, the crystallizable composition comprises equal volumes of a solution of a SMYD3 domain or a homologue thereof (10 mg/ml) in the presence of 1 mM adenosyl-ornithine, 100 mM MgCl₂ hexahydrate, 17% PEG 20K, and 100 mM Tris HCl pH 8.5. In other embodiments, the crystallizable composition comprises equal volumes of a solution of a SMYD3 domain or a homologue thereof (10 mg/ml) in the presence of 1 mM adenosyl-ornithine, 200 mM MgCl₂, 16% PEG 3350, and 100 mM HEPES pH 7.5.

According to another embodiment, the invention provides a crystal comprising a SMYD3 domain or its homologue. Preferably, the native crystal has a unit cell dimension of a=58.2 Å, b=118.1 Å, c=82.9 Å and belongs to space group P_{1 21 1}. It will be readily apparent to those skilled in the art that the unit cells of such a crystal composition may deviate ±2% from the above cell dimensions depending on the deviation in the unit cell calculations.

As used herein, the SMYD3 domain in the crystallizable compositions or crystals can be amino acids X-Y of SEQ ID NO:1, where X=1, 2, or 7 and Y=419 or 428 of SEQ ID NO:1. The homologue thereof can be any of the aforementioned amino acids with conservative substitutions, deletions or additions, to the extent that any substitutions, deletions or additions maintains a SMYD3 methyltransferase activity in the homologue; preferably the homologue with substitutions, deletions or additions is at least 70%, 80%, 90%, 95%, 96%, 97%, 98%, or 99% identical to one of the aforementioned. Preferably, the homologue with substitutions, deletions or additions is at least 80%, 90%, 95%, 96%, 97%, 98%, or 99% identical to one of the aforementioned.

| (SEQ ID NO:1) | | |
|---|---|---|
| 1 | MEPLKVEKFATANRGNGLRAVTPLRPGELLFRSDPLAYTVCKGSRGVVCDRCLLGKEKLMRCSQCRVAKY | 70 |
| 71 | CSAKCQKKAWPDHKRECKCLKSCKPRYPPDSVRLLGRVVFKLMDGAPSESEKLYSFYDLESNINKLTEDK | 140 |
| 141 | KEGLRQLVMTFQHFMREEIQDASQLPPAFDLFEAFAKVICNSFTICNAEMQEVGVGLYPSISLLNHSCDP | 210 |
| 211 | NCSIVFNGPHLLLRAVRDIEVGEELTICYLDMLMTSEERRKQLRDQYCFECDCFRCQTQDKDADMLTGDE | 280 |
| 281 | QVWKEVQESLKKIEELKAHWKWEQVLAMCQAIISSNSERLPDINIYQLKVLDCAMDACINLGLLEEALFY | 350 |
| 351 | GTRTMEPYRIFFPGSHPVRGVQVMKVGKLQLHQGMFPQAMKNLRLAFDIMRVTHGREHSLIEDLILLLEE | 420 |
| 421 | CDANIRAS | 428 |

The SMYD3 protein or its homologue may be produced by any well-known method, including synthetic methods, such as solid phase, liquid phase and combination solid phase/liquid phase syntheses; recombinant DNA methods, including cDNA cloning, optionally combined with site directed mutagenesis; and/or purification of the natural products.

Methods of Obtaining Crystals of a SMYD3 Domain or its Homologues

The invention also relates to a method of obtaining a crystal of a SMYD3 domain or homologue thereof, comprising the steps of:

a) optionally producing and purifying a SMYD3 domain or homologue thereof;

b) combining a crystallization solution with said SMYD3 domain or homologue thereof to produce a crystallizable composition; and

c) subjecting the composition to conditions which promote crystallization and obtaining said crystal.

In another embodiment, the invention provides methods of obtaining crystals of a SMYD3 domain protein, a homologue thereof, or complexes thereof using the steps set forth above. In one embodiment, step (b) is performed with a SMYD3 domain or homologue thereof bound to a chemical entity. In another embodiment, the above method further comprises the step of soaking said crystal in a solution comprising a chemical entity that binds to the SMYD3 domain or homologue thereof.

In certain embodiments, the method of making crystals of a SMYD3 domain, a homologue, or a SMYD3 domain protein or homologue complex includes the use of a device for promoting crystallizations. Devices for promoting crystallization can include but are not limited to the hanging-drop, sitting-drop, sandwich-drop, dialysis, microbatch or microtube batch devices (U.S. Pat. Nos. 4,886,646, 5,096,676, 5,130,105, 5,221,410 and 5,400,741; Pav, S., et al., Proteins Struct. Funct. Genet, 20: 98-102 (1994); Chayen, Acta. Cryst., D54: 8-15 (1998), Chayen, Structure, 5: 1269-1274 (1997), D'Arcy et al., J. Cryst. Growth, 168: 175-180 (1996) and Chayen, J. Appl. Cryst., 30: 198-202 (1997), incorporated herein by reference). The hanging-drop, sitting-drop and some adaptations of the microbatch methods (D'Arcy et al., J. Cryst. Growth, 168: 175-180 (1996) and Chayen, J. Appl. Cryst., 30: 198-202 (1997)) produce crystals by vapor diffusion. The hanging drop and sitting drop containing the crystallizable composition is equilibrated against a reservoir containing a higher or lower concentration of precipitant. As the drop approaches equilibrium with the reservoir, the saturation of protein in the solution leads to the formation of crystals.

Microseeding may be used to increase the size and quality of crystals. In this instance, microcrystals are crushed to yield a stock seed solution. The stock seed solution is diluted in series. Using a needle, glass rod, micro-pipet, micro-loop or strand of hair, a small sample from each diluted solution is added to a set of equilibrated drops containing a protein concentration equal to or less than a concentration needed to create crystals without the presence of seeds. The aim is to end up with a single seed crystal that will act to nucleate crystal growth in the drop.

It would be readily apparent to one of skill in the art to vary the crystallization conditions disclosed above to identify other crystallization conditions that would produce crystals of SMYD3 protein, SMYD3 protein complex, SMYD3 domain protein complex or homologue thereof, or SMYD3 domain homologue. Such variations include, but are not limited to, adjusting pH, protein concentration and/or crystallization temperature, changing the identity or concentration of salt and/or precipitant used, using a different method for crystallization, or introducing additives such as detergents (e.g., TWEEN 20 (monolaurate), LDOA, Brji 30 (4 lauryl ether)), sugars (e.g., glucose, maltose), organic compounds (e.g., dioxane, dimethylformamide), lanthanide ions, or poly-ionic compounds that aid in crystallizations. High throughput crystallization assays may also be used to assist in finding or optimizing the crystallization condition.

In certain embodiments, the crystal comprising a domain of a SMYD3 methyltransferase protein or a homologue thereof diffract X-rays to a resolution of at least 1.5 Å. In other embodiments, the crystal comprising a domain of a SMYD3 domain, a homologue, or a SMYD3 domain protein or homologue complex diffract X-rays to a resolution of at least 5.0 Å, at least 3.5 Å, at least 2.5 Å, at least 2.0 Å, or at least 1.7 Å.

In certain embodiments, the crystal comprising a domain of a SMYD3 methyltransferase protein, a homologue thereof, or complexes thereof can produce an electron density map having resolution of at least 1.5 Å. In other embodiments, the crystal comprising a domain of a SMYD3 domain, a homologue, or a SMYD3 domain protein or homologue complex can produce an electron density map having resolution of at least 5.0 Å, at least 3.5 Å, at least 2.5 Å, at least 2.0 Å, or at least 1.7 Å.

In certain embodiments, the electron density map produced above is sufficient to determine the atomic coordinates a domain of a SMYD3 methyltransferase protein or a homologue thereof.

Binding Pockets of SMYD3 Protein or its Homologues

As disclosed herein, applicants have provided the first three-dimensional X-ray structure of SMYD3. The atomic coordinate data is presented in FIG. 1A.

To use the structure coordinates generated for the SMYD3 domain or one of its binding pockets or a SMYD3-like binding pocket, it may be necessary to convert the structure coordinates, or portions thereof, into a three-dimensional shape (i.e., a three-dimensional representation of these proteins and binding pockets). This is achieved through the use of a computer comprising commercially available software that is capable of generating three-dimensional representations or structures of molecules or molecular complexes, or portions thereof, from a set of structure coordinates. These three-dimensional representations may be displayed on a computer screen.

Binding pockets, also referred to as binding sites in the present invention, are of significant utility in fields such as drug discovery. The association of natural ligands or substrates with the binding pockets of their corresponding receptors or enzymes is the basis of many biological mechanisms of action. Similarly, many drugs exert their biological effects through association with the binding pockets of receptors and enzymes. Such associations may occur with all or part of the binding pocket. An understanding of such associations will help lead to the design of drugs having more favorable associations with their target receptor or enzyme, and thus, improved biological effects. Therefore, this information is valuable in designing potential binders of the binding pockets of biologically important targets. The binding pockets of this invention are useful and important for drug design.

The conformations of SMYD3 and other proteins at a particular amino acid site, along the polypeptide backbone, can be compared using well-known procedures for performing sequence alignments of the amino acids. Such sequence alignments allow for the equivalent sites on these proteins to be compared. Such methods for performing sequence alignment include, but are not limited to, the “bestfit” program and CLUSTAL W Alignment Tool, Higgins et al., supra.

The SAM binding pocket comprises the amino acid residues found within the near vicinity of the adenosyl-ornithine bound to SMYD3.

In one embodiment, the SAM binding pocket comprises amino acid residues T11, N13, R14, G15, N16, G17, Y124, D128, L129, E130, N132, K135, C180, N181, S202, L203, L204, N205, H206, S207, T236, Y239, Q256, Y257, C258, F259, E260, C261, D262, and C263, according to the structure of SMYD3 in FIG. 1A. The above-identified amino acid residues were within 5 Å (“5 Å sphere amino acids”) of the adenosyl-ornithine bound to SMYD3. These residues were identified using the program Sybyl (Tripos Associates, St. Louis, Mo.), which allow the display of the structure, and a software program to calculate the residues within 5 Å of adenosyl-ornithine bound to SMYD3. QUANTA (Accelrys ©2001, 2002), O (T. A. Jones et al., Acta Cryst., A47: 110-119 (1991)) and RIBBONS (Carson, J. Appl. Cryst., 24: 958-961 (1991)) may also be used to obtain the above residues.

In another embodiment, the SAM binding pocket comprises amino acids K8, F9, A10, T11, N13, R14, G15, N16, G17, L18, Y124, S125, D128, L129, E130, S131, N132, K135, L136, A176, K177, V178, I179, C180, N181, S182, F183, L197, Y198, P199, S200, I201, S202, L203, L204, N205, H206, S207, C208, D209, E234, L235, T236, I237, C238, Y239, Q252, L253, R254, D255, Q256, Y257, C258, F259, E260, C261, D262, C263, and C266 according to the structure of SMYD3 protein in FIG. 1A. These amino acid residues were within 8 Å (“8 Å sphere amino acids”) of the adenosyl-ornithine bound to SMYD3. These residues were identified using the program Sybyl (Tripos Associates, St. Louis, Mo.). QUANTA, O and RIBBONS, supra may also be used to obtain the above residues.

In another embodiment, the SAM binding pocket comprises amino acid residues R14, G15, N16, G17, Y124, E130, N132, K135, C180, N181, S202, L203, L204, N205, H206, Y239, Y257, F259, C261, and D262, according to the structure of SMYD3 protein in FIG. 1A. These amino acid residues are within 3.8 Å of the adenosyl-ornithine bound to SMYD3. These residues were identified using the program Sybyl (Tripos Associates, St. Louis, Mo.).

In another embodiment, the SAM binding pocket comprises amino acids R14, N16, Y124, E130, N132, N181, N205, H206, and F259 according to the structure of SMYD3 protein in FIG. 1A. These amino acid residues make contacts less than 3.8 Å in length with adenosyl-ornithine bound to SMYD3 (F259 makes primarily hydrophobic interactions or van der Waals contacts; and R14, N16, Y124, E130, N132, N181, N205, and H206 form direct or indirect hydrogen bonds). These residues were identified using the program Sybyl (Tripos Associates, St. Louis, Mo.).

In another embodiment, the SAM binding pocket comprises amino acids K135, C180, S202, L203, L204, Y239, Y257, C261, and D262 according to the structure of SMYD3 protein in FIG. 1A.

In another embodiment, the SAM binding pocket comprises amino acids I179, S182, F183, S202, I214, F216, L223, I237, Y239, L240, Q252, Y257, according to the structure of SMYD3 protein in FIG. 1A.

In another embodiment, the SAM binding pocket comprises amino acids R14, N132, Y124, and N205 according to the structure of SMYD3 protein in FIG. 1A.

It will be readily apparent to those of skill in the art that the numbering of amino acid residues in homologues of human SMYD3 may be different than that set forth for human SMYD3. Corresponding amino acid residues in homologues of SMYD3 are easily identified by visual inspection of the amino acid sequences or by using commercially available homology software programs. Homologues of SMYD3 include, for example, SMYD3 from other species, such as non-humans primates, mouse, rat, etc.

Those of skill in the art understand that a set of structure coordinates for an enzyme or an enzyme-complex, or a portion thereof, is a relative set of points that define a shape in three dimensions. Thus, it is possible that an entirely different set of coordinates could define a similar or identical shape. Moreover, slight variations in the individual coordinates will have little effect on overall shape. In terms of binding pockets, these variations would not be expected to significantly alter the nature of ligands that could associate with those pockets.

The variations in coordinates discussed above may be generated because of mathematical manipulations of the SMYD3 structure coordinates. For example, the structure coordinates set forth in FIG. 1A could undergo crystallographic permutations, fractionalization, integer additions or subtractions, inversion, or any combination of the above.

Alternatively, modifications in the crystal structure due to mutations, additions, substitutions, and/or deletions of amino acids, or other changes in any of the components that make up the crystal could also account for variations in structure coordinates. If such variations are within a certain root mean square deviation as compared to the original coordinates, the resulting three-dimensional shape is considered encompassed by this invention. Thus, for example, a ligand that bound to the binding pocket of SMYD3 would also be expected to bind to another binding pocket whose structure coordinates defined a shape that fell within the acceptable root mean square deviation.

Various computational analyses may be necessary to determine whether a molecule or the binding pocket or portion thereof is sufficiently similar to the SMYD3 binding pockets described above. Such analyses may be carried out using well known software applications, such as ProFit (A.C.R. Martin, SciTech Software, ProFit version 1.8, University College London, http://www.bioinf.org.uk/software), Swiss-Pdb Viewer (Guex et al., Electrophoresis, 18: 2714-2723 (1997)), the Molecular Similarity application of QUANTA (Accelrys, Inc., San Diego, Calif. ©1998, 2000; Accelrys ©2001, 2002) and as described in the accompanying User's Guide, which are incorporated herein by reference.

The above programs permit comparisons between different structures, different conformations of the same structure, and different parts of the same structure. The procedure used in QUANTA (Accelrys, Inc., San Diego, Calif. ©1998, 2000; Accelrys ©2001, 2002) and Swiss-Pdb Viewer to compare structures is divided into four steps: 1) load the structures to be compared; 2) define the atom equivalences in these structures; 3) perform a fitting operation on the structures; and 4) analyze the results.

The procedure used in ProFit to compare structures includes the following steps: 1) load the structures to be compared; 2) specify selected residues of interest; 3) define the atom equivalences in the selected residues; 4) perform a fitting operation on the selected residues; and 5) analyze the results.

Each structure in the comparison is identified by a name. One structure is identified as the target (i.e., the fixed structure); all remaining structures are working structures (i.e., moving structures). Since atom equivalency within QUANTA (Accelrys ©2001, 2002) is defined by user input, for the purpose of this invention we will define equivalent atoms as protein backbone atoms N, C, O and Cα for all corresponding amino acids between the two structures being compared.

The corresponding amino acids may be identified by sequence alignment programs such as the “bestfit” program available from the Genetics Computer Group which uses the local homology algorithm described by Smith and Waterman in Advances in Applied Mathematics 2, 482-489 (1981), which is incorporated herein by reference. A suitable amino acid sequence alignment will require that the proteins being aligned share a minimum percentage of identical amino acids. Generally, a first protein being aligned with a second protein should share in excess of about 35% identical amino acids (Hanks, S. K., et al., Science, 241, 42-52 (1988); Hanks, S. K. and Quinn, A. M. Methods in Enzymology, 200: 38-62 (1991)). The identification of equivalent residues can also be assisted by secondary structure alignment, for example, aligning the α-helices, β-sheets in the structure. The program Swiss-Pdb Viewer has its own best fit algorithm that is based on secondary sequence alignment.

When a rigid fitting method is used, the working structure is translated and rotated to obtain an optimum fit with the target structure. The fitting operation uses an algorithm that computes the optimum translation and rotation to be applied to the moving structure, such that the root mean square difference of the fit over the specified pairs of equivalent atom is an absolute minimum. This number, given in angstroms, is reported by the above programs. The Swiss-Pdb Viewer program sets an RMSD cutoff for eliminating pairs of equivalent atoms that have high RMSD values. An RMSD cutoff value can be used to exclude pairs of equivalent atoms with extreme individual RMSD values. In the program ProFit, the RMSD cutoff value can be specified by the user.

For the purpose of this invention, any molecule, molecular complex, binding pocket, motif, domain thereof or portion thereof that is within a root mean square deviation for backbone atoms (N, Cα, C, O) when superimposed on the relevant backbone atoms described by structure coordinates listed in FIG. 1A are encompassed by this invention.

One embodiment of this invention provides a crystalline molecule comprising a protein defined by structure coordinates of a set of amino acid residues that are identical to SMYD3 amino acid residues according to FIG. 1A, wherein the RMSD between said set of amino acid residues and said SMYD3 amino acid residues is not more than about 5.0 Å. In other embodiments, the RMSD between said set of amino acid residues and said SMYD3 amino acid residues is not greater than about 4.0 Å, not greater than about 3.0 Å, not greater than about 2.0 Å, not greater than about 1.5 Å, not greater than about 1.0 Å, or not greater than about 0.5 Å.

In one embodiment, the present invention provides a crystalline molecule comprising all or part of a binding pocket defined by a set of amino acid residues comprising at least six amino acid residues which are identical to human SMYD3 amino acid residues R14, G15, N16, G17, Y124, E130, N132, K135, C180, N181, S182, F183, T184, I201, S202, L203, L204, N205, H206, S207, C208, I214, I237, C238, Y239, L240, D241, R249, L253, Q256, Y257, F259, C261, D262, C263, R265, C266 according to FIG. 1A, wherein the RMSD of the backbone atoms between said SMYD3 amino acid residues and said at least six amino acid residues which are identical is not greater than about 3.0 Å. In other embodiments, the RMSD is not greater than about 2.0 Å, 1.0 Å, 0.8, 0.5 Å, 0.3 Å, or 0.2 Å. In other embodiments, the binding pocket is defined by a set of amino acid residues comprising at least four, six, eight, twelve, or fifteen amino acid residues which are identical to said SMYD3 amino acid residues.

In one embodiment, the present invention provides a crystalline molecule comprising all or part of a binding pocket defined by a set of amino acid residues which are identical to human SMYD3 amino acid residues R14, N16, Y124, E130, N132, N181, N205, H206, and F259 according to FIG. 1A, wherein the RMSD of the backbone atoms between said SMYD3 amino acid residues and said set of amino acid residues which are identical is not greater than about 3.0 Å. In other embodiments, the RMSD is not greater than about 2.0 Å, 1.0 Å, 0.8, 0.5 Å, 0.3 Å, or 0.2 Å. In other embodiments, the binding pocket is defined by a set of amino acid residues comprising at least four, five, six, or seven amino acid residues identical to said SMYD3 amino acid residues.

In one embodiment, the present invention provides a crystalline molecule comprising all or part of a binding pocket defined by a set of amino acid residues comprising a set of amino acid residues which are identical to human SMYD3 amino acid residues R14, N132, Y124, and N205 according to FIG. 1A, wherein the RMSD of the backbone atoms between said SMYD3 amino acid residues and said set of amino acid residues which are identical is not greater than about 3.0 Å. In other embodiments, the RMSD is not greater than about 2.0 Å, 1.0 Å, 0.8, 0.5 Å, 0.3 Å, or 0.2 Å.

In one embodiment, the above molecule is SMYD3 protein, SMYD3 domain or homologues thereof. In another embodiment, the above molecules are in crystalline form. A SMYD3 protein may be human SMYD3. Homologues of human SMYD3 can be SMYD3 from another species, such as a mouse, a rat or a non-human primate.

Computer Systems

According to another embodiment, this invention provides a machine-readable data storage medium, comprising a data storage material encoded with machine-readable data, wherein said data defines the above-mentioned molecules or molecular complexes or binding pockets thereof. In one embodiment, the data defines the above-mentioned binding pockets by comprising the structure coordinates of said amino acid residues according to FIG. 1A. To use the structure coordinates generated for SMYD3, homologues thereof, or one of its binding pockets, it is at times necessary to convert them into a three-dimensional shape or to extract three-dimensional structural information from them. This is achieved through the use of commercially or publicly available software that is capable of generating a three-dimensional structure or a three-dimensional representation of molecules or portions thereof from a set of structure coordinates. In one embodiment, three-dimensional structure or representation may be displayed graphically.

Therefore, according to another embodiment, this invention provides a machine-readable data storage medium comprising a data storage material encoded with machine-readable data. In one embodiment, a machine programmed with instructions for using said data is capable of generating a three-dimensional structure or three-dimensional representation of any of the molecules, or molecular complexes or binding pockets thereof, which are described herein.

This invention also provides a computer comprising:

(a) a machine-readable data storage medium, comprising a data storage material encoded with machine-readable data, wherein said data defines any one of the above molecules or molecular complexes;

(b) a working memory for storing instructions for processing said machine-readable data;

(c) a central processing unit (CPU) coupled to said working memory and to said machine-readable data storage medium for processing said machine readable data and means for generating three-dimensional structural information of said molecule or molecular complex; and

(d) output hardware coupled to said central processing unit for outputting three-dimensional structural information of said molecule or molecular complex, or information produced by using said three-dimensional structural information of said molecule or molecular complex.

In one embodiment, the data defines the binding pocket of the molecule or molecular complex.

Three-dimensional data generation may be provided by an instruction or set of instructions, such as a computer program or commands for generating a three-dimensional structure or graphical representation from structure coordinates, or by subtracting distances between atoms, calculating chemical energies for a SMYD3 molecule or molecular complex or homologues thereof, or calculating or minimizing energies for an association of a SMYD3 molecule or molecular complex or homologues thereof to a chemical entity. The graphical representation can be generated or displayed by commercially available software programs. Examples of software programs include but are not limited to QUANTA (Accelrys ©2001, 2002), O (Jones et al., Acta Crystallogr. A47: 110-119 (1991)) and RIBBONS (Carson, J. Appl. Crystallogr., 24: 9589-961 (1991)), which are incorporated herein by reference. Certain software programs may imbue this representation with physico-chemical attributes which are known from the chemical composition of the molecule, such as residue charge, hydrophobicity, torsional and rotational degrees of freedom for the residue or segment, etc. Examples of software programs for calculating chemical energies are described in the Rational Drug Design section.

Information about said binding pocket or information produced by using said binding pocket can be outputted through display terminals, touchscreens, facsimile machines, modems, CD-ROMs, printers, a CD or DVD recorder, ZIP™ or JAZ™ drives or disk drives. The information can be in graphical or alphanumeric form.

In one embodiment, the computer is executing an instruction such as a computer program for generating three-dimensional structure or docking. In another embodiment, the computer further comprises a commercially available software program to display the information as a graphical representation. Examples of software programs include but as not limited to, QUANTA (Accelrys ©2001, 2002), O (Jones et al., Acta Crystallogr. A47: 110-119 (1991)) and RIBBONS (Carson, J. Appl. Crystallogr., 24: 9589-961 (1991)), all of which are incorporated herein by reference.

FIG. 5 demonstrates one version of these embodiments. System (10) includes a computer (11) comprising a central processing unit (“CPU”) (20), a working memory (22) which may be, e.g., RAM (random-access memory) or “core” memory, mass storage memory (24) (such as one or more disk drives, CD-ROM drives or DVD-ROM drives), one or more cathode-ray tube (“CRT”) display terminals (26), one or more keyboards (28), one or more input lines (30), and one or more output lines (40), all of which are, interconnected by a conventional bi-directional system bus (50).

Input hardware (35), coupled to computer (11) by input lines (30), may be implemented in a variety of ways. Machine-readable data of this invention may be inputted via the use of a modem or modems (32) connected by a telephone line or dedicated data line (34). Alternatively or additionally, the input hardware (35) may comprise CD-ROM or DVD-ROM drives or disk drives (24). In conjunction with display terminal (26), keyboard (28) may also be used as an input device.

Output hardware (46), coupled to computer (11) by output lines (40), may similarly be implemented by conventional devices. By way of example, output hardware (46) may include CRT display terminal (26) for displaying a graphical representation of a binding pocket of this invention using a program such as QUANTA (Accelrys ©2001, 2002) as described herein. Output hardware may also include a printer (42), so that hard copy output may be produced, or a disk drive (24), to store system output for later use. Output hardware may also include a display terminal, touchscreens, facsimile machines, modems, a CD or DVD recorder, ZIP™ or JAZ™ drives, disk drives, or other machine-readable data storage device.

In operation, CPU (20) coordinates the use of the various input and output devices (35), (46), coordinates data accesses from mass storage (24) and accesses to and from working memory (22), and determines the sequence of data processing steps. A number of programs may be used to process the machine-readable data of this invention. Such programs are discussed in reference to the computational methods of drug discovery as described herein. Specific references to components of the hardware system (10) are included as appropriate throughout the following description of the data storage medium.

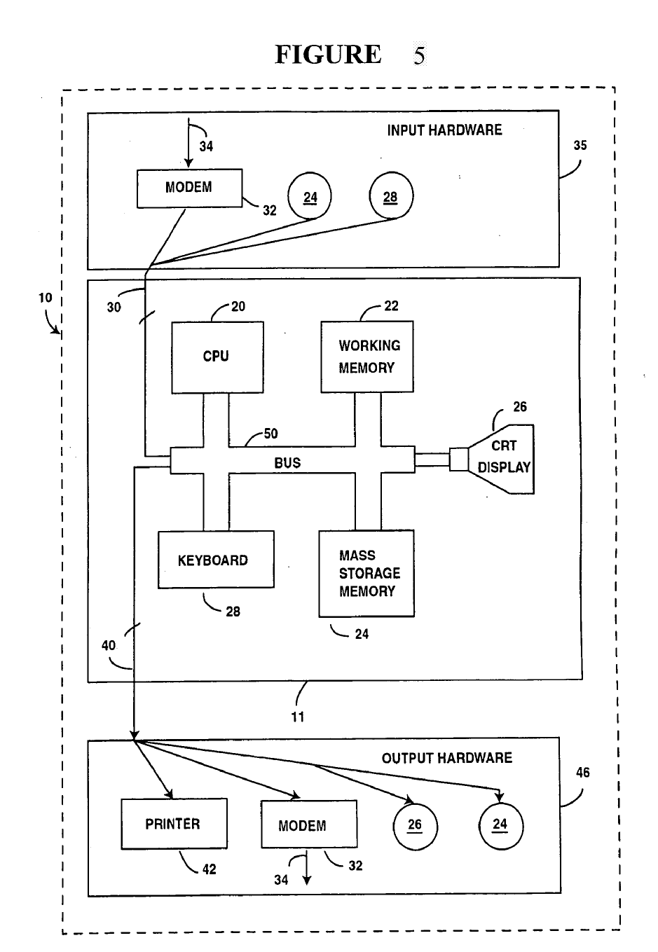

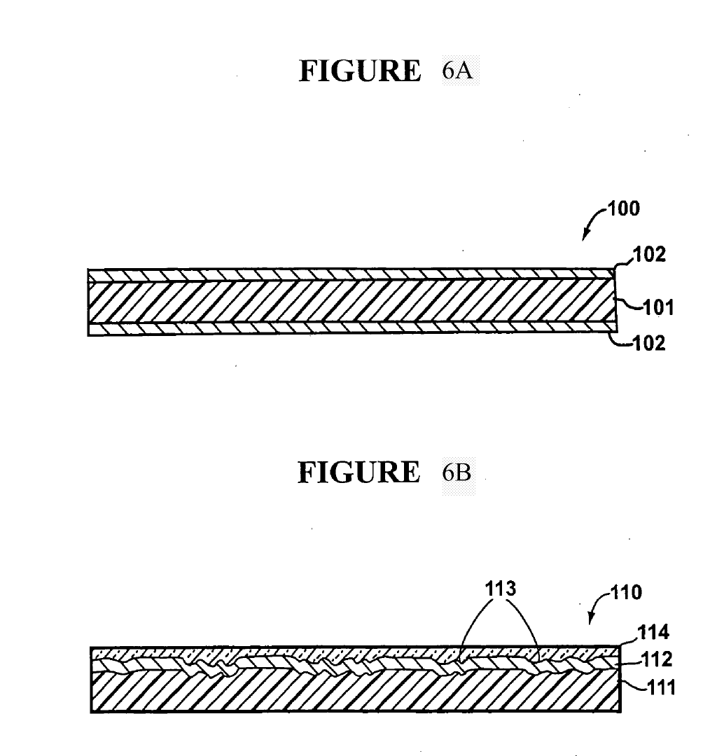

FIG. 6B shows a cross section of a magnetic data storage medium (100) that can be encoded with a machine-readable data that can be carried out by a system such as system (10) of FIG. 5. Medium (100) can be a conventional floppy diskette or hard disk, having a suitable substrate (101), which may be conventional, and a suitable coating (102), which may be conventional, on one or both sides, containing magnetic domains (not visible) whose polarity or orientation can be altered magnetically. Medium (100) may also have an opening (not shown) for receiving the spindle of a disk drive or other data storage device (24).

The magnetic domains of coating (102) of medium (100) are polarized or oriented so as to encode in manner which may be conventional, machine readable data such as that described herein, for execution by a system such as system (10) of FIG. 5.

FIG. 6B shows a cross section of an optically-readable data storage medium (110) which also can be encoded with such a machine-readable data, or set of instructions, which can be carried out by a system such as system (10) of FIG. 5. Medium (110) can be a conventional compact disk read only memory (CD-ROM) or a rewritable medium such as a magneto-optical disk which is optically readable and magneto-optically writable. Medium (100) preferably has a suitable substrate (111), which may be conventional, and a suitable coating (112), which may be conventional, usually of one side of substrate (111).

In the case of CD-ROM, as is well known, coating (112) is reflective and is impressed with a plurality of pits (113) to encode the machine-readable data. The arrangement of pits is read by reflecting laser light off the surface of coating (112). A protective coating (114), which preferably is substantially transparent, is provided on top of coating (112).

In the case of a magneto-optical disk, as is well known, coating (112) has no pits (113), but has a plurality of magnetic domains whose polarity or orientation can be changed magnetically when heated above a certain temperature, as by a laser (not shown). The orientation of the domains can be read by measuring the polarization of laser light reflected from coating (112). The arrangement of the domains encodes the data as described above.

In one embodiment, the structure coordinates of said molecules or molecular complexes or binding pockets are produced by homology modeling of at least a portion of the structure coordinates of FIG. 1A. Homology modeling can be used to generate structural models of SMYD3 homologues or other homologous proteins based on the known structure of SMYD3 domain. This can be achieved by performing one or more of the following steps: performing sequence alignment between the amino acid sequence of a molecule (possibly an unknown molecule) against the amino acid sequence of SMYD3; identifying conserved and variable regions by sequence or structure; generating structure coordinates for structurally conserved residues of the unknown structure from those of SMYD3; generating conformations for the structurally variable residues in the unknown structure; replacing the non-conserved residues of SMYD3 with residues in the unknown structure; building side chain conformations; and refining and/or evaluating the unknown structure.

Software programs that are useful in homology modeling include XALIGN (Wishart, D. S., et al., Comput. Appl. Biosci., 10: 687-88 (1994)) and CLUSTAL W Alignment Tool, Higgins et al., supra. See also, U.S. Pat. No. 5,884,230. These references are incorporated herein by reference.

To perform the sequence alignment, programs such as the “bestfit” program available from the Genetics Computer Group (Waterman in Advances in Applied Mathematics 2, 482 (1981), which is incorporated herein by reference) and CLUSTAL W Alignment Tool (Higgins et al., supra, which is incorporated by reference) can be used. To model the amino acid side chains of homologous molecules, the amino acid residues in SMYD3 can be replaced, using a computer graphics program such as “O” (Jones et al, (1991) Acta Cryst. Sect. A, 47: 110-119), by those of the homologous protein, where they differ. The same orientation or a different orientation of the amino acid can be used. Insertions and deletions of amino acid residues may be necessary where gaps occur in the sequence alignment. However, certain portions of the active site of SMYD3 and its homologues are highly conserved with essentially no insertions and deletions.

Homology modeling can be performed using, for example, the computer programs SWISS-MODEL available through Glaxo Wellcome Experimental Research in Geneva, Switzerland; WHATIF available on EMBL servers; Schnare et al., J. Mol. Biol, 256: 701-719 (1996); Blundell et al., Nature 326: 347-352 (1987); Fetrow and Bryant, Bio/Technology 11:479-484 (1993); Greer, Methods in Enzymology 202: 239-252 (1991); and Johnson et al, Crit. Rev. Biochem. Mol. Biol. 29:1-68 (1994). An example of homology modeling can be found, for example, in Szklarz G. D., Life Sci. 61: 2507-2520 (1997). These references are incorporated herein by reference.

Thus, in accordance with the present invention, data capable of generating the three-dimensional structure or three-dimensional representation of the above molecules or molecular complexes, or binding pockets thereof, can be stored in a machine-readable storage medium, which is capable of displaying structural information or a graphical three-dimensional representation of the structure. In one embodiment, means of generating three-dimensional information is provided by means for generating a three-dimensional structural representation of the binding pocket or protein or protein complex.

Rational Drug Design

The SMYD3 structure coordinates or the three-dimensional graphical representation generated from these coordinates may be used in conjunction with a computer for a variety of purposes, including drug discovery.

For example, the structure encoded by the data may be computationally evaluated for its ability to associate with chemical entities. Chemical entities that associate with SMYD3 may inhibit or activate SMYD3 or its homologues, and are potential drug candidates. Alternatively, the structure encoded by the data may be displayed in a graphical three-dimensional representation on a computer screen. This allows visual inspection of the structure, as well as visual inspection of the structure's association with chemical entities.

In one embodiment, the invention provides a method of using a computer for selecting an orientation of a chemical entity that interacts favorably with a binding pocket or domain comprising the steps of:

(a) providing the structure coordinates of said binding pocket or domain on a computer comprising means for generating three-dimensional structural information from said structure coordinates;

(b) employing computational means to dock a first chemical entity in the binding pocket or domain;

(c) quantifying the association between said chemical entity and all or part of the binding pocket or domain for different orientations of the chemical entity; and

(d) selecting the orientation of the chemical entity with the most favorable interaction based on said quantified association.

In one embodiment, the docking is facilitated by said quantified association.

In one embodiment, the above method further comprises the following steps before step (a):

(e) producing a crystal of a molecule or molecular complex comprising a SMYD3 domain or homologue thereof;

(f) determining the three-dimensional structure coordinates of the molecule or molecular complex by X-ray diffraction of the crystal; and

(g) identifying all or part of a binding pocket that corresponds to said binding pocket

Three-dimensional structural information in step (a) may be generated by instructions such as a computer program or commands that can generate a three-dimensional representation; subtract distances between atoms; calculate chemical energies for a SMYD3 molecule, molecular complex or homologues thereof; or calculate or minimize the chemical energies of an association of SMYD3 molecule, molecular complex or homologues thereof to a chemical entity. These types of computer programs are known in the art. The graphical representation can be generated or displayed by commercially available software programs. Examples of software programs include but are not limited to QUANTA (Accelrys ©2001, 2002), O (Jones et al., Acta Crystallogr. A47: 110-119 (1991)) and RIBBONS (Carson, J. Appl. Crystallogr., 24: 9589-961 (1991)), which are incorporated herein by reference. Certain software programs may imbue this representation with physico-chemical attributes which are known from the chemical composition of the molecule, such as residue charge, hydrophobicity, torsional and rotational degrees of freedom for the residue or segment, etc. Examples of software programs for calculating chemical energies are described below.

Optionally, the above methods may further comprise the following step after step (d): outputting said quantified association to a suitable output hardware, such as a CRT display terminal, a CD or DVD recorder, ZIP™ or JAZ™ drive, a disk drive, or other machine-readable data storage device, as described previously. The method may further comprise generating a three-dimensional structure, graphical representation thereof, or both, of the protein, binding pocket, molecule or molecular complex prior to step (b).

One embodiment of this invention provides the above method, wherein energy minimization, molecular dynamics simulations, rigid body minimizations combinations thereof, or similar induced-fit manipulations are performed simultaneously with or following step (b).

The above method may further comprise the steps of:

(e) repeating steps (b) through (d) with a second chemical entity; and

(f) selecting of at least one of said first or second chemical entity that interacts more favorably with said-binding pocket or domain based on said quantified association of said first or second chemical entity.

In another embodiment, the invention provides the method of using a computer for selecting an orientation of a chemical entity with a favorable shape complementarity in a binding pocket comprising the steps of:

(a) providing the structure coordinates of said binding pocket and all or part of the SAM binding motif bound therein on a computer comprising means for generating three-dimensional structural information from said structure coordinates;

(b) employing computational means to dock a first chemical entity in the binding pocket;

(c) quantitating the contact score of said chemical entity in different orientations in the binding pocket; and

(d) selecting an orientation with the highest contact score.

In one embodiment, the docking is monitored and directed or facilitated by the contact score.

The method above may further comprise the step of generating a three-dimensional graphical representation of the binding pocket and all or part of the SAM binding motif bound therein prior to step (b).

The method above may further comprise the steps of:

(e) repeating steps (b) through (d) with a second chemical entity; and

(f) selecting at least one of said first or second chemical entity that has a higher contact score based on said quantitated contact score of said first or second chemical entity.

In another embodiment, the invention provides a method for screening a plurality of chemical entities to associate at a deformation energy of binding of no greater than 7 kcal/mol with said binding pocket:

(a) employing computational means, which utilize said structure coordinates to dock one of said chemical entities from the plurality of chemical entities and said binding pocket;

(b) quantifying the deformation energy of binding between the chemical entity and the binding pocket;

(c) repeating steps (a) and (b) for each remaining chemical entity; and

(d) outputting a set of chemical entities that associate with the binding pocket at a deformation energy of binding of not greater than 7 kcal/mol to a suitable output hardware.

In another embodiment, the method comprises the steps of:

(a) constructing a computer model of a binding pocket of a molecule or molecular complex;

(b) selecting a chemical entity to be evaluated by a method selected from the group consisting of assembling said chemical entity; selecting a chemical entity from a small molecule database; de novo ligand design of said chemical entity; and modifying a known binder, or a portion thereof, of a SMYD3 protein, or homologue thereof to produce said chemical entity;

(c) employing computational means to dock said chemical entity to be evaluated in said binding pocket in order to provide an energy-minimized configuration of said chemical entity in the binding pocket; and

(d) evaluating the results of said docking to quantify the association between said chemical entity and the binding pocket Alternatively, the structure coordinates of the SMYD3 binding pockets may be utilized in a method for identifying a candidate binder of a molecule or molecular complex comprising a binding pocket of SMYD3. This method comprises the steps of:

(a) using a three-dimensional structure of the binding pocket or domain of SMYD3 to design, select or optimize a plurality of chemical entities;

(b) contacting each chemical entity with the molecule and molecular complex;

(c) monitoring the change in the catalytic activity of the molecule or molecular complex by the chemical entity; and

(d) selecting a chemical entity based on the effect of the chemical entity on the activity of the molecule or molecular complex.

In one embodiment, step (a) is carried out using a three-dimensional structure of the binding pocket or domain or portion thereof of the molecule or molecular complex. In another embodiment, the three-dimensional structure is displayed as a graphical representation.

In another embodiment, the method comprises the steps of:

(a) constructing a computer model of a binding pocket of the molecule or molecular complex;

(b) selecting a chemical entity to be evaluated by a method selected from the group consisting of assembling said chemical entity; selecting a chemical entity from a small molecule database; de novo ligand design of said chemical entity; and modifying a known binder, or a portion thereof, of a SMYD3 protein or homologue thereof to produce said chemical entity;

(c) employing computational means to dock said chemical entity to be evaluated and said binding pocket in order to provide an energy-minimized configuration of said chemical entity in the binding pocket; and

(d) evaluating the results of said docking to quantify the association between said chemical entity and the binding pocket;

(e) synthesizing said chemical entity; and

(f) contacting said chemical entity with said molecule or molecular complex to determine the ability of said chemical entity to activate or inhibit said molecule.

In one embodiment, the invention provides a method of designing a compound or complex that associates with all or part of the binding pocket of a domain of a SMYD3 protein comprising the steps of:

(a) providing the structure coordinates of said binding pocket or domain on a computer comprising means for generating three-dimensional structural information from said structure coordinates;

(b) using the computer to dock a first chemical entity in part of the binding pocket or domain;

(c) docking a second chemical entity in another part of the binding pocket or domain;

(d) quantifying the association between the first and second chemical entity and part of the binding pocket or domain;

(e) repeating steps (b) to (d) with another first and second chemical entity and selecting a first and a second chemical entity based on said quantified association of all of said first and second chemical entity;

(f) optionally, visually inspecting the relationship of the first and second chemical entity to each other in relation to the binding pocket or domain on a computer screen using the three-dimensional graphical representation of the binding pocket or domain and said first and second chemical entity; and

(g) assembling the first and second chemical entity into a compound or complex that interacts with said binding pocket by model building.

For the first time, the present invention permits the use of molecular design techniques to identify, select and design chemical entities, including inhibitory compounds, capable of binding to SMYD3 or SMYD3-like binding pockets and domains.

Applicants' elucidation of binding pockets of SMYD3 provides the necessary information for designing new chemical entities and compounds that may interact with SMYD3 substrate, active site, SAM binding pockets or SMYD3-like substrate, active site or SAM binding pockets, in whole or in part.

Throughout this section, discussions about the ability of a chemical entity to bind to, interact with or inhibit SMYD3 binding pockets refer to features of the entity alone.

The design of compounds that bind to or inhibit SMYD3 binding pockets according to this invention generally involves consideration of two factors. First, the chemical entity must be capable of physically and structurally associating with parts or all of the SMYD3 binding pockets. Non-covalent molecular interactions important in this association include hydrogen bonding, van der Waals interactions, hydrophobic interactions and electrostatic interactions.

Second, the chemical entity must be able to assume a conformation that allows it to associate with the SMYD3 binding pockets directly. Although certain portions of the chemical entity will not directly participate in these associations, those portions of the chemical entity may still influence the overall conformation of the molecule. This, in turn, may have a significant impact on potency. Such conformational requirements include the overall three-dimensional structure and orientation of the chemical entity in relation to all or a portion of the binding pocket, or the spacing between functional groups of a chemical entity comprising several chemical entities that directly interact with the SMYD3 or SMYD3-like binding pockets.

The potential effect of a chemical entity on SMYD3 binding pockets may be analyzed prior to its actual synthesis and testing by the use of computer modeling techniques. If the theoretical structure of the given entity suggests insufficient interaction and association between it and the SMYD3 binding pockets, testing of the entity is obviated. However, if computer modeling indicates a strong interaction, the molecule may then be synthesized and tested for its ability to bind to a SMYD3 binding pocket This may be achieved by testing the ability of the molecule to bind SMYD3 using the assays described herein.

A potential binder of a SMYD3 binding pocket may be computationally evaluated by means of a series of steps in which chemical entities or fragments are screened and selected for their ability to associate with the SMYD3 binding pockets.

One skilled in the art may use one of several methods to screen chemical entities or fragments or moieties thereof for their ability to associate with the binding pockets described herein. This process may begin by visual inspection of, for example, any of the binding pockets on the computer screen based on the SMYD3 structure coordinates FIG. 1A, or other coordinates which define a similar shape generated from the machine-readable storage medium. Selected chemical entities, or fragments or moieties thereof may then be positioned in a variety of orientations, or docked, within that binding pocket as defined supra. Docking may be accomplished using software such as QUANTA (Accelrys ©2001, 2002) and Sybyl (Tripos Associates, St. Louis, Mo.), followed by, or performed simultaneously with, energy minimization, rigid-body minimization (Gshwend, supra) and molecular dynamics with standard molecular mechanics force fields, such as CHARMM and AMBER.

Specialized computer programs may also assist in the process of selecting fragments or chemical entities. These include:
1. GRID (Goodford, P. J., “A Computational Procedure for Determining Energetically Favorable Binding Sites on Biologically Important Macromolecules”, J. Med. Chem., 28: 849-857 (1985)). GRID is available from Oxford University, Oxford, UK.
2. MCSS (Miranker, A., et al., “Functionality Maps of Binding Sites: A Multiple Copy Simultaneous Search Method.” Proteins Struct. Funct. Genet, 11: 29-34 (1991)). MCSS is available from Accelrys, San Diego, Calif.
3. AUTODOCK (Goodsell, D. S., et al., “Automated Docking of Substrates to Proteins by Simulated Annealing”, Proteins Struct., Funct., and Genet, 8: 195-202 (1990)). AUTODOCK is available from Scripps Research Institute, La Jolla, Calif.
4. DOCK (Kuntz, I. D., et al., “A Geometric Approach to Macromolecule-Ligand Interactions”, J. Mol. Biol., 161: 269-288 (1982)). DOCK is available from University of California, San Francisco, Calif.

Once suitable chemical entities or fragments have been selected, they can be assembled into a single compound or complex. Assembly may be preceded by visual inspection of the relationship of the fragments to each other on the three-dimensional image displayed on a computer screen in relation to the structure coordinates of SMYD3. This would be followed by manual model building using software such as QUANTA (Accelrys ©2001, 2002) or Sybyl (Tripos Associates, St. Louis, Mo.).

Useful programs to aid one of skill in the art in connecting the individual chemical entities or fragments include:
1. CAVEAT (Bartlett, P. A., et al., “CAVEAT: A Program to Facilitate the Structure-Derived Design of Biologically Active Molecules”, in Molecular Recognition in Chemical and Biological Problems, S. M. Roberts, Ed., Royal Society of Chemistry, Special Publication No. 78: pp. 182-196 (1989); Lauri, G. and Bartlett, P. A., “CAVEAT: A Program to Facilitate the Design of Organic Molecules”, J. Comp. Aid. Molec. Design, 8: 51-66 (1994)). CAVEAT is available from the University of California, Berkeley, Calif.
2. 3D Database systems such as ISIS (MDL Information Systems, San Leandro, Calif.). This area is reviewed in Martin, Y. C., “3D Database Searching in Drug Design”, J. Med. Chem., 35: 2145-2154 (1992).
3. HOOK (Eisen, M. B., et al., “HOOK: A Program for Finding Novel Molecular Architectures that Satisfy the Chemical and Steric Requirements of a Macromolecule Binding Site”, Proteins Struct., Funct., Genet, 19: 199-221 (1994)). HOOK is available from Accelrys, San Diego, Calif.

Instead of proceeding to build an binder of a SMYD3 binding pocket in a step-wise fashion one fragment or chemical entity at a time as described above, inhibitory or other SMYD3 binding compounds may be designed as a whole or “de novo” using either an empty binding pocket or optionally including some portion(s) of a known binder(s). There are many de novo ligand design methods including:
1. LUDI (Bohm, H.-J., “The Computer Program LUDI: A New Method for the De Novo Design of Enzyme Inhibitors”, J. Comp. Aid. Molec. Design, 6: pp. 61-78 (1992)). LUDI is available from Accelrys Incorporated, San Diego, Calif.
2. LEGEND (Nishibata, Y., et al., Tetrahedron, 47: 8985-8990 (1991)). LEGEND is available from Accelrys Incorporated, San Diego, Calif.
3. LeapFrog (available from Tripos Associates, St. Louis, Mo.).
4. SPROUT (Gillet, V., et al., “SPROUT: A Program for Structure Generation)”, J. Comp. Aid. Molec. Design, 7: 127-153 (1993)). SPROUT is available from the University of Leeds, UK.

Other molecular modeling techniques may also be employed in accordance with this invention (see, e.g., Cohen, N. C., et al., “Molecular Modeling Software and Methods for Medicinal Chemistry, J. Med. Chem., 33: 883-894 (1990); see also, Navia, M. A. and Murcko, M. A., “The Use of Structural Information in Drug Design”, Current Opinions in Structural Biology, 2: 202-210 (1992); Balbes, L. M., et al., “A Perspective of Modern Methods in Computer-Aided Drug Design”, in Reviews in Computational Chemistry, K. B. Lipkowitz and D. B. Boyd, Eds., VCH Publishers, New York, 5: pp. 337-379 (1994); see also, Guida, W. C., “Software For Structure-Based Drug Design”, Curr. Opin. Struct. Biology, 4: 777-781 (1994); Sherman, W., et al., “Novel Procedure for Modeling Ligand/Receptor Induced Fit Effects”, J. Med. Chem., 49: 534-553 (2006)).

Once a chemical entity has been designed or selected by the above methods, the efficiency with which that entity may bind to any of the above binding pockets may be tested and optimized by computational evaluation. For example, an effective binding pocket binder must preferably demonstrate a relatively small difference in energy between its bound and free states (i.e., a small deformation energy of binding). Thus, the most efficient binding pocket binders should preferably be designed with a magnitude of deformation energy of binding of not greater than about 10 kcal/mole, more preferably, not greater than 7 kcal/mole. Binding pocket binders may interact with the binding pocket in more than one conformation that is similar in overall binding energy. In those cases, the deformation energy of binding is taken to be the difference between the energy of the free entity and the average energy of the conformations observed when the binder binds to the protein.

A chemical entity designed or selected as binding to any one of the above binding pockets may be further computationally optimized so that in its bound state it would preferably lack repulsive electrostatic interaction with the target enzyme and with the surrounding water molecules. Such non-complementary electrostatic interactions include repulsive charge-charge, dipole-dipole and charge-dipole interactions.

Specific computer software is available in the art to evaluate compound deformation energy and electrostatic interactions. Examples of programs designed for such

uses include: Gaussian 94, revision C (M. J. Frisch, Gaussian, Inc., Pittsburgh, Pa. ©1995); AMBER, version 4.1 (P. A. Kollman, University of California at San Francisco, ©1995); QUANTA/CHARMM (Accelrys ©2001, 2002); Insight II/Discover (Accelrys, Inc., San Diego, Calif. ©1998); DelPhi (Accelrys, Inc., San Diego, Calif. ©1998); and AMSOL (Quantum Chemistry Program Exchange, Indiana University). These programs may be implemented, for instance, using a Silicon Graphics workstation such as an Indigo2 with “IMPACT” graphics. Other hardware systems and software packages will be known to those skilled in the art.

Another approach enabled by this invention is the computational screening of small molecule databases for chemical entities or compounds that can bind in whole, or in part, to any of the above binding pocket. In this screening, the quality of fit of such entities to the binding pocket may be judged either by shape complementarity or by estimated interaction energy (Meng, E. C., et al., J. Comp. Chem., 13: 505-524 (1992)).

According to another embodiment, the invention provides chemical entities that associate with a SMYD3 binding pocket produced or identified by the method set forth above.

Another particularly useful drug design technique enabled by this invention is iterative drug design. Iterative drug design is a method for optimizing associations between a protein and a chemical entity by determining and evaluating the three-dimensional structures of successive sets of protein/chemical entity complexes.

In iterative drug design, crystals of a series of protein or protein complexes are obtained and then the three-dimensional structures of each crystal is solved. Such an approach provides insight into the association between the proteins and compounds of each complex. This is accomplished by selecting compounds with binding capacity, obtaining crystals of this new protein/compound complex, solving the three-dimensional structure of the complex, and comparing the associations between the new protein/compound complex and previously solved protein/compound complexes. By observing how changes in the compound affected the protein/compound associations, these associations may be optimized.

In some cases, iterative drug design is carried out by forming successive protein-compound complexes and then crystallizing each new complex. High throughput crystallization assays may be used to find a new crystallization condition or to optimize the original protein crystallization condition for the new complex. Alternatively, a pre-formed protein crystal may be soaked in the presence of a binder, thereby forming a protein/compound complex and obviating the need to crystallize each individual protein/compound complex.

Any of the above methods may be used to design peptide or small molecule mimics of the SAM binding motif which may have effects on the activity of full-length SMYD3 protein or fragments thereof, or on the activity of full-length but mutated SMYD3 protein or fragments of the mutated protein thereof.

In one embodiment, the present invention provides a method for identifying a candidate binder that interacts with a binding site of a SMYD3 methyltransferase protein or a homologue thereof, comprising the steps of:

(a) obtaining a crystal comprising a domain of said SMYD3 methyltransferase protein or said homologue thereof, wherein the crystal is characterized with space group P_{1 21 1} and has unit cell parameters of a=58.175 Å, b=118.073 Å, c=82.901 Å α=90.00, β=91.58, γ=90.00;

(b) obtaining the structure coordinates of amino acids of the crystal of step (a), wherein the structure coordinates are set forth in FIG. 1A-1 to 1A-129;

(c) generating a three-dimensional model of the domain of said SMYD3 methyltransferase protein or said homologue thereof using the structure coordinates of the amino acids generated in step (b), a root mean square deviation from backbone atoms of said amino acids of not more than ±2.0 Å;

(d) determining a binding site of the domain of said SMYD3 methyltransferase protein or said homologue thereof from said three-dimensional model; and

(e) performing computer fitting analysis to identify the candidate binder which interacts with said binding site.

In one embodiment, the present invention provides the method for identifying a candidate binder that interacts with a binding site of a SMYD3 methyltransferase protein or a homologue thereof, further comprising the step of: (f) contacting the identified candidate binder with the domain of said SMYD3 methyltransferase protein or said homologue thereof in order to determine the effect of the binder on SMYD3 methyltransferase protein activity.

In one embodiment, the present invention provides the method for identifying a candidate binder that interacts with a binding site of a SMYD3 methyltransferase protein or a homologue thereof, wherein the binding site of the domain of said SMYD3 methyltransferase protein or said homologue thereof determined in step (d) comprises the structure coordinates according to FIG. 1A-1 to 1A-129 of amino acid residues R14, N132, Y124, and N205, wherein the root mean square deviation from the backbone atoms of said amino acids is not more than ±2.0 Å.

In one embodiment, the present invention provides the method for identifying a candidate binder that interacts with a binding site of a SMYD3 methyltransferase protein or a homologue thereof, wherein the binding site of the domain of said SMYD3 methyltransferase protein or said homologue thereof determined in step (d) comprises the structure coordinates according to FIG. 1A-1 to 1A-129 of amino acid residues R14, N16, Y124, E130, N132, N181, N205, H206, and F259, wherein the root mean square deviation from the backbone atoms of said amino acids is not more than ±2.0 Å.

In one embodiment, the present invention provides the method for identifying a candidate binder that interacts with a binding site of a SMYD3 methyltransferase protein or a homologue thereof, wherein the binding site of the domain of said SMYD3 methyltransferase protein or said homologue thereof determined in step (d) comprises the structure coordinates according to FIG. 1A-1 to 1A-129 of amino acid residues R14, G15, N16, G17, Y124, E130, N132, K135, C180, N181, S182, F183, T184, I201, S202, L203, L204, N205, H206, S207, C208, I214, I237, C238, Y239, L240, D241, R249, L253, Q256, Y257, F259, C261, D262, C263, R265, and C266, wherein the root mean square deviation from the backbone atoms of said amino acids is not more than ±2.0 Å.

In one embodiment, the present invention provides a method for identifying a candidate binder that interacts with a binding site of a domain of a SMYD3 methyltransferase protein or a homologue thereof, comprising the steps of:

(a) obtaining a crystal comprising the domain of said SMYD3 methyltransferase protein or said homologue thereof, wherein the crystal is characterized with space group P_{1 21 1} and has unit cell parameters of a=58.175 Å, b=118.073 Å, c=82.901 Å α=90.00, β=91.58, γ=90.00;

(b) obtaining the structure coordinates of amino acids of the crystal of step (a);

(c) generating a three-dimensional model of said SMYD3 methyltransferase protein or said homologue thereof using the structure coordinates of the amino acids generated in step (b), a root mean square deviation from backbone atoms of said amino acids of not more than ±2.0 Å;

(d) determining a binding site of the domain of said SMYD3 methyltransferase protein or said homologue thereof from said three-dimensional model; and

(e) performing computer fitting analysis to identify the candidate binder which interacts with said binding site. In one embodiment, the step of obtaining a crystal is optional.

In one embodiment, the present invention provides the method for identifying a candidate binder that interacts with a binding site, further comprising the step of:

(f) contacting the identified candidate binder with the domain of said SMYD3 methyltransferase protein or said homologue thereof in order to determine the effect of the binder on SMYD3 methyltransferase activity.

One embodiment of this invention provides the method for identifying a candidate binder that interacts with a binding site, wherein the binding site of the domain of said SMYD3 methyltransferase protein or said homologue thereof determined in step (d) comprises the structure coordinates according to FIG. 1A-1 to 1A-129 of amino acid residues R14, N132, Y124, and N205, wherein the root mean square deviation from the backbone atoms of said amino acids is not more than ±2.0 Å.

One embodiment of this invention provides the method for identifying a candidate binder that interacts with a binding site, wherein the binding site of the domain of said SMYD3 methyltransferase protein or said homologue thereof determined in step (d) comprises the structure coordinates according to FIG. 1A-1 to 1A-129 of amino acid residues R14, N16, Y124, E130, N132, N181, N205, H206, and F259, wherein the root mean square deviation from the backbone atoms of said amino acids is not more than ±2.0 Å.

In one embodiment, the present invention provides the method for identifying a candidate binder that interacts with a binding site, wherein the binding site of the domain of said SMYD3 methyltransferase protein or said homologue thereof determined in step (d) comprises the structure coordinates according to FIG. 1A-1 to 1A-129 of amino acid residues R14, G15, N16, G17, Y124, E130, N132, K135, C180, N181, S182, F183, T184, I201, S202, L203, L204, N205, H206, S207, C208, I214, I237, C238, Y239, L240, D241, R249, L253, Q256, Y257, F259, C261, D262, C263, R265, and C266, wherein the root mean square deviation from the backbone atoms of said amino acids is not more than ±2.0 Å.

In one embodiment, the present invention provides a method for identifying a candidate binder that interacts with a binding site of a domain of a SMYD3 methyltransferase protein or a homologue thereof, comprising the step of determining a binding site of the domain of said SMYD3 methyltransferase protein or the homologue thereof from a three-dimensional model to design or identify the candidate binder which interacts with said binding site.

In one embodiment, the present invention provides the method for identifying a candidate binder that interacts with a binding site of a domain of a SMYD3 methyltransferase protein or a homologue thereof, wherein the binding site of the domain of said SMYD3 methyltransferase protein or said homologue thereof determined comprises the structure coordinates according to FIG. 1A-1 to 1A-129 of amino acid residues R14, N132, Y124, and N205, wherein the root mean square deviation from the backbone atoms of said amino acids is not more than ±2.0 Å.

In one embodiment, the present invention provides the method for identifying a candidate binder that interacts with a binding site of a domain of a SMYD3 methyltransferase protein or a homologue thereof, wherein the binding site of the domain of said SMYD3 methyltransferase protein or said homologue thereof determined comprises the structure coordinates according to FIG. 1A-1 to 1A-129 of amino acid residues R14, N16, Y124, E130, N132, N181, N205, H206, and F259, wherein the root mean square deviation from the backbone atoms of said amino acids is not more than ±2.0 Å.

In one embodiment, the present invention provides the method for identifying a candidate binder that interacts with a binding site of a domain of a SMYD3 methyltransferase protein or a homologue thereof, wherein the binding site of the domain of said SMYD3 methyltransferase protein or said homologue thereof determined comprises the structure coordinates according to FIG. 1A-1 to 1A-129 of amino acid residues R14, G15, N16, G17, Y124, E130, N132, K135, C180, N181, S182, F183, T184, I201, S202, L203, L204, N205, H206, S207, C208, I214, I237, C238, Y239, L240, D241, R249, L253, Q256, Y257, F259, C261, D262, C263, R265, C266, wherein the root mean square deviation from the backbone atoms of said amino acids is not more than ±2.0 Å.

One embodiment of this invention provides a method for identifying a candidate binder of a molecule or molecular complex comprising a binding pocket or domain selected from the group consisting of:

(i) a set of amino acid residues which are identical to human SMYD3 methyltransferase amino acid residues R14, N132, Y124, and N205 according to FIG. 1A, wherein the root mean square deviation of the backbone atoms between the set of amino acid residues and the SMYD3 amino acid residues is not greater than about 2.0 Å;

(ii) a set of amino acid residues comprising at least three amino acid residues which are identical to human SMYD3 methyltransferase amino acid residues R14, N16, Y124, E130, N132, N181, N205, H206, and F259 according to FIG. 1A, wherein the root mean square deviation of the backbone atoms between the at least three amino acid residues and the SMYD3 amino acid residues which are identical is not greater than about 2.0 Å;

(iii) a set of amino acid residues comprising at least five amino acid residues which are identical to human SMYD3 methyltransferase amino acid residues R14, N16, Y124, E130, N132, N181, N205, H206, and F259 according to FIG. 1A, wherein the root mean square deviation of the backbone atoms between the at least five amino acid residues and the SMYD3 amino acid residues which are identical is not greater than about 2.0 Å;

(iv) a set of amino acid residues comprising at least five amino acid residues which are identical to human SMYD3 methyltransferase amino acid residues R14, G15, N16, G17, Y124, E130, N132, K135, C180, N181, S182, F183, T184, I201, S202, L203, L204, N205, H206, S207, C208, I214, I237, C238, Y239, L240, D241, R249, L253, Q256, Y257, F259, C261, D262, C263, R265, C266 according to FIG. 1A, wherein the root mean square deviation of the backbone atoms between the at least five amino acid residues and the SMYD3 amino acid residues which are identical is not greater than about 2.0 Å; and

(v) a set of amino acid residues comprising at least six amino acid residues which are identical to human SMYD3 methyltransferase amino acid residues R14, G15, N16, G17, Y124, E130, N132, K135, C180, N181, S182, F183, T184, I201, S202, L203, L204, N205, H206, S207, C208, I214, I237, C238, Y239, L240, D241, R249, L253, Q256, Y257, F259, C261, D262, C263, R265, C266 according to FIG. 1A, wherein the root mean square deviation of the backbone atoms between the at least six amino acid residues and the SMYD3 amino acid residues which are identical is not greater than about 2.0 Å; and

(vi) a set of amino acid residues that are identical to SMYD3 amino acid residues according to FIG. 1A, wherein the root mean square deviation between the set of amino acid residues and the SMYD3 amino acid residues is not more than about 2.0 Å;

(vii) a set of amino acid residues that are identical to SMYD3 amino acid residues according to FIG. 1A, wherein the root mean square deviation between the set of amino acid residues and the SMYD3 amino acid residues is not more than about 3.0 Å;

comprising the steps of:

(a) using a three-dimensional structure of the binding pocket or domain to design, select or optimize a plurality of chemical entities; and

(b) selecting said candidate binder based on the effect of said chemical entities on said domain of said SMYD3 methyltransferase protein or said domain of said SMYD3 methyltransferase protein homologue on the catalytic activity of the molecule.

In one embodiment, the present invention provides a method of using a crystal of a domain of said SMYD3 methyltransferase protein or a homologue in a binder screening assay comprising:

(a) selecting a potential binder by performing rational drug design with a three-dimensional structure determined for the crystal, wherein said selecting is performed in conjunction with computer modeling;

(b) contacting the potential binder with a methyltransferase; and

(c) detecting the ability of the potential binder to modulate the activity of the methyltransferase.

In certain embodiments, the ability of the potential binder for modulating the methyltransferase is assessed using an enzyme inhibition assay. In other embodiments, the ability of the potential binder for modulating the methyltransferase is performed using a cellular-based assay. In other embodiments, the ability of the potential binder for interacting with the methyltransferase is performed using affinity-selection-mass-spectrometry.

In one embodiment, the present invention provides a method for identifying a candidate binder that interacts with a binding site of a SMYD3 methyltransferase protein or a homologue thereof comprising:

(a) obtaining a crystal of a SMYD3 methyltransferase protein or a homologue thereof;

(b) obtaining the atomic coordinates of the crystal; and

(c) using the atomic coordinates and one or more molecular modeling techniques to identify the candidate binder that interacts with a binding site of a SMYD3 methyltransferase protein or a homologue thereof. In certain embodiments, the crystal comprises a domain of a SMYD3 methyltransferase protein or a homologue thereof. In one embodiment, the step of obtaining a crystal is optional.

In one embodiment, the present invention provides the method for identifying a candidate binder that interacts with a binding site of a SMYD3 methyltransferase protein or a homologue thereof, wherein the one or more molecular modeling techniques are selected from the group consisting of graphic molecular modeling and computational chemistry.

In one embodiment, the present invention provides the method for identifying a candidate binder that interacts with a binding site of a SMYD3 methyltransferase protein or a homologue thereof, further comprising the candidate binder with the SMYD3 methyltransferase protein or the homologue and detecting binding of the candidate binder to the SMYD3 methyltransferase protein or the homologue.

In one embodiment, the present invention provides a method of struture-based identification of candidate compounds for binding to a SMYD3 methyltransferase protein or a homologue thereof, comprising:

(a) constructing a three-dimensional structure of the SMYD3 methyltransferase protein or a homologue thereof;

(b) performing computer-assisted structure-based drug design with said structure of the SMYD3 methyltransferase protein or a homologue; and

(c) identifying at least one candidate binder that is predicted to have a compatible conformation with a binding site of the structure of the SMYD3 methyltransferase protein or a homologue.

In certain embodiments, the present invention provides for methods wherein the three-dimensional structure is visualized as a computer image generated when said atomic coordinates determined by X-ray diffraction are analyzed on a computer using a graphical display software program to create an electronic file of the image and visualizing the electronic file on a computer capable of representing the electronic file as a three-dimensional image.

Structure Determination of Other Molecules

The structure coordinates set forth in FIG. 1A can also be used in obtaining structural information about other crystallized molecules or molecular complexes. This may be achieved by any of a number of well-known techniques, including molecular replacement.

According to one embodiment, the machine-readable data storage medium comprises a data storage material encoded with a first set of machine readable data which comprises the Fourier transform of at least a portion of the structure coordinates set forth in FIG. 1A or homology model thereof, and which, when using a machine programmed with instructions for using said data, can be combined with a second set of machine readable data comprising the X-ray diffraction pattern of a molecule or molecular complex to determine at least a portion of the structure coordinates corresponding to the second set of machine readable data.

In another embodiment, the invention provides a computer for determining at least a portion of the structure coordinates corresponding to X-ray diffraction data obtained from a molecule or molecular complex having an unknown structure, wherein said computer comprises:

(a) a machine-readable data storage medium comprising a data storage material encoded with machine-readable data, wherein said data comprises at least a portion of the structure coordinates of SMYD3 according to FIG. 1A or a homology model thereof;

(b) a machine-readable data storage medium comprising a data storage material encoded with machine-readable data, wherein said data comprises X-ray diffraction data obtained from said molecule or molecular complex having an unknown structure; and

(c) instructions for performing a Fourier transform of the machine-readable data of (a) and for processing said machine-readable data of (b) into structure coordinates.

For example, the Fourier transform of at least a portion of the structure coordinates set forth in FIG. 1A or homology model thereof may be used to determine at least a portion of the structure coordinates of the molecule or molecular complex.

Therefore, another embodiment this invention provides a method of utilizing molecular replacement to obtain structural information about a molecule or a molecular complex of unknown structure wherein the molecule or molecular complex is sufficiently homologous to SMYD3, comprising the steps of:

(a) crystallizing said molecule or molecular complex of unknown structure;

(b) generating an X-ray diffraction pattern from said crystallized molecule or molecular complex;

(c) applying at least a portion of the SMYD3 structure coordinates set forth in one of FIG. 1A or a homology model thereof to the X-ray diffraction pattern to generate a three-dimensional electron density map of at least a portion of the molecule or molecular complex whose structure is unknown; and

(d) generating a structural model of the molecule or molecular complex from the three-dimensional electron density map.

In one embodiment, the method is performed using a computer. In another embodiment, the molecule is selected from the group consisting of SMYD3 protein and SMYD3 domain homologues. In another embodiment, the molecular complex is SMYD3 domain complex or homologue thereof.

By using molecular replacement, all or part of the structure coordinates of SMYD3 as provided by this invention (and set forth in FIG. 1A) can be used to determine the structure of a crystallized molecule or molecular complex whose structure is unknown more quickly and efficiently than attempting to determine such information ab initio.

Molecular replacement provides an accurate estimation of the phases for an unknown structure. Phases are a factor in equations used to solve crystal structures that cannot be determined directly. Obtaining accurate values for the phases, by methods other than molecular replacement, is a time-consuming process that involves iterative cycles of approximations and refinements and greatly hinders the solution of crystal structures. However, when the crystal structure of a protein containing at least a homologous portion has been solved, the phases from the known structure may provide a satisfactory estimate of the phases for the unknown structure.

Thus, this method involves generating a preliminary model of a molecule or molecular complex whose structure coordinates are unknown, by orienting and positioning the relevant portion of SMYD3 protein according to FIG. 1A within the unit cell of the crystal of the unknown molecule or molecular complex so as best to account for the observed X-ray diffraction pattern of the crystal of the molecule or molecular complex whose structure is unknown. Phases can then be calculated from this model and combined with the observed X-ray diffraction pattern amplitudes to generate an electron density map of the structure whose coordinates are unknown. This, in turn, can be subjected to any well-known model building and structure refinement techniques to provide a final, accurate structure of the unknown crystallized molecule or molecular complex (E. Lattman, “Use of the Rotation and Translation Functions”, in Meth. Enzymol., 115: 55-77 (1985); M. G. Rossmann, ed., “The Molecular Replacement Method”, Int. Sci. Rev. Ser., No. 13, Gordon & Breach, New York (1972)).

The structure of any portion of any crystallized molecule or molecular complex that is sufficiently homologous to any portion of the structure of human SMYD3 protein can be resolved by this method.

In one embodiment, the method of molecular replacement is utilized to obtain structural information about a SMYD3 homologue. The structure coordinates of SMYD3 as provided by this invention are particularly useful in solving the structure of SMYD3 complexes that are bound by ligands, substrates and binders.

Furthermore, the structure coordinates of SMYD3 as provided by this invention are useful in solving the structure of SMYD3 proteins that have amino acid substitutions, additions and/or deletions (referred to collectively as “SMYD3 mutants”, as compared to naturally occurring SMYD3). These SMYD3 mutants may optionally be crystallized in co-complex with a chemical entity. The crystal structures of a series of such complexes may then be solved by molecular replacement and compared with that of wild-type SMYD3. Potential sites for modification within the various binding pockets of the enzyme may thus be identified. This information provides an additional tool for determining the most efficient binding interactions, for example, increased hydrophobic interactions, between SMYD3 and a chemical entity or compound.

The structure coordinates are also particularly useful in solving the structure of crystals of the domain of SMYD3 or homologues co-complexed with a variety of chemical entities. This approach enables the determination of the optimal sites for interaction between chemical entities, including candidate SMYD3 binders. For example, high resolution X-ray diffraction data collected from crystals exposed to different types of solvent allows the determination of where each type of solvent molecule resides. Small molecules that bind tightly to those sites can then be designed and synthesized and tested for their SMYD3 modulatory activity.

All of the molecules and complexes referred to above may be studied using well-known X-ray diffraction techniques and may be refined using 1.5-3.4 Å resolution X-ray data to an R value of about 0.30 or less using computer software, such as X-PLOR (Yale University, ©1992, distributed by Accelrys, Inc.; see, e.g., Blundell & Johnson, supra; Meth. Enzymol., vol. 114 & 115, H. W. Wyckoff et al., eds., Academic Press (1985)) or CNS (Brunger et al., Acta Cryst., D54: 905-921, (1998)).

In order that this invention be more fully understood, the following examples are set forth. These examples are for the purpose of illustration only and are not to be construed as limiting the scope of the invention in any way.

### EXAMPLE 1

### SMYD3 Expression and Purification

The full length SMYD3 protein (GenBank accession no. AAH3 1010; SEQ ID NO:1) was expressed in insect cells. SMYD3 (full length sequence, amino acid residues 1 to 218; was cloned from cDNA bone marrow library (Clonetech, CA, USA).]. (See, Hamamoto et al., (2004) Nature Cell Biology 6: 731-740) The expressed full length protein was engineered to contain a C-terminal hexa-histidine tag. The expressed SMYD3 protein has 3 amino acids added to its N-terminal end (MetAlaLeu) and 8 amino acids added to the C-terminal end (GluGlyHisHisHisHisHisHis). The full length protein of Hsp90 was cloned from Hep G2 cells [ATCC HB-8065]. The expressed Hsp90 protein has 3 amino acids added to its N-terminal end (MetAlaLeu). Sequence verified clones were each transformed into DH10 BAC chemically competent cells (Invitrogen Corporation, Cat#10361012). The transformation was then plated on selective media. 1-2 colonies were picked into minipreps and bacmid DNA isolated.

The bacmids were transfected and expressed in Spotoptera frugiperda (SF9) cells using the following standard Bac to Bac protocol (Invitrogen Corporation, Cat.#10359-016) to generate viruses for protein expression. SF9 cells were used for 48 hr expressions in SF-900 II media. The chaperone HSP90 was co-expressed with SMYD3 by co-infection with virus for each. Cells were collected by centrifugation and frozen pellets were used for purification of full length SMYD3.

Frozen cells were lysed in buffer, (50 mM Tris-HCl pH7.7, 250 mM NaCl with protease inhibitor cocktail (Roche Applied Science, Cat.#11-873-580-001)) and centrifuged to remove cell debris. The soluble fraction was purified over an IMAC column charged with nickel (GE Healthcare, NJ), and eluted under native conditions with a step gradient of 10 mM, then 500 mM imidazole. The protein was then further purified by gel filtration using a Superdex 200 column (GE Healthcare, NJ), into 25 mM Tris HCl pH7.6, 150 mM NaCl, and 1 mM TCEP. Protein was pooled based on SDS-PAGE and concentrated to 10 mg/ml.

### EXAMPLE 2

### Protein Crystallization for Native SMYD3

It has been found that a hanging drop or sitting drop containing 0.75 μl of protein 10 mg/mL and 1 mM Sinefungin in 25 mM Tris HCl pH7.6, 150 mM NACl, 1 mM TCEP and 0.75 μL reservoir solution: 100 mM Tris HCl pH 8.5, 17% PEG 20K, 100 mM Magnesium Chloride hexahydrate in a sealed container containing 500 μL reservoir solution, incubated overnight at 21° C. provides diffraction quality crystals. Crystals have also been grown with a reservoir solution of 100 mM HEPES pH 7.5, 16% PEG 3350, 200 mM Magnesium Chloride.

### EXAMPLE 3

### X-Ray Diffraction and Structure Determination of SMYD3

The crystals were individually harvested from their trays and transferred to a cryoprotectant consisting of 75-80% reservoir solution plus 20-25% glycerol or PEG400. After about 2 minutes the crystal was collected and transferred into liquid nitrogen. The crystals were then transferred in liquid nitrogen to the Advanced Photon Source (Argonne National Laboratory) where a two wavelength MAD experiment was collected, a Zn peak wavelength and a high energy remote wavelength.

X-ray diffraction data were indexed and integrated using the program MOSFLM (Collaborative Computational Project, Number 4 (1994) Acta. Cryst. D50, 760-763; http://www.ccp4.ac.uk/main.html) and then merged using the program SCALA ((Collaborative Computational Project, Number 4 (1994) Acta. Cryst. D50, 760-763; http://www.ccp4.ac.uk/main.html). The subsequent conversion of intensity data to structure factor amplitudes was carried out using the program TRUNCATE (Collaborative Computational Project, Number 4 (1994) Acta. Cryst. D50, 760-763; http://www.ccp4.ac.uk/main.html). The program SnB (Weeks, C. M. & Miller, R. (1999) J. Appl. Cryst. 32, 120-124; http://www.hwi.buffalo.edu/SnB/) was used to determine the location of Zn sites in the protein using the Bijvoet differences in data collected at the Zn peak wavelength. The refinement of the Zn sites and the calculation of the initial set of phases were carried out using the program MLPHARE (Collaborative Computational Project, Number 4 (1994) Acta. Cryst. D50, 760-763; http://www.ccp4.ac.uk/main.html). The electron density map resulting from this phase set was improved by density modification using the program DM (Collaborative Computational Project, Number 4 (1994) Acta. Cryst. D50, 760-763; http://www.ccp4.ac.uk/main.html). The initial protein model was built into the resulting map using the program ARP/wARP (Perrakis, A., Morris, R. J., Lamzin, V. S. (1999) Nature Struct. Biol. 6, 453-463; http://www.embl-hamburg.de/ARP/ and XTALVIEW/XFIT (McRee, D. E. J. Structural Biology (1993) 125:156-65; available from CCMS (San Diego Super Computer Center) CCMS-request sdsc.edu.). This model was refined using the program REFMAC (Collaborative Computational Project, Number 4 (1994) Acta. Cryst. D50, 760-763; http://www.ccp4.ac.uk/main.html) with interactive refitting carried out using the program XTALVIEW/XFIT (McRee, D. E. J. Structural Biology (1993) 125:156-65; available from CCMS (San Diego Super Computer Center) CCMS-request@sdsc.edu).

The electron density corresponding to side chains absent from the search model was generally clear and unambiguous in the methyltransferase domain.

The final SMYD3 structure contains two copies of the MYND domain (residues 49-87), the SET methyltransferase domain (residues 148 to 239), with one andenosyl ornithine and three zincs bound in each copy, and 482 water molecules. During the course of the refinement, the electron density corresponding to residues 2-4 in both chains and 423-428 in chain B was poor and did not improve. Consequently, these residues were removed from the final model. Crystallographic refinement statistics are provided in Table 1.

TABLE 1
SMYD3 Data Collection Statistics

| Space group | P 1 21 1 | |
|---|---|---|
| Cell dimensions | a = 58.2 | Å |
| | b = 118.1 | Å |
| | c = 82.9 | Å |
| | 1. = 90° | |
| | a. = 91.6° | |
| | γ = 90° | |
| Wavelength λ | 1.2815 | Å |
| Overall Resolution | 21.83 | Å |
| limits | 1.85 | Å |

| | |
|---|---|
| Number of reflections collected | 696882 |
| Number of unique reflections | 94957 |
| Overall Redundancy of data | 7.3 |
| Overall Completeness of data | 99.9% |
| Completeness of data in last data shell | 99.9% |
| Overall R_{SYM} | 0.08 |
| R_{SYM} in last resolved shell | 0.374 |
| Overall I/sigma (I) | 15.5 |
| I/sigma (I) in last shell | 4.1 |

### EXAMPLE 4

### Overview of SMYD3 Structure

The principal features of the SMYD3 structure include a complex β-sheet motif and a set of loosely defined helical bundles which constitute the SAM binding site. While the SAM binding site loosely resembles those of other lysine methyltransferases, the overall structure of the protein is unlike any other in the PDB currently. The unique fold derives mainly from the insert in the middle of the SET domain. Adenosyl-ornithine rests within the fairly exposed SAM binding pocket. Key hydrogen bonds exist between adenosyl-ornithine and the pocket. For example, the 6-amino of adenosyl-ornithine donates a proton to the backbone carbonyl of H206. The N9 position of adenosyl-ornithine accepts a proton from the backbone N of H206. The guanido group of R14 can make charge-dipole interactions with the NI position of adenosyl-ornithine. The side chain of N132 both donates and accepts a proton to the pair of ribose hydroxyls. The basic amine of adenosyl-ornithine interacts with the furanyl oxygen, a nearby water, the backbone carbonyl of N16, the sidechain oxygen of N205, and the acid of adenosyl-ornithine. The acid not only interacts with the basic amine of adenosyl-ornithine, but also with the backbone NH of N16, Y124's hydroxyl, and a water that interacts with the backbone NH of E130 and with the sidechain carbonyl of N181. In addition, the phenyl ring of F259 makes a pi-pi aromatic-aromatic interaction with the purine ring system. These exposed interactions feature relatively high desolvation costs, suggesting a less potent binding mode, consistent with experiment. The opening to the substrate binding cleft is maintained, even in the absence of the substrate, facilitating the design of binders to the peptide binding site if so desired.

TABLE 2
Secondary structure elements

| Secondary | | |
|---|---|---|
| Structure | Starting | Ending |
| Type | residue | residue |
| HELIX | ALA73 | HIS83 |
| HELIX | CYS87 | CYS93 |
| HELIX | ASP100 | LYS111 |
| HELIX | SER118 | LYS122 |
| HELIX | SER125 | LEU129 |
| HELIX | ASN132 | LEU136 |
| HELIX | GLU138 | PHE154 |
| HELIX | ASP161 | LEU165 |
| HELIX | LEU171 | ASN181 |
| HELIX | SER200 | LEU204 |
| HELIX | SER246 | TYR257 |
| HELIX | PHE264 | GLN267 |
| HELIX | ASP272 | MET275 |
| HELIX | GLU280 | ALA298 |
| HELIX | TRP302 | ASN316 |
| HELIX | ILE325 | ASN340 |
| HELIX | LEU344 | PHE361 |
| HELIX | PRO367 | HIS382 |
| HELIX | PHE386 | THR403 |
| HELIX | SER409 | ALA427 |
| SHEET | VAL6 | ALA10 |
| SHEET | ASN16 | ALA20 |
| SHEET | LEU29 | SER33 |
| SHEET | ALA37 | VAL40 |
| SHEET | MET60 | ARG61 |
| SHEET | LYS69 | TYR70 |
| SHEET | PHE183 | CYS186 |
| SHEET | GLU192 | LEU197 |
| SHEET | ASN205 | HIS206 |
| SHEET | CYS212 | ASN217 |
| SHEET | HIS220 | ALA225 |
| SHEET | GLU234 | ILE237 |

### EXAMPLE 5

### Docking to the SMYD3 Structure

In order to establish the utility of the structure to find chemical matter capable of binding to SMYD3, collection of about 150,000 compounds from a variety of sources screened on a cluster of Linux boxes using the structure from FIG. 1A in the software FlexX (BioSolveIT, GmbH, Sankt Augustin, Germany) with default parameters. Compounds were ranked according to their FlexX scores. The top 5,000 compounds were grouped into 25 pools of 200 for deployment in an affinity selection mass spectrometry experiment. Hits from the pools were then run in singlicates to eliminate artifacts from the pools. Analogs of hits were selected via substructure searching of the core, defined based on the docking mode in the structure.

Shown below are the structures of the compounds identified by the methods described above.

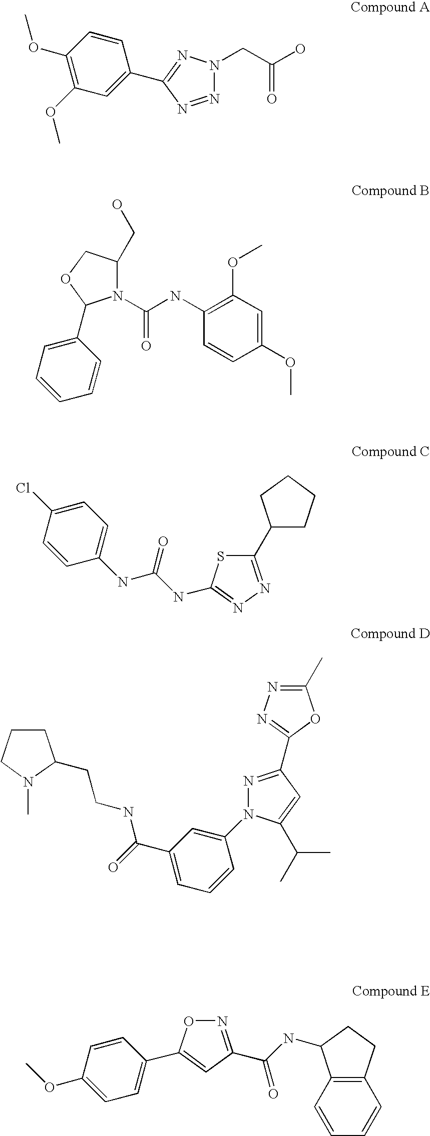

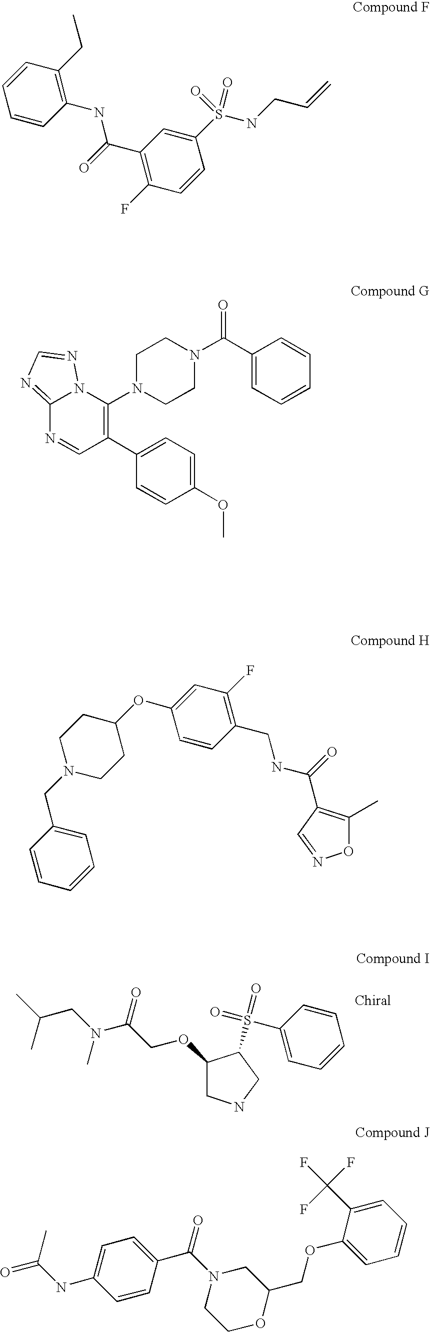

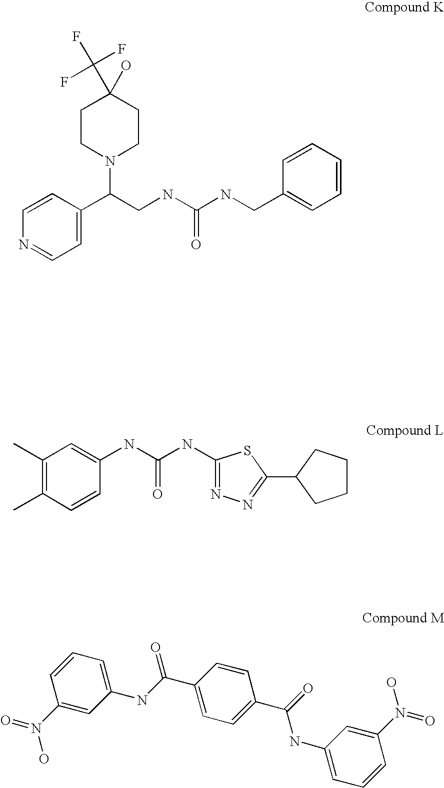

### INCORPORATION BY REFERENCE

All patents, published patent applications and other references disclosed herein are hereby expressly incorporated herein by reference.

### EQUIVALENTS

Those skilled in the art will recognize, or be able to ascertain, using no more than routine experimentation, many equivalents to specific embodiments of the invention described specifically herein. Such equivalents are intended to be encompassed in the scope of the following claims.

What is claimed is:

1. A crystal comprising a domain of a SMYD3 methyltransferase protein or a homologue thereof, wherein said domain of said SMYD3 methyltransferase protein is selected from the group consisting of amino acid residues X-Y of SEQ ID NO:1, where X=1, 2, or 7 and Y=419 or 428, and optionally other chemical entities are present.

2. The crystal according to claim 1, wherein said domain of said SMYD3 methyltransferase comprises amino acid residues 1-428 of SEQ ID NO:1, and optionally other chemical entities are present.

3. A crystallizable composition comprising a domain of a SMYD3 methyltransferase protein or a homologue thereof, wherein said domain of said SMYD3 methyltransferase is selected from the group consisting of amino acid residues X-Y of SEQ ID NO:1, where X=1, 2, or 7 and Y=419 or 428 of SEQ ID NO:1.

4. The crystallizable composition according to claim 3, wherein said domain of said SMYD3 methyltransferase protein comprises amino acid residues 1-428 of SEQ ID NO:1.

5. A computer comprising:
(a) a machine-readable data storage medium, comprising a data storage material encoded with machine-readable data, wherein said data defines a binding pocket or domain selected from the group consisting of:
(i) a set of amino acid residues which are identical to human SMYD3 methyltransferase amino acid residues R14, N132, Y124, and N205 according to FIG. 1A, wherein the root mean square deviation of the backbone atoms between the set of amino acid residues and the SMYD3 amino acid residues is not greater than about 2.0 Å;
(ii) a set of amino acid residues comprising at least three amino acid residues which are identical to human SMYD3 methyltransferase amino acid residues R14, N16, Y124, E130, N132, N181, N205, H206, and F259 according to FIG. 1A, wherein the root mean square deviation of the backbone atoms between the at least three amino acid residues and the SMYD3 amino acid residues which are identical is not greater than about 2.0 Å;
(iii) a set of amino acid residues comprising at least five amino acid residues which are identical to human SMYD3 methyltransferase amino acid residues R14, N16, Y124, E130, N132, N181, N205, H206, and F259 according to FIG. 1A, wherein the root mean square deviation of the backbone atoms between the at least five amino acid residues and the SMYD3 amino acid residues which are identical is not greater than about 2.0 Å;
(iv) a set of amino acid residues comprising at least five amino acid residues which are identical to human SMYD3 methyltransferase amino acid residues R14, G15, N16, G17, Y124, E130, N132, K135, C180, N181, S182, F183, T184, I201, S202, L203, L204, N205, H206, S207, C208, I214, I237, C238, Y239, L240, D241, R249, L253, Q256, Y257, F259, C261, D262, C263, R265, C266 according to FIG. 1A, wherein the root mean square deviation of the backbone atoms between the at least five amino acid residues and the SMYD3 amino acid residues which are identical is not greater than about 2.0 Å; and
(v) a set of amino acid residues comprising at least six amino acid residues which are identical to human SMYD3 methyltransferase amino acid residues R14, G15, N16, G17, Y124, E130, N132, K135, C180, N181, S182, F183, T184, I201, S202, L203, L204, N205, H206, S207, C208, I214, I237, C238, Y239, L240, D241, R249, L253, Q256, Y257, F259, C261, D262, C263, R265, C266 according to FIG. 1A, wherein the root mean square deviation of the backbone atoms between the at least six amino acid residues and the SMYD3 amino acid residues which are identical is not greater than about 2.0 Å; and
(vi) a set of amino acid residues that are identical to SMYD3 amino acid residues according to FIG. 1A, wherein the root mean square deviation between the set of amino acid residues and the SMYD3 amino acid residues is not more than about 2.0 Å;
(vii) a set of amino acid residues that are identical to SMYD3 amino acid residues according to FIG. 1A, wherein the root mean square deviation between the set of amino acid residues and the SMYD3 amino acid residues is not more than about 3.0 Å;
(b) a working memory for storing instructions for processing said machine-readable data;
(c) a central processing unit coupled to said working memory and to said machine-readable data storage medium for processing said machine-readable data and a means for generating three-dimensional structural information of said binding pocket or domain; and
(d) output hardware coupled to said central processing unit for outputting said three-dimensional structural information of said binding pocket or domain, or information produced using said three-dimensional structural information of said binding pocket or domain.

6. The computer according to claim 5, wherein the binding pocket is produced by homology modeling of the structure coordinates of said SMYD3 methyltransferase amino acid residues according to FIG. 1A.

7. The computer according to claim 5, wherein said means for generating three-dimensional structural information is provided by means for generating a three-dimensional graphical representation of said binding pocket or domain.

8. The computer according to claim 5, wherein said output hardware is a display terminal, a printer, CD or DVD recorder, ZIP™ or JAZ™ drive, a disk drive, or other machine-readable data storage device.

9. A method of using a computer for selecting an orientation of a chemical entity that interacts favorably with a binding pocket or domain selected from the group consisting of:
(i) a set of amino acid residues which are identical to human SMYD3 methyltransferase amino acid residues R14, N132, Y124, and N205 according to FIG. 1A, wherein the root mean square deviation of the backbone atoms between the set of amino acid residues and the SMYD3 amino acid residues is not greater than about 2.0 Å;
(ii) a set of amino acid residues comprising at least three amino acid residues which are identical to human SMYD3 methyltransferase amino acid residues R14, N16, Y124, E130, N132, N181, N205, H206, and F259 according to FIG. 1A, wherein the root mean square deviation of the backbone atoms between the at least three amino acid residues and the SMYD3 amino acid residues which are identical is not greater than about 2.0 Å;
(iii) a set of amino acid residues comprising at least five amino acid residues which are identical to human SMYD3 methyltransferase amino acid residues R14, N16, Y124, E130, N132, N181, N205, H206, and F259 according to FIG. 1A, wherein the root mean square deviation of the backbone atoms between the at least five amino acid residues and the SMYD3 amino acid residues which are identical is not greater than about 2.0 Å;
(iv) a set of amino acid residues comprising at least five amino acid residues which are identical to human SMYD3 methyltransferase amino acid residues R14, G15, N16, G17, Y124, E130, N132, K135, C180, N181, S182, F183, T184, I201, S202, L203, L204, N205, H206, S207, C208, I214, I237, C238, Y239, L240, D241, R249, L253, Q256, Y257, F259, C261, D262, C263, R265, C266 according to FIG. 1A, wherein the root mean square deviation of the backbone atoms between the at least five amino acid residues and the SMYD3 amino acid residues which are identical is not greater than about 2.0 Å; and
(v) a set of amino acid residues comprising at least six amino acid residues which are identical to human SMYD3 methyltransferase amino acid residues R14, G15, N16, G17, Y124, E130, N132, K135, C180, N181, S182, F183, T184, I201, S202, L203, L204, N205, H206, S207, C208, I214, I237, C238, Y239, L240, D241, R249, L253, Q256, Y257, F259, C261, D262, C263, R265, C266 according to FIG. 1A, wherein the root mean square deviation of the backbone atoms between the at least six amino acid residues and the SMYD3 amino acid residues which are identical is not greater than about 2.0 Å; and
(vi) a set of amino acid residues that are identical to SMYD3 amino acid residues according to FIG. 1A, wherein the root mean square deviation between the set of amino acid residues and the SMYD3 amino acid residues is not more than about 2.0 Å;
(vii) a set of amino acid residues that are identical to SMYD3 amino acid residues according to FIG. 1A, wherein the root mean square deviation between the set of amino acid residues and the SMYD3 amino acid residues is not more than about 3.0 Å;
said method comprising the steps of
(a) providing the structure coordinates of said binding pocket or domain on a computer comprising means for generating three-dimensional structural information from said structure coordinates;
(b) employing computational means to dock a first chemical entity in the binding pocket or domain;
(c) quantifying the association between said chemical entity and all or part of the binding pocket or domain for different orientations of the chemical entity; and
(d) selecting the orientation of the chemical entity with the most favorable interaction based on said quantified association.

10. The method according to claim 9, further comprising the step of: (e) generating a three-dimensional graphical representation of the binding pocket or domain prior to step (b).

11. The method according to claim 9, wherein energy minimization, molecular dynamics simulations, rigid-body minimizations, combinations thereof, or similar induced-fit manipulations are performed simultaneously with or following step (b).

12. The method according to claim 9, further comprising the steps of: (e) repeating steps (b) through (d) with a second chemical entity; and (f) selecting of at least one of said first or second chemical entity that interacts more favorably with said-binding pocket or domain based on said quantified association of said first or second chemical entity.

13. A method of using a computer for selecting an orientation of a chemical entity with a favorable shape complementarity in a binding pocket selected from the group consisting of:
(i) a set of amino acid residues which are identical to human SMYD3 methyltransferase amino acid residues R14, N132, Y124, and N205 according to FIG. 1A, wherein the root mean square deviation of the backbone atoms between the set of amino acid residues and the SMYD3 amino acid residues is not greater than about 2.0 Å;
(ii) a set of amino acid residues comprising at least three amino acid residues which are identical to human SMYD3 methyltransferase amino acid residues R14, N16, Y124, E130, N132, N181, N205, H206, and F259 according to FIG. 1A, wherein the root mean square deviation of the backbone atoms between the at least three amino acid residues and the SMYD3 amino acid residues which are identical is not greater than about 2.0 Å;
(iii) a set of amino acid residues comprising at least five amino acid residues which are identical to human SMYD3 methyltransferase amino acid residues R14, N16, Y124, E130, N132, N181, N205, H206, and F259 according to FIG. 1A, wherein the root mean square deviation of the backbone atoms between the at least five amino acid residues and the SMYD3 amino acid residues which are identical is not greater than about 2.0 Å;
(iv) a set of amino acid residues comprising at least five amino acid residues which are identical to human SMYD3 methyltransferase amino acid residues R14, G15, N16, G17, Y124, E130, N132, K135, C180, N181, S182, F183, T184, I201, S202, L203, L204, N205, H206, S207, C208, I214, I237, C238, Y239, L240, D241, R249, L253, Q256, Y257, F259, C261, D262, C263, R265, C266 according to FIG. 1A, wherein the root mean square deviation of the backbone atoms between the at least five amino acid residues and the SMYD3 amino acid residues which are identical is not greater than about 2.0 Å; and
(v) a set of amino acid residues comprising at least six amino acid residues which are identical to human SMYD3 methyltransferase amino acid residues R14, G15, N16, G17, Y124, E130, N132, K135, C180, N181, S182, F183, T184, I201, S202, L203, L204, N205, H206, S207, C208, I214, I237, C238, Y239, L240, D241, R249, L253, Q256, Y257, F259, C261, D262, C263, R265, C266 according to FIG. 1A, wherein the root mean square deviation of the backbone atoms between the at least six amino acid residues and the SMYD3 amino acid residues which are identical is not greater than about 2.0 Å; and
(vi) a set of amino acid residues that are identical to SMYD3 amino acid residues according to FIG. 1A, wherein the root mean square deviation between the set of amino acid residues and the SMYD3 amino acid residues is not more than about 2.0 Å;
(vii) a set of amino acid residues that are identical to SMYD3 amino acid residues according to FIG. 1A, wherein the root mean square deviation between the set of amino acid residues and the SMYD3 amino acid residues is not more than about 3.0 Å;
said method comprising the steps of:
(a) providing the structure coordinates of said binding pocket on a computer comprising means for generating three-dimensional structural information from said structure coordinates;
(b) employing computational means to dock a first chemical entity in the binding pocket;
(c) quantitating the contact score of said chemical entity in different orientations; and
(d) selecting an orientation with the highest contact score.

14. The method according to claim 13, further comprising the step of: (e) generating a three-dimensional graphical representation of the binding pocket prior to step (b).

15. The method according to claim 13, further comprising the steps of: (e) repeating steps (b) through (d) with a second chemical entity; and (f) selecting of at least one of said first or second chemical entity that has a higher contact score based on said quantitated contact score of said first or second chemical entity.

16. A method for identifying a candidate binder of a molecule or molecular complex comprising a binding pocket or domain selected from the group consisting of:
(i) a set of amino acid residues which are identical to human SMYD3 methyltransferase amino acid residues R14, N132, Y124, and N205 according to FIG. 1A, wherein the root mean square deviation of the backbone atoms between the set of amino acid residues and the SMYD3 amino acid residues is not greater than about 2.0 Å;
(ii) a set of amino acid residues comprising at least three amino acid residues which are identical to human SMYD3 methyltransferase amino acid residues R14, N16, Y124, E130, N132, N181, N205, H206, and F259 according to FIG. 1A, wherein the root mean square deviation of the backbone atoms between the at least three amino acid residues and the SMYD3 amino acid residues which are identical is not greater than about 2.0 Å;
(iii) a set of amino acid residues comprising at least five amino acid residues which are identical to human SMYD3 methyltransferase amino acid residues R14, N16, Y124, E130, N132, N181, N205, H206, and F259 according to FIG. 1A, wherein the root mean square deviation of the backbone atoms between the at least five amino acid residues and the SMYD3 amino acid residues which are identical is not greater than about 2.0 Å;
(iv) a set of amino acid residues comprising at least five amino acid residues which are identical to human SMYD3 methyltransferase amino acid residues R14, G15, N16, G17, Y124, E130, N132, K135, C180, N181, S182, F183, T184, I201, S202, L203, L204, N205, H206, S207, C208, I214, I237, C238, Y239, L240, D241, R249, L253, Q256, Y257, F259, C261, D262, C263, R265, C266 according to FIG. 1A, wherein the root mean square deviation of the backbone atoms between the at least five amino acid residues and the SMYD3 amino acid residues which are identical is not greater than about 2.0 Å; and
(v) a set of amino acid residues comprising at least six amino acid residues which are identical to human SMYD3 methyltransferase amino acid residues R14, G15, N16, G17, Y124, E130, N132, K135, C180, N181, S182, F183, T184, I201, S202, L203, L204, N205, H206, S207, C208, I214, I237, C238, Y239, L240, D241, R249, L253, Q256, Y257, F259, C261, D262, C263, R265, C266 according to FIG. 1A, wherein the root mean square deviation of the backbone atoms between the at least six amino acid residues and the SMYD3 amino acid residues which are identical is not greater than about 2.0 Å; and
(vi) a set of amino acid residues that are identical to SMYD3 amino acid residues according to FIG. 1A, wherein the root mean square deviation between the set of amino acid residues and the SMYD3 amino acid residues is not more than about 2.0 Å;
(vii) a set of amino acid residues that are identical to SMYD3 amino acid residues according to FIG. 1A, wherein the root mean square deviation between the set of amino acid residues and the SMYD3 amino acid residues is not more than about 3.0 Å;
comprising the steps of:
(a) using a three-dimensional structure of the binding pocket or domain to design, select or optimize a plurality of chemical entities;
(b) contacting each chemical entity with the molecule or the molecular complex;
(c) monitoring an effect on the catalytic activity of the molecule or molecular complex by each chemical entity; and
(d) selecting a chemical entity based on the magnitude of observed desired effect of the chemical entity on the catalytic activity of the molecule or molecular complex.

17. A method of designing a compound or complex that interacts with a binding pocket or domain selected from the group consisting of:
(i) a set of amino acid residues which are identical to human SMYD3 methyltransferase amino acid residues R14, N132, Y124, and N205 according to FIG. 1A, wherein the root mean square deviation of the backbone atoms between the set of amino acid residues and the SMYD3 amino acid residues is not greater than about 2.0 Å;
(ii) a set of amino acid residues comprising at least three amino acid residues which are identical to human SMYD3 methyltransferase amino acid residues R14, N16, Y124, E130, N132, N181, N205, H206, and F259 according to FIG. 1A, wherein the root mean square deviation of the backbone atoms between the at least three amino acid residues and the SMYD3 amino acid residues which are identical is not greater than about 2.0 Å;
(iii) a set of amino acid residues comprising at least five amino acid residues which are identical to human SMYD3 methyltransferase amino acid residues R14, N16, Y124, E130, N132, N181, N205, H206, and F259 according to FIG. 1A, wherein the root mean square deviation of the backbone atoms between the at least five amino acid residues and the SMYD3 amino acid residues which are identical is not greater than about 2.0 Å;
(iv) a set of amino acid residues comprising at least five amino acid residues which are identical to human SMYD3 methyltransferase amino acid residues R14, G15, N16, G17, Y124, E130, N132, K135, C180, N181, S182, F183, T184, I201, S202, L203, L204, N205, H206, S207, C208, I214, I237, C238, Y239, L240, D241, R249, L253, Q256, Y257, F259, C261, D262, C263, R265, C266 according to FIG. 1A, wherein the root mean square deviation of the backbone atoms between the at least five amino acid residues and the SMYD3 amino acid residues which are identical is not greater than about 2.0 Å; and
(v) a set of amino acid residues comprising at least six amino acid residues which are identical to human SMYD3 methyltransferase amino acid residues R14, G15, N16, G17, Y124, E130, N132, K135, C180, N181, S182, F183, T184, I201, S202, L203, L204, N205, H206, S207, C208, I214, I237, C238, Y239, L240, D241, R249, L253, Q256, Y257, F259, C261, D262, C263, R265, C266 according to FIG. 1A, wherein the root mean square deviation of the backbone atoms between the at least six amino acid residues and the SMYD3 amino acid residues which are identical is not greater than about 2.0 Å; and
(vi) a set of amino acid residues that are identical to SMYD3 amino acid residues according to FIG. 1A, wherein the root mean square deviation between the set of amino acid residues and the SMYD3 amino acid residues is not more than about 2.0 Å;
(vii) a set of amino acid residues that are identical to SMYD3 amino acid residues according to FIG. 1A, wherein the root mean square deviation between the set of amino acid residues and the SMYD3 amino acid residues is not more than about 3.0 Å;
comprising the steps of:
(a) providing the structure coordinates of said binding pocket or domain on a computer comprising means for generating three-dimensional structural information from said structure coordinates;
(b) using the computer to dock a first chemical entity in part of the binding pocket or domain;
(c) docking at least a second chemical entity in another part of the binding pocket or domain;
(d) quantifying the association between the first or second chemical entity and part of the binding pocket or domain;
(e) repeating steps (b) to (d) with another first and second chemical entity;
(f) selecting a first and a second chemical entity based on said quantified association of both said first and second chemical entity;
(g) optionally, visually inspecting the relationship of the first and second chemical entity to each other in relation to the binding pocket or domain on a computer screen using the three-dimensional graphical representation of the binding pocket or domain and said first and second chemical entity; and
(h) assembling the first and second chemical entity into a compound or complex that interacts with said binding pocket or domain by model building.

18. A method of utilizing molecular replacement to obtain structural information about a molecule or a molecular complex of unknown structure, wherein the molecule is sufficiently homologous to a domain of a SMYD3 methyltransferase protein or a homologue thereof, comprising the steps of:
(a) crystallizing said molecule or molecular complex;
(b) generating an X-ray diffraction pattern from said crystallized molecule or molecular complex;
(c) applying at least a portion of the structure coordinates set forth in FIG. 1A or a homology model thereof to the X-ray diffraction pattern to generate a three-dimensional electron density map of at least a portion of the molecule or molecular complex of unknown structure; and
(d) generating a structural model of the molecule or molecular complex from the three-dimensional electron density map.

19. The method according to claim 18, wherein the molecule is selected from the group consisting of said domain of said SMYD3 methyltransferase protein, and said domain of said SMYD3 methyltransferase protein homologue.

20. The method according to claim 18, wherein the molecular complex is selected from the group consisting of said domain of said SMYD3 methyltransferase protein complex and said domain of said SMYD3 methyltransferase protein homologue complex.

21. A method for identifying a candidate binder that interacts with a binding site of a SMYD3 methyltransferase protein or a homologue thereof, comprising the steps of:
(a) obtaining a crystal comprising a domain of said SMYD3 methyltransferase protein or said homologue thereof, wherein the crystal is characterized with space group P_{1 21 1} and has unit cell parameters of a=58.175 Å, b=118.073 Å, c=82.901 Å, α=90.00, β=91.58, γ=90.00;
(b) obtaining the structure coordinates of amino acids of the crystal of step (a), wherein the structure coordinates are set forth in FIG. 1A-1 to 1A-129;
(c) generating a three-dimensional model of the domain of said SMYD3 methyltransferase protein or said homologue thereof using the structure coordinates of the amino acids obtained in step (b), a root mean square deviation from backbone atoms of said amino acids of not more than ±2.0 Å;
(d) determining a binding site of the domain of said SMYD3 methyltransferase protein or said homologue thereof from said three-dimensional model; and
(e) performing computer fitting analysis to identify the candidate binder which interacts with said binding site.

22. The method according to claim 21, further comprising the step of: (f) contacting the identified candidate binder with the domain of said SMYD3 methyltransferase protein or said homologue thereof in order to determine the effect of the binder on SMYD3 methyltransferase protein activity.

23. The method according to claim 21, wherein the binding site of the domain of said SMYD3 methyltransferase protein or said homologue thereof determined in step (d) comprises the structure coordinates according to FIG. 1A-1 to 1A-129 of amino acid residues R14, N132, Y124, and N205, wherein the root mean square deviation from the backbone atoms of said amino acids is not more than ±2.0 Å.

24. The method according to claim 21, wherein the binding site of the domain of said SMYD3 methyltransferase protein or said homologue thereof determined in step (d) comprises the structure coordinates according to FIG. 1A-1 to 1A-129 of amino acid residues R14, N16, Y124, E130, N132, N181, N205, H206, and F259, wherein the root mean square deviation from the backbone atoms of said amino acids is not more than ±2.0 Å.

25. The method according to claim 21, wherein the binding site of the domain of said SMYD3 methyltransferase protein or said homologue thereof determined in step (d) comprises the structure coordinates according to FIG. 1A-1 to 1A-129 of amino acid residues R14, G15, N16, G17, Y124, E130, N132, K135, C180, N181, S182, F183, T184, I201, S202, L203, L204, N205, H206, S207, C208, I214, I237, C238, Y239, L240, D241, R249, L253, Q256, Y257, F259, C261, D262, C263, R265, and C266, wherein the root mean square deviation from the backbone atoms of said amino acids is not more than ±2.0 Å.

26. A method for identifying a candidate binder that interacts with a binding site of a domain of a SMYD3 methyltransferase protein or a homologue thereof, comprising the steps of:
(a) obtaining a crystal comprising the domain of said SMYD3 methyltransferase protein or said homologue thereof, wherein the crystal is characterized with space group P_{1 21 1} and has unit cell parameters of a=58.175 Å, b=118.073 Å, c=82.901 Å, α=90.00, β=91.58, γ=90.00;
(b) obtaining the structure coordinates of amino acids of the crystal of step (a);
(c) generating a three-dimensional model of said SMYD3 methyltransferase protein or said homologue thereof using the structure coordinates of the amino acids generated in step (b), a root mean square deviation from backbone atoms of said amino acids of not more than ±2.0 Å;
(d) determining a binding site of the domain of said SMYD3 methyltransferase protein or said homologue thereof from said three-dimensional model; and
(e) performing computer fitting analysis to identify the candidate binder which interacts with said binding site.

27. The method according to claim 26, further comprising the step of: (f) contacting the identified candidate binder with the domain of said SMYD3 methyltransferase protein or said homologue thereof in order to determine the effect of the binder on SMYD3 methyltransferase protein activity.

28. The method according to claim 26, wherein the binding site of the domain of said SMYD3 methyltransferase protein or said homologue thereof determined in step (d) comprises the structure coordinates according to FIG. 1A-1 to 1A-129 of amino acid residues R14, N132, Y124, and N205, wherein the root mean square deviation from the backbone atoms of said amino acids is not more than ±2.0 Å.

29. The method according to claim 26, wherein the binding site of the domain of said SMYD3 methyltransferase protein or said homologue thereof determined in step (d) comprises the structure coordinates according to FIG. 1A-1 to 1A-129 of amino acid residues R14, N16, Y124, E130, N132, N181, N205, H206, and F259, wherein the root mean square deviation from the backbone atoms of said amino acids is not more than ±2.0 Å.

30. The method according to claim 26, wherein the binding site of the domain of said SMYD3 methyltransferase protein or said homologue thereof determined in step (d) comprises the structure coordinates according to FIG. 1A-1 to 1A-129 of amino acid residues R14, G15, N16, G17, Y124, E130, N132, K135, C180, N181, S182, F183, T184, I201, S202, L203, L204, N205, H206, S207, C208, I214, I237, C238, Y239, L240, D241, R249, L253, Q256, Y257, F259, C261, D262, C263, R265, and C266, wherein the root mean square deviation from the backbone atoms of said amino acids is not more than ±2.0 Å.

31. A method for identifying a candidate binder that interacts with a binding site of a domain of a SMYD3 methyltransferase protein or a homologue thereof, comprising the step of determining a binding site of the domain of said SMYD3 methyltransferase protein or the homologue thereof from a three-dimensional model to design or identify the candidate binder which interacts with said binding site.

32. The method according to claim 31, wherein the binding site of the domain of said SMYD3 methyltransferase protein or said homologue thereof determined comprises the structure coordinates according to FIG. 1A-1 to 1A-129 of amino acid residues R14, N132, Y124, and N205, wherein the root mean square deviation from the backbone atoms of said amino acids is not more than ±2.0 Å.

33. The method according to claim 31, wherein the binding site of the domain of said SMYD3 methyltransferase protein or said homologue thereof determined comprises the structure coordinates according to FIG. 1A-1 to 1A-129 of amino acid residues R14, N16, Y124, E130, N132, N181, N205, H206, and F259, wherein the root mean square deviation from the backbone atoms of said amino acids is not more than ±2.0 Å.

34. The method according to claim 31, wherein the binding site of the domain of said SMYD3 methyltransferase protein or said homologue thereof determined comprises the structure coordinates according to FIG. 1A-1 to 1A-129 of amino acid residues R14, G15, N16, G17, Y124, E130, N132, K135, C180, N181, S182, F183, T184, I201, S202, L203, L204, N205, H206, S207, C208, I214, I237, C238, Y239, L240, D241, R249, L253, Q256, Y257, F259, C261, D262, C263, R265, and C266, wherein the root mean square deviation from the backbone atoms of said amino acids is not more than ±2.0 Å

35. A method for identifying a candidate binder of a molecule or molecular complex comprising a binding pocket or domain selected from the group consisting of:
(i) a set of amino acid residues which are identical to human SMYD3 methyltransferase amino acid residues R14, N132, Y124, and N205 according to FIG. 1A, wherein the root mean square deviation of the backbone atoms between the set of amino acid residues and the SMYD3 amino acid residues is not greater than about 2.0 Å;
(ii) a set of amino acid residues comprising at least three amino acid residues which are identical to human SMYD3 methyltransferase amino acid residues R14, N16, Y124, E130, N132, N181, N205, H206, and F259 according to FIG. 1A, wherein the root mean square deviation of the backbone atoms between the at least three amino acid residues and the SMYD3 amino acid residues which are identical is not greater than about 2.0 Å;
(iii) a set of amino acid residues comprising at least five amino acid residues which are identical to human SMYD3 methyltransferase amino acid residues R14, N16, Y124, E130, N132, N181, N205, H206, and F259 according to FIG. 1A, wherein the root mean square deviation of the backbone atoms between the at least five amino acid residues and the SMYD3 amino acid residues which are identical is not greater than about 2.0 Å;
(iv) a set of amino acid residues comprising at least five amino acid residues which are identical to human SMYD3 methyltransferase amino acid residues R14, G15, N16, G17, Y124, E130, N132, K135, C180, N181, S182, F183, T184, I201, S202, L203, L204, N205, H206, S207, C208, I214, I237, C238, Y239, L240, D241, R249, L253, Q256, Y257, F259, C261, D262, C263, R265, C266 according to FIG. 1A, wherein the root mean square deviation of the backbone atoms between the at least five amino acid residues and the SMYD3 amino acid residues which are identical is not greater than about 2.0 Å; and
(v) a set of amino acid residues comprising at least six amino acid residues which are identical to human SMYD3 methyltransferase amino acid residues R14, G15, N16, G17, Y124, E130, N132, K135, C180, N181, S182, F183, T184, I201, S202, L203, L204, N205, H206, S207, C208, I214, I237, C238, Y239, L240, D241, R249, L253, Q256, Y257, F259, C261, D262, C263, R265, C266 according to FIG. 1A, wherein the root mean square deviation of the backbone atoms between the at least six amino acid residues and the SMYD3 amino acid residues which are identical is not greater than about 2.0 Å; and
(vi) a set of amino acid residues that are identical to SMYD3 amino acid residues according to FIG. 1A, wherein the root mean square deviation between the set of amino acid residues and the SMYD3 amino acid residues is not more than about 2.0 Å;
(vii) a set of amino acid residues that are identical to SMYD3 amino acid residues according to FIG. 1A, wherein the root mean square deviation between the set of amino acid residues and the SMYD3 amino acid residues is not more than about 3.0 Å;
comprising the steps of:
(a) using a three-dimensional structure of the binding pocket or domain to design, select or optimize a plurality of chemical entities; and
(b) selecting said candidate binder based on the effect of said chemical entities on a domain of a SMYD3 methyltransferase protein or a domain of a SMYD3 methyltransferase protein homologue on the catalytic activity of the molecule or molecular complex.

36. A method of using the crystal of claim 1 or 2 in an binder screening assay comprising:
(a) selecting a potential binder by performing rational drug design with a three-dimensional structure determined for the crystal, wherein said selecting is performed in conjunction with computer modeling;
(b) contacting the potential binder with a methyltransferase; and
(c) detecting the ability of the potential binder for modulating the methyltransferase activity.

37. A method of preparing the crystals of claim 1 or 2, comprising the steps of
a) combining a crystallization solution with a SMYD3-like methyltransferase protein or homologue thereof to produce a crystallizable composition; and
b) subjecting the composition to conditions which promote crystallization and obtaining said crystal.

38. A set of coordinates as described in FIG. 1A defining the 3-dimensional structure of the protein SMYD3 with the amino acid sequence 1-428.

39. A compound having the following formula:

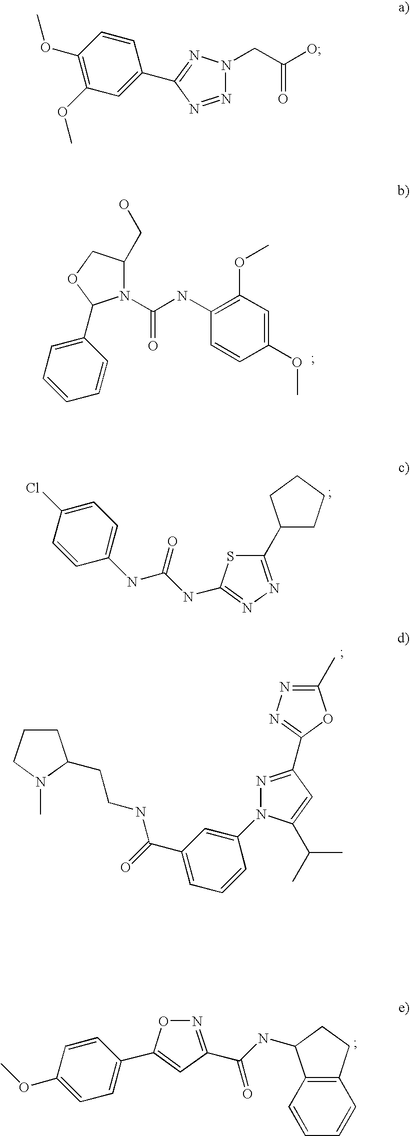

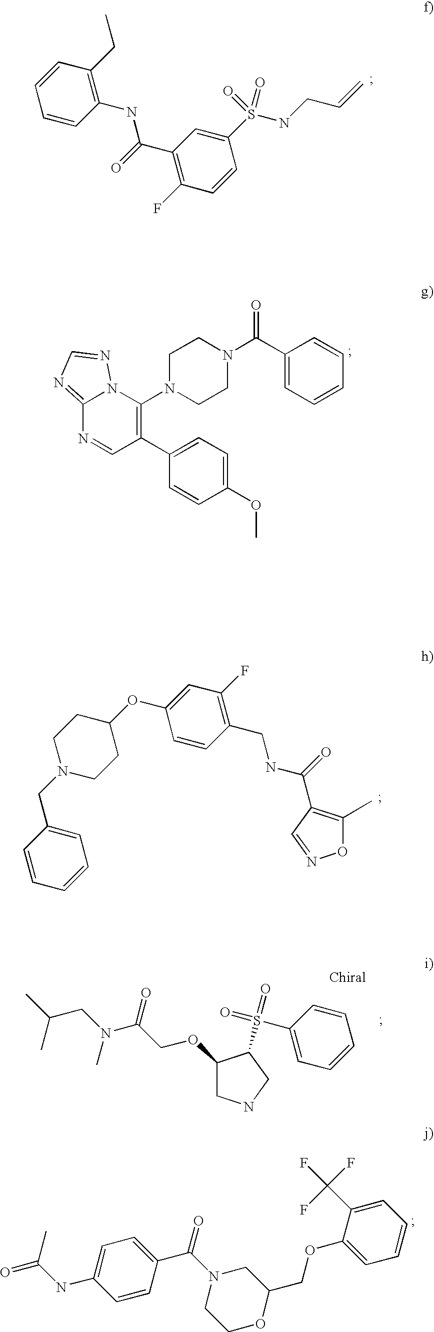

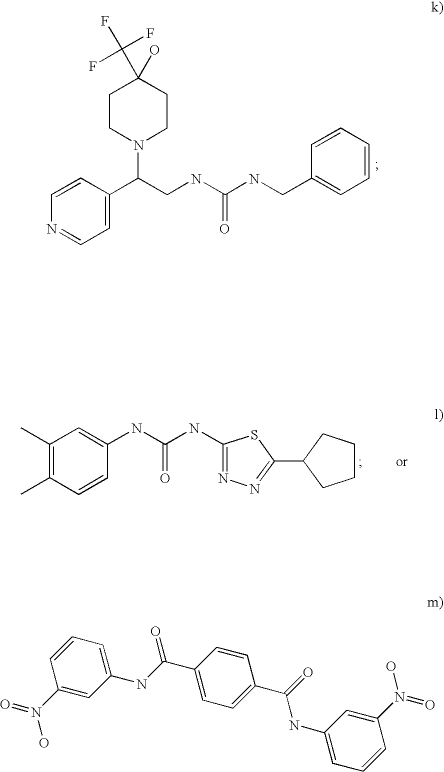

40. A method of treating cancer or male infertility in a patient by administering one or more of compounds in claim 39.

41. The method of claim 40, further comprising administering an additional treatment.

42. The method of claim 41, wherein said additional treatment is an anticancer treatments or an antidiabetic treatment.

43. A method for determining SMYD3 binding of any potential SMYD3 binder, comprising the steps
(iii) contacting a SMYD3 protein with a test compound;
(iv) detecting binding of said test compound and said SMYD3 protein.


SEQUENCE LISTING (1 sequence)

SEQ ID NO: 1 - protein, 428 aa, Homo sapiens
MEPLKVEKFATANRGNGLRAVTPLRPGELLFRSDPLAYTVCKGSRGVVCDRCLLGKEKLMRCSQCRVAKYCSAKCQKKAWPDHKRECKCLKSCKPRYPPDSVRLLGRVVFKLMDGAPSESEKLYSFYDLESNINKLTEDKKEGLRQLVMTFQHFMREEIQDASQLPPAFDLFEAFAKVICNSFTICNAEMQEVGVGLYPSISLLNHSCDPNCSIVFNGPHLLLRAVRDIEVGEELTICYLDMLMTSEERRKQLRDQYCFECDCFRCQTQDKDADMLTGDEQVWKEVQESLKKIEELKAHWKWEQVLAMCQAIISSNSERLPDINIYQLKVLDCAMDACINLGLLEEALFYGTRTMEPYRIFFPGSHPVRGVQVMKVGKLQLHQGMFPQAMKNLRLAFDIMRVTHGREHSLIEDLILLLEECDANIRAS